SEQUENCE LISTING (3 sequences)

SEQ ID NO: 1 - DNA, 38 bp, Artificial Sequence
  Description of Artificial Sequence Synthetic primer
agcggatccgccatgaattattcaagctacctcttagc

SEQ ID NO: 2 - DNA, 37 bp, Artificial Sequence
  Description of Artificial Sequence Synthetic primer
tatctctagattactgtgtgatttttccttggctttt

SEQ ID NO: 3 - protein, 9 aa, Artificial Sequence
  Description of Artificial Sequence Synthetic peptide
CARSKNKDC

(19) United States
(12) Patent Application Publication
(10) Pub. No.: US 2023/0193205 A1
(43) Pub. Date: Jun. 22, 2023

(54) GENE MODIFIED FIBROBLASTS FOR THERAPEUTIC APPLICATIONS

(71) Applicant: FIGENE, LLC, Houston, TX (US)

(72) Inventors: Thomas ICHIM, San Diego, CA (US); Pete O'HEERON, Houston, TX (US)

(21) Appl. No.: 17/996,109

(22) PCT Filed: Apr. 19, 2021
(86) PCT No.: PCT/US2021/027990

§ 371 Date: Oct. 13, 2022

Related U.S. Application Data
(60) Provisional application No. 63/012,202, filed on Apr. 19, 2020.

(51) Int. Cl. C12N 5/077; C12N 15/85; A61K 35/33

(52) U.S. Cl. CPC C12N 5/0656; C12N 15/85; A61K 35/33; C12N 2500/60; C12N 2800/107

(57) ABSTRACT

Disclosed are compositions of matter, cells, protocols and procedures useful for augmentation of one or more therapeutic activities of fibroblast cellular populations. In one embodiment, fibroblasts are cultured in an acidic pH incubation medium before genetic modification, after genetic modification, or both. In another embodiment, fibroblasts are cultured under hypoxic conditions. In another embodiment, fibroblasts are treated with mitogenic factor-comprising composition(s) to increase the efficacy of genetic modification, wherein the mitogenic factor(s) may be cytokines, peptides, and/or proteins. In another embodiment, a therapeutically effective amount of the genetically modified fibroblasts are provided as a therapeutic application to an individual in need thereof. In another embodiment, the therapeutic application is treatment of fibrosis, treatment of inflammation, acceleration of healing, treatment of clotting disorders, or promotion of angiogenesis.

This application claims priority to U.S. Provisional Patent Application Ser. No. 63/012,202, filed Apr. 19, 2020, which is incorporated by reference herein in its entirety.

### TECHNICAL FIELD

Embodiments of the field of the present disclosure include at least the fields of molecular biology, cell biology, cell therapy, and medicine.

### BACKGROUND

The disclosure provides means of generating therapeutic fibroblasts using gene modification. Provided within the disclosure are means of growing, expanding, and generating fibroblasts that possess features allowing them to be superior cells to unmanipulated cells for purposes of gene modification. Gene manipulation according to the disclosure may be performed in order to enhance various properties of the fibroblasts. For example, in embodiments in which enhanced angiogenesis is desired, transfection of fibroblasts with proangiogenic genes is disclosed. In embodiments in which enhanced protection from inflammation is desired, transfection with genes known to inhibit inflammation is disclosed. In situations in which suppression of cancer is desired, transfection with cytotoxic genes, and/or immunomodulatory genes is disclosed. One of the key aspects of the disclosure is the unexpected finding that conditions such as acidosis enhance the ability of fibroblasts to be more efficiently transfected and provide enhanced viability, both in vitro and in vivo.

### BRIEF SUMMARY

The present disclosure is directed to means of generating therapeutic fibroblasts and fibroblast progenitor cells through gene manipulation. In one embodiment, fibroblasts are cultured under acidic conditions, allowing for enhanced activity subsequent to gene modification. In some embodiments, gene modification comprises addition of one or more genes through means of transfection of the fibroblasts. In other embodiments, the disclosure provides for ideal cells for gene modification, wherein said gene modification encompasses gene silencing or gene deletion. In some embodiments, gene modification involves either inhibition or overexpression of one or more genes through manipulation of one or more transcription factors.

In specific embodiments, methods are provided for preparing gene modified fibroblasts comprising the steps of culturing fibroblasts in an acidic pH incubation medium before gene modification, concurrent with gene modification, after gene modification, or a combination thereof. In specific embodiments, fibroblasts are cultured in an acidic pH incubation medium before gene modification. In specific embodiments, fibroblasts are cultured in an acidic pH incubation medium concurrent with gene modification. In specific embodiments, fibroblasts are cultured in an acidic pH incubation medium after gene modification. In specific embodiments, fibroblasts are cultured in an acidic pH incubation medium before and after gene modification.

In specific embodiments, the acidic pH is a pH of 4.6-6.5, 4.8-6.5, 5-6.5, 5.2-6.5, 5.4-6.5, 5.6-6.5, 5.8-6.5, 6-6.5, 4.6-6.4, 4.8-6.4, 5-6.4, 5.2-6.4, 5.4-6.4, 5.6-6.4, 5.8-6.4, 6-6.4, 4.6-6.3, 4.8-6.3, 5-6.3, 5.2-6.3, 5.4-6.3, 5.6-6.3, 5.8-6.3, 6-6.3, 4.6-6.2, 4.8-6.2, 5-6.2, 5.2-6.2, 5.4-6.2, 5.6-6.2, 5.8-6.2, 6-6.2, 4.6-6.1, 4.8-6.1. 5-6.1, 5.2-6.1, 5.4-6.1, 5.6-6.1, 5.8-6.1, 4.6-6, 4.8-6, 5.0-6, 5.2-6, 5.4-6, 5.6-6, or 5.8-6. In specific embodiments, the acidic pH is a pH of 5-6.5. In specific embodiments, the acidic pH is a pH of 5, 5.1, 5.2, 5.3, 5.4, 5.5, 5.6, 5.7, 5.8, 5.9, 6, 6.1, 6.2, 6.3, 6.4, or 6.5. In specific embodiments, the acidic pH is a pH of 5.8-6.2. In specific cases, the acidic pH is a pH of 5.8, 5.9, 6, 6.1, or 6.2.

In specific embodiments, fibroblasts are cultured in an acidic pH incubation medium for 1 second-180 minutes, 1 second-8 minutes, 1 second-6 minutes, 1 second-4 minutes, 1-10 minutes, 1-8 minutes, 1-6 minutes, 1-4 minutes, 2-10 minutes, 2-8 minutes, 2-6 minutes, 2-4 minutes, 4-10 minutes, 4-8 minutes, or 4-6 minutes. In specific embodiments, fibroblasts are cultured in an acidic pH incubation medium for 10-180, 10-150, 10-120, 10-90, 10-60, 15-180, 15-150, 15-120, 15-90, 15-60, 20-180, 20-150, 20-120, 20-90, 20-60, 25-180, 25-150, 25-120, 25-90, 25-60, 30-180, 30-150, 30-120, 30-90, or 30-60, minutes. In specific embodiments, fibroblasts are cultured in an acidic pH incubation medium for 15-120 minutes. In specific embodiments, fibroblasts are cultured for 15, 20, 30, 40, 50, 60, 70, 80, 90, 100, 110, or 120 minutes.

In specific embodiments, methods are provided for preparing gene modified fibroblasts comprising the steps of culturing fibroblasts in a first acidic pH incubation medium and a second nonacidic pH incubation medium. In specific embodiments, fibroblasts are cultured in the first acidic pH incubation medium and the second nonacidic pH incubation medium before gene modification, concurrent with gene modification, after gene modification, or a combination thereof.

In some embodiments, the incubating in a second nonacidic incubation medium is performed for 10 minutes to 3 weeks, 10 minutes to 2 weeks, 10 minutes to 1 week, 10 minutes to four days, 10 minutes to 2 days, 10 minutes to 1 day, 1 hour to 3 weeks, 1 hour to 2 weeks, 1 hour to 1 week, 1 hour to four days, 1 hour to two days, 1 hour to 1 day, 12 hours to 3 weeks, 12 hours to 2 weeks, 12 hours to 1 week, 12 hours to four days, 12 hours to 2 days, 12 hours to 1 day, 1 day to 3 weeks, 1 day to 2 weeks, 1 day to 1 week, three days to 3 weeks, 3 days to 2 weeks, 3 days to 1 week, 3 days to 4 days, 1 week to 3 weeks, 1 week to 2 weeks, or 2 weeks to three weeks. In some embodiments, the incubating in a second nonacidic incubation medium is performed for 10 minutes to 3 weeks.

In specific embodiments, fibroblasts are cultured in a hypoxic condition. In specific embodiments, the hypoxic condition is at or below 4.5%, 4%, 3.5%, 3%, or 2.5%, oxygen. In specific embodiments, the hypoxic condition is at 1-4.5%, 1-4%, 1-3.5%, 1-3%, 1.5-4.5%, 1.5-4%, 1.5-3.5%, 1.5-3%, 2-4.5%, 2-4%, 2-3.5%, 2-3%, 2.5-4.5%, 2.5-4%, 2.5-3.5%, or 2.5-3%, oxygen. In specific embodiments, the hypoxic condition is at 1-4.5% oxygen. In specific embodiments, the hypoxic condition is at 2-4% oxygen. In specific embodiments, the hypoxic condition is at 2, 2.5, 3, 3.5, or 4%, oxygen. In specific embodiments, the hypoxic condition is at or below 3% oxygen.

In specific embodiments, the incubation medium is DMEM, RPMI, F12, IMDM, MEM, or a combination thereof. In specific embodiments, the incubation medium comprises less than 5 g/L, 4 g/L, 3 g/L, 2 g/L, 1 g/L, or 0.5 g/L, glucose. In specific embodiments, the incubation medium comprises less than 5 g/L glucose.

In specific embodiments, the incubation medium further comprises one or more mitogenic factors. In specific embodiments, the mitogenic factor is EGF, one or more FGFs, one or more cytokines, one or more ROCK inhibitors, one or more GSK3 inhibitors, one or more SHH inhibitors, one or more ERK activators, one or more MAO inhibitors, one or more PKC activators, one or more histone modifying enzyme inhibitors, Y-27632, CHlR99021, tranylcypromine, PMA, 3-deazaneplanocin A, 5-aza-2′-deoxycytidine, 4-phenylbutyrate, one or more PKC alpha activators, one or more PKC epsilon activators, one or more ERK activators, valproic acid, insulin, or a combination thereof.

In specific embodiments, the mitogenic factor is EGF. In specific embodiments, the incubation medium comprises at least 1, 2, 5, 7, 10, 12, or 15 ng/nl EGF. In specific embodiments, the incubation medium comprises at most 5, 7, 10, 12, 15, 20, or 25 ng/nl EGF. In specific embodiments, the incubation medium comprises 1-20, 2-20, 5-20, 7-20, 10-20, 12-20, 1-15, 2-15, 5-15, 7-15, 10-15, 12-15, 1-12, 2-12, 5-12, 7-12, or 10-12 ng/nl EGF. In specific embodiments, the incubation medium comprises 5-20 ng/nl EGF.

In specific embodiments, the said mitogenic factor is one or more FGFs. In specific embodiments, the incubation medium comprises at least 1, 2, 5, 7, 10, 12, or 15 ng/nl of one or more FGFs. In specific embodiments, the incubation medium comprises at most 5, 7, 10, 12, 15, 20, or 25 ng/nl of one or more FGFs. In specific embodiments, the incubation medium comprises 1-20, 2-20, 5-20, 7-20, 10-20, 12-20, 1-15, 2-15, 5-15, 7-15, 10-15, 12-15, 1-12, 2-12, 5-12, 7-12, or 10-12 ng/nl of one or more FGFs. In specific embodiments, the incubation medium comprises 5-20 ng/nl of one or more FGFs.

In specific embodiments, the mitogenic factor is one or more cytokines. In specific embodiments, the cytokine is one or more pro-inflammatory cytokines, one or more chemokines, one or more pro-fibrotic cytokines, or a combination thereof. In specific embodiments, the cytokine is IGF-1, IL-6, TGF-beta, TNF-alpha, CTGF, SPARC, SDF1, or a combination thereof. In specific embodiments, the incubation medium comprises 0.1-100 ng/nl of one or more cytokines. In specific embodiments, the incubation medium comprises 10-50 ng/nl IL-6. In specific embodiments, the incubation medium comprises 0.1-30 ng/nl TGF-beta. In specific embodiments, the incubation medium comprises 25-80 ng/nl TNF-alpha. In specific embodiments, the incubation medium comprises 1-50 ng/nl CTGF. In specific embodiments, the incubation medium comprises 10-50 ng/nl SPARK. In specific embodiments, the said incubation medium comprises 1-50 ng/nl SDF1.

In specific embodiments, the mitogenic factor is one or more ROCK inhibitors. In specific embodiments, the ROCK inhibitor is Y-27632, thiazovivin, fasudil, GSK429286A, or a combination thereof. In specific embodiments, the mitogenic factor is one or more GSK3 inhibitors. In specific embodiments, the said GSK3 inhibitor is CHIR99021, kenpaullone, SB-216763, indirubin, hymenidin, aloinin A, lithium chloride, or a combination thereof. In specific embodiments, the mitogenic factor is one or more MAO inhibitors. In specific embodiments, the MAO inhibitor is tranylcypromine, 2-propynal, clorgyline hydrochloride, ethyl homovanillate, phenelzine sulfate salt, rasagiline, pimprinine, or a combination thereof. In specific embodiments, the mitogenic factor is one or more PKC activators. In specific embodiments, the PKC activator is PKC alpha, PKC epsilon, PMA, alpha-amyloid precursor protein, phorbol-12, 13-dibutyrate, mezerein, ingenol 3-angelate, phorbol 12, 13-dihexanoate, or a combination thereof. In specific embodiments, the mitogenic factor is one or more ERK activators. In specific embodiments, the ERK activator is Ceramide C6.

In specific embodiments, the mitogenic factor is one or more histone modifying enzyme inhibitors. In specific embodiments, the histone modifying enzyme inhibitor is 3-deazaneplanocin A, 5-aza-2′-deoxycytidine, 4-phenylbutyrate, TC-H 106, valproic acid, insulin, SIRT1 activator 3, or a combination thereof.

In specific embodiments, the mitogenic factor is Y-27632, CHIR99021, tranylcypromine, PMA, 3-deazaneplanocin A, 5-aza-2′-deoxycytidine, 4-phenylbutyrate, one or more PKC alpha activators, one or more PKC epsilon activators, valproic acid, insulin, or a combination thereof. In specific embodiments, the incubation medium further comprises 0.1-100 ng/ml of Y-27632, CHIR99021, tranylcypromine, PMA, 3-deazaneplanocin A, 5-aza-2′-deoxycytidine, 4-phenylbutyrate, one or more PKC alpha activators, one or more PKC epsilon activators, valproic acid, insulin, or a combination thereof. In specific embodiments, the mitogenic factor is PMA. In specific embodiments, the incubation medium further comprises 1-50 ng/ml PMA.

In specific embodiments, the incubation medium comprises an effective amount of an inhibitor to reduce expression or function of a protein or complex from 10 to 99%. In specific embodiments, the incubation medium comprises an effective amount of an activator to increase expression or function of a protein or complex by 10 to 99%.

In specific embodiments, methods are provided further comprising the step of genetically modifying the fibroblasts. In specific embodiments, the gene modification comprises transfecting the fibroblasts with one or more genes. In specific embodiments, a therapeutically effective amount of the genetically modified fibroblasts are provided as a therapeutic application to an individual in need thereof. In specific embodiments, the therapeutic application is treatment of fibrosis, treatment of inflammation, acceleration of healing, treatment of clotting disorders, or promotion of angiogenesis.

In specific embodiments, the therapeutic application comprises treatment of fibrosis, wherein said one or more genes comprises one or more anti-fibrotic factors. In specific embodiments, the antifibrotic factor is one or more cathepsins, calpin 1, calpin 2, chondroitinase ABC, chondroitinase AC, pancreatic elastase, elastase-2a, elastase-2b, neutrophil elastase, proteinase-3, endogenous vascular elastase, mast cell chymase, mast cell tryptase, plasmin, thrombin, granzyme B, hyaluronidase, chymopapain, chymotrypsin, legumain, collagenase, one or more matrix metalloproteinases, one or more aggrecanases, papain, subtilisin, subtilisin A, heparanase, one or more TGF-beta receptor antagonists, one or more angiotensin inhibitors, one or more angiotensin-converting enzyme inhibitors, one or more angiotensin-II receptor antagonists, one or more cannabinoid receptor, one or more interleukins, one or more interferons, one or more cytokines, one or more chemokines, one or more chemotactic molecules, relaxin, one or more hormones, or a combination thereof.

In specific embodiments, the antifibrotic factor is one or more cathepsins. In specific embodiments, the cathepsin is cathepsin D, cathepsin E, cathepsin S, cathepsin K, cathepsin L, cathepsin B, cathepsin C, cathepsin H, cathepsin F, cathepsin G, cathepsin O, cathepsin R, cathepsin V, cathepsin W, cathepsin Z, or a combination thereof.

In specific embodiments, the antifibrotic factor is one or more metalloproteinases. In specific embodiments, the metalloproteinase is MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-23, MMP-24, MMP-25, MMP-26, MMP-23, MMP-27, MMP-28, or a combination thereof. In specific embodiments, the anti-fibrotic factor is one or more aggrecanases. In specific embodiments, the aggrecanase is ADAMTS-1, ADAMTS-2, ADAMTS-3, ADAMTS-4, ADAMTS-5, ADAMTS-14, or a combination thereof.

In specific embodiments, the anti-fibrotic factor is one or more TGF-beta receptor antagonists. In specific embodiments, the TGF-beta receptor antagonist is decorin, one or more decorin-fusion proteins, one or more anti-TGF-beta receptor antibodies, one or more anti-TGF-beta receptor oligonucleotides, or a combination thereof. In specific embodiments, the TGF-beta receptor antagonist is one or more decorin-fusion proteins. In specific embodiments, the decorin-fusion protein is CAR-decorin.

In specific embodiments, the TGF-beta receptor antagonist is one or more anti-TGF-beta receptor antibodies. In specific embodiments, the anti-TGF-beta receptor antibody is fresolimumab, lerdelimumab, metelimumab, one or more monoclonal antibodies, one or more humanized antibodies, one or more human antibodies, one or more antibody fragments, or a combination thereof. In specific embodiments, the TGF-beta receptor antagonist is one or more anti-TGF-beta receptor oligonucleotides. In specific embodiments, the anti-TGF-beta receptor oligonucleotide is trabedersen, one or more siRNAs targeting TGF-beta, or a combination thereof.

In specific embodiments, the therapeutic application comprises treatment of inflammation, wherein said one or more genes comprises one or more anti-inflammatory factors. In specific embodiments, the anti-inflammatory factor is one or more inflammatory cytokine antagonists, one or more anti-microbial factors, one or more antibodies, one or more non-antibody anti-inflammatory factors, one or more soluble receptor fusion proteins, or a combination thereof.

In specific embodiments, the anti-inflammatory factor is one or more inflammatory cytokine antagonists. In specific embodiments, the inflammatory cytokine antagonist is one or more IL-6 antagonists, one or more IL-10 antagonists, IL-10, LL-37, thymosin beta 4, or a combination thereof. In specific embodiments, the anti-inflammatory factor is one or more anti-microbial factors. In specific embodiments, the anti-microbial factor is LL-37, thymosin beta 4, or a combination thereof.

In specific embodiments, the said anti-inflammatory factor is one or more antibodies. In specific embodiments, the antibody is one or more anti-TNF-alpha antibodies, one or more anti-IL-6 antibodies, one or more anti-IL-10 antibodies, one or more monoclonal antibodies, one or more humanized antibodies, one or more human antibodies, one or more antibody fragments, or a combination thereof. In specific embodiments, the antibody is one or more anti-TNF-alpha antibodies. In specific embodiments, the anti-TNF-alpha antibody is infliximab, adalimumab, certolizumab pegol, golimumab, or combinations thereof. In specific embodiments, the anti-inflammatory factor is one or more soluble receptor fusion proteins. In specific embodiments, the soluble receptor fusion protein is etanercept.

In specific embodiments, the anti-inflammatory factor is one or more non-antibody anti-inflammatory factors. In specific embodiments, the non-antibody anti-inflammatory factor excludes antibodies. In specific embodiments, the non-antibody anti-inflammatory factor excludes monoclonal antibodies, human antibodies, humanized antibodies, or a combination thereof. In specific embodiments, the non-antibody anti-inflammatory factor excludes TNF antibodies, alemtuzumab, afelimomab, aselizumab, atlizumab, atorolimumab, basiliximab, belimumab, bertilimumab, cedelizumab, clenoliximab, daclizumab, dorlimomab aritox, dorlixizumab, eculizumab, efalizumab, elsilimomab, erlizumab, faralimomab, fontolizumab, galiximab, gantenerumab, gavilimomab, golimumab, gomiliximab, ibalizumab, inolimomab, ipilimumab, keliximab, lebrilizumab, lerdelimumab, lumiliximab, maslimomab, mepolizumab, metelimumab, morolimumab, muromonab-CD3, natalizumab, nerelimomab, ocrelizumab, odulimomab, omalizumab, otelixizumab, pascolizumab, pexelizumab, rituxumab, reslizumab, rovelizumab, ruplizumab, siplizumab, talizumab, telimomab aritox, teneliximab, teplizumab, tocilizumab, toralizumab, vapaliximab, vepalimomab, visilizumab, zanolimumab, ziralimumab, zolimomab aritox, or a combination thereof.

In specific embodiments, the therapeutic application comprises acceleration of healing, wherein said one or more genes comprises one or more extracellular matrix factors. In specific embodiments, the extracellular matrix factor is decorin, fibronectin, vitronectin, laminin, COM, tenascin-C, tenascin-X, elastin, TIMP-1, albumin, osteonectin, one or more collagens, one or more keratins, one or more thrombospondins, one or more proteoglycans, one or more glycosaminoglycans, one or more integrins, or a combination thereof.

In specific embodiments, the extracellular matrix factor is one or more collagens. In specific embodiments, the collagen is collagen-I, collagen-III, collagen-VI, or a combination thereof. In specific embodiments, the extracellular matrix factor is one or more keratins. In specific embodiments, the keratin is K6, K16, or a combination thereof. In specific embodiments, the extracellular matrix factor is one or more thrombospondins. In specific embodiments, the thrombospondin is thrombospondin-1, thrombospondin-2, or a combination thereof.

In specific embodiments, the extracellular matrix factor is one or more proteoglycans. In specific embodiments, the proteoglycan is versican, syndecan, glypicans, perlecan, lumican, heparin sulfate, or a combination thereof. In specific embodiments, the extracellular matrix factor is one or more glycosaminoglycans. In specific embodiments, the glycosaminoglycan is a combination of hyaluronan and hyaluronic acid.

In specific embodiments, the therapeutic application comprises treatment of one or more clotting disorders, wherein said one or more genes comprises one or more clotting factors. In specific embodiments, the clotting factor is factor I, factor II, factor III, factor V, factor VII, factor VIII, factor IX, factor X, factor XI, factor XII, factor XIII, von Willebrand factor, prekallikrein, HMWK, fibronectin, antithrombin III, heparin cofactor II, protein C, protein S, protein Z, plasminogen, tPA, urokinase, or a combination thereof.

In specific embodiments, the therapeutic application comprises promotion of angiogenesis, wherein said one or more genes comprises one or more angiogenic factors. In specific embodiments, the angiogenic factor is one or more VEGFs, one or more angiopoietins, one or more angiopoietin-like proteins, one or more FGFs, one or more TGFs, one or more TNFs, one or more CSFs, one or more NOSs, EGF, or a combination thereof.

In specific embodiments, the angiogenic factor is one or more VEGFs. In specific embodiments, the VEGF is VEGF-A, VEGF-B, VEGF-C, VEGF-D, VEGF-E, PIGF, or a combination thereof. In specific embodiments, the angiogenic factor is one or more angiopoietins. In specific embodiments, the angiopoietin is ANGPT1, ANGPT2, ANGPT4, or a combination thereof. In specific embodiments, the angiogenic factor is one or more angiopoietin-like proteins. In specific embodiments, the angiopoietin-like protein is ANGPTL1, ANGPTL2, ANGPTL3, ANGPTL4, ANGPTL5, ANGPTL6, ANGTPL7, or a combination thereof.

In specific embodiments, the angiogenic factor is one or more FGFs. In specific embodiments, the FGF is FGF-1, FGF-2, or a combination thereof. In specific embodiments, the angiogenic factor is one or more TGFs. In specific embodiments, the TGF is TGF-alpha, TGF-beta, or combinations thereof. In specific embodiments, the angiogenic factor is one or more TNFs. In specific embodiments, the TNF is TNF-alpha. In specific embodiments, the angiogenic factor is one or more CSFs. In specific embodiments, the CSF is GM-CSF, M-CSF, or combinations thereof.

In specific embodiments, the fibroblasts are autologous, allogeneic, or xenogeneic with respect to a recipient. In specific embodiments, the source of the fibroblasts comprises: a) fallopian tube; b) menstrual blood; c) endometrium; d) skin; e) nail cuticle; f) deciduous tooth; g) hair follicle; h) tonsil; i) omentum; j) bone marrow; k) placenta; l) umbilical cord; m) peripheral blood; n) adipose tissue; o) amniotic fluid; p) umbilical cord blood; q) embryonic fibroblasts; r) plastic surgery related by-product; or s) nail matrix. In specific embodiments, the peripheral blood is extracted after activation of fibroblasts. In specific embodiments, the activation is achieved by administration of G-CSF. In specific embodiments, the activation is achieved by administration of M-CSF. In specific embodiments, the activation is achieved by administration of GM-CSF. In specific embodiments, the activation is achieved by administration of Flt3 ligand.

In specific embodiments, fibroblasts are transfected with one or more genes selected from the group consisting of Lin-28, STAT3, NFKB, CEBP/B, SOX2, OCT4, WNT5A, LIF, COX2, RUNX2, NANOG, and a combination thereof. In specific embodiments, the fibroblasts endogenously express at least one of CD184, CD193, CD235a, CD318, CD255, CD268, fMLP, ITGA2, or ITGA4.

In specific embodiments, the fibroblasts are transfected with and/or express one or more genes selected from the group consisting of 37135 (Sep-01); 37500 (Sep-02); 37865 (Sep-03); 38231 (Sep-04); 38596 (Sep-05); 38961 (Sep-06); 39326 (Sep-07); 40057 (Sep-09); 40422 (Sep-10); 40787 (Sep-11); 41153 (Sep-12); 41883 (Sep-14); A1BG; A1CF; A2M; A2ML1; A4GNT; AAAS; AACS; AADAC; AAGAB; AAK1; AANAT; AARD; AARS2; AARS; AASDH; AASDHPPT; AASS; AATF; AATK; ABAT; ABCA12; ABCA13; ABCA1; ABCA2; ABCA3; ABCA5; ABCA6; ABCA7; ABCA9; ABCB11; ABCB1; ABCB4; ABCB5; ABCB6; ABCB7; ABCB8; ABCB9; ABCC10; ABCC11; ABCC12; ABCC1; ABCC2; ABCC3; ABCC4; ABCC5; ABCC6; ABCC8; ABCC9; ABCD1; ABCD2; ABCD3; ABCD4; ABCE1; ABCF1; ABCF2; ABCG1; ABCG2; ABCG4; ABCG5; ABCG8; ABHD12B; ABHD12; ABHD16A; ABHD17B; ABHD2; ABHDS; ABHD6; ABI1; ABI2; ABI3BP; ABI3; ABL1; ABL2; ABLIM1; ABLIM2; ABO; ABRA; ABR; ABT1; ACAA1; ACAA2; ACACA; ACACB; ACAD10; ACAD8; ACAD9; ACADL; ACADSB; ACADS; ACADVL; ACAN; ACAT1; ACAT2; ACBD3; ACBD4; ACBD5; ACBD6; ACCS; ACD; ACE2; ACE; ACER2; ACER3; ACHE; ACINI; ACKR1; ACKR2; ACKR3; ACKR4; ACLY; ACMSD; ACO1; ACO2; ACOT13; ACOT1; ACOT2; ACOT9; ACOX1; ACOX2; ACOX3; ACOXL; ACP1; ACP2; ACP5; ACP6; ACPP; ACRBP; ACRC; ACR; ACRV1; ACSBG1; ACSBG2; ACSF3; ACSL1; ACSL3; ACSL5; ACSL6; ACSM1; ACSM2B; ACSM3; ACSS1; ACSS2; ACSS3; ACTA1; ACTA2; ACTB; ACTBL2; ACTC1; ACTG1; ACTG2; ACTL6A; ACTL6B; ACTL8; ACTL9; ACTN1; ACTN2; ACTN3; ACTN4; ACTR1A; ACTR1B; ACTR2; ACTR3B; ACTR3; ACTR5; ACTRT1; ACVR1B; ACVR1C; ACVR1; ACVR2A; ACVR2B; ACVRL1; ACYL; ACY3; ACYP2; ADAD1; ADA; ADAM10; ADAM11; ADAM12; ADAM15; ADAM17; ADAM19; ADAM20; ADAM21; ADAM22; ADAM23; ADAM28; ADAM29; ADAM2; ADAM33; ADAM7; ADAMS; ADAMS; ADAMDEC1; ADAMTS10; ADAMTS12; ADAMTS13; ADAMTS14; ADAMTS15; ADAMTS16; ADAMTS17; ADAMTS18; ADAMTS19; ADAMTS1; ADAMTS20; ADAMTS2; ADAMTS3; ADAMTS4; ADAMTS5; ADAMTS6; ADAMTS7; ADAMTS8; ADAMTS9; ADAMTSL1; ADAMTSL2; ADAMTSL3; ADAMTSL4; ADAMTSL5; ADAP1; ADAP2; ADARB1; ADARB2; ADAR; ADAT2; ADAT3; ADCK3; ADCK4; ADCY10; ADCY1; ADCY2; ADCY3; ADCYS; ADCY6; ADCY7; ADCY8; ADCY9; ADCYAP1; ADCYAP1R1; ADD1; ADD2; ADD3; ADGB; ADGRA1; ADGRA2; ADGRA3; ADGRB1; ADGRB2; ADGRB3; ADGRD1; ADGRD2; ADGRE1; ADGRE2; ADGRE5; ADGRF5; ADGRG1; ADGRG2; ADGRG3; ADGRG6; ADGRG7; ADGRL1; ADGRL3; ADGRV1; ADH1A; ADH1B; ADH4; ADH5; ADH6; ADH7; ADHFE1; AM; ADIG; ADIPOQ; ADIPOR1; ADIPOR2; ADIRF; ADK; ADM2; ADM; ADNP; ADO; ADORAL; ADORA2A; ADORA2B; ADPGK; ADPRH; ADPRHL1; ADPRHL2; ADRA1A; ADRA1B; ADRA1D; ADRA2A; ADRA2B; ADRA2C; ADRB1; ADRB2; ADRB3; ADRBK1; ADRBK2; ADRM1; ADSL; ADSS; ADSSL1; ADTRP; AEBP2; AEN; AES; AFAP1; AFAP1L1; AFAP1L2; AFF1; AFF2; AFF3; AFF4; AFG3L2; AFM; AFP; AGA; AGAP1; AGAP2; AGAP3; AGAP4; AGBL1; AGBL2; AGBL3; AGBL4; AGER; AGFG1; AGFG2; AGGF1; AGK; AGMO; AGO2; AGPAT1; AGPAT2; AGPAT3; AGPAT4; AGPAT9; AGPS; AGR2; AGR3; AGRN; AGRP; AGT; AGTPBP1; AGTR1; AGTR2; AGXT2; AGXT; AHCY; AHCYL2; AHI1; AHNAK; AHR; AHRR; AHSA1; AHSA2; AHSG; AICDA; AIDA; AIF1; AIFM1; AIFM2; AIFM3; AIG1; AIM1; AIM2; AIMP1; AIMP2; AIP; AIPL1; AIRE; AJAP1; AJUBA; AK1; AK3; AK6; AK7; AK8; AK9; AKAP10; AKAP12; AKAP13; AKAP17A; AKAP1; AKAP2; AKAP3; AKAP4; AKAP5; AKAP6; AKAP7; AKAP9; AKIP1; AKIRIN2; AKNA; AKR1B10; AKR1B1; AKR1C1; AKR1C2; AKR1C3; AKR1C4; AKR1D1; AKR1E2; AKR7A3; AKT1; AKT1S1; AKT2; AKT3; AKTIP; ALAD; ALAS1; ALAS2; ALB; ALCAM; ALDH16A1; ALDH18A1; ALDH1A1; ALDH1A2; ALDH1A3; ALDH1B1; ALDH1L1; ALDH2; ALDH3A1; ALDH3A2; ALDH3B1; ALDH4A1; ALDH5A1; ALDH6A1; ALDH7A1; ALDH9A1; ALDOA; ALDOB; ALDOC; ALG10B; ALG10; ALG11; ALG12; ALG13; ALG1; ALG1L; ALG2; ALG3; ALG8; ALG9; ALKBH1; ALKBH2; ALKBH3; ALKBH7; ALKBH8; ALK; ALLC; ALMS1; ALOX12B; ALOX12; ALOX15B; ALOX15; ALOX5AP; ALOX5; ALOXE3; ALPI; ALPK1; ALPK2; ALPK3; ALPP; ALPPL2; ALS2CL; ALS2CR12; ALS2; ALX1; ALX4; ALYREF; AMACR; AMBN; AMBP; AMBRA1; AMD1; AMELX; AMELY; AMER1; AMER2; AMER3; AMFR; AMH; AMHR2; AMICA1; AMIGO2; AMMECR1; AMN; AMOT; AMOTL1; AMPD2; AMPD3; AMPH; AMT; AMZ1; ANAPC10; ANAPC11; ANAPC13; ANAPC1; ANAPC2; ANAPC4; ANAPC5; ANAPC7; ANG; ANGPT1; ANGPT2; ANGPT4; ANGPTL1; ANGPTL2; ANGPTL4; ANGPTL6; ANK1; ANK2; ANK3; ANKFN1; ANKFY1; ANKHD1-EIF4EBP3; ANKHD1; ANKH; ANKK1; ANKLET; ANKLE2; ANKMY1; ANKRD10; ANKRD11; ANKRD12; ANKRD18A; ANKRD1; ANKRD23; ANKRD26; ANKRD28; ANKRD2; ANKRD30A; ANKRD36B; ANKRD36; ANKRD37; ANKRD44; ANKRD45; ANKRD46; ANKRD50; ANKRD55; ANKRD6; ANKRD7; ANKS1A; ANKS1B; ANKS4B; ANKS6; ANLN; AN010; ANO1; ANO2; ANO3; ANO4; ANDS; ANO6; ANO7; ANP32A; ANP32B; ANP32D; ANPEP; ANTXR1; ANTXR2; ANXA10; ANXA11; ANXA13; ANXA1; ANXA2; ANXA2R; ANXA3; ANXA4; ANXA5; ANXA6; ANXA7; ANXA8; ANXA8L1; AOAH; AOC1; AOC2; AOC3; AOX1; AP1AR; AP1B1; AP1G1; AP1M1; AP1M2; AP1S1; AP1S2; AP1S3; AP2A1; AP2B1; AP2M1; AP2S1; AP3B1; AP3D1; AP3M2; AP3S1; AP3S2; AP4B1; AP4E1; AP4M1; AP4S1; AP5M1; AP5Z1; APAF1; APBA1; APBA2; APBA3; APBB1; APBB1IP; APBB2; APBB3; APC2; APCDD1; APCDD1L; APC; APCS; APEH; APEX1; APEX2; APH1B; APIS; APIP; APLN; APLNR; APLP1; APLP2; APOA1BP; APOA2; APOA4; APOA5; APOBEC1; APOBEC2; APOBEC3A_B; APOBEC3B; APOBEC3C; APOBEC3F; APOBEC3G; APOBEC3H; APOB; APOBR; APOC1; APOC2; APOC3; APOC4; APOD; APOE; APOF; APOH; APOL1; APOL2; APOL3; APOL4; APOL6; APOLD1; APOM; APOO; APOPT1; APPBP2; APP; APPL1; APPL2; APRT; APTX; AQP10; AQP1; AQP2; AQP3; AQP4; AQP5; AQP6; AQP7; AQP8; AQP9; ARAF; ARAP1; ARAP3; ARC; ARCN1; AREG; ARF1; ARF3; ARF4; ARF6; ARFGAP1; ARFGAP2; ARFGAP3; ARFGEF1; ARFGEF2; ARFGEF3; ARFIP1; ARFRP1; ARG1; ARGLU1; ARHGAP10; ARHGAP11A; ARHGAP11B; ARHGAP15; ARHGAP18; ARHGAP1; ARHGAP20; ARHGAP21; ARHGAP22; ARHGAP23; ARHGAP24; ARHGAP25; ARHGAP26; ARHGAP27; ARHGAP28; ARHGAP30; ARHGAP31; ARHGAP32; ARHGAP35; ARHGAP42; ARHGAP4; ARHGAP5; ARHGAP6; ARHGAP9; ARHGD1A; ARHGD1B; ARHGEF10; ARHGEF10L; ARHGEF11; ARHGEF12; ARHGEF15; ARHGEF16; ARHGEF17; ARHGEF1; ARHGEF25; ARHGEF26; ARHGEF28; ARHGEF2; ARHGEF38; ARHGEF3; ARHGEF4; ARHGEF5; ARHGEF6; ARHGEF7; ARHGEF9; AR; ARID1B; ARID2; ARID3A; ARID3B; ARID4A; ARID4B; ARID5B; ARIH1; ARL11; ARL13A; ARL13B; ARL14EP; ARL14; ARL15; ARL1; ARL2BP; ARL2; ARL3; ARL4A; ARL4C; ARL4D; ARL5A; ARL5B; ARL6; ARL6IP1; ARL6IP5; ARMC10; ARMC1; ARMC2; ARMC3; ARMC4; ARMC5; ARMC8; ARMC9; ARMCX1; ARMS2; ARMT1; ARNT2; ARNT; ARNTL2; ARNTL; ARPC1A; ARPC1B; ARPC2; ARPC3; ARPC5; ARPIN; ARPP21; ARR3; ARRB1; ARRB2; ARRDC2; ARRDC3; ARRDC4; ARSA; ARSB; ARSD; ARSE; ARSF; ARSG; ARSH; ARSI; ARSJ; ARSK; ART1; ART3; ART4; ARVCF; ARX; AS3MT; ASAH1; ASAH2; ASAP1; ASAP2; ASB10; ASB13; ASB15; ASB18; ASB1; ASB2; ASB6; ASB7; ASCC1; ASCC2; ASCC3; ASCL1; ASCL2; ASCL4; ASF1A; ASF1B; ASH1L; ASH2L; ASIC1; ASIC2; ASIC3; ASIC4; ASIC5; ASIP; ASL; ASMT; ASMTL; ASNA1; ASNS; ASPA; ASPG; ASPH; ASPM; ASPN; ASPRV1; ASPSCR1; ASRGL1; ASS1; ASTN2; ASUN; ASXL1; ASXL2; ASXL3; ASZ1; ATAD2; ATAD3B; ATAD3C; ATAD5; ATAT1; ATCAY; ATE1; ATFL; ATF2; ATF3; ATF4; ATF5; ATF6B; ATF6; ATF7; ATF7IP; ATG10; ATG12; ATG16L1; ATG2B; ATG3; ATG4A; ATG4B; ATG4C; ATG5; ATG7; ATG9A; ATIC; ATL1; ATL2; ATL3; ATM; ATN1; ATOH1; ATOH7; ATOX1; ATP10A; ATP10B; ATP10D; ATP11A; ATP11AUN; ATP11B; ATP12A; ATP13A3; ATP13A4; ATP1A2; ATP1A3; ATP1A4; ATP1B1; ATP1B2; ATP2A1; ATP2A2; ATP2A3; ATP2B1; ATP2B2; ATP2B3; ATP2B4; ATP2C1; ATP2C2; ATP4A; ATP4B; ATP5A1; ATP5B; ATP5C1; ATP5D; ATP5E; ATP5G1; ATP5G2; ATP5G3; ATP5H; ATP5J2; ATP5J; ATP5L; ATP5O; ATP6AP1; ATP6AP1L; ATP6AP2; ATP6V0A1; ATP6V0A2; ATP6V0A4; ATP6V0C; ATP6V0D1; ATP6V0E1; ATP6V0E2; ATP6V1B1; ATP6V1B2; ATP6V1C1; ATP6V1D; ATP6V1E1; ATP6V1F; ATP6V1G1; ATP6V1G2; ATP6V1G3; ATP7A; ATP7B; ATP8A1; ATP8A2; ATP8B1; ATP8B3; ATP8B4; ATP9B; ATPAF2; ATRAID; ATR; ATRIP; ATRN; ATRNL1; ATRX; ATXN10; ATXN1; ATXN1L; ATXN2; ATXN2L; ATXN3; ATXN7; ATXN7L3B; AUH; AURKA; AURKB; AURKC; AUTS2; AVEN; AVP; AVPI1; AVPR1A; AVPR1B; AVPR2; AWAT1; AXDND1; AXIN1; AXIN2; AXL; AZGP1; AZI2; AZIN1; AZIN2; AZUL; B2M; B3GALNT1; B3GALNT2; B3GALT2; B3GALT4; B3GALT5; B3GALTL; B3GAT1; B3GAT2; B3GAT3; B3GNT2; B3GNT3; B3GNT5; B3GNT6; B3GNT8; B3GNTL1; B4GALNT1; B4GALNT2; B4GALNT3; B4GALT1; B4GALT3; B4GALT4; B4GALT5; B4GALT6; B4GALT7; B4GAT1; B9D1; B9D2; BAALC; BAAT; BABAM1; BACE1; BACE2; BACH1; BACH2; BAD; BAG1; BAG3; BAG4; BAG5; BAG6; BAIAP2L1; BAIAP3; BAK1; BAMBI; BANF1; BANK1; BANP; BAP1; BARD1; BARHL1; BARX1; BARX2; BASP1; BATF2; BATF; BAX; BAZ1A; BAZ1B; BAZ2A; BAZ2B; BBC3; BBIP1; BBOX1; BBS10; BBS12; BBS1; BBS2; BBS4; BBS5; BBS7; BBS9; BBX; BCAM; BCAN; BCAP29; BCAP31; BCAR1; BCAS1; BCAS3; BCAS4; BCAT1; BCAT2; BCCIP; BCDIN3D; BCHE; BCKDHA; BCKDHB; BCKDK; BCL10; BCL11A; BCL11B; BCL2A1; BCL2; BCL2L10; BCL2L11; BCL2L12; BCL2L13; BCL2L14; BCL2L1; BCL2L2; BCL2L2-PABPN1; BCL3; BCL6B; BCL6; BCL7A; BCL7B; BCL7C; BCL9L; BCLAF1; BCO1; BCO2; BCOR; BCORL1; BCR; BCS1L; BDH1; BDH2; BDKRB1; BDKRB2; BDNF; BDP1; BEAN1; BEGAIN; BEND3; BEND4; BEST1; BEST2; BET1; BET1L; BEX1; BEX2; BEX4; BFAR; BFSP1; BFSP2; BGLAP; BGN; BHLHA15; BHLHA9; BHLHB9; BHLHE22; BHLHE23; BHLHE40; BHLHE41; BHMT2; BHMT; BICC1; BICD1; BICD2; BID; BIN1; BIN2; BIN3; BIRC2; BIRC3; BIRC5; BIRC6; BIRC7; BIVM; BLCAP; BLID; BLK; BLMH; BLM; BLNK; BLOC1S2; BLOC1S3; BLOC1S4; BLOC1S5; BLOC1S6; BLVRA; BLVRB; BLZF1; BMF; BMI1; BMP10; BMP15; BMP1; BMP2; BMP2K; BMP3; BMP4; BMP5; BMP6; BMP7; BMP8B; BMPER; BMPR1A; BMPR1B; BMPR2; BMS1; BMX; BNC1; BNC2; BNIP1; BNIP2; BNIP3; BNIP3L; BNIPL; BOC; BOD1L2; BOK; BOLA3; BOLL; BOP1; BORA; BPGM; BPIFA1; BPIFA2; BPIFA3; BPIFB1; BPIFB2; BPIFC; BPI; BPNT1; BPTF; BPY2; BRAF; BRAP; BRAT1; BRCA1; BRCA2; BRCC3; BRD1; BRD2; BRD3; BRD4; BRD7; BRD8; BRE; BRF1; BRF2; BRI3BP; BRI3; BRINP1; BRINP2; BRINP3; BRIP1; BRK1; BRMS1; BRMS1L; BRS3; BRSK1; BRSK2; BRWD1; BRWD3; BSCL2; BSG; BSN; BSPH1; BSPRY; BST1; BST2; BSX; BTBD10; BTBD11; BTBD16; BTBD1; BTBD2; BTBD3; BTBD9; BTC; BTD; BTF3; BTG1; BTG2; BTG3; BTG4; BTK; BTLA; BTN1A1; BTN2A1; BTN2A2; BTN3A1; BTN3A2; BTN3A3; BTNL2; BTRC; BUB1B; BUB1; BUB3; BUD13; BUD31; BVES; BYSL; BZRAP1; BZW1; C10orf107; C10orf10; C10orf113; C10orf11; C10orf2; C10orf32; C10orf35; C10orf54; C10orf67; C10orf82; C10orf88; C10orf90; C11orf21; C11orf30; C11orf53; C11orf65; C11orf68; C11orf73; C11orf74; C11orf80; C11orf87; C11orf95; C12orf10; C12orf42; C12orf43; C12orf50; C12orf57; C12orf5; C12orf65; C12orf66; C12orf75; C12orf77; C14orf166; C14orf177; C14orf1; C15orf32; C15orf41; C15orf48; C15orf53; C15orf59; C16orf72; C16orf74; C16orf78; C16orf95; C17orf51; C17orf53; C17orf64; C17orf96; C18orf54; C18orf8; C19orf12; C19orf18; C19orf24; C19orf26; C19orf33; C19orf40; C19orf45; C19orf48; C19orf57; C19orf68; C1D; C1GALT1C1; C1GALT1; C1orf106; C1orf109; C1orf110; C1orf112; C1orf115; C1orf127; C1orf141; C1orf167; C1orf204; C1orf226; C1orf228; C1orf27; C1orf61; C1orf86; C1QB; C1QBP; C1QL1; C1QL3; C1QTNF1; C1QTNF3; C1QTNF5; C1QTNF6; C1QTNF7; C1QTNF9B-AS1; C1R; C1RL; C1S; C20orf194; C20orf196; C20orf27; C20orf85; C21orf2; C21orf33; C21orf59; C21orf62; C21orf91; C22orf29; C2CD3; C2CD4A; C2CD4B; C2CD5; C2; C2orf16; C2orf40; C2orf43; C2orf47; C2orf57; C2orf61; C3AR1; C3; C3orf17; C3orf18; C3orf20; C3orf35; C3orf56; C3orf58; C3orf67; C3orf79; C4A; C4B_2; C4B; C4BPA; C4BPB; C4orf22; C4orf26; C4orf27; C4orf32; C4orf33; C4orf36; C4orf48; C4orf51; C5AR1; C5AR2; C5; C5orf22; C5orf30; C5orf34; C5orf38; C5orf42; C5orf63; C6; C6orf106; C6orf10; C6orf15; C6orf25; C6orf47; C6orf48; C6orf89; C7; C7orf49; C7orf57; C7orf60; C7orf62; C7orf65; C7orf69; C7orf72; C8orf34; C8orf37; C8orf46; C8orf48; C8orf4; C8orf86; C9; C9orf106; C9orf135; C9orf152; C9orf156; C9orf170; C9orf171; C9orf3; C9orf43; C9orf66; C9orf72; C9orf85; C9orf91; C9orf9; CA10; CA11; CA12; CA13; CA1; CA2; CA3; CA4; CASA; CA6; CA8; CAB39; CAB39L; CABIN1; CABLES1; CABP2; CABP4; CABS1; CABYR; CACNA1A; CACNA1B; CACNA1C; CACNA1D; CACNA1E; CACNA1F; CACNA1G; CACNA1H; CACNA1I; CACNA1S; CACNA2D1; CACNA2D2; CACNA2D3; CACNA2D4; CACNB1; CACNB2; CACNB3; CACNB4; CACNG2; CACNG3; CACNG4; CACNG5; CACNG6; CACUL1; CACYBP; CAD; CADM1; CADM2; CADM3; CADM4; CADPS2; CADPS; CAGE1; CALB1; CALB2; CALCA; CALCB; CALCOCO1; CALCOCO2; CALCR; CALCRL; CALD1; CALHM1; CALHM2; CALHM3; CALM2; CALML3; CALML5; CALN1; CALR3; CALR; CALU; CALY; CAMK1D; CAMK1G; CAMK1; CAMK2A; CAMK2B; CAMK2D; CAMK2G; CAMK4; CAMKK1; CAMKK2; CAMKMT; CAMLG; CAMP; CAMSAP1; CAMSAP2; CAMTA1; CAND1; CAND2; CANT1; CANX; CAP1; CAP2; CAPG; CAPN10; CAPN13; CAPN14; CAPN1; CAPN2; CAPN3; CAPN5; CAPN6; CAPN7; CAPN9; CAPNS1; CAPRIN1; CAPRIN2; CAPS2; CAPS; CAPSL; CAPZA2; CAPZA3; CARD10; CARD11; CARD14; CARD16; CARD6; CARDS; CARDS; CARF; CARKD; CARM1; CARS; CARTPT; CASC1; CASC3; CASC4; CASC5; CASD1; CASK; CASP10; CASP14; CASP1; CASP2; CASP3; CASP4; CASP5; CASP6; CASP7; CASP8AP2; CASP8; CASP9; CASQ1; CASR; CASS4; CAST; CASZ1; CAT; CATSPER1; CATSPER2; CAV1; CAV2; CAV3; CBFA2T2; CBFA2T3; CBFB; CBLB; CBL; CBLL1; CBLN1; CBLN2; CBLN4; CBR1; CBR3; CBR4; CBS; CBX1; CBX2; CBX3; CBX4; CBX5; CBX6; CBX7; CBX8; CBY1; CC2D1A; CC2D1B; CC2D2A; CCAR1; CCAR2; CCBE1; CCBL1; CCDC101; CCDC102B; CCDC103; CCDC105; CCDC108; CCDC114; CCDC115; CCDC121; CCDC122; CCDC129; CCDC130; CCDC134; CCDC136; CCDC140; CCDC141; CCDC148; CCDC151; CCDC167; CCDC169-SOHLH2; CCDC170; CCDC171; CCDC175; CCDC176; CCDC178; CCDC180; CCDC181; CCDC185; CCDC22; CCDC28A; CCDC39; CCDC3; CCDC40; CCDC42B; CCDC42; CCDC50; CCDC54; CCDC60; CCDC62; CCDC63; CCDC65; CCDC66; CCDC67; CCDC68; CCDC6; CCDC78; CCDC80; CCDC83; CCDC85A; CCDC86; CCDC88A; CCDC88C; CCDC8; CCDC91; CCDC94; CCDC97; CCHCR1; CCKAR; CCKBR; CCK; CCL11; CCL13; CCL14; CCL15; CCL16; CCL17; CCL18; CCL19; CCL1; CCL20; CCL21; CCL22; CCL23; CCL24; CCL25; CCL26; CCL27; CCL28; CCL2; CCL3; CCL3L3; CCL4; CCL4L1; CCL4L2; CCL5; CCL7; CCL8; CCM2; CCNA1; CCNA2; CCNB1; CCNB2; CCNB3; CCNC; CCND1; CCND2; CCND3; CCNDBP1; CCNE1; CCNE2; CCNF; CCNG1; CCNG2; CCNH; CCNI; CCNJ; CCNJL; CCNK; CCNL1; CCNL2; CCNO; CCNT1; CCNY; CCP110; CCR10; CCR1; CCR2; CCR3; CCR4; CCR5; CCR6; CCR7; CCR8; CCR9; CCRL2; CCRN4L; CCSER1; CCS; CCT2; CCT3; CCT4; CCT5; CCT6A; CCT6B; CCT7; CD109; CD14; CD151; CD163; CD163L1; CD164; CD177; CD180; CD19; CD1A; CD1B; CD1C; CD1D; CD1E; CD200; CD200R1; CD207; CD209; CD226; CD22; CD244; CD247; CD248; CD24; CD274; CD276; CD27; CD28; CD2AP; CD300A; CD300C; CD300LF; CD302; CD320; CD33; CD34; CD36; CD37; CD38; CD30; CD3EAP; CD3E; CD3G; CD40; CD40LG; CD44; CD46; CD47; CD48; CD4; CD55; CD59; CD5; CD5L; CD63; CD68; CD69; CD6; CD72; CD74; CD79A; CD79B; CD7; CD80; CD81; CD82; CD83; CD84; CD86; CD8A; CD8B; CD93; CD96; CD99; CD9; CDADC1; CDAN1; CDC123; CDC14A; CDC14B; CDC16; CDC20B; CDC23; CDC25A; CDC25B; CDC25C; CDC27; CDC34; CDC37; CDC37L1; CDC42BPA; CDC42BPB; CDC42BPG; CDC42EP1; CDC42EP3; CDC42SE2; CDC45; CDC5L; CDC6; CDC73; CDC7; CDCA2; CDCA3; CDCA5; CDCA7; CDCA7L; CDCP1; CDH10; CDH11; CDH13; CDH15; CDH16; CDH17; CDH18; CDH19; CDH1; CDH20; CDH22; CDH23; CDH26; CDH2; CDH3; CDH4; CDH5; CDH6; CDH7; CDH8; CDH9; CDHR1; CDHR2; CDHR3; CDHR5; CDIPT; CDK10; CDK11B; CDK12; CDK13; CDK14; CDK15; CDK16; CDK17; CDK18; CDK19; CDK1; CDK20; CDK2AP1; CDK2AP2; CDK2; CDK3; CDK4; CDK5; CDK5R1; CDK5R2; CDK5RAP1; CDK5RAP2; CDK5RAP3; CDK6; CDK7; CDK8; CDK9; CDKAL1; CDKL1; CDKL2; CDKL3; CDKL4; CDKL5; CDKN1A; CDKN1B; CDKN1C; CDKN2A; CDKN2AIP; CDKN2B; CDKN2C; CDKN20; CDKN3; CDNF; CDO1; CDON; CDR1; CDR2; CDS2; CDT1; CDV3; CDX1; CDX2; CDX4; CDY1B; CDY2B; CDYL2; CDYL; CEACAM16; CEACAM19; CEACAM1; CEACAM21; CEACAM3; CEACAM4; CEACAM5; CEACAM6; CEACAM7; CEACAM8; CEBPA; CEBPB; CEBPD; CEBPE; CEBPG; CEBPZ; CECR1; CECR2; CELA1; CELA3B; CELF1; CELF2; CELF4; CELF5; CELF6; CEL; CELSR1; CELSR3; CEMIP; CEMP1; CEND1; CENPA; CENPB; CENPC; CENPE; CENPF; CENPH; CENPJ; CENPK; CENPN; CENPO; CENPQ; CENPU; CENPV; CENPW; CEP112; CEP120; CEP128; CEP131; CEP135; CEP152; CEP162; CEP164; CEP170; CEP192; CEP19; CEP250; CEP290; CEP41; CEP55; CEP57; CEP63; CEP68; CEP72; CEP76; CEP83; CEP85L; CEP89; CERT; CERK; CERKL; CERS1; CERS2; CERS3; CERS4; CERS6; CES1; CES2; CES3; CETN1; CETN2; CETN3; CETP; CFAP126; CFAP36; CFAP44; CFAP52; CFAP53; CFAP57; CFAP58; CFAP61; CFAP69; CFAP97; CFB; CFC1B; CFC1; CFD; CFDP1; CFH; CFHR1; CFHR2; CFHR3; CFHR4; CFHR5; CFI; CFL1; CFL2; CFLAR; CFP; CFTR; CGA; CGB1; CGB2; CGB5; CGB7; CGN; CGNL1; CGRRF1; CH.sub.25H; CHAC1; CHAD; CHAF1A; CHAF1B; CHAMP1; CHAT; CHCHD10; CHCHD1; CHCHD3; CHCHD5; CHCHD6; CHCHD7; CHD1; CHD2; CHD3; CHD4; CHD6; CHD7; CHD8; CHDH; CHEK1; CHEK2; CHERP; CHFR; CHGA; CHGB; CHI3L1; CHIC1; CHIC2; CHIT1; CHKA; CHKB; CHL1; CHM; CHML; CHMP1A; CHMP1B; CHMP2B; CHMP3; CHMP4A; CHMP4B; CHMP4C; CHMP5; CHN1; CHN2; CHODL; CHORDC1; CHP1; CHP2; CHPF; CHPT1; CHRAC1; CHRD; CHRDL1; CHRFAM7A; CHRM1; CHRM2; CHRM3; CHRM4; CHRM5; CHRNA10; CHRNA1; CHRNA2; CHRNA3; CHRNA4; CHRNA5; CHRNA6; CHRNA7; CHRNB1; CHRNB2; CHRNB3; CHRNB4; CHRND; CHRNE; CHRNG; CHST10; CHST11; CHST12; CHST13; CHST14; CHST15; CHST1; CHST2; CHST3; CHST4; CHST5; CHST6; CHST8; CHST9; CHSY1; CHSY3; CHTF18; CHTOP; CHURC1; CIAO1; CIAPIN1; CIB1; CIB2; CIC; CIDEB; CIDEC; CITTA; CILP2; CILP; CINP; CIPC; CIR1; CIRBP; CIRH1A; CISD1; CISD2; CISH; CITED1; CITED2; CIT; CIZ1; CKAP2; CKAP2L; CKAP4; CKAP5; CKB; CKLF; CKM; CKMT1A; CKMT1B; CKMT2; CKS1B; CKS2; CLASP1; CLASP2; CLCA2; CLCA4; CLCF1; CLC; CLCN1; CLCN2; CLCN3; CLCN4; CLCN5; CLCN6; CLCN7; CLCNKB; CLDN10; CLDN11; CLDN14; CLDN15; CLDN16; CLDN18; CLDN1; CLDN23; CLDN2; CLDN5; CLDN6; CLDN7; CLDN8; CLDN9; CLEC10A; CLEC11A; CLEC12A; CLEC14A; CLEC16A; CLEC1B; CLEC2A; CLEC2B; CLEC20; CLEC2L; CLEC3B; CLEC4A; CLEC4C; CLEC40; CLEC4E; CLEC4G; CLEC4M; CLEC5A; CLEC6A; CLEC7A; CLEC9A; CLECL1; CLGN; CLIC1; CLIC3; CLIC5; CLIC6; CLINT1; CLIP1; CLIP2; CLK1; CLK2; CLLU1; CLMN; CLMP; CLN3; CLN5; CLN6; CLN8; CLNK; CLNS1A; CLOCK; CLP1; CLPP; CLPS; CLPTM1; CLPTM1L; CLRN1; CLSPN; CLSTN1; CLSTN2; CLTA; CLTC; CLTCL1; CLUAP1; CLU; CLUL1; CLVS1; CLYBL; CMA1; CMAS; CMCJ; CMC2; CMC4; CMIP; CMKLR1; CMPK2; CMSS1; CMTM3; CMTM5; CMTM7; CMTM8; CMTR1; CMTR2; CMYA5; CNBD1; CNBP; CNDP1; CNDP2; CNGA1; CNGA3; CNGB1; CNGB3; CNIH3; CNKSR1; CNKSR2; CNKSR3; CNN1; CNN2; CNNM1; CNNM2; CNNM4; CNOT1; CNOT2; CNOT3; CNOT4; CNOT6; CNOT6L; CNOT7; CNOT8; CNP; CNPY2; CNPY3; CNR1; CNR2; CNRIP1; CNST; CNTF; CNTFR; CNTLN; CNTN1; CNTN2; CNTN3; CNTN4; CNTN5; CNTN6; CNTNAP1; CNTNAP2; CNTNAP3; CNTNAP4; CNTNAP5; CNTRL; CNTROB; COAL; COA5; COASY; COBL; COBLL1; COCH; COG1; COG2; COG3; COG4; COG5; COG6; COG7; COG5; COIL; COL10A1; COL11A1; COL11A2; COL12A1; COL13A1; COL14A1; COL15A1; COL16A1; COL17A1; COL18A1; COL1A1; COL1A2; COL20A1; COL21A1; COL22A1; COL23A1; COL24A1; COL25A1; COL26A1; COL27A1; COL28A1; COL2A1; COL3A1; COL4A1; COL4A2; COL4A3BP; COL4A3; COL4A4; COL4A5; COL4A6; COL5A1; COL5A2; COL5A3; COL6A1; COL6A2; COL6A3; COL6A5; COL7A1; COL8A1; COL8A2; COL9A1; COL9A2; COL9A3; COLCA1; COLCA2; COLEC10; COLEC11; COLEC12; COLGALT2; COLQ; COMMD10; COMMD1; COMMD3-BMI1; COMMD5; COMMD7; COMP; COMT; COPA; COPB1; COPB2; COPE; COPG2; COPRS; COPS2; COPS3; COPS4; COPS5; COPSE; COPS7A; COPSE; COPZ2; COQ2; COQ3; COQ4; COQ5; COQ6; COQ7; COQ9; CORIN; CORO1A; CORO1B; CORO1C; CORO2A; CORO2B; CORO6; CORO7; CORO7-PAM16; COTL1; COX10; COX11; COX14; COX15; COX16; COX17; COX18; COX19; COX4I1; COX4I2; COX5A; COX5B; COX6A1; COX6A2; COX6B1; COX6C; COX7A1; COX7A2; COX7A2L; COX7B2; COX7B; COX7C; COX8A; CPA1; CPA2; CPA3; CPA4; CPA6; CPAMD8; CPB1; CPB2; CPD; CPEB1; CPEB3; CPEB4; CPED1; CPE; CP; CPLX1; CPLX2; CPLX3; CPLX4; CPM; CPN1; CPN2; CPNE1; CPNE2; CPNE3; CPNE4; CPNE7; CPNE8; CPO; CPDX; CPPED1; CPQ; CPS1; CPSF1; CPSF2; CPSF3; CPSF3L; CPSF4; CPSF6; CPSF7; CPT1A; CPT1B; CPT1C; CPVL; CPXCR1; CPZ; CR1; CR2; CRABP1; CRABP2; CRACR2A; CRADD; CRAMP1L; CRAT; CRB1; CRB2; CRB3; CRBN; CRCP; CRCT1; CREB1; CREB3; CREB3L1; CREB3L2; CREB3L3; CREB3L4; CREB5; CREBBP; CREBRF; CREBZF; CREG1; CRELD1; CRELD2; CREM; CRHBP; CRH; CRHR1; CRHR2; CRIM1; CRIP2; CRIP3; CRIPAK; CRIPT; CRISP1; CRISP2; CRISP3; CRISPLD1; CRISPLD2; CRK; CRKL; CRLF1; CRLF2; CRLF3; CRLS1; CRMP1; CRNKL1; CRNN; CROCC; CROT; CRP; CRTAC1; CRTAM; CRTAP; CRTC1; CRTC2; CRTC3; CRX; CRY1; CRY2; CRYAB; CRYBA1; CRYBA2; CRYBA4; CRYBB1; CRYBB2; CRYBB3; CRYGB; CRYGC; CRYGD; CRYGS; CRYL1; CRYM; CRYZL1; CSAD; CSAG3; CSE1L; CSF1R; CSF2; CSF2RA; CSF2RB; CSF3; CSGALNACT1; CSGALNACT2; CSH1; CSH2; CSHL1; CS; CSK; CSMD1; CSMD2; CSMD3; CSN1S1; CSN2; CSN3; CSNK1A1; CSNK1D; CSNK1G3; CSNK2A1; CSNK2A2; CSNK2B; CSPG4; CSPG5; CSPP1; CSRNP1; CSRNP3; CSRP1; CSRP2BP; CSRP2; CSRP3; CST1; CST2; CST3; CST4; CST5; CST6; CST7; CST8; CST9; CST9L; CSTA; CSTB; CSTF1; CSTF2; CSTF2T; CSTL1; CT45A1; CT55; CT83; CTAG1A; CTAG2; CTAGE1; CTAGE5; CTBP1; CTBP2; CTC1; CTCF; CTCFL; CTDP1; CTDSP1; CTDSP2; CTDSPL; CTF1; CTGF; CTHRC1; CTIF; CTLA4; CTNNA1; CTNNA2; CTNNA3; CTNNAL1; CTNNB1; CTNNBL1; CTNND1; CTNND2; CTNS; CTPS1; CTR9; CTRB1; CTRB2; CTRC; CTRL; CTSA; CTSB; CTSC; CTSD; CTSE; CTSF; CTSG; CTSH; CTSL; CTSO; CTSV; CTSW; CTSZ; CTTNBP2; CTTN; CTU1; CTXN3; CUBN; CUEDC1; CUEDC2; CUL1; CUL2; CUL3; CUL4A; CUL4B; CUL5; CULT; CUL9; CUTA; CUX1; CUX2; CUZD1; CWC22; CWC27; CWF19L1; CWF19L2; CWH43; CX3CL1; CX3CR1; CXADR; CXCL10; CXCL11; CXCL12; CXCL13; CXCL14; CXCL16; CXCL17; CXCL1; CXCL2; CXCL3; CXCL5; CXCL6; CXCL8; CXCL9; CXCR1; CXCR2; CXCR3; CXCR4; CXCR5; CXCR6; CXorf36; CXorf66; CXorf67; CXXC1; CXXC4; CYB561D2; CYB561; CYB5A; CYB5B; CYB5R3; CYB5R4; CYBA; CYBB; CYBRD1; CYC1; CYCS; CYFIP1; CYFIP2; CYGB; CYLC1; CYLC2; CYLD; CYP11A1; CYP11B1; CYP11B2; CYP17A1; CYP19A1; CYP1A1; CYP1A2; CYP1B1; CYP21A2; CYP24A1; CYP26A1; CYP26B1; CYP26C1; CYP27A1; CYP27B1; CYP27C1; CYP2A13; CYP2A6; CYP2A7; CYP2B6; CYP2C18; CYP2C19; CYP2C8; CYP2C9; CYP2D6; CYP2E1; CYP2F1; CYP2R1; CYP2S1; CYP2U1; CYP2W1; CYP39A1; CYP3A43; CYP3A4; CYP3A5; CYP3A7-CYP3A51P; CYP3A7; CYP46A1; CYP4A11; CYP4B1; CYP4F11; CYP4F12; CYP4F22; CYP4F2; CYP4F3; CYP4F8; CYP4V2; CYP51A1; CYP7A1; CYP7B1; CYP8B1; CYST; CYSLTR1; CYTH1; CYTH3; CYTIP; CYTL1; CYYR1; D2HGDH; DAAM1; DAAM2; DAB2; DAB2IP; DACH1; DACH2; DACT1; DACT2; DACT3; DAD1; DAG1; DAND5; DAOA; DAO; DAP3; DAP; DAPK1; DAPK2; DAPK3; DARS2; DARS; DAW1; DAXX; DAZ1; DAZ2; DAZ3; DAZ4; DAZAP1; DAZAP2; DAZL; DBF4; DBH; DBI; DBN1; DBNL; DBP; DBR1; DBX1; DCAF12; DCAF13; DCAF17; DCAF4; DCAF5; DCAF6; DCAF7; DCAF8; DCANP1; DCBLD1; DCBLD2; DCC; DCDC2C; DCDC2; DCD; DCHS1; DCHS2; DCK; DCLK1; DCLK2; DCLK3; DCLRE1A; DCLRE1C; DCN; DCP1A; DCP1B; DCPS; DCST1; DCST2; DCSTAMP; DCTD; DCT; DCTN1; DCTN2; DCTN3; DCTN4; DCTN5; DCTN6; DCUN101; DCX; DCXR; DDAH1; DDAH2; DDB1; DDB2; DDC; DDHD1; DDHD2; DDI1; DDIAS; DDIT3; DDIT4; DDIT4L; DDN; DDO; DDR1; DDR2; DDRGK1; DDT; DDTL; DDX10; DDX11; DDX17; DDX18; DDX19A; DDX1; DDX21; DDX25; DDX27; DDX28; DDX31; DDX39A; DDX39B; DDX3X; DDX3Y; DDX41; DDX42; DDX43; DDX46; DDX4; DDX50; DDX51; DDX52; DDX53; DDX54; DDX56; DDX58; DDX59; DDX5; DDX60; DDX6; DEAF1; DEC1; DECR1; DEDD; DEF6; DEFA1B; DEFA3; DEFA4; DEFA5; DEFA6; DEFB103B; DEFB104B; DEFB105A; DEFB106A; DEFB108B; DEFB112; DEFB125; DEFB126; DEFB1; DEFB4A; DEGS1; DEGS2; DEK; DENND1A; DENND1B; DENND2A; DENND4A; DENR; DEPDC1B; DEPDC5; DEPTOR; DERA; DERL1; DERL2; DERL3; DES; DESI2; DEXI; DFFB; DFNA5; DFNB31; DFNB59; DGAT1; DGAT2; DGCR2; DGCR6L; DGCR8; DGKA; DGKB; DGKD; DGKE; DGKG; DGKH; DGKI; DGKK; DGKQ; DGKZ; DGUOK; DHCR7; DHDH; DHFR; DHFRL1; DHH; DHODH; DHPS; DHRS11; DHRS2; DHRS4; DHRS7C; DHRS9; DHTKD1; DHX15; DHX16; DHX32; DHX34; DHX36; DHX38; DHX40; DHX58; DHX8; DHX9; DIABLO; DIAPH1; DIAPH2; DIAPH3; DICER1; DIDO1; DIEXF; DIP2A; DIP2B; DIP2C; DIRAS1; DIRAS2; DIRC1; DIRC2; DIS3; DIS3L2; DIS3L; DISCI; DISP1; DIXDC1; DKC1; DKK1; DKK2; DKK3; DKK4; DKKL1; DLAT; DLC1; DLD; DLEC1; DLEU7; DLG1; DLG2; DLG3; DLG4; DLG5; DLGAP1; DLGAP2; DLGAP3; DLGAP5; DLK1; DLL1; DLL3; DLL4; DLST; DLX1; DLX2; DLX3; DLX4; DLX5; DLX6; DMBT1; DMC1; DMD; DMGDH; DMKN; DMP1; DMPK; DMRT1; DMRT2; DMRT3; DMRTA1; DMTF1; DMTN; DMWD; DMXL1; DMXL2; DNA2; DNAAF1; DNAAF2; DNAAF3; DNAAF5; DNAH11; DNAH12; DNAH17; DNAH1; DNAH2; DNAH3; DNAH5; DNAH6; DNAH7; DNAH8; DNAH9; DNAI1; DNAI2; DNAJA1; DNAJA2; DNAJA3; DNAJB11; DNAJB13; DNAJB1; DNAJB2; DNAJB5; DNAJB6; DNAJB7; DNAJB8; DNAJB9; DNAJC10; DNAJC12; DNAJC13; DNAJC14; DNAJC15; DNAJC18; DNAJC19; DNAJC1; DNAJC27; DNAJC28; DNAJC2; DNAJC3; DNAJC5; DNAJC7; DNAL1; DNASE1; DNASE1L2; DNASE1L3; DNASE2B; DNASE2; DND1; DNER; DNHD1; DNLZ; DNM1; DNM1L; DNM2; DNM3; DNMBP; DNMT1; DNMT3A; DNMT3B; DNMT3L; DNPEP; DNTT; DOC2A; DOC2B; DOCK10; DOCK11; DOCK1; DOCK2; DOCK3; DOCK4; DOCK5; DOCK6; DOCK7; DOCK8; DOCK9; DOHH; DOK1; DOK2; DOK3; DOK4; DOK5; DOK6; DOK7; DOLK; DONSON; DOPEY2; DOT1L; DPAGT1; DPCD; DPCR1; DPEP1; DPEP2; DPEP3; DPF1; DPF3; DPH1; DPH3; DPH6; DPH7; DPMI; DPM2; DPM3; DPP10; DPP3; DPP4; DPP6; DPP7; DPP8; DPP9; DPPA2; DPPA3; DPPA4; DPT; DPY19L2; DPY19L3; DPY30; DPYS; DPYSL2; DPYSL3; DPYSL4; DPYSL5; DRAM1; DRAP1; DRC1; DRD1; DRD2; DRD3; DRD4; DRD5; DRG1; DRGX; DROSHA; DRP2; DSC1; DSC2; DSC3; DSCAM; DSCAML1; DSCC1; DSCR4; DSE; DSEL; DSG1; DSG2; DSG3; DSG4; DSP; DSPP; DST; DSTN; DSTYK; DTD1; DTHD1; DTL; DTNA; DTNB; DTNBP1; DTX1; DTX2; DTX3; DTX4; DTYMK; DUOX1; DUOX2; DUOXA1; DUOXA2; DUPD1; DUS2; DUSP10; DUSP11; DUSP12; DUSP13; DUSP14; DUSP15; DUSP16; DUSP18; DUSP1; DUSP21; DUSP22; DUSP23; DUSP27; DUSP28; DUSP2; DUSP3; DUSP4; DUSP5; DUSP6; DUSP7; DUSP8; DUSP9; DUT; DUX4; DVL2; DVL3; DXO; DYM; DYNAP; DYNC1H1; DYNC1I1; DYNC1I2; DYNC1LI1; DYNC2H1; DYNLL1; DYNLL2; DYNLRB1; DYNLT1; DYNLT3; DYRK1A; DYRK1B; DYRK2; DYRK3; DYSF; DYX1C1; DZIP1; E2F1; E2F3; E2F4; E2F5; E2F6; E2F7; E2F8; E4F1; EAF1; EAF2; EARS2; EBAG9; EBF1; EBF2; EBF3; EBF4; EBI3; EBP; EBPL; ECD; ECE2; ECEL1; ECHDC1; ECHDC3; ECHS1; ECI1; ECI2; ECSCR; ECSIT; ECT2; EDA2R; EDA; EDARADD; EDAR; EDC4; EDEM1; EDIL3; EDN1; EDN3; EDNRA; EDNRB; EEA1; EED; EEF1A1; EEF1A2; EEF1B2; EEF1D; EEF1E1; EEF1G; EEF2; EEFSEC; EFCAB11; EFCAB1; EFEMP1; EFEMP2; EFHB; EFHC1; EFHC2; EFHD1; EFNA1; EFNA2; EFNA3; EFNA4; EFNA5; EFNB1; EFNB2; EFNB3; EFR3A; EFR3B; EFS; EFTUD1; EFTUD2; EGF; EGFL6; EGFL7; EGFL8; EGFLAM; EGFR; EGLN1; EGLN2; EGLN3; EGR1; EGR2; EGR3; EGR4; EHBP1; EHD1; EHD2; EHD3; EHD4; EHF; EHHADH; EHMT1; EHMT2; EI24; EID1; EIF1AD; EIF1AX; EIF1AY; EIF1; EIF2A; EIF2AK1; EIF2AK2; EIF2AK3; EIF2AK4; EIF2B1; EIF2B2; EIF2B3; EIF2B4; EIF2B5; EIF2S1; EIF2S2; EIF2S3; EIF3A; EIF3B; EIF3C; EIF3E; EIF3F; EIF3H; EIF3J; EIF3K; EIF3M; EIF4A1; EIF4A2; EIF4A3; EIF4B; EIF4E2; EIF4E3; EIF4EBP1; EIF4EBP2; EIF4EBP3; EIF4E; EIF4EIF1; EIF4G1; EIF4G2; EIF4H; EIF5A2; EIF5A; EIF5; EIF6; ELAC1; ELAC2; ELANE; ELAVL1; ELAVL2; ELAVL3; ELAVL4; ELF1; ELF2; ELF3; ELF4; ELF5; ELK1; ELK3; ELK4; ELL2; ELL; ELMO1; ELMO2; ELMOD1; ELMOD2; ELMOD3; ELMSAN1; ELN; ELOF1; ELOVL2; ELOVL4; ELOVL5; ELOVL6; ELOVL7; ELP2; ELP3; ELP4; ELP6; ELSPBP1; EMB; EMC10; EMC2; EMC3; EMC7; EMC8; EMCN; EMD; EME1; EMG1; EMILIN1; EMILIN2; EMILIN3; EML1; EML2; EML4; EML5; EML6; EMP1; EMP2; EMX1; EMX2; EN1; EN2; ENAH; ENAM; ENC1; ENDOG; ENDOU; ENDOV; ENGASE; ENG; ENHO; ENO2; ENO3; ENO4; ENOPH1; ENOSF1; ENOX1; ENOX2; ENPEP; ENPP1; ENPP2; ENPP3; ENPP5; ENPP7; ENTHD2; ENTPD1; ENTPD2; ENTPD4; ENTPD5; ENTPD6; ENTPD7; EOGT; EOMES; EP300; EP400; EPAS1; EPB41L1; EPB41L2; EPB41L3; EPB41L4A; EPB41L4B; EPB42; EPC1; EPC2; EPCAM; EPDR1; EPG5; EPGN; EPHA1; EPHA3; EPHA4; EPHA5; EPHA6; EPHA7; EPHB1; EPHB3; EPHB4; EPHB6; EPHX1; EPHX2; EPHX3; EPM2A; EPM2AIP1; EPN1; EPO; EPOR; EPPIN; EPPIN-WFDC6; EPPK1; EPRS; EPS15L1; EPS8; EPS8L2; EPSTI1; EPX; EPYC; ERAL1; ERAP1; ERAP2; ERAS; ERBB2; ERBB2IP; ERBB3; ERBB4; ERCT; ERC2; ERCC1; ERCC2; ERCC3; ERCC4; ERCC5; ERCC6; ERCC6L2; ERCC6-PGBD3; ERCC8; EREG; ERF; ERG; ERGIC1; ERGIC2; ERGIC3; ERI3; ERICH5; ERICH6B; ERLEC1; ERLIN1; ERLIN2; ERMAP; ERMARD; ERMP1; ERN1; ERN2; ERO1LB; ERO1L; ERP29; ERP44; ERV3-1; ERVW-1; ESAM; ESCO1; ESCO2; ESD; ESF1; ESM1; ESPL1; ESPN; ESR1; ESR2; ESRP1; ESRP2; ESRRA; ESRRB; ESRRG; ESX1; ESYT1; ESYT2; ESYT3; ETAA1; ETF1; ETFA; ETFB; ETFDH; ETHE1; ETNK1; ETNK2; ETNPPL; ETS1; ETS2; ETV1; ETV3; ETV4; ETV5; ETV6; ETV7; EVA1A; EVA1C; EVC2; EVC; EVI2A; EVI2B; EVI5; EVL; EVPL; EVX1; EVX2; EWSR1; EXD2; EXD3; EXO1; EXOC1; EXOC2; EXOC3L1; EXOC3L2; EXOC4; EX005; EXOC7; EXOG; EXOSC1; EXOSC2; EXOSC3; EXOSC4; EXOSC5; EXOSC6; EXOSC7; EXOSC8; EXPH5; EXT1; EXT2; EXTL2; EXTL3; EYA1; EYA2; EYA4; EYS; EZH1; EZH2; EZR; F10; F11; F11R; F12; F13A1; Fl3B; F2; F2R; F2RL1; F2RL2; F2RL3; F5; F7; F8; F9; FA2H; FAAH2; FABP1; FABP2; FABP4; FABP5; FABP6; FABP7; FABP9; FADD; FADS1; FADS2; FADS3; FAF2; FAHD2A; FAH; FAIM2; FAIM3; FAM102A; FAM103A1; FAM105A; FAM107A; FAM107B; FAM109A; FAM110A; FAM110B; FAM110C; FAM111B; FAM114A1; FAM120B; FAM120C; FAM124B; FAM126A; FAM129A; FAM129B; FAM134B; FAM135A; FAM135B; FAM136A; FAM13A; FAM13C; FAM149A; FAM150B; FAM155A; FAM160B1; FAM161A; FAM163A; FAM163B; FAM167A; FAM168A; FAM168B; FAM169B; FAM170A; FAM172A; FAM173B; FAM174A; FAM175A; FAM175B; FAM177A1; FAM177B; FAM178A; FAM178B; FAM179B; FAM180A; FAM184A; FAM184B; FAM187B; FAM188A; FAM188B; FAM189B; FAM193B; FAM196A; FAM196B; FAM198B; FAM19A1; FAM19A2; FAM19A4; FAM19A5; FAM204A; FAM205A; FAM20A; FAM20B; FAM20C; FAM210B; FAM213A; FAM214A; FAM216A; FAM220A; FAM227B; FAM32A; FAM3A; FAM3B; FAM3C; FAM3D; FAM46A; FAM46D; FAM49A; FAM49B; FAM50B; FAM53B; FAM57A; FAM58A; FAM60A; FAM63B; FAM64A; FAM65B; FAM69C; FAM71F1; FAM71F2; FAM72B; FAM78B; FAM81B; FAM83A; FAM83B; FAM83D; FAM83H; FAM84A; FAM84B; FAM89A; FAM92A1; FAM92B; FAM9B; FAN1; FANCA; FANCB; FANCC; FANCD2; FANCE; FANCF; FANCG; FANCI; FANCL; FANCM; FANK1; FAP; FAR1; FARP1; FARP2; FARS2; FARSA; FAS; FASLG; FASN; FASTKD2; FASTK; FAT1; FAT2; FAT3; FAT4; FATE1; FAU; FBF1; FBL; FBLIM1; FBLN1; FBLN2; FBLN5; FBN1; FBN2; FBN3; FBP1; FBP2; FBRS; FBXL15; FBXL17; FBXL19; FBXL20; FBXL2; FBXL3; FBXL4; FBXL5; FBXL7; FBXO10; FBXO11; FBXO15; FBXO17; FBXO18; FBXO28; FBXO30; FBXO31; FBXO32; FBXO33; FBXO38; FBXO3; FBXO40; FBXO47; FBXO4; FBXO5; FBXO7; FBXO8; FBXO9; FBXW11; FBXW4; FBXW7; FBXW8; FCAR; FCER1A; FCER1G; FCER2; FCGBP; FCGR2A; FCGR2B; FCGR3A; FCGR3B; FCGRT; FCHO1; FCHO2; FCHSD2; FCN1; FCN2; FCRL1; FCRL2; FCRL3; FCRL4; FCRL5; FCRL6; FCRLA; FCRLB; FDFT1; FDPS; FDX1; FDX1L; FDXR; FECH; FEM1A; FEM1B; FEM1C; FEN1; FERD3L; FER; FERMT1; FERMT2; FERMT3; FES; FEV; FEZ1; FEZ2; FEZF1; FEZF2; FFAR1; FFAR2; FFAR3; FFAR4; FGA; FGB; FGD1; FGD2; FGD3; FGD4; FGD5; FGD6; FGF10; FGF11; FGF12; FGF13; FGF14; FGF16; FGF17; FGF18; FGF19; FGF1; FGF20; FGF21; FGF23; FGF2; FGF3; FGF4; FGF5; FGF6; FGF7; FGF8; FGF9; FGFBP1; FGFBP2; FGFBP3; FGFR1; FGFR10P2; FGFR10P; FGFR2; FGFR3; FGFR4; FGFRL1; FGG; FGGY; FGL1; FGL2; FGR; FHDC1; FH; FHIT; FHL1; FHL2; FHL5; FHOD1; FHOD3; FIBP; FIG4; FIGF; FIGLA; FIGN; FILIP1; FILIP1L; FIP1L1; FIST; FITM1; FITM2; FJX1; FKBP10; FKBP11; FKBP14; FKBP15; FKBP1A; FKBP1B; FKBP2; FKBP3; FKBP4; FKBP5; FKBP6; FKBP7; FKBP8; FKBP9; FKBPL; FKRP; FKTN; FLAD1; FLCN; FLG2; FLG; FLIT; FLII; FLNA; FLNB; FLNC; FLOT1; FLOT2; FLRT2; FLRT3; FLT1; FLT3; FLT3LG; FLT4; FLVCR1; FLVCR2; FMN1; FMN2; FMNL1; FMNL2; FMNL3; FM01; FMO2; FMO3; FMO4; FMOD; FMR1; FN1; FN3K; FN3KRP; FNBP1; FNDC1; FNDC3A; FNDC3B; FNDC4; FNTA; FNTB; FOCAD; FOLH1; FOLR1; FOLR2; FOLR3; FOPNL; FOSB; FOS; FOSL1; FOSL2; FOXA1; FOXA2; FOXA3; FOXB1; FOXC1; FOXC2; FOXD1; FOXE1; FOXF1; FOXF2; FOXG1; FOXH1; FOXI1; FOXJ1; FOXJ2; FOXK1; FOXK2; FOXL1; FOXL2; FOXM1; FOXN1; FOXN2; FOXN3; FOXO1; FOXO3; FOXO4; FOXO6; FOXP1; FOXP2; FOXP3; FOXP4; FOXQ1; FOXR1; FOXR2; FOXRED1; FPGS; FPR1; FPR2; FPR3; FRAS1; FRAT1; FRAT2; FREM1; FREM2; FREM3; FRG1; FRG2; FRK; FRMD3; FRMD4A; FRMD4B; FRMD5; FRMD6; FRMD7; FRMPD1; FRMPD2; FRMPD4; FRRS1L; FRS2; FRS3; FRY; FRYL; FRZB; FSBP; FSCB; FSCN1; FSCN2; FSD1; FSD1L; FSD2; FSHB; FSHR; FSIP1; FST; FSTL1; FSTL3; FSTL4; FSTL5; FTCD; FTH1; FTL; FTMT; FTO; FTSJ1; FTSJ2; FTSJ3; FUBP1; FUBP3; FUCA1; FUCA2; FUNDC1; FUNDC2; FURIN; FUS; FUT10; FUT11; FUT1; FUT2; FUT3; FUT4; FUT5; FUT6; FUT7; FUT8; FUT9; FUZ; FXN; FXR1; FXR2; FXYD1; FXYD2; FXYD3; FXYD5; FXYD6; FYB; FYCO1; FYN; FZD10; FZD1; FZD2; FZD3; FZD4; FZD5; FZD6; FZD7; FZD8; FZD9; FZR1; GOS2; G2E3; G3BP1; G6PC2; G6PC3; G6PC; G6PD; GAA; GAB1; GAB2; GAB3; GABARAP; GABARAPL1; GABARAPL2; GABBR1; GABBR2; GABPA; GABPB1; GABRA1; GABRA2; GABRA3; GABRA4; GABRA5; GABRA6; GABRB1; GABRB2; GABRB3; GABRE; GABRG1; GABRG2; GABRG3; GABRP; GABRQ; GABRR1; GABRR2; GAD1; GAD2; GADD45B; GADD45G; GADD45GIP1; GADL1; GAGE10; GAGE1; GAK; GALC; GAL; GALK1; GALK2; GALM; GALNS; GALNT12; GALNT13; GALNT14; GALNT15; GALNT18; GALNT1; GALNT2; GALNT3; GALNT4; GALNT5; GALNT6; GALNT7; GALNT8; GALNT9; GALNTL6; GALP; GALR1; GALR2; GALR3; GALT; GAMT; GANAB; GANC; GAN; GAP43; GAPDH; GAPDHS; GAR1; GAREM; GARNL3; GARS; GART; GAS1; GAS2; GAS2L1; GAS6; GAS7; GASB; GAST; GATA1; GATA2; GATA3; GATA4; GATA5; GATA6; GATAD1; GATAD2A; GATAD2B; GATB; GATM; GBA2; GBA; GBAS; GBE1; GBF1; GBGT1; GBX1; GBX2; GCA; GCC1; GCDH; GCFC2; GCG; GCGR; GCH1; GCHFR; GC; GCK; GCKR; GCLC; GCM1; GCM2; GCN1L1; GCNT1; GCNT2; GCNT3; GCNT7; GCOM1; GCSAM; GCSAML; GCSH; GDA; GDAP1; GDE1; GDF10; GDF11; GDF15; GDF1; GDF2; GDF3; GDF5; GDF6; GDF7; GDF9; GDI1; GDI2; GDNF; GDPD3; GDPD5; GEM; GEMIN2; GEMIN4; GEMIN6; GEN1; GET4; GFAP; GFER; GFI1B; GFI1; GFM1; GFPT1; GFPT2; GFRA1; GFRA2; GFRA3; GFRA4; GGA1; GGA3; GGACT; GGCT; GGCX; GGH; GGNBP2; GGN; GGPS1; GGT1; GGT2; GGT5; GGTLC1; GH1; GH2; GHITM; GHRH; GHR; GHRHR; GHRL; GHSR; GIDS; GIF; GIGYF1; GIGYF2; GIMAP5; GIMAP7; GIMAP8; GIN1; GINS1; GINS2; GIPC1; GIPC3; GIP; GIPR; GIT1; GIT2; GJA1; GJA3; GJA8; GJB1; GJB2; GJB6; GJC1; GJC2; GJC3; GJD2; GJD3; GK; GKN1; GKN2; GLA; GLB1; GLCCI1; GLCE; GLDC; GLDN; GLE1; GLG1; GLI1; GLI2; GLI3; GLIPR1; GLIPR2; GLIS2; GLIS3; GLMN; GL01; GLOD4; GLP1R; GLP2R; GLRA1; GLRA2; GLRA3; GLRB; GLRX2; GLRX3; GLRX5; GLRX; GLS2; GLS; GLT1D1; GLT6D1; GLT8D1; GLTSCR1; GLTSCR2; GLUD1; GLUD2; GLUL; GLYAT; GLYATL3; GLYCTK; GM2A; GMCL1; GMDS; GMFB; GMFG; GMIP; GML; GMNN; GMPPA; GMPPB; GMPR2; GMPR; GMPS; GNA11; GNA12; GNA13; GNA14; GNA15; GNAI1; GNAI2; GNAL; GNAO1; GNAQ; GNAS; GNAT1; GNAT3; GNAZ; GNB1L; GNB2L1; GNB3; GNB4; GNB5; GNE; GNG10; GNG11; GNG2; GNG4; GNG7; GNG8; GNGT1; GNGT2; GNL1; GNL3; GNL3L; GNLY; GNMT; GNPAT; GNPDA1; GNPDA2; GNPTAB; GNPTG; GNRH1; GNRH2; GNRHR; GNS; GOLGA1; GOLGA2; GOLGA3; GOLGA4; GOLGA5; GOLGA8B; GOLGB1; GOLM1; GOLPH3; GOLT1A; GOLT1B; GON4L; GOPC; GORAB; GORASP1; GORASP2; GOSR1; GOSR2; GOT1; GOT2; GP1BA; GP1BB; GP2; GPS; GP6; GP9; GPA33; GPAA1; GPALPP1; GPAM; GPANK1; GPAT2; GPATCH1; GPATCH2; GPATCH2L; GPATCH8; GPBAR1; GPC1; GPC2; GPC3; GPC4; GPC5; GPC6; PCPD1; GPD1; GPD1L; GPD2; GPER1; GPHA2; GPHN; GPIHBP1; GPI; GPKOW; GPLD1; GPM6A; GPM6B; GPN1; GPNMB; GPR101; GPR119; GPR12; GPR132; GPR135; GPR137C; GPR139; GPR143; GPR148; GPR149; GPR150; GPR151; GPR152; GPR153; GPR155; GPR156; GPR158; GPR15; GPR160; GPR161; GPR162; GPR171; GPR174; GPR176; GPR179; GPR17; GPR180; GPR182; GPR183; GPR19; GPR1; GPR20; GPR22; GPR26; GPR34; GPR35; GPR37; GPR37L1; GPR39; GPR4; GPR50; GPR52; GPR55; GPR65; GPR68; GPR6; GPR75; GPR78; GPR83; GPR87; GPRASP1; GPRC5A; GPRC5B; GPRC5C; GPRC5D; GPRC6A; GPRIN1; GPRIN2; GPRIN3; GPS2; GPSM1; GPSM2; GPSM3; GPT2; GPT; GPX5; GRAMD1B; GRAMD3; GRAMD4; GRAP2; GRAP; GRASP; GRB10; GRB14; GRB2; GRB7; GREB1; GREM1; GREM2; GRHL1; GRHL2; GRHPR; GRIA1; GRIA2; GRIA3; GRIA4; GRID1; GRID2; GRIK1; GRIK2; GRIK4; GRIK5; GRIN1; GRIN2A; GRIN2B; GRIN2C; GRIN2D; GRIN3A; GRIN3B; GRINA; GRIP1; GRK1; GRK4; GRK5; GRK6; GRK7; GRM1; GRM2; GRM3; GRM4; GRM5; GRM6; GRM7; GRM8; GRN; GRPEL1; GRP; GRPR; GRSF1; GRXCR1; GRXCR2; GSC2; GSC; GSDMA; GSDMB; GSDMC; GSDMD; GSE1; GSG1L; GSK3A; GSK3B; GSN; GSPT1; GSPT2; GSR; GSS; GSTA1; GSTA2; GSTA4; GSTA5; GSTCD; GSTK1; GSTM3; GSTO1; GSTO2; GSTP1; GSTT1; GSTT2B; GSTT2; GSTZ1; GSX1; GSX2; GTDC1; GTF2A1; GTF2A1L; GTF2A2; GTF2E1; GTF2E2; GTF2F1; GTF2F2; GTF2H1; GTF2H2C_2; GTF2H2C; GTF2H2; GTF2H3; GTF2H4; GTF2H5; GTF2I; GTF2IRD1; GTF2IRD2; GTF3A; GTF3C1; GTPBP1; GTPBP3; GTPBP4; GTSF1; GUCA1A; GUCA1B; GUCD1; GUCY1A2; GUCY1A3; GUCY1B3; GUCY2C; GUCY2D; GUCY2F; GUK1; GULP1; GUSB; GYG1; GYG2; GYLTL1B; GYPA; GYPB; GYPC; GYPE; GYS1; GYS2; GZF1; GZMA; GZMB; GZMH; GZMK; GZMM; H1F0; H1FX; H2AFJ; H2AFX; H2AFY2; H2AFY; H2AFZ; H2BFWT; H3F3B; HAAO; HABP2; HABP4; HACD1; HACD2; HACD4; HACE1; HACL1; HADHA; HADHB; HADH; HAGH; HAL; HAMP; HAND1; HAND2; HAO1; HAO2; HAP1; HAPLN1; HAPLN4; HARS2; HARS; HAST; HAS2; HAS3; HAT1; HAVCR1; HAVCR2; HAX1; HBA2; HBB; HBD; HBE1; HBEGF; HBG1; HBG2; HBM; HBP1; HBQ1; HBS1L; HBZ; HCAR1; HCAR2; HCAR3; HCCS; HCFC1; HCFC2; HCK; HCLS1; HCN1; HCN2; HCN3; HCN4; HCRT; HCRTR2; HCST; HDAC10; HDAC11; HDAC1; HDAC2; HDAC3; HDAC4; HDAC5; HDAC6; HDAC7; HDAC8; HDAC9; HDC; HDDC2; HDGF; HDGFL1; HDGFRP3; HDHD1; HDLBP; HEATR1; HEATR3; HEATR5B; HEATR6; HEBP1; HEBP2; HECA; HECTD2; HECTD4; HECW1; HECW2; HEG1; HELLS; HELQ; HELT; HELZ2; HELZ; HEMGN; HEPACAM; HEPH; HEPN1; HERC1; HERC2; HERC3; HERC5; HERC6; HERPUD1; HES1; HES2; HES6; HEST; HESX1; HEXA; HEXB; HEXDC; HEXIM1; HEY1; HEY2; HFE; HFM1; HGD; HGFAC; HGF; HGH1; HGS; HGSNAT; HHAT; HHATL; HHEX; HHIP; HHIPL1; HHLA1; HHLA2; HIAT1; HIBADH; HIBCH; HIC1; HIC2; HID1; HIF1A; HIF1AN; HIF3A; HIGD1A; HIGD1C; HIGD2A; HILPDA; HINFP; HINT1; HINT2; HIP1; HIP1R; HIPK1; HIPK2; HIPK3; HIRA; HIRIP3; HIST1H1A; HIST1H1B; HIST1H1C; HIST1H1D; HIST1H1E; HIST1H1T; HIST1H2AE; HIST1H2AH; HIST1H2BG; HIST1H2BH; HIST1H2BM; HIST1H3G; HIST1H4D; HIST3H3; HIVEP1; HIVEP2; HJURP; HK1; HK2; HK3; HKDC1; HLA-A; HLA-B; HLA-C; HLA-DMA; HLA-DMB; HLA-DOA; HLA-DOB; HLA-DPA1; HLA-DPB1; HLA-DQA1; HLA-DQA2; HLA-DQB1; HLA-DQB2; HLA-DRA; HLA-DRB1; HLA-DRB3; HLA-DRB4; HLA-DRB5; HLA-E; HLA-F; HLA-G; HLCS; HLF; HLTF; HLX; HM13; HMBOX1; HMBS; HMCN1; HMG20A; HMG20B; HMGA1; HMGA2; HMGB1; HMGB2; HMGB3; HMGCR; HMGCS1; HMGN1; HMGN4; HMGN5; HMGXB3; HMGXB4; HMHA1; HMMR; HMOX1; HMOX2; HMP19; HMSD; HMX1; HN1; HN1L; HNF1A; HNF1B; HNF4A; HNF4G; HNMT; HNRNPAO; HNRNPA1; HNRNPA2B1; HNRNPA3; HNRNPAB; HNRNPC; HNRNPD; HNRNPDL; HNRNPF; HNRNPH1; HNRNPH2; HNRNPK; HNRNPL; HNRNPM; HNRNPR; HNRNPU; HNRNPUL1; HOGA1; HOMER1; HOMER2; HOMER3; HOMEZ; HOOK2; HOOK3; HOPX; HORMAD2; HOXA10; HOXA11; HOXA13; HOXA1; HOXA2; HOXA3; HOXA4; HOXA5; HOXA6; HOXA7; HOXA9; HOXB13; HOXB1; HOXB2; HOXB3; HOXB4; HOXB5; HOXB6; HOXB7; HOXB8; HOXB9; HOXC10; HOXC11; HOXC12; HOXC13; HOXC4; HOXC5; HOXC6; HOXC8; HOXC9; HOXD10; HOXD11; HOXD12; HOXD13; HOXD1; HOXD3; HOXD4; HOXD8; HOXD9; HP1BP3; HPCAL1; HPD; HPGD; HPGDS; HP; HPN; HPR; HPRT1; HPS1; HPS3; HPS4; HPS5; HPS6; HPSE2; HPSE; HPX; HRAS; HRASLS; HRC; HRG; HRH1; HRH2; HRH3; HRH4; HR; HRK; HRNR; HRSP12; HS1BP3; HS3ST1; HS3ST2; HS3ST3A1; HS3ST3B1; HS3ST4; HS3ST5; HS3ST6; HS6ST1; HS6ST2; HS6ST3; HSBP1; HSD11B1; HSD11B1L; HSD11B2; HSD17B10; HSD17B11; HSD17B12; HSD17B13; HSD17B14; HSD17B1; HSD17B2; HSD17B3; HSD17B4; HSD17B6; HSD17B7; HSD17B8; HSD3B1; HSD3B7; HSDL1; HSDL2; HSF1; HSF2; HSF4; HSF5; HSFY2; HSH2D; HSP90AA1; HSP90AB1; HSP90B1; HSPA12A; HSPA12B; HSPA13; HSPA14; HSPA1A; HSPA1L; HSPA2; HSPA4; HSPA4L; HSPA5; HSPA6; HSPA8; HSPA9; HSPB1; HSPB2; HSPB3; HSPB6; HSPB7; HSPB8; HSPB9; HSPBAP1; HSPBP1; HSPD1; HSPE1; HSPG2; HSPH1; HTATIP2; HTN1; HTN3; HTR1A; HTR1B; HTR1E; HTR1F; HTR2A; HTR2B; HTR2C; HTR3A; HTR3B; HTR3C; HTR3D; HTR3E; HTR4; HTR5A; HTR7; HTRA1; HTRA2; HTRA3; HTRA4; HTT; HUNK; HUS1B; HUS1; HUWE1; HVCN1; HYAL1; HYAL2; HYAL3; HYAL4; HYDIN; HYKK; HYLS1; HYOU1; HYPM; IAPP; IARS2; IARS; IBA57; TBSP; IBTK; ICA1; ICAM1; ICAM2; ICAM3; ICAM4; ICAM5; ICE1; ICE2; ICK; ICMT; ICOS; ICOSLG; ID1; ID2; ID4; IDE; IDH1; IDH2; IDH3B; IDNK; IDOL; ID02; IDS; IDUA; IER2; IER3; IER3IP1; IER5; IFF01; IFF02; IFI16; IFI27; IFI30; IFI35; IFI44; IFIH1; IFIT1; IFIT2; IFIT3; IFIT5; IFITM1; IFITM2; IFITM3; IFITM5; IFNA10; IFNA13; IFNA14; IFNA16; IFNA17; IFNA1; IFNA21; IFNA2; IFNA4; IFNA5; IFNA6; IFNA7; IFNA8; IFNAR1; IFNAR2; IFNB1; IFNE; IFNG; IFNGR1; IFNGR2; IFNK; IFNL1; IFNL2; IFNL3; IFNW1; IFRD1; IFT122; IFT140; IFT172; IFT27; IFT43; IFT57; IFT74; IFT80; IFT88; IGBP1; IGDCC3; IGF1; IGF1R; IGF2BP1; IGF2BP2; IGF2BP3; IGF2; IGF2R; IGFALS; IGFBP1; IGFBP2; IGFBP3; IGFBP4; IGFBP5; IGFBP6; IGFBP7; IGFBPL1; IGFL1; IGFLR1; IGHMBP2; IGJ; IGLL1; IGSF10; IGSF11; IGSF1; IGSF5; IGSF6; IGSF8; IHH; IKBKAP; IKBKB; IKBKE; IKBKG; IK; IKZF1; IKZF2; IKZF3; IKZF4; IL10; IL10RA; IL10RB; IL11; IL11RA; IL12A; IL12B; IL12RB1; IL13; IL13RA1; IL13RA2; IL15; IL15RA; IL16; IL17A; IL17B; IL17C; IL170; IL17F; IL17RA; IL17RB; IL17RC; IL17RD; IL17RE; IL17REL; IL18BP; IL18; IL18R1; IL18RAP; IL19; IL1A; IL1B; IL1F10; IL1R1; IL1R2; IL1RAP; IL1RAPL1; IL1RAPL2; IL1RL1; IL1RL2; IL1RN; IL20; IL20RA; IL20RB; IL21; IL21R; IL22; IL22RA2; IL23A; IL23R; IL24; IL25; IL26; IL27; IL27RA; IL2; IL2RA; IL2RB; IL2RG; IL31; IL31RA; IL32; IL33; IL34; IL36A; IL36B; IL36G; IL36RN; IL37; IL3; IL3RA; IL4; IL4I1; IL4R; ILS; IL5RA; IL6; IL6R; IL6ST; IL7; IL7R; IL9; IL9R; ILDR1; ILDR2; ILF2; ILF3; ILK; IMMP1L; IMMP2L; IMMT; IMP3; IMPA1; IMPA2; IMPACT; IMPAD1; IMPDH1; IMPDH2; IMPG1; IMPG2; INADL; INA; INCENP; INF2; ING1; ING2; ING3; ING4; ING5; INHA; INHBA; INHBB; INHBC; INHBE; INO80B; INO80D; INO80; INPP1; INPP4A; INPP4B; INPP5A; INPP5B; INPP5D; INPP5E; INPP5F; INPP5K; INPPL1; INSC; INS; INSIG1; INSIG2; INS-IGF2; INSL3; INSL4; INSL6; INSM1; INSM2; INSR; INSRR; INTS12; INTS1; INTS2; INTS3; INTS4; INTS5; INTS6; INTS7; INTS8; INTU; INVS; IP6K1; IP6K2; IP6K3; IPCEF1; IPMK; IP011; IP013; IP07; IP08; IP09; IQCB1; IQCG; IQCH; IQCJ; IQCJ-SCHIP1; IQCK; IQGAP1; IQGAP2; IQGAP3; IQSEC1; IQSEC2; IQSEC3; IRAK1BP1; IRAK1; IRAK2; IRAK3; IRAK4; IREB2; IRF1; IRF2BP2; IRF2BPL; IRF2; IRF3; IRF4; IRF5; IRF6; IRF7; IRF8; IRF9; IRG1; IRGM; IRS1; IRS2; IRS4; IRX1; IRX2; IRX3; IRX4; IRX5; ISCU; ISG20; ISL1; ISM1; ISM2; ISPD; IST1; ISX; ISYNA1; ITCH; ITFG1; ITFG3; ITGA10; ITGA11; ITGA1; ITGA2B; ITGA2; ITGA3; ITGA4; ITGA5; ITGA6; ITGA7; ITGA8; ITGA9; ITGAD; ITGAE; ITGAL; ITGAM; ITGAV; ITGAX; ITGB1BP1; ITGB1; ITGB2; ITGB3; ITGB4; ITGB5; ITGB6; ITGB7; ITGB8; ITGBL1; ITIH1; ITIH2; ITIH3; ITIH4; ITIH5; ITK; ITLN1; ITLN2; ITM2A; ITM2B; ITM2C; ITPA; ITPK1; ITPKA; ITPKC; ITPR1; ITPR2; ITPR3; ITSN1; ITSN2; IVD; IVL; IVNS1ABP; IYD; IZUMO1; JADE1; JADE2; JAG1; JAG2; JAGN1; JAK2; JAK3; JAKMIP1; JAKMIP2; JAKMIP3; JAM2; JAM3; JARID2; JAZF1; JDP2; JMJD1C; JMJD6; JMY; JPH2; JPH3; JPH4; JRK; JSRP1; JTB; JUNB; JUND; JUP; KAL1; KALRN; KANK1; KANK2; KANK4; KANSL1; KANSL3; KARS; KAT2A; KAT2B; KAT5; KAT6A; KAT6B; KAT7; KATE; KATNA1; KATNAL2; KATNB1; KAZALD1; KAZN; KBTBD11; KBTBD13; KBTBD8; KCMF1; KCNA1; KCNA4; KCNA5; KCNA6; KCNA7; KCNAB1; KCNAB2; KCNAB3; KCNB1; KCNB2; KCNC1; KCNC2; KCNC3; KCND2; KCNE1; KCNE2; KCNE3; KCNE4; KCNE5; KCNG3; KCNG4; KCNH1; KCNH2; KCNH3; KCNH4; KCNH5; KCNH6; KCNH7; KCNH8; KCNIP1; KCNIP2; KCNIP3; KCNIP4; KCNJ10; KCNJ11; KCNJ12; KCNJ13; KCNJ14; KCNJ15; KCNJ16; KCNJ18; KCNJ1; KCND2; KCNJ3; KCNJ4; KCNJ5; KCNJ6; KCNJ8; KCNJ9; KCNK10; KCNK12; KCNK16; KCNK17; KCNK18; KCNK1; KCNK2; KCNK3; KCNK5; KCNK6; KCNK9; KCNMA1; KCNMB1; KCNMB2; KCNMB3; KCNMB4; KCNN1; KCNN2; KCNN3; KCNN4; KCNQ1; KCNQ2; KCNQ3; KCNQ5; KCNRG; KCNS1; KCNS3; KCNT1; KCNT2; KCNU1; KCNV1; KCNV2; KCTD10; KCTD11; KCTD12; KCTD13; KCTD15; KCTD16; KCTD1; KCTD21; KCTD2; KCTD3; KCTD6; KCTD7; KCTD9; KDELC1; KDELR1; KDM2A; KDM2B; KDM3A; KDM3B; KDM4B; KDM4C; KDM5A; KDM5B; KDM5C; KDM50; KDM6A; KDM6B; KDR; KDSR; KEAP1; KEL; KERA; KHDC3L; KHDRBS2; KHDRBS3; KHK; KHSRP; KIAA0020; KIAA0040; KIAA0100; KIAA0101; KIAA0196; KIAA0226; KIAA0226L; KIAA0232; KIAA0319; KIAA0319L; KIAA0355; KIAA0391; KIAA0586; KIAA0825; KIAA0907; KIAA0922; KIAA1024; KIAA1033; KIAA1109; KIAA1147; KIAA1161; KIAA1211; KIAA1211L; KIAA1217; KIAA1279; KIAA1324; KIAA1324L; KIAA1377; KIAA1456; KIAA1462; KIAA1468; KIAA1524; KIAA1549; KIAA1551; KIAA1598; KIAA1715; KIAA1804; KIAA1841; KIAA1919; KIAA2022; KIDINS220; KIF11; KIF13A; KIF14; KIF15; KIF16B; KIF17; KIF18A; KIF1A; KIF1C; KIF20A; KIF20B; KIF21A; KIF21B; KIF22; KIF23; KIF24; KIF25; KIF26A; KIF26B; KIF2A; KIF2B; KIF2C; KIF3A; KIF3B; KIF3C; KIF4A; KIF4B; KIF5A; KIF5B; KIF5C; KIF6; KIF7; KIF9; KIFAP3; KIFC1; KIFC3; KIN; KIR2DL1; KIR2DL2; KIR2DL3; KIR2DL4; KIR2DL5A; KIR2DL5B; KIR2DS1; KIR2DS2; KIR2DS3; KIR2DS4; KIR3DL1; KIR3DL2; KIR3DL3; KIRREL2; KIRREL3; KIRREL; KISS1; KISS1R; KIT; KITLG; KLB; KLC1; KLF10; KLF11; KLF12; KLF13; KLF14; KLF15; KLF16; KLF1; KLF2; KLF3; KLF4; KLF5; KLF6; KLF7; KLF8; KLF9; KLHDC1; KLHDC2; KLHDC8A; KLHDC8B; KLHL10; KLHL12; KLHL1; KLHL20; KLHL25; KLHL26; KLHL29; KLHL2; KLHL31; KLHL35; KLHL3; KLHL40; KLHL41; KLHL42; KLHL5; KLHL6; KLHL7; KLHL9; KL; KLK10; KLK11; KLK12; KLK13; KLK14; KLK15; KLK1; KLK2; KLK3; KLK4; KLK5; KLK6; KLK7; KLK8; KLK9; KLKB1; KLLN; KLRB1; KLRC1; KLRC2; KLRC4; KLRC4-KLRK1; KLRD1; KLRG1; KLRG2; KMO; KMT2A; KMT2B; KMT2C; KMT20; KMT2E; KNG1; KNSTRN; KNTC1; KPNA1; KPNA2; KPNA3; KPNA4; KPNA6; KPNA7; KPNB1; KPRP; KPTN; KRAS; KRBOX4; KREMEN1; KRIT1; KRT10; KRT12; KRT13; KRT14; KRT15; KRT16; KRT17; KRT18; KRT19; KRT1; KRT20; KRT23; KRT2; KRT31; KRT32; KRT34; KRT35; KRT3; KRT4; KRT5; KRT6A; KRT6B; KRT6C; KRT71; KRT72; KRT74; KRT75; KRT76; KRT78; KRT7; KRT80; KRT81; KRT82; KRT83; KRT85; KRT86; KRT8; KRT9; KRTAP11-1; KRTAP5-1; KRTAP5-9; KSR1; KSR2; KTN1; KYNU; L1CAM; L2HGDH; L3MBTL1; L3MBTL2; L3MBTL3; L3MBTL4; LACC1; LACE1; LACRT; LACTB; LAD1; LAG3; LAIR1; LAIR2; LALBA; LAMA1; LAMA2; LAMA3; LAMA4; LAMAS; LAMB1; LAMB2; LAMB3; LAMB4; LAMC1; LAMC2; LAMC3; LAMP1; LAMP2; LAMP3; LAMTOR1; LAMTOR2; LAMTOR3; LANCL1; LANCL2; LAP3; LAPTM4B; LARGE; LARP1; LARP4; LARP6; LARP7; LARS2; LARS; LAS1L; LASP1; LAT2; LAT; LATS1; LATS2; LAX1; LAYN; LBH; LBP; LBR; LBX1; LBX2; LCA5; LCAT; LCE1C; LCE3A; LCE3B; LCE3C; LCE30; LCE3E; LCE5A; LCK; LCLAT1; LCMT1; LCN1; LCN2; LCOR; LCP1; LCP2; LCT; LDB1; LDB2; LDB3; LDHA; LDHAL6B; LDHB; LDHC; LDHD; LDLRAD3; LDLRAD4; LDLR; LDOC1; LDOC1L; LEAP2; LECT1; LECT2; LEF1; LEFTY1; LEFTY2; LEKR1; LEMD2; LEMD3; LE01; LEP; LEPROTL1; LETM1; LETMD1; LEUTX; LFNG; LGALS12; LGALS13; LGALS14; LGALS16; LGALS1; LGALS2; LGALS3BP; LGALS3; LGALS4; LGALS7B; LGALS8; LGALS9; LGALSL; LGI1; LGI2; LGI4; LGMN; LGR4; LGR5; LGR6; LGSN; LHB; LHCGR; LHFP; LHFPL1; LHFPL2; LHFPL3; LHFPL4; LHFPL5; LHPP; LHX1; LHX2; LHX3; LHX4; LHX5; LHX6; LHX9; LIAS; LIF; LIFR; LIG1; LIG3; LIG4; LILRA1; LILRA2; LILRA3; LILRA4; LILRA5; LILRB1; LILRB2; LILRB3; LILRB4; LILRB5; LIM2; LIMA1; LIMCH1; LIMD1; LIMD2; LIMK1; LIMK2; LIMS1; LIMS2; LIN28B; LIN52; LIN7A; LIN7B; LIN7C; LIN9; LING01; LINGO2; LINGO4; LINS; LIPA; LIPC; LIPE; LIPF; LIPG; LIPH; LIPI; LIPN; LIPT1; LIPT2; LITAF; LIX1; LLGL1; LLGL2; LMAN1; LMAN1L; LMAN2L; LMBR1; LMBRD1; LMBRD2; LMCD1; LMF1; LMLN; LMNA; LMNB1; LMNB2; LMNTD1; LMO1; LMO2; LMO3; LMO7; LMOD1; LMOD3; LMTK2; LMTK3; LMX1A; LMX1B; LNPEP; LNX1; LNX2; LOC100288966; LOC101060321; LOC102723475; LOC102723996; LOC102724127; LOC102724560; LOC102724770; LOC102725016; LOC102725035; LOC400499; LOC400927-CSNK1E; LOC645177; LONP1; LONRF1; LOR; LOXHD1; LOX; LOXL1; LOXL2; LOXL3; LOXL4; LPA; LPAR1; LPAR2; LPAR3; LPAR6; LPCAT1; LPCAT2; LPCAT3; LPCAT4; LPGAT1; LPIN1; LPIN2; LPL; LPO; LPP; LPPR2; LPPR4; LPXN; LRAT; LRBA; LRCH1; LRCH4; LRFN2; LRFN5; LRG1; LRGUK; LRIF1; LRIG1; LRIG3; LRIT1; LRIT3; LRMP; LRP10; LRP12; LRP1B; LRP1; LRP2BP; LRP2; LRP4; LRP5; LRP6; LRPAP1; LRPPRC; LRR1; LRRC15; LRRC16A; LRRC16B; LRRC17; LRRC18; LRRC1; LRRC20; LRRC26; LRRC30; LRRC32; LRRC37A; LRRC37B; LRRC39; LRRC49; LRRC4B; LRRC4C; LRRC4; LRRC52; LRRC59; LRRC61; LRRC63; LRRC69; LRRC6; LRRC74A; LRRC7; LRRC8A; LRRC8C; LRRCC1; LRRFIP1; LRRFIP2; LRRK1; LRRK2; LRRN1; LRRN2; LRRN3; LRRN4; LRRTM1; LRRTM2; LRRTM3; LRRTM4; LRSAM1; LRTM1; LRTOMT; LSAMP; LSM11; LSM1; LSM2; LSM3; LSM4; LSM5; LSM6; LSM7; LSP1; LSR; LSS; LST1; LTA4H; LTA; LTB4R2; LTB4R; LTB; LTBP1; LTBP2; LTBP3; LTBP4; LTBR; LTC4S; LTF; LTK; LUC7L3; LUC7L; LUM; LURAP1L; LUZP4; LUZP6; LVRN; LXN; LY6D; LY6E; LY6G5B; LY6G5C; LY6G6C; LY6G6F; LY6K; LY75-CD302; LY75; LY86; LY96; LY9; LYL1; LYN; LYNX1; LYPD1; LYPD2; LYPD3; LYPD4; LYPD5; LYPD6; LYPD8; LYPLA1; LYPLAL1; LYRM1; LYRM4; LYRM7; LYRM9; LYST; LYVE1; LYZ; LYZL1; LYZL2; LYZL6; LZTFL1; LZTR1; LZTS1; M6PR; MAATS1; MAB21L1; MAB21L2; MAB21L3; MACC1; MACROD1; MACROD2; MAD1L1; MAD2L1BP; MAD2L1; MAD2L2; MADCAM1; MADD; MAEA; MAEL; MAF1; MAFA; MAFB; MAFF; MAFG; MAF; MAFK; MAGEA10; MAGEA11; MAGEA12; MAGEA1; MAGEA2B; MAGEA3; MAGEA4; MAGEA6; MAGEA9; MAGEB17; MAGEB1; MAGEB2; MAGEB6; MAGEC1; MAGEC2; MAGEC3; MAGED1; MAGED2; MAGED4B; MAGED4; MAGEE1; MAGEH1; MAGEL2; MAG; MAGI1; MAGI2; MAGI3; MAGT1; MAK16; MAK; MAL; MALL; MALRD1; MALT1; MAML1; MAML2; MAML3; MAMLD1; MAN1A1; MAN1B1; MAN2A1; MAN2A2; MAN2B1; MAN2C1; MANBA; MANEA; MANF; MANSC1; MAOA; MAOB; MAP10; MAP1A; MAP1B; MAP1LC3A; MAP1LC3B2; MAP1LC3B; MAP1S; MAP2; MAP2K1; MAP2K2; MAP2K3; MAP2K4; MAP2K5; MAP2K6; MAP2K7; MAP3K10; MAP3K11; MAP3K12; MAP3K13; MAP3K14; MAP3K19; MAP3K1; MAP3K2; MAP3K3; MAP3K4; MAP3K5; MAP3K6; MAP3K7CL; MAP3K7; MAP3K8; MAP3K9; MAP4; MAP4K1; MAP4K2; MAP4K3; MAP4K4; MAP4K5; MAP6; MAP7; MAPS; MAPK10; MAPK11; MAPK12; MAPK13; MAPK14; MAPK15; MAPK1; MAPK3; MAPK6; MAPK7; MAPK8; MAPK8IP1; MAPK8IP2; MAPK8IP3; MAPK9; MAPKAP1; MAPKAPK2; MAPKAPK3; MAPKBP1; MAPRE1; MAPRE2; MAPRE3; MAPT; MARC1; MARC2; MARCH1; MARCH2; MARCH5; MARCH6; MARCH7; MARCH8; MARCKS; MARCO; MARK1; MARK2; MARK3; MARK4; MARS2; MARS; MARVELD1; MARVELD2; MARVELD3; MAST; MASTL; MASP1; MAST2; MAST4; MASTL; MAT1A; MAT2A; MAT2B; MATK; MATN2; MATN3; MATR3; MAU2; MAVS; MAX; MAZ; MB21D1; MB21D2; MBD1; MBD2; MBD3; MBD3L2; MBD4; MBD5; MBD6; MB; MBIP; MBL2; MBNL1; MBNL2; MBOAT1; MBOAT2; MBOAT4; MBP; MBTD1; MBTPS1; MBTPS2; MC1R; MC2R; MC3R; MC4R; MC5R; MCAM; MCAT; MCCC1; MCCC2; MCCD1; MCC; MCEE; MCF2; MCF2L2; MCF2L; MCFD2; MCHR1; MCHR2; MCIDAS; MCM10; MCM2; MCM3AP; MCM3; MCM4; MCM5; MCM6; MCMI; MCM8; MCM9; MCMBP; MCOLN1; MCOLN3; MCPH1; MCRS1; MCTP1; MCTP2; MCTS1; MCU; MCUR1; MDC1; MDFIC; MDFI; MDGA1; MDGA2; MDH1; MDH2; MDK; MDM1; MDM2; MDM4; ME1; ME2; ME3; MEA1; MECOM; MECP2; MED12; MED12L; MED13; MED13L; MED14; MED15; MED16; MED17; MED19; MEDT; MED22; MED23; MED24; MED25; MED28; MED29; MED30; MED4; MED6; MEDS; MEF2A; MEF2B; MEF2BNB-MEF2B; MEF2C; MEF2D; MEFV; MEGF10; MEGF11; MEGF8; MEGF9; MEI1; MEIS1; MEIS2; MELK; MEMO1; MEN1; MEOX1; MEOX2; MEP1A; MEPE; MERTK; MESDC1; MESDC2; MESP2; MEST; METAP1D; METAP1; METAP2; MET; METRN; METTL13; METTL16; METTL17; METTL18; METTL1; METTL21A; METTL21B; METTL21C; METTL24; METTLE; METTL7A; METTL9; MEX3B; MEX3C; MEX3D; MFAP3; MFAP4; MFAP5; MFGE8; MFHAS1; MFI2; MFN1; MFRP; MFSD11; MFSD12; MFSD1; MFSD6; MFSD7; MFSD8; MGA; MGAM; MGARP; MGAT1; MGAT2; MGAT3; MGAT4A; MGAT4B; MGAT4C; MGAT5B; MGAT5; MGEA5; MGLL; MGME1; MGMT; MGP; MGRN1; MGST1; MGST2; MGST3; MIA2; MIA3; MIA; MIB1; MIB2; MICA; MICAL2; MICALCL; MICB; MICU1; MICU3; MID1; MID2; MIEN1; MIER2; MIER3; MIF; MILR1; MINA; MINK1; MINPP1; MIOX; MIPEP; MIP; MIPOL1; MIR1-1HG; MIS18BP1; MITF; MIXL1; MKI67; MKKS; MKL1; MKL2; MKLN1; MKNK1; MKNK2; MKRN1; MKRN3; MKS1; MLANA; MLC1; MLEC; MLF1; MLF2; MLH1; MLH3; MLKL; MLLT10; MLLT1; MLLT3; MLLT4; MLLT6; MLN; MLNR; MLPH; MLST8; MLX; MLXIP; MLXIPL; MLYCD; MMAA; MMAB; MMADHC; MMD2; MMD; MME; MMEL1; MMP10; MMP11; MMP12; MMP13; MMP14; MMP15; MMP16; MMP17; MMP19; MMPI; MMP20; MMP21; MMP24; MMP25; MMP26; MMP28; MMP2; MMP3; MMPI; MMP8; MMP9; MMRN1; MMRN2; MMS19; MMS22L; MN1; MNAT1; MNDA; MNS1; MNT; MNX1; MOAP1; MOB1A; MOB1B; MOB2; MOB3B; MOB4; MOBP; MOCOS; MOCS1; MOCS2; MOGAT1; MOGAT2; MOGAT3; MOG; MOGS; MOK; MON1A; MON1B; MON2; MORC3; MORF4L1; MORN1; MORN2; MORNS; MOS; MOV10L1; MPC1; MPC2; MPDU1; MPDZ; MPEG1; MPG; MPHOSPH10; MPHOSPH6; MPHOSPH8; MPHOSPH9; MPI; MPLKIP; MPO; MPP1; MPP2; MPP3; MPP5; MPP7; MPPE1; MPPED2; MPRIP; MPST; MPV17; MPV17L2; MPZ; MPZL1; MPZL2; MPZL3; MR1; MRAP2; MRAP; MRAS; MRC1; MRC2; MRE11A; MREG; MRFAP1; MRGBP; MRGPRF; MRGPRX1; MRGPRX3; MRGPRX4; MRI1; MRM1; MROH2B; MRO; MRPL10; MRPL11; MRPL13; MRPL15; MRPL17; MRPL19; MRPL1; MRPL23; MRPL28; MRPL33; MRPL36; MRPL3; MRPL40; MRPL41; MRPL44; MRPL49; MRPL52; MRPL9; MRPS11; MRPS12; MRPS16; MRPS18B; MRPS22; MRPS23; MRPS28; MRPS30; MRPS31; MRPS33; MRPS6; MRPS7; MRPS9; MRRF; MRS2; MRVI1; MS4Al2; MS4A1; MS4A2; MS4A3; MS4A4A; MS4A6A; MS4A8; MSANTD3-TMEFF1; MSC; MSGN1; MSH2; MSH3; MSHS; MSH6; MSI1; MSI2; MSLN; MSMB; MSMO1; MSMP; MSN; MSR1; MSRA; MSRB2; MSRB3; MST1; MST1R; MSTN; MSTO1; MSX1; MSX2; MT1A; MT1B; MT1E; MT1F; MT1G; MT1H; MT1M; MT1X; MT2A; MT3; MT4; MTA1; MTA2; MTA3; MTAP; MTBP; MTCH1; MTCH2; MTCL1; MTCP1; MTDH; MTERF1; MTF1; MTFMT; MTFP1; MTG1; MTHFD1; MTHFD1L; MTHFD2; MTHFR; MTHFS; MTIF2; MTIF3; MTL5; MTM1; MTMR11; MTMR12; MTMR14; MTMR1; MTMR2; MTMR3; MTMR4; MTMR6; MTMR7; MTMR8; MTMR9; MTNR1A; MTNR1B; MT01; MTPAP; MTPN; MTRF1; MTR; MTRNR2L7; MTRR; MTSS1; MTTP; MTURN; MTUS1; MTUS2; MTX1; MTX2; MUC12; MUC13; MUC15; MUC17; MUC19; MUC1; MUC20; MUC21; MUC22; MUC2; MUC3A; MUC4; MUC5AC; MUC5B; MUC6; MUC7; MUCL1; MUM1; MURC; MUS81; MUSK; MUT; MUTYH; MVB12B; MVD; MVK; MVP; MX1; MX2; MXD1; MXD3; MXD4; MXI1; MXRA5; MYADM; MYBBP1A; MYB; MYBL1; MYBL2; MYBPC1; MYBPC2; MYBPC3; MYBPH; MYCBP2; MYCBPAP; MYC; MYCN; MYCT1; MYD88; MYDGF; MYEF2; MYEOV; MYF5; MYF6; MYH10; MYH11; MYH13; MYH14; MYH15; MYH1; MYH2; MYH3; MYH4; MYH6; MYH7B; MYH7; MYH8; MYH9; MYL12A; MYL12B; MYL1; MYL2; MYL3; MYL4; MYL6; MYL7; MYL9; MYLIP; MYLK2; MYLK3; MYLK; MYLPF; MYNN; MYO10; MYO15A; MYO16; MYO18B; MYO1A; MYO1B; MY01C; MYOCD; MYO1E; MYO1F; MYO3A; MYO3B; MYO5A; MYO5B; MY05C; MYO6; MYO7A; MY07B; MYO9A; MYO9B; MYOCD; MYOC; MYOD1; MYOF; MYOG; MYOM1; MYOM2; MYOT; MYOZ1; MYOZ2; MYOZ3; MYPN; MYRF; MYRFL; MYRIP; MYT1; MYT1L; MYZAP; MZB1; MZF1; N4BP1; N4BP2L1; N4BP2L2; NAA10; NAA15; NAA16; NAA20; NAA25; NAA30; NAA35; NAA40; NAA50; NAA60; NAAA; NAALAD2; NAALADL1; NAALADL2; NAB1; NAB2; NABP1; NABP2; NACA; NACC1; NACC2; NADSYN1; NAE1; NAF1; NAGA; NAGK; NAGLU; NAGPA; NAGS; NAIP; NALCN; NAMPT; NANOG; NANOS1; NANOS2; NANOS3; NANS; NAP1L1; NAP1L3; NAP1L4; NAP1L5; NAPA; NAPEPLD; NAPG; NAPRT; NAPSA; NARFL; NARR; NARS; NAT10; NAT14; NAT1; NAT2; NAT6; NAT8B; NAT8; NAT8L; NAV1; NAV2; NAV3; NBAS; NBEA; NBEAL1; NBEAL2; NBN; NBPF3; NBR1; NCALD; NCAM1; NCAM2; NCAN; NCAPD2; NCAPD3; NCAPG2; NCAPG; NCBP2; NCEH1; NCF1; NCF2; NCF4; NCK1; NCK2; NCKAP1; NCKAP1L; NCKAP5; NCKIPSD; NCL; NCOA1; NCOA2; NCOA3; NCOA4; NCOA5; NCOA6; NCOA7; NCOR1; NCOR2; NCR1; NCR2; NCR3; NCR3LG1; NCS1; NCSTN; NDC80; NDE1; NDEL1; NDFIP1; NDFIP2; NDNF; NDN; NDP; NDRG1; NDRG2; NDRG3; NDRG4; NDST1; NDST2; NDST3; NDST4; NDUFA10; NDUFA11; NDUFA12; NDUFA13; NDUFA1; NDUFA2; NDUFA5; NDUFA6; NDUFA9; NDUFAB1; NDUFAF1; NDUFAF2; NDUFAF3; NDUFAF4; NDUFAF5; NDUFAF6; NDUFB10; NDUFB11; NDUFB2; NDUFB3; NDUFB4; NDUFB5; NDUFB6; NDUFB8; NDUFB9; NDUFC2; NDUFS1; NDUFS2; NDUFS3; NDUFS4; NDUFS6; NDUFS7; NDUFS8; NDUFV1; NDUFV2; NDUFV3; NEB; NEBL; NECAB1; NECAB3; NECAP1; NEDD1; NEDD4; NEDD4L; NEDD8; NEDD9; NEFH; NEFM; NEIL1; NEIL2; NEIL3; NEK10; NEK11; NEK1; NEK2; NEK3; NEK4; NEK6; NEK7; NEK8; NEK9; NELFA; NELFB; NELFCD; NELFE; NELL1; NELL2; NEMF; NE01; NES; NETT; NET01; NETO2; NEU1; NEU3; NEURL1; NEURL2; NEUROD1; NEUROD2; NEUROD4; NEUROD6; NEUROG1; NEUROG2; NEUROG3; NF1; NF2; NFAM1; NFASC; NFAT5; NFATC1; NFATC2; NFATC3; NFATC4; NFE2; NFE2L1; NFE2L2; NFE2L3; NFIA; NFIB; NFIC; NFIL3; NFIX; NFKB1; NFKB2; NFKBIA; NFKBIB; NFKBIE; NFKBIL1; NFKBIZ; NFRKB; NFS1; NFU1; NFYA; NFYB; NFYC; NGB; NGDN; NGEF; NGF; NGFRAP1; NGFR; NGLY1; NHEJ1; NHLH1; NHLRC1; NHLRC3; NHP2; NHP2L1; NHS; NHSL1; NICN1; NID1; NID2; NIF3L1; NIM1K; NIN; NINJ1; NINJ2; NINL; NIPA1; NIPA2; NIPAL3; NIPAL4; NIPBL; NIPSNAP1; NIPSNAP3B; NISCH; NIT1; NIT2; NKAIN2; NKAIN3; NKAP; NKD1; NKD2; NKG7; NKIRAS1; NKIRAS2; NKRF; NKTR; NKX1-1; NKX1-2; NKX2-1; NKX2-2; NKX2-3; NKX2-5; NKX2-6; NKX2-8; NKX3-1; NKX6-1; NKX6-2; NLE1; NLGN1; NLGN2; NLGN3; NLGN4X; NLGN4Y; NLK; NLN; NLRC3; NLRC4; NLRC5; NLRP10; NLRP11; NLRP12; NLRP13; NLRP14; NLRP1; NLRP2; NLRP3; NLRP4; NLRP5; NLRP6; NLRP7; NLRP8; NLRP9; NMB; NMBR; NME1; NME1-NME2; NME2; NME3; NME4; NME5; NME6; NME7; NME8; NME9; NMI; NMNAT1; NMNAT2; NMNAT3; NMS; NMT1; NMT2; NMU; NMUR1; NMUR2; NNAT; NNMT; NNT; NOA1; NOB1; NOBOX; NOC3L; NOD1; NOD2; NODAL; NOG; NOL11; NOL3; NOL4; NOL6; NOL8; NOLC1; NOMI; NOMO1; NONO; NOP10; NOP14; NOP16; NOP2; NOP56; NOP9; NOS1AP; NOS1; NOS2; NOS3; NOSIP; NOSTRIN; NOTCH1; NOTCH2; NOTCH3; NOTCH4; NOTUM; NOVA1; NOVA2; NOV; NOX1; NOX3; NOX4; NOX5; NOXA1; NOXO1; NPAP1; NPAS2; NPAS3; NPAS4; NPAT; NPB; NPBWR1; NPC1; NPC1L1; NPC2; NPDC1; NPEPL1; NPEPPS; NPFF; NPFFR2; NPHP1; NPHP3; NPHP4; NPHS1; NPHS2; NPL; NPLOC4; NPM1; NPM2; NPNT; NPPC; NPR1; NPR2; NPR3; NPRL2; NPRL3; NPS; NPSR1; NPTN; NPTX1; NPTX2; NPTXR; NPVF; NPW; NPY1R; NPY2R; NPY5R; NPY; NQO1; NQO2; NROB1; NR101; NR102; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRAP; NRARP; NRBP1; NRBP2; NRCAM; NRD1; NRDE2; NREP; NRF1; NRG1; NRG2; NRG3; NRG4; NRGN; NRIP1; NRIP2; NRK; NRL; NRM; NRN1; NRP1; NRP2; NRSN1; NRSN2; NRTN; NRXN1; NRXN2; NRXN3; NSA2; NSD1; NSDHL; NSF; NSFL1C; NSG1; NSMAF; NSMCE2; NSMF; NSRP1; NSUN2; NSUN3; NSUN5; NSUN7; NT5C1B; NT5C1B-RDH14; NT5C2; NT5C3A; NT5C; NT5DC1; NT5DC3; NT5E; NT5M; NTAN1; NTF3; NTF4; NTHL1; NTM; NTN1; NTN4; NTNG1; NTNG2; NTPCR; NTRK1; NTRK2; NTRK3; NTS; NTSR1; NTSR2; NUAK1; NUAK2; NUB1; NUBP1; NUBP2; NUBPL; NUCB1; NUCB2; NUCKS1; NUDCD1; NUDCD3; NUDC; NUDT10; NUDT11; NUDT15; NUDT19; NUDT1; NUDT21; NUDT2; NUDT3; NUDT6; NUDT7; NUF2; NUFIP1; NUFIP2; NUGGC; NUMA1; NUMB; NUMBL; NUP153; NUP155; NUP205; NUP210; NUP214; NUP35; NUP37; NUP43; NUP50; NUP62; NUP85; NUP88; NUP93; NUP98; NUPL2; NUPR1; NUS1; NUSAP1; NUTF2; NUTM1; NUTM2A; NUTM2B; NVL; NWD1; NXF1; NXF2B; NXF3; NXF5; NXN; NXNL1; NXNL2; NXPE1; NXPE2; NXPE4; NXPH1; NXPH2; NXT1; NXT2; NYAP2; NYX; OARD1; OAS1; OAS2; OAS3; OASL; OAT; OAZ1; OAZ2; OBFC1; OBP2A; OBSCN; OBSL1; OC90; OCA2; OCIAD1; OCIAD2; OCLM; OCLN; OCM2; OCM; OCRL; ODAM; ODC1; ODF1; ODF3B; ODF4; OFCC1; OFD1; OGDH; OGDHL; OGFOD1; OGFR; OGG1; OGN; OGT; OIP5; OIT3; OLA1; OLFM1; OLFM2; OLFM4; OLFML2B; OLIG1; OLIG2; OLIG3; OLR1; OMD; OMG; OMP; ONECUT1; ONECUT2; OPA1; OPA3; OPCML; OPHN1; OPN1LW; OPN1MW2; OPN1SW; OPN3; OPN4; OPN5; OPRK1; OPRL1; OPRM1; OPTC; OPTN; OR10A2; OR10A4; OR10C1; OR10J1; OR10J3; OR10J5; OR10K1; OR10K2; OR10R2; OR10T2; OR10X1; OR10Z1; OR11A1; OR12D2; OR12D3; OR13C3; OR13C4; OR13F1; OR13G1; OR13J1; OR14J1; OR1A1; OR1C1; OR1D2; OR1E1; OR1E2; OR1J2; OR1K1; OR1L8; OR1M1; OR1N1; OR1N2; OR2A25; OR2AG1; OR2AK2; OR2AT4; OR2B2; OR2B3; OR2B6; OR2C1; OR2F2; OR2G2; OR2G3; OR2H2; OR2J2; OR2J3; OR2L2; OR2M3; OR2M4; OR2M7; OR2S2; OR2T10; OR2T12; OR2T1; OR2T2; OR2T33; OR2T4; OR2T5; OR2W1; OR2Y1; OR2Z1; OR3A1; OR4A15; OR4C12; OR4C13; OR4C6; OR4D10; OR4K13; OR4P4; OR4S2; OR51A2; OR51A7; OR51E1; OR51E2; OR51F2; OR51V1; OR52B2; OR52B4; OR52D1; OR52E6; OR5211; OR5212; OR52K1; OR52K2; OR52M1; OR5D18; OR5H2; OR5H6; OR5K1; OR5K2; OR5V1; OR6B1; OR6B2; OR6C1; OR6F1; OR6K2; OR6K3; OR6K6; OR6N1; OR6N2; OR6X1; OR6Y1; OR7C1; OR7D2; OR7E24; OR8S1; OR9K2; OR9Q2; ORAI1; ORAI3; ORA0V1; ORC2; ORC3; ORC4; ORC5; ORC6; ORM1; ORM2; ORMDL3; OS9; OSBP2; OSBP; OSBPL10; OSBPL11; OSBPL1A; OSBPL2; OSBPL3; OSBPL5; OSBPL6; OSBPL8; OSBPL9; OSCAR; OSER1; OSGEP; OSGIN1; OSM; OSMR; OSR1; OSR2; OSTF1; OSTM1; OTC; OTOA; OTOF; OTOG; OTOGL; OTOL1; OTOP1; OTOR; OTP; OTUB1; OTUD1; OTUD4; OTUD7A; OTUD7B; OTULIN; OTX1; OTX2; OVCA2; OVOL1; OVOL2; OXA1L; OXCT1; OXER1; OXGR1; OXR1; OXSR1; OXT; OXTR; P2RX1; P2RX2; P2RX3; P2RX4; P2RX5; P2RX6; P2RX7; P2RY11; P2RY12; P2RY13; P2RY14; P2RY1; P2RY2; P2RY4; P2RY6; P2RY8; P3H2; P3H3; P3H4; P4HA1; P4HA2; P4HB; P4HTM; PA2G4; PABPC1; PABPC3; PABPC4L; PABPN1; PACRG; PACS1; PACS2; PACSIN1; PACSIN2; PADI4; PAEP; PAF1; PAFAH1B1; PAFAH1B2; PAFAH2; PAG1; PAGE1; PAGE4; PAGES; PAGR1; PAH; PAICS; PAIP1; PAIP2; PAK1; PAK1IP1; PAK2; PAK3; PAK4; PAK6; PAK7; PALB2; PALD1; PALLD; PALM2-AKAP2; PAM16; PAM; PAMR1; PAN2; PAN3; PANK1; PANK2; PANX1; PANX2; PAOX; PAPD7; PAPL; PAPOLA; PAPOLG; PAPPA2; PAPPA; PAPSS1; PAPSS2; PAQR3; PAQR5; PAQR7; PARD3B; PARD3; PARD6A; PARD6B; PARG; PARK2; PARL; PARM1; PARN; PARP12; PARP14; PARP15; PARP1; PARP2; PARP3; PARP4; PARP6; PARP9; PARPBP; PARS2; PARVA; PARVB; PARVG; PASD1; PASK; PATE1; PATZ1; PAWR; PAX1; PAX2; PAX3; PAX4; PAX5; PAX6; PAX8; PAX9; PAXIP1; PBK; PBLD; PBOV1; PBRM1; PBX1; PBX2; PBX3; PBX4; PCBD1; PCBD2; PCBP1; PCBP2; PCBP3; PCBP4; PCCA; PCCB; PCDH10; PCDH11X; PCDH11Y; PCDH15; PCDH17; PCDH18; PCDH19; PCDH1; PCDH20; PCDH7; PCDH8; PCDH9; PCDHA1; PCDHA4; PCDHA6; PCDHB1; PCDHB2; PCDHB3; PCDHB8; PCDHGA11; PCDHGA3; PCDHGB4; PCDHGB6; PCDHGC3; PCED1B; PCF11; PCGF1; PCGF2; PCGF3; PCGF6; PC; PCID2; PCK1; PCK2; PCLO; PCM1; PCMT1; PCMTD1; PCNA; PCNT; PCNXL2; PCNXL4; PCOLCE2; PCOLCE; PCP2; PCP4; PCSK1; PCSK1N; PCSK2; PCSK4; PCSK5; PCSK6; PCSK7; PCTP; PCYT1A; PCYT1B; PDAP1; PDCD10; PDCD1; PDCD1LG2; PDCD2; PDCD4; PDCD5; PDCD6; PDCD6IP; PDCD7; PDC; PDCL2; PDCL3; PDE10A; PDE11A; PDE12; PDE1A; PDE1B; PDE1C; PDE2A; PDE3A; PDE3B; PDE4A; PDE4D; PDE5A; PDE6A; PDE6B; PDE6C; PDE6D; PDE6G; PDE6H; PDE7A; PDE7B; PDE8A; PDE8B; PDE9A; PDF; PDGFA; PDGFB; PDGFC; PDGFD; PDGFRA; PDGFRB; PDGFRL; PDHA1; PDHB; PDHX; PDIA2; PDIA3; PDIA4; PDIA5; PDIA6; PDK1; PDK2; PDK3; PDK4; PDLIM1; PDLIM2; PDLIM3; PDLIM4; PDLIM5; PDLIM7; PDP1; PDP2; PDPK1; PDPR; PDRG1; PDS5A; PDS5B; PDSS1; PDSS2; PDX1; PDXDC1; PDXK; PDXP; PDYN; PDZD2; PDZD4; PDZD7; PDZD8; PDZK1; PDZRN3; PDZRN4; PEA15; PEAK1; PEARL; PEBP1; PEBP4; PECAM1; PECR; PEG10; PEG3; PELI1; PELI2; PELP1; PEMT; PENK; PEPD; PER1; PER2; PER3; PERM1; PERP; PES1; PET100; PET117; PEX11A; PEX12; PEX13; PEX14; PEX16; PEX19; PEX1; PEX26; PEX2; PEX3; PEX5; PEX5L; PEX6; PEX7; PF4; PF4V1; PFDN4; PFDN5; PFDN6; PFKFB1; PFKFB2; PFKFB3; PFKFB4; PFKL; PFKM; PFKP; PFN1; PFN2; PGA3; PGA4; PGA5; PGAM1; PGAM2; PGAM4; PGAP1; PGAP2; PGAP3; PGBD1; PGBD5; PGC; PGD; PGF; PGGT1B; PGK1; PGK2; PGLS; PGLYRP1; PGLYRP2; PGLYRP3; PGLYRP4; PGM3; PGPEP1; PGP; PGR; PGRMC1; PGRMC2; PHACTR1; PHACTR2; PHACTR3; PHB2; PHB; PHC1; PHC2; PHC3; PHEX; PHF10; PHF11; PHF12; PHF19; PHF1; PHF20; PHF21A; PHF23; PHF2; PHF3; PHF5A; PHF6; PHF8; PHGDH; PHIP; PHKA1; PHKA2; PHKB; PHKG1; PHKG2; PHLDA1; PHLDA2; PHLDA3; PHLDB1; PHLPP1; PHLPP2; PHOX2A; PHOX2B; PHPT1; PHRF1; PHTF1; PHTF2; PHYH; PHYHIP; PHYKPL; PI15; PI16; PI3; PI4K2A; PI4K2B; PI4KA; PI4KB; PIAS1; PIAS2; PIAS4; PIBF1; PICALM; PICK1; PID1; PIDD1; PIEZO1; PIEZO2; PIF1; PIFO; PIGA; PIGF; PIGG; PIGL; PIGM; PIGN; PIGO; PIGP; PIGQ; PIGR; PIGT; PIGU; PIGW; PIGY; PIH101; PIK3AP1; PIK3C2A; PIK3C2B; PIK3C2G; PIK3C3; PIK3CA; PIK3CB; PIK3CD; PIK3CG; PIK3IP1; PIK3R1; PIK3R2; PIK3R3; PIK3R4; PIK3R5; PIKFYVE; PILRA; PIM1; PIM2; PIM3; PINI; PIN4; PINX1; PIP4K2A; PIP4K2B; PIP4K2C; PIPSK1A; PIPSK1B; PIPSK1C; PIPSKL1; PIP; PIPDX; PIR; PISD; PITPNA; PITPNM1; PITPNM3; PITRM1; PITX1; PITX2; PITX3; PIWIL1; PIWIL2; PIWIL3; PIWIL4; PJA1; PJA2; PKD1; PKD1L1; PKD1L2; PKD1L3; PKD2; PKD2L1; PKDCC; PKDREJ; PKHD1; PKIA; PKIB; PKLR; PKM; PKMYT1; PKN1; PKN2; PKN3; PKNOX1; PKNOX2; PKP1; PKP2; PKP3; PKP4; PLAZA; PLA2G10; PLA2G12A; PLA2G12B; PLA2G15; PLA2G16; PLA2G1B; PLA2G3; PLA2G4A; PLA2G4B; PLA2G4C; PLA2G4D; PLA2G5; PLA2G6; PLA2G7; PLA2R1; PLAA; PLAC1; PLACE; PLAG1; PLAGL1; PLAGL2; PLAT; PLAU; PLAUR; PLB1; PLBD1; PLCB1; PLCB2; PLCB3; PLCB4; PLCD1; PLCD3; PLCD4; PLCE1; PLCG1; PLCG2; PLCH1; PLCL1; PLCL2; PLCXD2; PLCXD3; PLCZ1; PLD1; PLD2; PLD3; PLD4; PLD5; PLEC; PLEK2; PLEKHA1; PLEKHA2; PLEKHAS; PLEKHA6; PLEKHA7; PLEKHB1; PLEKHD1; PLEKHF1; PLEKHF2; PLEKHG1; PLEKHG2; PLEKHG3; PLEKHG4; PLEKHG6; PLEKHH2; PLEKHM1; PLEKHO1; PLEKHG2; PLEK; PLG; PLGLB2; PLIN1; PLIN2; PLIN3; PLIN4; PLIN5; PLK1; PLK2; PLK4; PLK5; PLLP; PLN; PLOD2; PLP1; PLP2; PLRG1; PLS1; PLS3; PLSCR1; PLSCR3; PLSCR4; PLSCR5; PLTP; PLVAP; PLXDC1; PLXDC2; PLXNA1; PLXNA2; PLXNA3; PLXNA4; PLXNB1; PLXNB3; PLXNC1; PLXND1; PM20D1; PMAIP1; PMCH; PMEL; PMEPA1; PMF1-BGLAP; PMF1; PML; PMM1; PMM2; PMP22; PMPCA; PMPCB; PMS1; PMS2; PMVK; PNCK; PNKD; PNKP; PNLDC1; PNLIP; PNLIPRP2; PNMA1; PNMA2; PNMT; PNN; PNO1; PNOC; PNP; PNPLA1; PNPLA2; PNPLA3; PNPLA4; PNPLA5; PNPLA6; PNPLA8; PNPO; PNPT1; PNRC1; POC1A; POC1B; P005; PODXL; POF1B; POFUT1; POFUT2; POGK; POGLUT1; POLA1; POLA2; POLB; POLD1; POLD2; POLD3; POLD4; POLDIP3; POLE2; POLE3; POLE4; POLE; POLG2; POLG; POLH; POLI; POLK; POLL; POLM; POLN; POLQ; POLR1A; POLR1B; POLR1C; POLR1D; POLR2A; POLR2B; POLR2C; POLR2D; POLR2E; POLR2F; POLR2G; POLR2H; POLR2J; POLR2K; POLR2M; POLR3A; POLR3B; POLR3E; POLR3K; POLRMT; POM121; POMC; POMGNT1; POMGNT2; POMK; POMP; POMT1; POMT2; POMZP3; PON1; PON2; PON3; POP1; POP4; POPDC3; PORCN; POR; POSTN; POT1; POTED; POTEF; POTEG; POTEH; POTEM; POU1F1; POU2AF1; POU2F1; POU2F2; POU2F3; POU3F2; POU3F3; POU3F4; POU4F1; POU4F2; POU4F3; POU5F1B; POU5F1; POU6F1; POU6F2; PPA1; PPA2; PPAP2A; PPAP2C; PPAPDC1B; PPAPDC2; PPAPDC3; PPARA; PPARD; PPARGC1A; PPARGC1B; PPARG; PPAT; PPBP; PPCDC; PPEF1; PPEF2; PPFIA1; PPFIA2; PPFIA4; PPFIBP1; PPFIBP2; PPIA; PPIB; PPIC; PPID; PPIF; PPIG; PPIL1; PPIL2; PPIL3; PPIP5K1; PPL; PPM1A; PPM1B; PPM1D; PPM1E; PPM1F; PPM1G; PPM1H; PPM1K; PPM1L; PPM1M; PPME1; PPDX; PPP1CA; PPP1CC; PPP1R10; PPP1R11; PPP1R12A; PPP1R12B; PPP1R12C; PPP1R13B; PPP1R13L; PPP1R14A; PPP1R14B; PPP1R14C; PPP1R15A; PPP1R15B; PPP1R17; PPP1R18; PPP1R1A; PPP1R1B; PPP1R2; PPP1R3A; PPP1R3B; PPP1R3C; PPP1R42; PPP1R7; PPP1R9A; PPP2CA; PPP2CB; PPP2R1A; PPP2R1B; PPP2R2A; PPP2R2B; PPP2R2C; PPP2R3A; PPP2R3B; PPP2R4; PPP2R5A; PPP2R5B; PPP2R5C; PPP2R5D; PPP2R5E; PPP3CA; PPP3CB; PPP3CC; PPP3R1; PPP3R2; PPP4C; PPP4R1; PPP5C; PPP6C; PPP6R2; PPP6R3; PPRC1; PPT2; PPY; PQBP1; PQLC3; PRAC1; PRAC2; PRADC1; PRAF2; PRAM1; PRAME; PRAP1; PRB1; PRB2; PRB3; PRB4; PRC1; PRCC; PRCD; PRCP; PRDM10; PRDM11; PRDM13; PRDM14; PRDM1; PRDM4; PRDM5; PRDM6; PRDM7; PRDM8; PRDM9; PRDX2; PRDX3; PRDX4; PRDX5; PRDX6; PREB; PRELP; PREP; PREPL; PREX1; PREX2; PRF1; PRG2; PRG4; PRH1; PRH2; PRICKLE1; PRICKLE2; PRICKLE4; PRIM1; PRIMAL; PRIMPOL; PRKAA1; PRKAB1; PRKACA; PRKACB; PRKACG; PRKAG1; PRKAG2; PRKAG3; PRKAR1A; PRKAR1B; PRKAR2A; PRKAR2B; PRKCA; PRKCB; PRKCDBP; PRKCD; PRKCE; PRKCG; PRKCH; PRKCI; PRKCQ; PRKCSH; PRKCZ; PRKD1; PRKD3; PRKDC; PRKG1; PRKG2; PRKRA; PRKRIR; PRKX; PRLH; PRL; PRLHR; PRLR; PRM1; PRM2; PRM3; PRMT1; PRMT2; PRMT3; PRMT5; PRMT8; PRND; PRNP; PROC; PROCR; PRODH; PROK2; PROKR1; PROKR2; PROL1; PROM1; PROM2; PROP1; PROS1; PROSER1; PROX1; PROZ; PRPF19; PRPF31; PRPF38B; PRPF3; PRPF40A; PRPF4B; PRPF4; PRPF6; PRPF8; PRPH2; PRPH; PRPS1; PRPS1L1; PRPS2; PRPSAP1; PRPSAP2; PRR11; PRR13; PRR15; PRR16; PRR34; PRR3; PRR5; PRR9; PRRC1; PRRC2A; PRRC2C; PRRG4; PRRT1; PRRT2; PRRX1; PRRX2; PRSS12; PRSS16; PRSS1; PRSS21; PRSS22; PRSS23; PRSS27; PRSS2; PRSS33; PRSS35; PRSS3; PRSS50; PRSS53; PRSS55; PRSS57; PRSS58; PRSS8; PRTFDC1; PRTG; PRTN3; PRUNE2; PRUNE; PRX; PRY; PSAP; PSAT1; PSCA; PSD3; PSD4; PSD; PSEN1; PSEN2; PSENEN; PSG1; PSG2; PSG5; PSG6; PSG8; PSG9; PSIP1; PSKH1; PSMA1; PSMA2; PSMA3; PSMA4; PSMA6; PSMA7; PSMB10; PSMB1; PSMB4; PSMB5; PSMB6; PSMB7; PSMB8; PSMB9; PSMC1; PSMC2; PSMC3; PSMC3IP; PSMC4; PSMC5; PSMC6; PSMD10; PSMD12; PSMD13; PSMD14; PSMD1; PSMD2; PSMD3; PSMD4; PSMD6; PSMD7; PSMD8; PSMD9; PSME1; PSME2; PSME3; PSME4; PSMF1; PSMG1; PSMG2; PSMG3; PSORS1C1; PSORS1C2; PSPC1; PSPH; PSPN; PSTPIP1; PSTPIP2; PTBP1; PTBP2; PTCD1; PTCH1; PTCHD1; PTCHD4; PTCRA; PTDSS1; PTEN; PTER; PTF1A; PTGDR2; PTGDR; PTGDS; PTGER1; PTGER2; PTGER3; PTGER4; PTGES2; PTGES3; PTGES; PTGIR; PTGIS; PTGR1; PTGS1; PTGS2; PTH1R; PTH2; PTH2R; PTH; PTHLH; PTK2B; PTK2; PTK6; PTK7; PTMA; PTMS; PTN; PTOV1; PTP4A1; PTP4A3; PTPMT1; PTPN11; PTPN12; PTPN13; PTPN14; PTPN18; PTPN1; PTPN21; PTPN22; PTPN23; PTPN2; PTPN3; PTPN4; PTPN5; PTPN6; PTPN7; PTPN9; PTPRA; PTPRB; PTPRCAP; PTPRD; PTPRE; PTPRG; PTPRH; PTPRJ; PTPRK; PTPRM; PTPRN2; PTPRN; PTPRO; PTPRQ; PTPRR; PTPRS; PTPRT; PTPRZ1; PTRF; PTRH1; PTRH2; PTRHD1; PTS; PTTG1; PTTG1IP; PTTG2; PTX3; PUF60; PUM2; PURA; PURB; PUS10; PUS1; PVALB; PVR; PVRL1; PVRL2; PVRL3; PVRL4; PWP2; PWWP2B; PXDN; PXDNL; PXK; PXMP2; PXN; PXT1; PYCARD; PYCR1; PYDC1; PYDC2; PYGB; PYGL; PYGM; PYG01; PYGO2; PYHIN1; PYROXD1; PYY; PZP; QARS; QDPR; QKI; QPCT; QPCTL; QPRT; QRFP; QRFPR; QRSL1; QSOX1; QSOX2; QTRT1; R3HCC1; R3HCC1L; R3HDM1; R3HDML; RAB11A; RAB11B; RAB11FIP1; RAB11FIP2; RAB11FIP3; RAB11FIP4; RAB11FIP5; RAB12; RAB14; RAB15; RAB18; RAB1A; RAB1B; RAB20; RAB21; RAB22A; RAB23; RAB24; RAB25; RAB27A; RAB27B; RAB28; RAB29; RAB2A; RAB31; RAB32; RAB33B; RAB34; RAB35; RAB36; RAB37; RAB38; RAB39A; RAB39B; RAB3A; RAB3D; RAB3GAP1; RAB3GAP2; RAB3IL1; RAB3IP; RAB40AL; RAB40B; RAB40C; RAB4A; RAB4B; RAB5A; RAB5B; RAB5C; RAB6A; RAB6B; RAB6C; RAB7A; RAB8A; RAB8B; RAB9A; RABAC1; RABEP1; RABEP2; RABEPK; RABGAP1L; RABGEF1; RABIF; RABL6; RAC1; RAC2; RAC3; RACGAP1; RAD17; RAD18; RAD1; RAD21; RAD21L1; RAD23A; RAD23B; RAD50; RAD51AP1; RAD51B; RAD51C; RAD51D; RAD51; RAD52; RAD54B; RAD9A; RAD9B; RAE1; RAET1E; RAET1L; RAFT; RAG1; RAG2; RAI14; RAI1; RAI2; RALA; RALB; RALBP1; RALGAPA1; RALGAPA2; RALGAPB; RALGDS; RALGPS1; RALY; RALYL; RAMP1; RAMP2; RAMP3; RANBP10; RANBP17; RANBP1; RANBP2; RANBP3; RANBP3L; RANBP6; RANBP9; RANGAP1; RANGRF; RAN; RAP1B; RAP1GAP2; RAP1GDS1; RAP2A; RAP2B; RAPGEF1; RAPGEF2; RAPGEF3; RAPGEF4; RAPGEF5; RAPH1; RAPSN; RARA; RARB; RARG; RARRES1; RARRES2; RARRES3; RARS2; RARS; RASA1; RASA2; RASAL1; RASAL2; RASD1; RASD2; RASEF; RASGEF1A; RASGEF1C; RASGRF1; RASGRF2; RASGRP1; RASGRP2; RASGRP3; RASGRP4; RASIP1; RASL10A; RASL10B; RASL11A; RASL11B; RASL12; RASSF1; RASSF2; RASSF3; RASSF4; RASSF5; RASSF6; RASSF7; RASSF8; RAX2; RAX; RB1CC1; RB1; RBAK; RBBP5; RBBP6; RBBP7; RBBP8; RBBP9; RBCK1; RBFOX1; RBFOX2; RBFOX3; RBL1; RBL2; RBM10; RBM12; RBM14; RBM14-RBM4; RBM15; RBM17; RBM20; RBM25; RBM26; RBM27; RBM28; RBM38; RBM39; RBM3; RBM45; RBM46; RBM47; RBM4; RBM5; RBM6; RBM7; RBMS1; RBMS2; RBMS3; RBMX2; RBMX; RBMXL2; RBMY1A1; RBP1; RBP2; RBP3; RBP4; RBPJ; RBPMS2; RBSN; RBX1; RC3H1; RCAN1; RCAN2; RCBTB1; RCBTB2; RCHY1; RCL1; RCN1; RCN2; RCOR1; RCSD1; RCVRN; RD3; RDH10; RDH11; RDH12; RDH14; RDH16; RDH5; RDH8; RDM1; RDX; REC8; RECK; RECQL5; RECQL; REEP1; REEP2; REEP3; REEP5; REEP6; REG1A; REG1B; REG3A; RELA; RELB; REL; RELN; REM1; RENBP; REN; REPS1; REPS2; RERG; RERGL; REST; RET; RETN; RETNLB; RETSAT; REV1; REV3L; REX01; REXO2; REXO4; RFC1; RFC2; RFC3; RFC4; RFC5; RFFL; RFK; RFPL1; RFT1; RFTN1; RFTN2; RFWD2; RFWD3; RFX1; RFX2; RFX3; RFX4; RFX5; RFX6; RFX8; RFXANK; RFXAP; RGCC; RGL1; RGL2; RGL4; RGMA; RGMB; RGN; RGPD2; RGR; RGS10; RGS11; RGS12; RGS13; RGS14; RGS16; RGS17; RGS18; RGS19; RGS1; RGS20; RGS21; RGS22; RGS2; RGS3; RGS4; RGS5; RGS6; RGS7BP; RGS7; RGS8; RGS9BP; RGS9; RGSL1; RHAG; RHBDD2; RHBDD3; RHBDF1; RHBDF2; RHBDL1; RHBDL2; RHCE; RHCG; RHEB; RHEBL1; RHNO1; RHOB; RHOBTB1; RHOBTB2; RHOBTB3; RHOD; RHOF; RHOG; RHOH; RHO; RHOJ; RHOQ; RHOT1; RHOU; RHOV; RHOXF1; RHOXF2; RHPN1; RHPN2; RIBC2; RIC1; RIC3; RICTOR; RIFT; RILP; RILPL1; RIMBP2; RIMBP3C; RIMS1; RIMS2; RIMS4; RINI1; RIN2; RIN3; RING1; RINT1; RIOK1; RIOK2; RIOK3; RIPK1; RIPK2; RIPK3; RIPK4; RIPPLY2; RIT1; RITZ; RITA1; RLBP1; RLF; RLIM; RLN1; RLN2; RLN3; RMDN1; RMDN2; RMDN3; RMI1; RMI2; RMND1; RNASE11; RNASE12; RNASE13; RNASE1; RNASE2; RNASE3; RNASE4; RNASE6; RNASE7; RNASE8; RNASE9; RNASEH1; RNASEH2A; RNASEH2B; RNASEH2C; RNASEK; RNASEL; RNASET2; RND1; RND3; RNF103-CHMP3; RNF103; RNF111; RNF112; RNF114; RNF123; RNF125; RNF128; RNF130; RNF135; RNF138; RNF139; RNF144A; RNF144B; RNF146; RNF149; RNF14; RNF150; RNF152; RNF157; RNF167; RNF168; RNF170; RNF180; RNF182; RNF19A; RNF207; RNF20; RNF213; RNF214; RNF216; RNF24; RNF2; RNF31; RNF34; RNF39; RNF40; RNF41; RNF43; RNF44; RNF4; RNF5; RNF6; RNF7; RNF8; RNGTT; RNH1; RNLS; RNMT; RNPEP; RNPEPL1; RNPS1; ROBOT; ROBO2; ROBO3; ROBO4; ROCK1; ROCK2; ROGDI; ROM1; ROPN1B; ROPN1; ROPN1L; ROR1; ROR2; RORA; RORB; RORC; ROS1; RP1; RP1L1; RP2; RP9; RPA1; RPA2; RPA3; RPA4; RPAIN; RPAP1; RPE; RPGR; RPGRIP1; RPGRIP1L; RPH3A; RPH3AL; RPIA; RPL10A; RPL10; RPL10L; RPL12; RPL13; RPL14; RPL15; RPL17-C18orf32; RPL17; RPL18; RPL19; RPL21; RPL23A; RPL23; RPL24; RPL27A; RPL29; RPL30; RPL31; RPL34; RPL35; RPL36A; RPL36AL; RPL37A; RPL38; RPL39; RPL39L; RPL3; RPL41; RPL4; RPL6; RPL7A; RPL7; RPLP0; RPLP1; RPN1; RPN2; RPP14; RPP21; RPP25; RPP38; RPP40; RPRD1A; RPRD1B; RPRD2; RPRM; RPS10; RPS14; RPS15A; RPS16; RPS18; RPS19BP1; RPS20; RPS24; RPS27A; RPS27; RPS27L; RPS29; RPS2; RPS3A; RPS3; RPS4X; RPS4Y1; RPS6KA2; RPS6KA3; RPS6KA4; RPS6KA5; RPS6KA6; RPS6KB1; RPS6KB2; RPS9; RPSA; RPTOR; RQCD1; RRAD; RRAGA; RRAS2; RRAS; RRBP1; RREB1; RRH; RRM1; RRM2B; RRM2; RRN3; RRNAD1; RRP1B; RRP1; RRP9; RRS1; RS1; RSAD2; RSF1; RSL101; RSL24D1; RSPH1; RSPH4A; RSPH9; RSPO2; RSPO3; RSPO4; RSRC1; RSRC2; RSRP1; RSU1; RTCB; RTEL1; RTKN2; RTKN; RTL1; RTN1; RTN2; RTN3; RTN4; RTN4IP1; RTN4R; RTP3; RTP4; RTTN; RUFY1; RUFY3; RUNDC3B; RUNX1; RUNX1T1; RUNX2; RUNX3; RUVBL1; RUVBL2; RXFP1; RXFP2; RXFP3; RXRA; RXRB; RXRG; RYBP; RYK; RYR1; RYR2; RYR3; S100A10; S100A11; S100Al2; S100A13; S100A14; S100A16; S100A1; S100A2; S100A3; S100A4; S100A5; S100A6; S100A7A; S100A7; S100A8; S100A9; S100B; S100P; S100Z; S1PR2; S1PR3; S1PR4; S1PR5; SAA1; SAA2; SAA4; SAAL1; SAC3D1; SACM1L; SACS; SAE1; SAFB2; SAFB; SAGE1; SAG; SALL1; SALL2; SALL3; SALL4; SAMD14; SAMD1; SAMD4A; SAMD5; SAMD9; SAMD9L; SAMHD1; SAMM50; SAMSN1; SAP30BP; SAP30L; SAPCD1; SAPCD2; SAR1A; SAR1B; SARDH; SARM1; SARNP; SARS2; SART1; SART3; SASH1; SASH3; SAT1; SAT2; SATB1; SATB2; SATL1; SAV1; SBDS; SBF1; SBF2; SBNO1; SBNO2; SBSN; SC50; SCAF11; SCAF1; SCAF4; SCAF8; SCAI; SCAMP2; SCAMPS; SCAPER; SCAP; SCARA3; SCARA5; SCARB1; SCARB2; SCARF2; SCCPDH; SCD5; SCD; SCFD1; SCFD2; SCG2; SCG3; SCG5; SCGB1A1; SCGB1D1; SCGB1D2; SCGB2A1; SCGB2A2; SCGB2B2; SCGB3A1; SCGB3A2; SCGN; SCHIP1; SCIN; SCLT1; SCLY; SCML2; SCML4; SCN10A; SCN11A; SCN1A; SCN1B; SCN2A; SCN2B; SCN3A; SCN3B; SCN4A; SCN4B; SCN5A; SCN7A; SCN8A; SCN9A; SCNM1; SCNN1A; SCNN1B; SCNN1D; SCNN1G; SCO1; SCO2; SCP2D1; SCPEP1; SCRIB; SCRN1; SCT; SCTR; SCUBE1; SCUBE2; SCUBE3; SCYL1; SCYL3; SDC1; SDC2; SDC3; SDC4; SDCBP2; SDCBP; SDCCAG3; SDCCAG8; SDF2; SDF2L1; SDF4; SDHAF1; SDHAF2; SDHAF4; SDHA; SDHC; SDHD; SDK1; SDK2; SDPR; SDR42E1; SDR9C7; SDS; SEC11A; SEC11C; SEC13; SEC14L1; SEC14L2; SEC14L3; SEC16B; SEC23A; SEC23B; SEC23IP; SEC24A; SEC24B; SEC24C; SEC31A; SEC61A1; SEC61G; SEC62; SEC63; SECISBP2; SECISBP2L; SECTM1; SEL1L; SELE; SELENBP1; SELL; SELP; SELPLG; SEMA3A; SEMA3B; SEMA3C; SEMA3D; SEMA3E; SEMA3F; SEMA3G; SEMA4A; SEMA4B; SEMA4D; SEMA4F; SEMA4G; SEMA5A; SEMA5B; SEMA6A; SEMA6B; SEMA6D; SEMA7A; SEMG1; SEMG2; SENP1; SENP2; SENP3; SENP5; SENP6; SENP8; SEPHS1; SEPSECS; SERAC1; SERF1A; SERGEF; SERINC1; SERINC3; SERINC5; SERP1; SERP2; SERPINA10; SERPINA11; SERPINA12; SERPINA1; SERPINA3; SERPINA4; SERPINA5; SERPINA6; SERPINA7; SERPINA9; SERPINB10; SERPINB13; SERPINB1; SERPINB2; SERPINB3; SERPINB4; SERPINB5; SERPINB6; SERPINB7; SERPINB8; SERPINB9; SERPINC1; SERPIND1; SERPINE1; SERPINE2; SERPINE3; SERPINF1; SERPINF2; SERPING1; SERPINH1; SERPINI1; SERPINI2; SERTAD1; SERTAD2; SESN1; SESN3; SETBP1; SETD1A; SETD1B; SETD2; SETD3; SETD5; SETD7; SETD8; SETDB2; SET; SETMAR; SETX; SEZ6; SEZ6L2; SEZ6L; SF1; SF3A1; SF3B1; SF3B2; SF3B6; SFI1; SFMBT1; SFMBT2; SFR1; SFRP1; SFRP2; SFRP4; SFRP5; SFSWAP; SFT2D2; SFT2D3; SFTA2; SFTA3; SFTPA1; SFTPA2; SFTPB; SFTPC; SFTPD; SFXN1; SFXN2; SFXN4; SGCA; SGCB; SGCD; SGCE; SGCG; SGCZ; SGIP1; SGK1; SGK223; SGK2; SGK3; SGMS1; SGMS2; SGOL1; SGPL1; SGPP1; SGPP2; SGSH; SGSM2; SGSM3; SGTA; SH2B1; SH2B2; SH2B3; SH2D1A; SH2D2A; SH2D3A; SH2D3C; SH2D4A; SH2D4B; SH3BGR; SH3BGRL2; SH3BGRL; SH3BP1; SH3BP2; SH3BP4; SH3BP5; SH3D19; SH3GL1; SH3GL2; SH3GL3; SH3KBP1; SH3PXD2A; SH3PXD2B; SH3RF1; SH3RF3; SH3TC2; SH3YL1; SHANK1; SHANK2; SHANK3; SHARPIN; SHBG; SHB; SHC1; SHC2; SHC3; SHC4; SHCBP1; SHF; SHFM1; SHH; SHISA2; SHISA3; SHISA6; SHISA9; SHMT1; SHMT2; SHOC2; SHOX2; SHOX; SHPK; SHPRH; SHQ1; SHROOM2; SHROOM3; SHROOM4; SIAE; SIAH1; SIAH2; SIDT1; SIGIRR; SIGLEC11; SIGLEC14; SIGLEC1; SIGLECS; SIGLEC7; SIGLEC8; SIGLEC9; SIGMAR1; SI; SIK1; SIK2; SIK3; Sil1; SIM1; SIM2; SIN3A; SIN3B; SIPA1; SIPA1L2; SIPA1L3; SIRPA; SIRPB1; SIRPG; SIRT1; SIRT2; SIRT3; SIRT4; SIRTS; SIRT6; SIRT7; SITZ; SIVA1; SIX1; SIX2; SIX3; SIX4; SIX5; SIX6; SKA1; SKA2; SKAP1; SKAP2; SKIL; SKIV2L2; SKIV2L; SKOR1; SKOR2; SKP1; SKP2; SLA2; SLA; SLAIN2; SLAMF1; SLAMF6; SLAMF7; SLAMF8; SLBP; SLC10A1; SLC10A2; SLC10A6; SLC10A7; SLC11A1; SLC11A2; SLC12A1; SLC12A2; SLC12A3; SLC12A4; SLC12A5; SLC12A6; SLC12A7; SLC12A8; SLC12A9; SLC13A1; SLC13A2; SLC13A3; SLC13A5; SLC14A1; SLC14A2; SLC15A1; SLC15A2; SLC15A4; SLC16A10; SLC16A11; SLC16Al2; SLC16A13; SLC16A1; SLC16A2; SLC16A3; SLC16A6; SLC16A7; SLC16A8; SLC16A9; SLC17A1; SLC17A2; SLC17A3; SLC17A4; SLC17A5; SLC17A6; SLC17A7; SLC17A8; SLC18A1; SLC18A2; SLC18A3; SLC19A1; SLC19A2; SLC19A3; SLC1A1; SLC1A2; SLC1A3; SLC1A4; SLC1A5; SLC1A6; SLC20A1; SLC20A2; SLC22A11; SLC22Al2; SLC22A13; SLC22A14; SLC22A16; SLC22A17; SLC22A18AS; SLC22A18; SLC22A1; SLC22A23; SLC22A24; SLC22A2; SLC22A3; SLC22A4; SLC22A5; SLC22A6; SLC22A7; SLC22A8; SLC23A1; SLC23A2; SLC24A1; SLC24A2; SLC24A3; SLC24A4; SLC24A5; SLC25A10; SLC25Al2; SLC25A13; SLC25A14; SLC25A15; SLC25A16; SLC25A18; SLC25A19; SLC25A1; SLC25A20; SLC25A21; SLC25A22; SLC25A23; SLC25A25; SLC25A27; SLC25A2; SLC25A36; SLC25A37; SLC25A38; SLC25A3; SLC25A40; SLC25A41; SLC25A42; SLC25A43; SLC25A45; SLC25A46; SLC25A47; SLC25A4; SLC25A52; SLC25A5; SLC25A6; SLC26A1; SLC26A2; SLC26A3; SLC26A4; SLC26A5; SLC26A6; SLC26A7; SLC26A8; SLC26A9; SLC27A1; SLC27A2; SLC27A3; SLC27A4; SLC27A5; SLC28A1; SLC28A2; SLC28A3; SLC29A1; SLC29A2; SLC29A3; SLC29A4; SLC2A10; SLC2A11; SLC2Al2; SLC2A13; SLC2A14; SLC2A2; SLC2A3; SLC2A4RG; SLC2A5; SLC2A6; SLC2A8; SLC2A9; SLC30A10; SLC30A1; SLC30A3; SLC30A4; SLC30A5; SLC30A6; SLC30A7; SLC30A8; SLC30A9; SLC31A1; SLC31A2; SLC32A1; SLC33A1; SLC34A1; SLC34A2; SLC34A3; SLC35A1; SLC35A2; SLC35A3; SLC35A4; SLC35B2; SLC35B4; SLC35C1; SLC35D3; SLC35F1; SLC35F2; SLC35F3; SLC35F4; SLC35F6; SLC35G1; SLC35G2; SLC35G5; SLC35G6; SLC36A1; SLC36A2; SLC37A1; SLC37A2; SLC37A4; SLC38A1; SLC38A2; SLC38A4; SLC38A5; SLC38A6; SLC38A7; SLC38A8; SLC38A9; SLC39A10; SLC39A11; SLC39Al2; SLC39A13; SLC39A14; SLC39A1; SLC39A2; SLC39A3; SLC39A4; SLC39A6; SLC39A7; SLC39A8; SLC39A9; SLC3A1; SLC3A2; SLC40A1; SLC41A1; SLC43A1; SLC43A2; SLC43A3; SLC44A1; SLC44A2; SLC44A4; SLC44A5; SLC45A2; SLC45A3; SLC45A4; SLC46A1; SLC46A2; SLC47A1; SLC48A1; SLC4A10; SLC4A11; SLC4A1AP; SLC4A1; SLC4A2; SLC4A3; SLC4A4; SLC4A5; SLC4A7; SLC4A9; SLC50A1; SLC51A; SLC51B; SLC52A1; SLC52A2; SLC52A3; SLC5A11; SLC5Al2; SLC5A1; SLC5A2; SLC5A3; SLC5A4; SLC5A5; SLC5A6; SLC5A7; SLC5A8; SLC6A11; SLC6Al2; SLC6A13; SLC6A14; SLC6A15; SLC6A18; SLC6A19; SLC6A1; SLC6A20; SLC6A2; SLC6A4; SLC6A5; SLC6A6; SLC6A7; SLC6A8; SLC6A9; SLC7A10; SLC7A11; SLC7A13; SLC7A14; SLC7A1; SLC7A2; SLC7A3; SLC7A4; SLC7A5; SLC7A6; SLC7A7; SLC7A8; SLC7A9; SLC8A1; SLC8A2; SLC8A3; SLC9A1; SLC9A2; SLC9A3; SLC9A3R1; SLC9A3R2; SLC9A4; SLC9A5; SLC9A6; SLC9A7; SLC9A8; SLC9A9; SLC9B1; SLC9B2; SLC9C1; SLC9C2; SLCO1A2; SLCO1B1; SLCO1B3; SLCO1B7; SLCO1C1; SLCO2A1; SLCO2B1; SLCO3A1; SLCO4A1; SLCO4C1; SLCO5A1; SLCO6A1; SLFN12; SLFN12L; SLFN14; SLFN5; SLIT1; SLIT2; SLIT3; SLITRK1; SLITRK2; SLITRK3; SLITRK5; SLITRK6; SLK; SLMAP; SLMO2; SLN; SLPI; SLTM; SLUT; SLURP1; SLX4; SLX4IP; SMAD1; SMAD2; SMAD3; SMAD4; SMAD5; SMAD6; SMAD7; SMAD9; SMAGP; SMAP1; SMARCA1; SMARCA2; SMARCA4; SMARCA5; SMARCAD1; SMARCAL1; SMARCB1; SMARCC1; SMARCC2; SMARCD1; SMARCD3; SMARCE1; SMC1A; SMC1B; SMC2; SMC3; SMC4; SMC5; SMC6; SMCHD1; SMCO4; SMCP; SMEK1; SMEK2; SMG1; SMG6; SMG8; SMIM15; SMIM19; SMIM20; SMIM21; SMIM23; SMIM5; SMN2; SMOC1; SMOC2; SMO; SMOX; SMPD1; SMPD2; SMPD3; SMPDL3A; SMPDL3B; SMPX; SMR3B; SMS; SMTN; SMTNL1; SMU1; SMUG1; SMURF1; SMURF2; SMYD1; SMYD2; SMYD3; SMYD4; SMYD5; SNAI1; SNAI2; SNAI3; SNAP23; SNAP25; SNAP29; SNAP47; SNAP91; SNAPC1; SNAPC4; SNAPC5; SNCA; SNCAIP; SNCB; SNCG; SND1; SNED1; SNF8; SNIP1; SNRK; SNRNP200; SNRNP27; SNRNP70; SNRPA; SNRPB; SNRPC; SNRPD1; SNRPD3; SNRPE; SNRPF; SNRPN; SNTA1; SNTB1; SNTG1; SNTG2; SNUPN; SNORF; SNW1; SNX10; SNX12; SNX14; SNX16; SNX18; SNX19; SNX1; SNX20; SNX24; SNX25; SNX29; SNX2; SNX30; SNX3; SNX5; SNX9; SOAT1; SOAT2; SOBP; SOCS1; SOCS2; SOCS3; SOCS4; SOCS5; SOCS6; SOCS7; SOD1; SOD2; SOD3; SOHLH1; SOHLH2; SON; SORBS1; SORBS2; SORBS3; SORCS1; SORCS2; SORCS3; SORD; SORL1; SOS1; SOS2; SOSTDC1; SOST; SOX10; SOX11; SOX12; SOX13; SOX14; SOX15; SOX17; SOX18; SOX1; SOX21; SOX2; SOX3; SOX4; SOX5; SOX6; SOX7; SOX8; SOX9; SP100; SP110; SP140; SP1; SP2; SP3; SP4; SP5; SP6; SP7; SP8; SPA17; SPACA1; SPACA3; SPAG11A; SPAG11B; SPAG16; SPAG1; SPAG4; SPAG5; SPAG6; SPAG7; SPAG8; SPAG9; SPAM1; SPANXA2; SPANXB1; SPANXD; SPANXN4; SPARC; SPARCL1; SPAST; SPATA13; SPATA16; SPATA17; SPATA18; SPATA19; SPATA20; SPATA21; SPATA22; SPATA25; SPATA2; SPATA5; SPATA7; SPATA8; SPATA9; SPATC1; SPC24; SPC25; SPCS3; SPDEF; SPDL1; SPDYA; SPECC1; SPECC1L; SPEF2; SPEG; SPESP1; SPG11; SPG20; SPG21; SPG7; SPHK1; SPHK2; SPHKAP; SPIT; SPIB; SPIC; SPIDR; SPIN1; SPIN2A; SPINK1; SPINK2; SPINK4; SPINK5; SPINK6; SPINK7; SPINT1; SPINT2; SPNS1; SPNS2; SP011; SPOCK1; SPOCK2; SPOCK3; SPON1; SPON2; SPOP; SPP1; SPP2; SPPL2A; SPPL2B; SPPL2C; SPPL3; SPRED1; SPRED2; SPRED3; SPR; SPRN; SPRR1A; SPRR1B; SPRR2A; SPRR2B; SPRR3; SPRTN; SPRY1; SPRY2; SPRY3; SPRY4; SPRYD7; SPSB3; SPSB4; SPTA1; SPTAN1; SPTB; SPTBN1; SPTBN2; SPTBN4; SPTBN5; SPTLC1; SPTLC2; SPTLC3; SPTSSB; SPTY2D1; SPZ1; SQLE; SQRDL; SQSTM1; SRA1; SRBD1; SRCAP; SRC; SRCIN1; SRD5A1; SRD5A2; SRD5A3; SREBF1; SREBF2; SREK1; SREK1IP1; SRFBP1; SRF; SRGAP1; SRGAP2; SRGAP3; SRGN; SRI; SRL; SRMS; SRP14; SRP19; SRP68; SRP72; SRP9; SRPK1; SRPK2; SRPRB; SRPR; SRPX2; SRPX; SRR; SRRM1; SRRM2; SRRM4; SRRT; SRSF10; SRSF11; SRSF12; SRSF1; SRSF2; SRSF3; SRSF4; SRSF5; SRSF6; SRSF7; SRSF9; SRXN1; SRY; SS18; SS18L1; SSB; SSBP1; SSBP2; SSFA2; SSH1; SSH2; SSMEM1; SSNA1; SSPN; SSPO; SSR1; SSR2; SSRP1; SSSCA1; SST; SSTR1; SSTR2; SSTR3; SSTR4; SSTR5; SSUH2; SSX1; SSX2B; SSX2IP; SSX4B; SSX5; ST13; ST14; ST18; ST20; ST3GAL1; ST3GAL2; ST3GAL4; ST3GAL5; ST3GAL6; ST5; ST6GAL1; ST6GAL2; ST6GALNAC1; ST6GALNAC2; ST6GALNAC4; ST6GALNAC6; ST7; ST7L; ST8SIA1; ST8SIA2; ST8SIA3; ST8SIA4; ST8SIA6; STAB1; STAB2; STAC3; STAC; STAG1; STAG2; STAG3; STAM2; STAMBP; STAMBPL1; STAM; STAP1; STAP2; STARD10; STARD13; STARD3; STARD3NL; STARD5; STARD7; STARD8; STARD9; STAR; STAT1; STAT2; STAT3; STAT4; STAT5A; STAT5B; STATE; STATH; STAU2; STBD1; STC1; STC2; STEAP1; STEAP2; STEAP3; STEAP4; STH; STIL; STIM1; STIM2; STIP1; STK10; STK11; STK11IP; STK17A; STK17B; STK19; STK24; STK25; STK26; STK31; STK32A; STK32B; STK32C; STK33; STK35; STK38L; STK39; STK3; STK4; STMN2; STMN3; STMN4; STOM; STOML1; STOML2; STOML3; STON1-GTF2A1L; STON1; STOX1; STRA13; STRA6; STRA8; STRADA; STRADB; STRAP; STRC; STRN3; STRN4; STRN; STS; STT3A; STT3B; STUB1; STX11; STX16; STX17; STX18; STX1A; STX1B; STX2; STX3; STX4; STX5; STX6; STX8; STXBP1; STXBP2; STXBP4; STXBP5; STXBP5L; STXBP6; STYK1; STYX; SUB1; SUCLA2; SUCLG1; SUCLG2; SUCNR1; SUCO; SUDS3; SUFU; SUGCT; SUGP1; SUGT1; SULF1; SULF2; SULT1A1; SULT1A2; SULT1A4; SULT1B1; SULT1C2; SULT1E1; SULT2A1; SULT2B1; SULT4A1; SUMF1; SUMF2; SUMO1; SUMO2; SUMO3; SUMO4; SUN1; SUN2; SUN3; SUOX; SUPT20H; SUPT3H; SUPT4H1; SUPT7L; SUPV3L1; SURF1; SURF4; SUSD1; SUSD2; SUSD4; SUSD6; SUV39H1; SUV39H2; SUV420H2; SUZ12; SV2B; SV2C; SVEP1; SVIL; SVIP; SVOP; SWAP70; SWT1; SYBU; SYCE1; SYCE1L; SYCP2; SYCP2L; SYCP3; SYK; SYMPK; SYN1; SYN2; SYN3; SYNCRIP; SYNDIG1; SYNE1; SYNE2; SYNE4; SYNGAP1; SYNGR1; SYNGR2; SYNJ1; SYNJ2BP; SYNJ2; SYNM; SYNPO2; SYNPO; SYNPR; SYP; SYPL1; SYPL2; SYT11; SYT12; SYT13; SYT14; SYT1; SYT4; SYT6; SYT9; SYTL1; SYTL2; SYTL5; SYVN1; TAAR1; TAAR2; TAAR5; TAAR6; TAB1; TAB2; TAB3; TACT; TAC3; TAC4; TACC1; TACC2; TACC3; TACO1; TACR1; TACR2; TACR3; TADA1; TADA2A; TADA3; TAF15; TAF1B; TAF1C; TAF1D; TAF1; TAF2; TAF3; TAF4B; TAF4; TAF5L; TAF6; TAF7; TAF7L; TAF8; TAF9; TAGAP; TAGLN2; TAGLN; TALI; TAL2; TALDO1; TAMM41; TANC1; TANC2; TANGO2; TANK; TAOK1; TAOK2; TAOK3; TAP1; TAP2; TAPBP; TAPBPL; TARBP1; TARBP2; TARP; TARS; TARSL2; TAS2R10; TAS2R13; TAS2R14; TAS2R16; TAS2R1; TAS2R38; TAS2R50; TAS2R60; TAS2R9; TASP1; TATDN1; TAT; TAX1BP1; TAX1BP3; TAZ; TBATA; TBC1D15; TBC1D16; TBC1D1; TBC1D20; TBC1D22A; TBC1D22B; TBC1D24; TBC1D25; TBC1D2; TBC1D32; TBC1D3C; TBC1D3F; TBC1D4; TBC1D5; TBC1D7; TBC1D8; TBC1D9; TBCA; TBCC; TBCD; TBCE; TBCEL; TBK1; TBKBP1; TBL1X; TBL1XR1; TBL1Y; TBL2; TBL3; TBP; TBPL1; TBPL2; TBR1; TBRG1; TBX10; TBX18; TBX19; TBX1; TBX20; TBX21; TBX22; TBX2; TBX3; TBX4; TBX5; TBX6; TBXA2R; TBXAS1; TCAIM; TCAP; TCEA1; TCEA2; TCEA3; TCEAL1; TCEAL2; TCEAL4; TCEAL7; TCEB1; TCEB2; TCEB3C; TCERG1; TCERG1L; TCF12; TCF15; TCF19; TCF20; TCF21; TCF25; TCF3; TCF4; TCF7; TCF7L1; TCF7L2; TCFL5; TCHH; TCHP; TCIRG1; TCL1A; TCL1B; TCN1; TCN2; TCOF1; TCP10; TCP10L2; TCP11L1; TCP1; TCTA; TCTE1; TCTN1; TCTN2; TCTN3; TDGF1; TDG; TDO2; TCP1; TCP2; TDRD1; TDRD3; TDRD5; TDRD6; TDRD7; TDRD9; TDRKH; TDRP; TEAD1; TEAD2; TEAD3; TEAD4; TEC; TECPR2; TECR; TECRL; TECTA; TEF; TEFM; TEK; TEKT1; TEKT5; TELO2; TENM1; TENM2; TENM3; TENM4; TEP1; TEPP; TERF1; TERF2; TERF2IP; TERT; TESC; TES; TESPA1; TET1; TET2; TET3; TEX101; TEX11; TEX14; TEX15; TEX264; TEX29; TEX30; TEX35; TEX40; TFAM; TFAP2A; TFAP2B; TFAP2C; TFAP4; TFB1M; TFB2M; TFCP2; TFDP1; TFDP2; TFDP3; TFE3; TFEB; TFEC; TFF1; TFF2; TFF3; TFG; TF; TFIP11; TFPI2; TFPI; TFPT; TFR2; TFRC; TGFA; TGFB1; TGFB1I1; TGFB2; TGFB3; TGFBI; TGFBR1; TGFBR2; TGFBRAP1; TG; TGIF1; TGIF2-C20orf24; TGIF2; TGIF2LX; TGM1; TGM2; TGM3; TGM4; TGM5; TGM6; TGM7; TGOLN2; TGS1; THADA; THAP10; THAP11; THAP1; THAP2; THAP6; THBD; THBS1; THBS2; THBS3; THBS4; THEG; THEM4; THEM5; THEME; THEMIS2; THEMIS; THG1L; TH; THNSL1; THNSL2; THOC1; THOC2; TH005; THOC6; T; THOP1; THPO; THRA; THRB; THRSP; THSD1; THSD4; THSD7A; THYN1; TIAL; TIAF1; TIAL1; TIAM1; TIAM2; TICAM1; TICAM2; TICRR; TIFA; TIGD2; TIGIT; TIMD4; TIMELESS; TIMM10; TIMM17A; TIMM21; TIMM22; TIMM23; TIMM44; TIMM50; TIMM8A; TIMM8B; TIMMDC1; TIMP1; TIMP2; TIMP3; TIMP4; TINAG; TINF2; TIPARP; TIPIN; TIPRL; TIRAP; TJP1; TJP2; TJP3; TK1; TK2; TKT; TKTL1; TKTL2; TLDC1; TLE1; TLE2; TLE3; TLE4; TLE6; TLK1; TLK2; TLL1; TLL2; TLN1; TLN2; TLR10; TLR1; TLR2; TLR3; TLR4; TLR5; TLR6; TLR7; TLR8; TLR9; TLX1; TLX2; TLX3; TM4SF1; TM4SF20; TM4SF4; TM4SF5; TM6SF2; TM7SF2; TM9SF2; TM9SF4; TMBIM4; TMBIM6; TMC1; TMC2; TMC3; TMC5; TMC6; TMC8; TMCC1; TMCC2; TMCC3; TMCO1; TMCO4; TMCO5A; TMED10; TMED1; TMED2; TMED3; TMED4; TMED7; TMED7-TICAM2; TMED9; TMEFF1; TMEFF2; TMEM100; TMEM101; TMEM105; TMEM106B; TMEM108; TMEM114; TMEM115; TMEM117; TMEM11; TMEM126A; TMEM127; TMEM128; TMEM132A; TMEM132B; TMEM132C; TMEM1320; TMEM132E; TMEM134; TMEM135; TMEM138; TMEM150B; TMEM151A; TMEM151B; TMEM154; TMEM158; TMEM160; TMEM161B; TMEM163; TMEM165; TMEM169; TMEM170A; TMEM171; TMEM173; TMEM175; TMEM176B; TMEM178A; TMEM182; TMEM183A; TMEM184C; TMEM185A; TMEM187; TMEM189; TMEM189-UBE2V1; TMEM18; TMEM199; TMEM200A; TMEM205; TMEM207; TMEM209; TMEM213; TMEM215; TMEM216; TMEM217; TMEM219; TMEM220; TMEM229A; TMEM231; TMEM233; TMEM237; TMEM241; TMEM244; TMEM245; TMEM259; TMEM25; TMEM261; TMEM27; TMEM2; TMEM30A; TMEM30B; TMEM37; TMEM38A; TMEM38B; TMEM39A; TMEM40; TMEM43; TMEM45A; TMEM47; TMEM50B; TMEM55A; TMEM57; TMEM5; TMEM60; TMEM62; TMEM63A; TMEM67; TMEM70; TMEM74B; TMEM79; TMEM87A; TMEM88; TMEM89; TMEM8B; TMEM95; TMEM97; TMEM98; TMF1; TMIE; TMIGD2; TMIGD3; TMLHE; TMOD1; TMOD2; TMOD3; TMOD4; TMPO; TMPRSS11A; TMPRSS11B; TMPRSS110; TMPRSS11E; TMPRSS13; TMPRSS15; TMPRSS2; TMPRSS3; TMPRSS4; TMPRSS6; TMPRSS7; TMPRSS9; TMSB10; TMSB15B; TMSB4X; TMTC1; TMTC2; TMTC3; TMX1; TMX2; TMX3; TNC; TNFAIP1; TNFAIP2; TNFAIP3; TNFAIP6; TNFAIP8; TNFAIP8L2; TNFAIP8L3; TNF; TNFRSF10A; TNFRSF10B; TNFRSF10C; TNFRSF100; TNFRSF11A; TNFRSF11B; TNFRSF12A; TNFRSF13B; TNFRSF13C; TNFRSF14; TNFRSF19; TNFRSF1A; TNFRSF1B; TNFRSF21; TNFRSF6B; TNFRSF8; TNFSF10; TNFSF11; TNFSF12; TNFSF12-TNFSF13; TNFSF13B; TNFSF13; TNFSF14; TNFSF15; TNFSF18; TNFSF4; TNFSF8; TNFSF9; TNIK; TNIP1; TNIP2; TNIP3; TNK1; TNK2; TNKS2; TNKS; TNMD; TNNC1; TNN; TNNI1; TNNI2; TNNI3; TNNT1; TNNT2; TNNT3; TNP1; TNP2; TNPO1; TNPO2; TNPO3; TNRC18; TNRC6A; TNRC6B; TNR; TNS1; TNS2; TNS3; TNS4; TNXB; TOB1; TOB2; TOLLIP; TOM1; TOM1L1; TOMM20; TOMM34; TOMM40; TOMM70A; TONSL; TOP1; TOP2A; TOP2B; TOP3A; TOP3B; TOPBP1; TOPORS; TOR1A; TOR1AIP1; TOR1AIP2; TOR1B; TOR2A; TOX2; TOX3; TOX4; TOX; TP53AIP1; TP53BP1; TP53BP2; TP53; TP53I11; TP53I13; TP53I3; TP53INP1; TP53INP2; TP53RK; TP53TG3C; TP63; TP73; TPBG; TPCN1; TPCN2; TPD52; TPD52L1; TPD52L2; TPGS2; TPH1; TPH2; TPI1; TPK1; TPM1; TPM2; TPM3; TPM4; TPMT; TPO; TPP1; TPP2; TPPP2; TPPP3; TPPP; TPRG1; TPR; TPRN; TPSAB1; TPSB2; TPSD1; TPSG1; TPT1; TPTE2; TPTE; TPX2; TRA2A; TRA2B; TRABD2A; TRABD; TRADD; TRAF1; TRAF2; TRAF3; TRAF3IP1; TRAF3IP2; TRAF4; TRAF5; TRAF6; TRAF7; TRAFD1; TRAK1; TRAK2; TRAM1; TRAM1L1; TRAM2; TRAP1; TRAPPC10; TRAPPC11; TRAPPC1; TRAPPC2; TRAPPC4; TRAPPC9; TRAT1; TRDMT1; TRDN; TREH; TREM1; TREM2; TREML1; TREML2; TRERF1; TREX1; TREX2; TRHDE; TRH; TRHR; TRIAP1; TRIB1; TRIB2; TRIB3; TRIM10; TRIM11; TRIM13; TRIM15; TRIM16; TRIM17; TRIM21; TRIM22; TRIM23; TRIM24; TRIM25; TRIM26; TRIM27; TRIM28; TRIM29; TRIM2; TRIM31; TRIM32; TRIM34; TRIM35; TRIM36; TRIM37; TRIM38; TRIM39; TRIM3; TRIM40; TRIM42; TRIM44; TRIM50; TRIM56; TRIM58; TRIM59; TRIM5; TRIM62; TRIM66; TRIM68; TRIM69; TRIM6-TRIM34; TRIM71; TRIM72; TRIM73; TRIM74; TRIM8; TRIM9; TRIOBP; TRIO; TRIP10; TRIP11; TRIP13; TRIP4; TRIPE; TRIQK; TRMT10A; TRMT12; TRMT1; TRMT44; TRMT5; TRMU; TRO; TROVE2; TRPA1; TRPC1; TRPC3; TRPC4AP; TRPC4; TRPC5; TRPC6; TRPC7; TRPM1; TRPM2; TRPM3; TRPM4; TRPM5; TRPM6; TRPM7; TRPM8; TRPS1; TRPV1; TRPV2; TRPV3; TRPV4; TRPV5; TRPV6; TRRAP; TSACC; TSC1; TSC22D1; TSC22D3; TSC22D4; TSC2; TSEN2; TSEN34; TSEN54; TSFM; TSG101; TSGA10; TSHR; TSHZ1; TSHZ2; TSHZ3; TSLP; TSNAX; TSN; TSPAN10; TSPAN11; TSPAN12; TSPAN13; TSPAN14; TSPAN16; TSPAN18; TSPAN31; TSPAN32; TSPAN33; TSPAN4; TSPAN6; TSPAN7; TSPAN8; TSPAN9; TSPEAR; TSPO2; TSPO; TSPY10; TSPY1; TSPY3; TSPY4; TSPYL1; TSPYL2; TSPYL4; TSPYL5; TSR1; TSSC1; TSSK1B; TSSK2; TSSK4; TSTA3; TSTD1; TST; TTBK1; TTBK2; TTC12; TTC17; TTC19; TTC1; TTC21B; TTC28; TTC29; TTC37; TTC39A; TTC39B; TTC3; TTC5; TTC6; TTC7A; TTC7B; TTC8; TTC9B; TTC9C; TTC9; TTF1; TTF2; TTI1; TTI2; TTK; TTL; TTLL10; TTLL11; TTLL12; TTLL1; TTLL3; TTLL4; TTLL5; TTLL6; TTLL7; TTLL8; TTLL9; TTPA; TTR; TTYH1; TTYH2; TUBA1A; TUBA1B; TUBA1C; TUBA30; TUBA4A; TUBAE; TUBB1; TUBB2A; TUBB2B; TUBB3; TUBB4A; TUBB4B; TUBB6; TUBB; TUBD1; TUBE1; TUBG1; TUBG2; TUBGCP2; TUBGCP3; TUBGCP4; TUBGCP5; TUBGCP6; TUB; TUFM; TUFT1; TULP1; TULP2; TULP3; TULP4; TUSC1; TUSC2; TUSC3; TUSC5; TUT1; TVP23B; TWF1; TWIST1; TWIST2; TWSG1; TXK; TXLNG; TXN2; TXNDC15; TXNDC16; TXNDC17; TXNDC5; TXN; TXNL1; TXNRD2; TXNRD3NB; TYK2; TYMP; TYMS; TYR; TYRO3; TYROBP; TYRP1; TYSND1; TYW1B; U2AF1; U2AF2; UACA; UAP1; UBA1; UBA2; UBA3; UBA7; UBAC1; UBAC2; UBAP1; UBAP2; UBASH3A; UBASH3B; UBB; UBC; UBD; UBE2A; UBE2B; UBE2C; UBE2D1; UBE2D2; UBE2D3; UBE2E1; UBE2E2; UBE2E3; UBE2G1; UBE2G2; UBE2H; UBE2I; UBE2J1; UBE2K; UBE2L3; UBE2L6; UBE2M; UBE2N; UBE2Q2; UBE2QL1; UBE2R2; UBE2S; UBE2T; UBE2U; UBE2V1; UBE2V2; UBE2Z; UBE3A; UBE3B; UBE3C; UBE4A; UBE4B; UBIAD1; UBL3; UBL4A; UBL5; UBL7; UBLCP1; UBN1; UBOX5; UBP1; UBQLN1; UBQLN2; UBQLNL; UBR1; UBR3; UBR4; UBR5; UBR7; UBTD2; UBTF; UBXN1; UBXN2A; UBXN2B; UBXN4; UCHL1; UCHL3; UCHL5; UCK1; UCK2; UCKL1; UCMA; UCN2; UCN3; UCN; UCP1; UCP2; UCP3; UEVLD; UFD1L; UFL1; UFM1; UGCG; UGDH; UGGT1; UGGT2; UGP2; UGT1A10; UGT1A1; UGT1A3; UGT1A4; UGT1A5; UGT1A6; UGT1A7; UGT1A8; UGT1A9; UGT2A1; UGT2A2; UGT2A3; UGT2B10; UGT2B11; UGT2B15; UGT2B28; UGT2B4; UGT2B7; UGT3A2; UGT8; UHMK1; UHRF1BP1; UHRF1; UHRF2; UIMC1; ULBP1; ULBP2; ULBP3; ULK1; ULK2; ULK3; ULK4; UMOD; UMODL1; UMPS; UNC119; UNC13A; UNC13B; UNC13C; UNC13D; UNC45A; UNC45B; UNC5A; UNC5B; UNC5C; UNC5D; UNC79; UNC93A; UNC93B1; UNG; UPB1; UPF1; UPF2; UPF3A; UPF3B; UPK1A; UPK1B; UPK2; UPK3A; UPP1; UPP2; UPRT; UQCC1; UQCC2; UQCRB; UQCRC1; UQCRC2; UQCRFS1; UQCRQ; URB2; URGCP; URI1; URM1; UROC1; UROD; UROS; USB1; USE1; USF1; USF2; USH1C; USH1G; USH2A; USO1; USP10; USP11; USP12; USP13; USP14; USP15; USP17L2; USP17L30; USP18; USP20; USP22; USP25; USP26; USP28; USP2; USP32; USP33; USP36; USP37; USP3; USP40; USP42; USP43; USP44; USP46; USP48; USP49; USP4; USP5; USP6; USP6NL; USP7; USPS; USP9X; USP9Y; USPL1; UST; UTF1; UTP14A; UTP14C; UTP20; UTRN; UTS2B; UTS2; UTS2R; UTY; UVRAG; UVSSA; UXS1; UXT; VAC14; VAMP1; VAMP2; VAMP4; VAMP7; VAMP8; VANGL2; VAPA; VAPB; VARS2; VARS; VASH1; VASH2; VASP; VAT1; VAT1L; VAV1; VAV2; VAV3; VAX1; VAX2; VBP1; VCAN; VCL; VCP; VCX2; VCX3A; VCX3B; VCX; VCY; VDAC1; VDAC2; VDR; VEGFA; VEGFB; VEGFC; VENTX; VEPH1; VEZT; VGF; VGLL1; VGLL2; VGLL3; VGLL4; VHL; VHLL; VIL1; VIM; VIPAS39; VIP; VIPR1; VIPR2; VIT; VKORC1; VLDLR; VMA21; VMO1; VMP1; VN1R2; VN1R4; VNN1; VNN2; VNN3; VOPP1; VPRBP; VPREB1; VPREB3; VPS11; VPS13A; VPS13B; VPS13C; VPS26A; VPS28; VPS33A; VPS33B; VPS35; VPS36; VPS37A; VPS37B; VPS37C; VPS39; VPS41; VPS4A; VPS4B; VPS51; VPS52; VPS53; VPS54; VPS72; VPS8; VRK1; VRK2; VSIG10; VSIG1; VSIG2; VSIG4; VSNL1; VSTM1; VSX1; VSX2; VTA1; VTCN1; VTI1A; VTI1B; VTN; VWA2; VWA3A; VWA3B; VWA5A; VWA5B1; VWA7; VWA8; VWCE; VWDE; VWF; WAPAL; WARS2; WARS; WASF1; WASF3; WASH1; WAS; WASL; WBP1L; WBP2; WBSCR17; WBSCR22; WDFY2; WDFY4; WDHD1; WDPCP; WDR11; WDR12; WDR17; WDR19; WDR1; WDR20; WDR26; WDR31; WDR34; WDR35; WDR36; WDR37; WDR43; WDR45B; WDR45; WDR46; WDR48; WDR49; WDR4; WDR55; WDR5; WDR60; WDR62; WDR64; WDR66; WDR70; WDR72; WDR74; WDR76; WDR78; WDR7; WDR81; WDR83; WDR86; WDR93; WEE1; WFDC1; WFDC2; WFS1; WHSC1; WHSC1L1; WIF1; WIPF1; WIPF2; WIPF3; WIPI1; WIPI2; WISP1; WISP2; WISP3; WLS; WNK1; WNK2; WNK3; WNK4; WNT10A; WNT10B; WNT11; WNT16; WNT1; WNT2B; WNT2; WNT3A; WNT3; WNT4; WNT5A; WNT5B; WNT6; WNT7A; WNT7B; WNT8A; WNT8B; WNT9A; WNT9B; WRAP53; WRB; WRN; WRNIP1; WSB1; WSCD1; WSCD2; WT1; WTAP; WTIP; WWC1; WWC2; WWOX; WWP1; WWP2; WWTR1; XAB2; XAF1; XAGE1B; XAGE1E; XBP1; XCL1; XCL2; XCR1; XDH; XG; XIAP; XIRP1; XIRP2; XK; XKR4; XKR6; XKR9; XPA; XPC; XPNPEP1; XPNPEP2; XPNPEP3; XPO1; XPO4; XPO5; XPO6; XPO7; XPR1; XRCC1; XRCC2; XRCC3; XRCC4; XRCC5; XRCC6BP1; XRCC6; XRN1; XRN2; XRRA1; XXYLT1; XYLB; XYLT1; XYLT2; YAE1D1; YAP1; YARS2; YBX2; YBX3; YDJC; YEATS4; YES1; YIF1A; YIPF1; YIPF3; YIPF5; YKT6; YLPM1; YME1L1; YPEL1; YPEL2; YPEL3; YPEL4; YPEL5; YTHDC1; YTHDC2; YWHAB; YWHAE; YWHAG; YWHAH; YWHAQ; YWHAZ; YY1AP1; YY1; ZACN; ZAK; ZAP70; ZAR1; ZAR1L; ZASP; ZBED1; ZBED4; ZBED5; ZBP1; ZBTB10; ZBTB12; ZBTB14; ZBTB16; ZBTB17; ZBTB18; ZBTB20; ZBTB21; ZBTB22; ZBTB24; ZBTB2; ZBTB32; ZBTB33; ZBTB34; ZBTB38; ZBTB41; ZBTB46; ZBTB48; ZBTB49; ZBTB4; ZBTB5; ZBTB7C; ZBTB9; ZC2HC1B; ZC3H10; ZC3H11A; ZC3H12C; ZC3H12D; ZC3H14; ZC3H15; ZC3H3; ZC3H4; ZC3H7A; ZC3H7B; ZC3HAV1; ZC3HC1; ZC4H2; ZCCHC11; ZCCHC12; ZCCHC14; ZCCHC2; ZCCHC3; ZCCHC6; ZCCHC8; ZCRB1; ZCWPW1; ZDBF2; ZDHHC11; ZDHHC12; ZDHHC13; ZDHHC14; ZDHHC15; ZDHHC17; ZDHHC1; ZDHHC2; ZDHHC7; ZDHHC8; ZDHHC9; ZEB1; ZEB2; ZFAND3; ZFAND5; ZFAND6; ZFAT; ZFC3H1; ZFHX2; ZFHX3; ZFHX4; ZFP1; ZFP30; ZFP36; ZFP36L1; ZFP36L2; ZFP37; ZFP42; ZFP57; ZFP64; ZFP82; ZFP91; ZFPM1; ZFPM2; ZFR2; ZFR; ZFX; ZFY; ZFYVE19; ZFYVE21; ZFYVE26; ZFYVE27; ZFYVE28; ZFYVE9; ZG16B; ZGLP1; ZGPAT; ZHX1; ZHX2; ZIC1; ZIC2; ZIC3; ZIC4; ZIC5; ZIK1; ZIM2; ZKSCAN1; ZKSCAN3; ZKSCAN7; ZMAT3; ZMAT4; ZMIZ1; ZMYM2; ZMYM3; ZMYM4; ZMYM5; ZMYND10; ZMYND11; ZMYND8; ZNF106; ZNF107; ZNF10; ZNF112; ZNF121; ZNF131; ZNF132; ZNF133; ZNF141; ZNF143; ZNF146; ZNF148; ZNF154; ZNF160; ZNF169; ZNF175; ZNF177; ZNF182; ZNF184; ZNF185; ZNF189; ZNF197; ZNF202; ZNF205; ZNF20; ZNF212; ZNF214; ZNF215; ZNF217; ZNF224; ZNF22; ZNF230; ZNF236; ZNF239; ZNF23; ZNF248; ZNF24; ZNF253; ZNF260; ZNF263; ZNF264; ZNF266; ZNF267; ZNF268; ZNF273; ZNF274; ZNF276; ZNF277; ZNF280B; ZNF280D; ZNF281; ZNF282; ZNF286B; ZNF296; ZNF2; ZNF300; ZNF311; ZNF318; ZNF320; ZNF322; ZNF32; ZNF330; ZNF331; ZNF334; ZNF335; ZNF343; ZNF350; ZNF354A; ZNF35; ZNF365; ZNF366; ZNF367; ZNF382; ZNF383; ZNF384; ZNF385A; ZNF385B; ZNF385D; ZNF391; ZNF395; ZNF398; ZNF407; ZNF410; ZNF415; ZNF419; ZNF41; ZNF423; ZNF430; ZNF432; ZNF433; ZNF438; ZNF443; ZNF444; ZNF44; ZNF451; ZNF45; ZNF462; ZNF469; ZNF483; ZNF490; ZNF492; ZNF496; ZNF501; ZNF507; ZNF512B; ZNF512; ZNF513; ZNF516; ZNF519; ZNF521; ZNF536; ZNF555; ZNF559; ZNF568; ZNF569; ZNF577; ZNF580; ZNF581; ZNF582; ZNF583; ZNF585B; ZNF592; ZNF596; ZNF606; ZNF607; ZNF608; ZNF615; ZNF618; ZNF627; ZNF629; ZNF639; ZNF644; ZNF645; ZNF646; ZNF652; ZNF654; ZNF664; ZNF667; ZNF668; ZNF674; ZNF676; ZNF678; ZNF683; ZNF687; ZNF689; ZNF703; ZNF704; ZNF706; ZNF711; ZNF716; ZNF717; ZNF746; ZNF74; ZNF750; ZNF763; ZNF764; ZNF765; ZNF76; ZNF774; ZNF776; ZNF778; ZNF784; ZNF79; ZNF7; ZNF800; ZNF804A; ZNF804B; ZNF80; ZNF812; ZNF813; ZNF816; ZNF81; ZNF823; ZNF827; ZNF829; ZNF831; ZNF91; ZNF92; ZNF93; ZNF98; ZNFX1; ZNHIT2; ZNHIT3; ZNRD1; ZNRF3; ZP1; ZP4; ZPBP2; ZPLD1; ZPR1; ZRANB3; ZRSR2; ZSCAN18; ZSCAN22; ZSCAN26; ZSCAN31; ZSCAN32; ZSCAN9; ZSWIM2; ZSWIM6; ZW10; ZWILCH; ZWINT; ZYX; ZZEF1; ZZZ3, and a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 demonstrates reduction of B16 melanoma growth by intravenous administration of Interferon Gamma-producing fibroblasts.

FIG. 2 demonstrates reduction of LLC lung cancer growth by intravenous administration of Interferon Gamma-producing fibroblasts.

### DETAILED DESCRIPTION

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

“Administering,” as used herein, refers to a method of giving a dosage of a composition described herein The compositions utilized in the methods described herein can be administered, for example, intravenously, intramuscularly, subcutaneously, orally, by inhalation, parenterally, intraperitoneally, intraarterially, transdermally, sublingually, nasally, transbuccally, liposomally, adiposally, ophthalmically, intraocularly, intrathecally, topically, or locally. Administration can be systemic or local. A chosen method of administration can vary depending on, for example, the components of the composition being administered and the severity of the condition (e.g., the wound) being treated.

“Antibody,” as used herein, includes whole antibodies or immunoglobulins and any antigen-binding fragment or single chains thereof. Antibodies, as used herein, can be mammalian (e.g., human or mouse), humanized, chimeric, recombinant, synthetically produced, or naturally isolated. Antibodies of the present invention include all known forms of antibodies and other protein scaffolds with antibody-like properties. For example, the antibody can be a human antibody, a humanized antibody, a bispecific antibody, a chimeric antibody, or a protein scaffold with antibody-like properties, such as fibronectin or ankyrin repeats. The antibody also can be a Fab, F(ab′).sub.2, scFv, SMIP, diabody, nanobody, aptamers, or a domain antibody. The antibody can have any of the following isotypes: IgG (e.g., IgG1, IgG2, IgG3, and IgG4), IgM, IgA (e.g., IgA1, IgA2, and IgAsec), IgD, or IgE.

“Antibody fragment,” as used herein, refers to one or more fragments of an antibody that retain the ability to specifically bind to an antigen. The antigen-binding function of an antibody can be performed by fragments of a full-length antibody. Examples of binding fragments encompassed within the term “antigen-binding portion” of an antibody include but are not limited to: (i) a Fab fragment, a monovalent fragment consisting of the V.sub.L, V.sub.H, C.sub.L, and C.sub.H1 domains; (ii) a F(ab′).sub.2 fragment, a bivalent fragment comprising two Fab fragments linked by a disulfide bridge at the hinge region; (iii) a Fd fragment consisting of the V.sub.H and C.sub.H1 domains; (iv) a Fv fragment consisting of the V.sub.L and V.sub.H domains of a single arm of an antibody, (v) a dAb including V.sub.H and V.sub.L domains; (vi) a dAb fragment (Ward et al., Nature 341:544-546 (1989)), which consists of a V.sub.H domain; (vii) a dAb which consists of a V.sub.H or a V.sub.L domain; (viii) an isolated complementarity determining region (CDR); and (ix) a combination of two or more isolated CDRs which may optionally be joined by a synthetic linker.

“Express” and “expression” refer to the process by which information (e.g., genetic and/or epigenetic information) is converted into the structures present in a cell or secreted therefrom. Accordingly, as used herein, “expression” may refer to transcription, translation, or polynucleotide and/or polypeptide modifications (e.g., posttranslational modification of a polypeptide).

“Genetically modified fibroblast” is a fibroblast cell that recombinantly expresses a modification to gene expression compared to its native state. Gene modification can be at least (1) enhanced expression of one or more genes endogenous to the cell (such as by modifying one or more regulatory sequences for the gene); (2) suppressed expression of one or more genes endogenous to the cell (such as through RNA interference or gene editing, for example with CRISPR); and/or (3) expression of one or more recombinant genes (whether or not the gene is also present as a genomic, endogenous copy in the cell), such as from a transfected vector that may or may not be integrating into the fibroblast genome.

“Parenteral” as used herein refers to subcutaneous, intracutaneous, intravenous, intraperitoneal, intramuscular, intraarticular, intraarterial, intrasynovial, intrasternal, intrathecal, intralesional, or intracranial administration (e.g., injection), as well as any suitable infusion technique.

“Pharmaceutical composition” as used herein refers to any composition that comprises one or more therapeutically or biologically active agents such as cells and/or conditioned media thereof.

“Treating” as used herein refers to a reduction (e.g., by at least about 10%, about 15%, about 20%, about 25%, about 30%, about 35%, about 40%, about 45%, about 50%, about 60%, about 70%, about 80%, about 90%, about 95%, about 99%, or even 100%) in the progression or severity of a disease or disorder (e.g., a wound), or in the progression, severity, or frequency of one or more symptoms of the disease or disorder (e.g., a wound) in a subject (e.g., a human), and including a delay in onset of the disease or disorder.

Embodiments of the disclosure encompass the manipulation of fibroblasts for the purpose of enhancing transfection of the fibroblasts and/or for the purpose of generating fibroblasts with enhanced activity following transfection. In specific embodiments, the fibroblasts are exposed to acidic conditions, in particular for the purpose of enhancing the transfection of the fibroblasts and/or activity of the gene modified fibroblasts. The methods result in gene modified fibroblasts. In specific embodiments, following the manipulation an effective amount of the fibroblasts are provided to an individual in need thereof. The exposure of the fibroblasts to the acidic pH conditions may be for an entire duration of culturing of the fibroblasts or for part of a entire duration of culturing of the fibroblasts, and in either case occurs before gene modification, concurrent with gene modification, after gene modification, or a combination thereof.

The disclosure provides for culture of fibroblasts in an acidic pH incubation medium before gene modification, concurrent with gene modification, after gene modification, or a combination thereof, in order to enhance therapeutic activity of gene modification. Within the practice of the disclosure, cells may be cultured at pH between 4.6-6.5, 4.8-6.5, 5.0-6.5, 5.2-6.5, 5.4-6.5, 5.6-6.5, 5.8-6.5, 6.0-6.5, 4.6-6.4, 4.8-6.4, 5.0-6.4, 5.2-6.4, 5.4-6.4, 5.6-6.4, 5.8-6.4, 6.0-6.4, 4.6-6.3, 4.8-6.3, 5.0-6.3, 5.2-6.3, 5.4-6.3, 5.6-6.3, 5.8-6.3, 6.0-6.3, 4.6-6.2, 4.8-6.2, 5.0-6.2, 5.2-6.2, 5.4-6.2, 5.6-6.2, 5.8-6.2, 6.0-6.2, 4.6-6.1, 4.8-6.1. 5.0-6.1, 5.2-6.1, 5.4-6.1, 5.6-6.1, 5.8-6.1, 4.6-6.0, 4.8-6.0, 5.0-6.0, 5.2-6.0, 5.4-6.0, 5.6-6.0, or 5.8-6.0. Each possibility represents a separate embodiment of the current disclosure. Furthermore, in some embodiments, the method comprises incubating said differentiated fibroblast cell in a an acidic pH incubation medium with a pH between 5 and 6.5. In some embodiments, the method comprises incubating said differentiated fibroblast cell in an acidic pH incubation medium with a pH between 5.8 and 6.2.

In specific embodiments, methods are provided for preparing gene modified fibroblasts comprising the steps of culturing fibroblasts in a first acidic pH incubation medium and a second nonacidic pH incubation medium. In specific embodiments, fibroblasts are cultured in the first acidic pH incubation medium and the second nonacidic pH incubation medium before gene modification, concurrent with gene modification, after gene modification, or a combination thereof.

The incubation step of culturing cells in acidic conditions may be performed for any one of 1 second-180 minutes, 1 second-8 minutes, 1 second-6 minutes, 1 second-4 minutes, 1-10 minutes, 1-8 minutes, 1-6 minutes, 1-4 minutes, 2-10 minutes, 2-8 minutes, 2-6 minutes, 2-4 minutes, 4-10 minutes, 4-8 minutes, or 4-6 minutes. The incubation step of culturing cells in acidic conditions may be performed for any one of 10-180, 10-150, 10-120, 10-90, 10-60, 15-180, 15-150, 15-120, 15-90, 15-60, 20-180, 20-150, 20-120, 20-90, 20-60, 25-180, 25-150, 25-120, 25-90, 25-60, 30-180, 30-150, 30-120, 30-90, or 30-60, minutes. Each possibility represents a separate embodiment of the current invention. In some embodiments, the incubating in acidic conditions is performed for 15-120 minutes.

In some embodiments, the incubating in a second nonacidic incubation medium is performed for 10 minutes to 3 weeks. In some embodiments, the incubating in a second nonacidic incubation medium is performed for 10 minutes to 3 weeks. Each possibility represents a separate embodiment of the current invention.

In some embodiments, the incubating in a media further comprises incubating in a hypoxic condition. In some embodiments, the hypoxic condition is at or below 4.5%, 4%, 3.5%, 3%, or 2.5% oxygen. Each possibility represents a separate embodiment of the current invention. In some embodiments, the hypoxic condition is at or below 3% oxygen. In some embodiments, the hypoxic condition is 1-4.5%, 1-4%, 1-3.5%, 1-3%, 1.5-4.5%, 1.5-4%, 1.5-3.5%, 1.5-3%, 2-4.5%, 2-4%, 2-3.5%, 2-3%, 2.5-4.5%, 2.5-4%, 2.5-3.5%, or 2.5-3% oxygen. Each possibility represents a separate embodiment of the current invention. In some embodiments, the hypoxic condition is 2-4% oxygen. Incubating in hypoxia or a hypoxic condition will be well known to a skilled artisan. Special hypoxic incubator chambers can be used for this purpose and are sold by purveyors of tissue culture chamber.

Media used for the incubation of regenerative cells will be well known to one skilled in the art [1-4]. Non-limiting examples include, DMEM, RPMI, F12, IMDM, and MEM. In some embodiments, any one of the incubation media of the methods provided herein has low glucose. In some embodiments, the media of incubation one or two or both has less than 5 g/L, 4 g/L, 3 g/L, 2 g/L, 1 g/L, or 0.5 g/L glucose. Each possibility represents a separate embodiment of the current invention.

In some embodiments, the medium is supplanted with one or more mitogenic factors. In some embodiments, the medium is supplanted with EGF at a concentration of at least 1, 2, 5, 7, 10, 12, or 15 ng/nl. In some embodiments, the medium is supplanted with EGF at a concentration of at most, 5, 7, 10, 12, 15, 20, or 25 ng/nl. In some embodiments, the medium is supplanted with EGF at a concentration of between 1-20, 2-20, 5-20, 7-20, 10-20, 12-20, 1-15, 2-15, 5-15, 7-15, 10-15, 12-15, 1-12, 2-12, 5-12, 7-12, or 10-12, ng/nl. In some embodiments, the medium is supplanted with EGF at a concentration of between 5-20 ng/nl.

In some embodiments, the medium is supplanted with FGF at a concentration of at least 1, 2, 5, 7, 10, 12, or 15 ng/nl. In some embodiments, the medium is supplanted with FGF at a concentration of at most, 5, 7, 10, 12, 15, 20, or 25 ng/nl. In some embodiments, the medium is supplanted with FGF at a concentration of between 1-20, 2-20, 5-20, 7-20, 10-20, 12-20, 1-15, 2-15, 5-15, 7-15, 10-15, 12-15, 1-12, 2-12, 5-12, 7-12, or 10-12, ng/nl. In some embodiments, the medium is supplanted with FGF at a concentration of between 5-20 ng/nl.

In some embodiments, the medium is further supplemented with at least one cytokine. In some embodiments, the cytokine is selected from the group consisting of: pro-inflammatory cytokines, chemokines, and pro-fibrotic cytokines. In some embodiments, the at least one cytokine is selected from the group consisting of: IL-6, TGF-beta, TNF-alpha, CTGF, SPARC, and SDF1. In some embodiments, the cytokine is supplemented at a concentration of 0.1-100, 0.1-90, 0.1-80, 0.1-70, 0.1-60, 0.1-50, 0.1-40, 0.1-30, 0.1-20, 0.1-10, 1-100, 1-90, 1-80, 1-70, 1-60, 1-50, 1-40, 1-30, 1-20, 1-10, 10-100, 10-90, 10-80, 10-70, 10-60, 10-50, 15-100, 15-90, 15-80, 15-70, 15-60, 15-50, 20-100, 20-90, 20-80, 20-70, 20-60, 20-50, 25-100, 25-90, 25-80, 25-70, 25-60, or 25-50, ng/ml. Each possibility represents a separate embodiment of the current invention. In some embodiments, TNF-alpha is supplemented at a concentration of 25-80 ng/ml. In some embodiments, IL-6 is supplemented at a concentration of 10-50 ng/ml. In some embodiments, SPARC is supplemented at a concentration of 10-50 ng/ml. In some embodiments, CTGF is supplemented at a concentration of 1-50 ng/ml. In some embodiments, SDF1 is supplemented at a concentration of 1-50 ng/ml. In some embodiments, TGF-beta is supplemented at a concentration of 0.1-30 ng/ml. In some embodiments, the medium is further supplemented with a supplement comprising at least one small molecule selected from the group consisting of: a Rho-associated protein kinase (ROCK) inhibitor, a GSK3 inhibitor, a sonic hedgehog (SHH) inhibitor, an ERK activator, a monoamine oxidase (MAO) inhibitor, a protein kinase C (PKC) activator, a histone modifying enzyme inhibitor, and a combination thereof.

In some embodiments, inhibitors reduce expression or function of a protein or complex by at least 10, 20, 30, 40, 50, 60, 70, 80, 90, 95, or 99%. Each possibility represents a separate embodiment of the current invention. A person skilled in the art will understand that each small molecule will be administered to cells at a concentration that is specific to that molecule and which is sufficient to cause inhibition. The manufacturer's guidelines should be followed when supplementing with these molecules unless otherwise specified herein below. In some embodiments, activators increase expression or function of a protein or complex by at least 10, 20, 30, 40, 50, 60, 70, 80, 90, 95, or 99%. Each possibility represents a separate embodiment of the current invention. A person skilled in the art will understand that each small molecule will be administered to cells at a concentration that is specific to that molecule and which is sufficient to cause activation. The manufacturer's guidelines should be followed when supplementing with these molecules unless otherwise specified herein below.

ROCK inhibitors, inhibit Rho-associated protein kinases and are well known in the art. Non-limiting examples include Y-27632, thiazovivin, fasudil, and GSK429286A. GSK inhibitors inhibit glycogen synthase kinase 3A, 3B, and 3C, and are well known in the art. Non-limiting examples include CHIR99021, kenpaullone, SB-216763, indirubin, hymenidin, aloinin A, and lithium chloride. MAO inhibitors inhibit monoamine oxidase A and B and are well known in the art. Non-limiting examples include tranylcypromine, 2-propynal, clorgyline hydrochloride, ethyl homovanillate, phenelzine sulfate salt, rasagiline, and pimprinine.

Protein kinase C (PKC) alpha and epsilon are signaling molecules that make up the PKC signaling complex and activators of the complex are well known in the art. Non-limiting examples include, PKC alpha, PKC epsilon, phorbol 12-myristate 13-acetate (PMA), alpha-amyloid precursor protein, phorbol-12, 13-dibutyrate, mezerein, ingenol 3-angelate, and phorbol 12, 13-dihexanoate. ERK activation is also known to activate PKC. In some embodiments, the ERK activator is Ceramide C6.

Histone modifying enzymes include, but are not limited to, histone acetyltransferases, histone deacetylases, histone methyltransferases, histone demethylases, histone phosphorylases, histone ubiquitinases, histone sumoylases, histone ADP ribosylases, and histone deiminases. Inhibitors of these enzymes are well known in the art and include but are not limited to: 3-deazaneplanocin A, 5-aza-2′-deoxycytidine, 4-phenylbutyrate, TC-H 106, valproic acid, insulin, and SIRT1 activator 3.

In some embodiments, the medium is further supplemented with a small molecule selected from the group consisting of: Y-27632, CHIR99021, tranylcypromine, PMA, 3-deazaneplanocin A, 5-aza-2′-deoxycytidine, 4-phenylbutyrate, one or more PKC alpha activators, one or more PKC epsilon activators, valproic acid, insulin, or a combination thereof. In specific embodiments, the incubation medium is further supplemented with 0.1-100 ng/ml of Y-27632, CHIR99021, tranylcypromine, PMA, 3-deazaneplanocin A, 5-aza-2′-deoxycytidine, 4-phenylbutyrate, one or more PKC alpha activators, one or more PKC epsilon activators, valproic acid, insulin, or a combination thereof. In specific embodiments, the mitogenic factor is PMA. In specific embodiments, the incubation medium further comprises 1-50 ng/ml PMA.

In some embodiments, the small molecule is supplemented at a concentration of 0.1-100, 0.1-90, 0.1-80, 0.1-70, 0.1-60, 0.1-50, 0.1-40, 0.1-30, 0.1-20, 0.1-10, 1-100, 1-90, 1-80, 1-70, 1-60, 1-50, 1-40, 1-30, 1-20, 1-10, 10-100, 10-90, 10-80, 10-70, 10-60, 10-50, 15-100, 15-90, 15-80, 15-70, 15-60, 15-50, 20-100, 20-90, 20-80, 20-70, 20-60, 20-50, 25-100, 25-90, 25-80, 25-70, 25-60, or 25-50 ng/ml. In some embodiments, PMA is supplemented at a concentration of 1-50 nm.

Gene modified fibroblasts may be used for a variety of conditions, including one or more therapeutic conditions. Each condition may utilize specialized fibroblasts. For example, in the embodiment of treatment of fibrosis, anti-fibrotic factors such as one or more TGF-beta antagonists (e.g., one or more TGF-beta 1 antagonists or one or more TGF-beta 2 antagonists) is transfected into fibroblasts. Throughout the disclosure, the term “transfection” may include constitutive expression of the transfected gene(s), or inducible expression of the transfected gene(s).

In some embodiments, TGF-beta antagonist(s) are utilized, and a TGF-beta antagonist may be decorin. In some embodiments, the decorin may be a part of a fusion protein, for example, CAR-decorin, which includes a wound homing peptide, CAR (CARSKNKDC, SEQ ID NO: 1), see, e.g., Jirvinen and Ruoslahti, U.S. Pat. No. 9,180,161. In other embodiments, a TGF-beta antagonist may be, for example, an anti-TGF-beta antibody (e.g., fresolimumab, lerdelimumab, and metelimumab) or an anti-TGF-beta oligonucleotide (e.g., trabedersen (an antisense oligonucleotide targeting TGF-beta 2) or an siRNA targeting TGF-beta). The anti-TGF-beta antibody may be a monoclonal antibody, a humanized antibody, or a human antibody. The anti-TGF-beta antibody may be an antibody fragment.

Anti-fibrotic factors known in the art can be transfected into cells, including, for example, cathepsin D, cathepsin E, cathepsin S, cathepsin K, cathepsin L, cathepsin B, cathepsin C, cathepsin H, cathepsin F, cathepsin G, cathepsin O, cathepsin R, cathepsin V (cathepsin 12), cathepsin W, cathepsin Z (cathepsin X), calpin 1, calpin 2, chondroitinase ABC, chondroitinase AC, pancreatic elastase, elastase-2a, elastase-2b, neutrophil elastase, proteinase-3, endogenous vascular elastase, mast cell chymase, mast cell tryptase, plasmin, thrombin, granzyme B, hyaluronidase, chymopapain, chymotrypsin, legumain, collagenase, matrix metalloproteinases (e.g., MMP-1 (collagenase-1), MMP-9, MMP-7 (matrilysin), MMP-8 (collagenase-2), MMP-13 (collagenase-3), MMP-18 (collagenase-4), MMP-2 (gelatinase a), MMP-9 (gelatinase b), MMP-3 (stromelysin-1), MMP-10 (stromelysin-2), MMP-11 (stromelysin-3), MMP-7 (matrilysin), MMP-26 (matrilysin), MMP-12 (metalloelastase), MMP-14 (MT1-MMP), MMP-15 (MT2-MMP), MMP-16 (MT3-MMP), MMP-17 (MT4-MMP), MMP-24 (MT5-MMP), MMP-25 (MT6-MMP), MMP-19, MMP-20 (enamelysin), MMP-x, MMP-23, MMP-27, and MMP-28 (epilysin)), ADAMTS -1, ADAMTS -2, ADAMTS -3, ADAMTS -4 (aggrecanase-1), ADAMTS -5 (aggrecanase-2), ADAMTS -14, papain, subtilisin, subtilisin A, heparanase, TGF-beta receptor antagonists (e.g., TGF-beta receptor antibodies), angiotensin inhibitors, angiotensin-converting enzyme inhibitors, angiotensin-II receptor antagonists, cannabinoid receptor, or a combination thereof. In some embodiments, the anti-fibrotic factor includes, but are not limited to, interleukins, interferons (e.g., interferon gamma), cytokines, chemokines, chemotactic molecules, relaxin, hormones (e.g., progesterone, estrogen, testosterone, growth hormone, thyroid hormone, parathyroid hormone, and the like) or a combination thereof.

In the treatment of conditions associated with inflammation, various “anti-inflammatory factors,” as used herein, may be transfected in order to endow fibroblasts with enhanced ability to reduce inflammation. An anti-inflammatory factor may include, for example, inflammatory cytokine antagonists (e.g., IL-6 antagonists and/or IL-10 antagonists) and anti-microbial factors. An “inflammatory cytokine antagonist” refers to any agent which decreases, blocks, inhibits, abrogates, or interferes with the pro-inflammatory cascade of cytokine proteins leading to an inflammatory response. Exemplary inflammatory cytokine inhibitors include IL-10 (which in some embodiments functions as an IL-6 antagonist and/or an IL-10 antagonist), LL-37, and thymosin beta 4. In some embodiments, the anti-microbial factor is LL-37 or thymosin beta 4. In some embodiments, an anti-inflammatory agent (e.g., an inflammatory cytokine antagonist) may be an antibody (e.g., an anti-TNFalpha antibody (e.g., infliximab, adalimumab, certolizumab pegol, and golimumab), an anti-IL-6 antibody, or an anti-IL-10 antibody). The antibody may be a monoclonal antibody, a humanized antibody, or a human antibody. The antibody may be an antibody fragment. In other instances, an anti-inflammatory factor (e.g., an inflammatory cytokine antagonist) may be a soluble receptor fusion protein, e.g., etanercept.

In some embodiments, the anti-inflammatory factor is a “non-antibody anti-inflammatory factor.” As used herein, this term specifically excludes antibodies (e.g., monoclonal antibodies, human antibodies, and humanized antibodies). In some embodiments, the term “non-inflammatory anti-inflammatory factor” specifically excludes anti-tumor necrosis factor (TNF) antibodies (e.g., infliximab, adalimumab, certolizumab pegol), alemtuzumab, afelimomab, aselizumab, atlizumab, atorolimumab, basiliximab, belimumab, bertilimumab, cedelizumab, clenoliximab, daclizumab, dorlimomab aritox, dorlixizumab, eculizumab, efalizumab, elsilimomab, erlizumab, faralimomab, fontolizumab, galiximab, gantenerumab, gavilimomab, golimumab, gomiliximab, ibalizumab, inolimomab, ipilimumab, keliximab, lebrilizumab, lerdelimumab, lumiliximab, maslimomab, mepolizumab, metelimumab, morolimumab, muromonab-CD3, natalizumab, nerelimomab, ocrelizumab, odulimomab, omalizumab, otelixizumab, pascolizumab, pexelizumab, rituxumab, reslizumab, rovelizumab, ruplizumab, siplizumab, talizumab, telimomab aritox, teneliximab, teplizumab, tocilizumab, toralizumab, vapaliximab, vepalimomab, visilizumab, zanolimumab, ziralimumab, and zolimomab aritox.

In some embodiments, fibroblasts are engineered (such as by transfection) to express higher levels of extracellular matrix proteins in order to promote acceleration of healing, and in some embodiments, healing without fibrosis. Non-limiting examples of extracellular matrix factors include collagen (e.g., collagen-I, collagen-III, and collagen-VI), decorin, fibronectin, vitronectin, laminin, cartilage oligomeric matrix protein (COMP), tenascin-C, tenascin-X, elastin, keratin (e.g., K6 and K16), tissue inhibitor of metalloproteinase-1 (TIMP-1), albumin, osteonectin, thrombospondin (e.g., thrombospondin-1 or thrombospondin-2), proteoglycans (e.g., versican, syndecan, glypicans, perlecan, lumican, and heparin sulfate), glycosaminoglycans (e.g., hyaluronan/hyaluronic acid), and integrins.

In other embodiments, clotting factors may be transfected in order to endow fibroblasts with enhanced ability to to treat conditions associated with clotting abnormalities. As used herein, the term clotting factors encompasses agents involved in platelet activation as well as the coagulation cascade (including both the intrinsic and extrinsic pathways) that leads to fibrin formation. Clotting factors include, but are not limited to, factor I (fibrinogen/fibrin), factor II (prothrombin), CD142 (also known as tissue factor, tissue thromboplastin, or factor III), factor V, factor VII, factor VIII, factor IX, factor X, factor XI, factor XII, factor XIII, von Willebrand factor, prekallikrein, high-molecular weight kininogen (HMWK), fibronectin, antithrombin III, heparin cofactor II, protein C, protein S, protein Z, plasminogen, tissue plasminogen activator (tPA), and urokinase.

In some embodiments of the disclosure, fibroblasts are gene-modified to express a biological agent that is involved in angiogenesis, the formation of new blood vessels. Angiogenic factors include, but are not limited to, vascular endothelial growth factors (VEGFs), including, e.g., VEGF-A, VEGF-B, VEGF-C, VEGF-D, VEGF-E, and placental growth factor (PIGF); angiopoietins and angiopoietin-like proteins (e.g., ANGPT1, ANGPT2, ANGPT4, ANGPTL1, ANGPTL2, ANGPTL3, ANGPTL4, ANGPTL5, ANGPTL6, and ANGTPL7); fibroblast growth factors (FGFs), including FGF-1 and FGF-2; epidermal growth factor (EGF); transforming growth factors (TGFs), including TGF-alpha and TGF-beta, tumor necrosis factors (TNFs), including TNF-alpha; colony stimulating factors (CSFs), including granulocyte macrophage colony-stimulating factor (GM-CSF) and macrophage colony stimulating factor (M-CSF, also known as CSF1); and nitric oxide synthase (NOS).

In some embodiments, the fibroblasts are rendered to possess enhanced migratory properties, enhanced ability to produce IGF, HGF, and FGF, as well as enhanced resistance to apoptosis by transfection with one or more genes selected from a group comprising: Lin-28, STATS, NFKB, CEBP/B, SOX2, OCT4, WNT5A, LIF, COX2, RUNX2 and NANOG.

Numerous genes may be utilized for various therapeutic applications wherein the encoded peptide or protein comprises a therapeutic protein or a fragment or variant thereof, including any gene listed herein.

The introduction of nucleic acids into cells can be accomplished by various means. In one embodiments, transduction is the infection of a target cell such as the fibroblast by a virus that promotes genetic modification of the target cell. Many viruses bind and infect mammalian cells and can be used to introduce genetic material (e.g., a donor gene, such as a gene encoding a wound healing agent) into the host cell as part of their replication cycle. In viruses modified for gene transfer, the donor gene (e.g., a gene encoding a wound healing agent) is inserted into the viral genome. Additional modifications may be made to the virus to improve infectivity or tropism (e.g., pseudotyping), to reduce or eliminate replicative competency, and/or to reduce immunogenicity. The newly-introduced donor gene will be expressed in the infected host cell or organism and, if replacing a defective host gene, can ameliorate conditions or diseases caused by the defective gene.

Numerous examples of viral vectors that can be used to deliver genetic material (e.g., a donor gene, such as a gene encoding a wound healing agent) include, but are not limited to, a retrovirus, adenovirus (e.g., Ad2, Ad5, Ad11, Ad12, Ad24, Ad26, Ad34, Ad35, Ad40, Ad48, Ad49, Ad50, and Pan9 (also known as AdC68)), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding a wound healing agent to a HUCPVC include Norwalk virus, togavirus, flavivirus, reoviruses, papovavirus, hepadnavirus, and hepatitis virus. Adenoviruses and retroviruses (including lentiviruses) are particularly attractive modalities for gene therapy applications, as discussed below, due to the ability to genetically modify and exploit the life cycle of these viruses.

Another means of viral introduction is the utilization of recombinant adenoviral vectors, which offer several significant advantages for the expression of a wound healing agent(s) in HUCPVCs. The viruses can be prepared at extremely high titer, infect non-replicating cells, and confer high-efficiency and high-level transduction of target cells in vivo after directed injection or perfusion. Furthermore, as adenoviruses do not integrate their DNA into the host genome, this gene therapy modality has a reduced risk of inducing spontaneous proliferative disorders. In animal models, adenoviral gene transfer has generally been found to mediate high-level expression for approximately one week. The duration of transgene expression may be prolonged, and ectopic expression reduced, by using tissue-specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a cre-lox. Examples of adenoviruses that can be used as a viral vector of the invention include those having, or derived from, the serotypes Ad2, Ad5, Ad11, Ad12, Ad24, Ad26, Ad34, Ad35, Ad40, Ad48, Ad49, Ad50, and Pan9 (also known as AdC68).

Recombinant adeno-associated viruses (rAAV), which are derived from non-pathogenic parvoviruses, can be used to express a donor gene, such as a gene encoding a wound healing agent(s), as these vectors evoke almost no cellular immune response, and produce transgene expression lasting months in most systems. The AAV genome is built of single stranded DNA, and includes inverted terminal repeats (ITRs) at both ends of the DNA strand, and two open reading frames: rep and cap, encoding replication and capsid proteins, respectively. A donor gene (e.g., a gene encoding a wound healing agent) can replace the native rep and cap genes. AAVs can be made with a variety of different serotype capsids which have varying tropism for different tissue types. Examples of AAV serotypes that can be used include but are not limited to AAV1, AAV2, AAV3, AAV4, AAV5, AAV6, AAV7, AAV8, AV9, and AAVrh10. AAV vectors can be produced, for example, by triple transfection of subconfluent HEK293 cells by three plasmids: AAV cis-plasmid containing the donor gene of interest (e.g., a gene encoding a wound healing agent), AAV trans-plasmid containing AAV rep and cap genes, and an adenovirus helper plasmid, e.g., pDF6. Incorporation of a tissue-specific promoter is, again, typically beneficial.

Another viral vector that can be used to deliver a genes into a subject or cells is a retrovirus, including a lentivirus. As opposed to adenoviruses, the genetic material in retroviruses is in the form of RNA molecules, while the genetic material of their hosts is in the form of DNA. When a retrovirus infects a host cell, it will introduce its RNA together with some enzymes into the cell. This RNA molecule from the retrovirus will produce a double-stranded DNA copy (provirus) from its RNA molecules through a process called reverse transcription. Following transport into the cell nucleus, the proviral DNA is integrated in a host chromosome, permanently altering the genome of the infected cell and any progeny cells that may arise. The ability to permanently introduce a gene encoding a polypeptide or oligonucleotide into a cell such as a HUCPVC is the defining characteristic of retroviruses used for gene therapy. Retroviruses include lentiviruses, a family of viruses including human immunodeficiency virus (HIV) that includes several accessory proteins to facilitate viral infection and proviral integration. Additional examples of retroviruses include: avian leukosis-sarcoma, mammalian C-type, B -type viruses, D-type viruses, HTLV-BLV group, and spumavirus.

A retrovirus for gene therapy may be one that is modified to direct the insertion of the donor gene incorporated in the genome of the virus into a non-arbitrary position in the genome of the host, e.g., using a zinc finger nuclease or by including sequences, such as the beta-globin locus control region, to direct the site of integration to specific chromosomal sites. Retroviruses and lentiviruses have considerable utility for gene therapy applications. Current, “third-generation” lentiviral vectors feature total replication incompetence, broad tropism, and increased gene transfer capacity for mammalian cells. Lentiviruses pseudotyped with, e.g., vesicular stomatitis virus glycoprotein (VSV-G) or feline endogenous virus RD114 envelope glycoprotein can be used to transduce HUCPVCs. U.S. Pat. Nos. 5,919,458, 5,994,136, and 7,198,950, hereby incorporated by reference, describe the production and use of lentiviruses to genetically modify target cells.

Besides adenoviral and retroviral vectors, other viral vectors and techniques are known in the art that can be used to transfer a donor gene encoding a desired polypeptide or oligonucleotide (e.g., a gene encoding a wound healing agent) into a subject or cells. These viruses include, e.g., poxviruses (e.g., vaccinia virus and modified vaccinia virus Ankara (MVA); see, e.g., U.S. Pat. Nos. 4,603,112 and 5,762,938), herpesviruses, togaviruses (e.g., Venezuelan Equine Encephalitis virus; see, e.g., U.S. Pat. No. 5,643,576), picornaviruses (e.g., poliovirus; see, e.g., U.S. Pat. No. 5,639,649), and baculoviruses. Other viruses useful for delivering donor genes include papovavirus, hepadnavirus, and hepatitis virus, for example.

Naked DNA or oligonucleotides (e.g., DNA vectors such as plasmids) encoding wound healing agents can also be used to genetically modify fibroblasts. This is the simplest method of non-viral transfection. Clinical trials carried out using intramuscular injection of a naked DNA plasmid have had some success; however expression has been low in comparison to other methods of transfection. Other efficient methods for delivery of naked DNA exist such as electroporation and the use of a “gene gun,” which shoots DNA-coated gold particles into the cell using high pressure gas.

To improve the delivery of a DNA vector (e.g., a plasmid) into fibroblasts, the DNA can be protected from damage and its entry into the cell facilitated. Lipoplexes and polyplexes have the ability to protect transfer DNA from undesirable degradation during the transfection process. Plasmid DNA can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also as a result of their charge they interact with the cell membrane, endocytosis of the lipoplex occurs, and the DNA is released into the cytoplasm. The cationic lipids also protect against degradation of the DNA by the cell. Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

In some embodiments gene editing is used to genetically modify fibroblasts. Broadly, gene editing approaches are based on precise, targeted changes to the genome of organisms. Gene editing may be used to alter the genome sequence (for example, by incorporation of point mutations, insertions, or deletions). Gene editing approaches can be used to ‘knock-in’ heterologous nucleic acid sequences into the genome at targeted locations. A variety of gene editing approaches are known in the art, including but not limited to clustered regularly interspaced short palindromic repeats (CRISPR)-Cas (e.g., Cas9) gene editing (see, e.g., U.S. Pat. Nos. 8,697,359 and 8,771,945), transcription activator-like effector based nuclease (TALEN) gene editing, zinc-finger nuclease (ZFN) gene editing, or meganuclease gene editing (see, e.g., U.S. Pat. No. 8,021,867).

The process of cell transfection may occur before, concurrently with, or after treatment of cells with acidic conditions. In one particular embodiment, cells are extracted by biopsy, cultured to expansion, such as 10-fold or more expansion, and subsequently exposed to acidic conditions, after which they are transfected.

In some embodiments, fibroblasts are utilized in an autologous manner. In another embodiment allogeneic fibroblasts are utilized for the practice of the disclosure. Various sources of fibroblasts may be used for the practice of the invention, these include at least: a) fallopian tube; b) menstrual blood; c) endometrium; d) skin; e) nail cuticle; f) deciduous tooth; g) hair follicle; h) tonsil; i) omentum; j) bone marrow; k) placenta; l) umbilical cord; m) peripheral blood; n) adipose tissue; o) amniotic fluid; p) umbilical cord blood; q) embryonic fibroblasts; r) plastic surgery related by-product; or s) nail matrix.

In one embodiment, fibroblasts are activated by administration of agents which act as “regenerative adjuvants” for said fibroblasts. The term “regenerative adjuvants” as used herein refers to agents which mobilize fibroblasts or enhance production of growth factors such as IGF, EGF, or PDGF. In specific embodiments, the activation is achieved by administration of G-CSF, M-CSF, GM-CSF, or Flt3 ligand. In specific embodiments, the peripheral blood is extracted after activation of fibroblasts. In specific embodiments, the activation is achieved by administration of G-CSF, M-CSF, GM-CSF, or Flt3 ligand.

The fibroblasts utilized in the disclosure may be generated, in one embodiment, by outgrowth from a biopsy of the recipient's own skin (in the case of autologous preparations), or skin of healthy donors (for allogeneic preparations). In some embodiments fibroblasts are used from young donors. Skin tissue (dermis and epidermis layers) may be biopsied from a subject's post-auricular area.

In one embodiment, the starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±0.2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Heidelberg, Germany).

In one embodiment fibroblasts are transfected with genes prior to expansion in culture to allow for enhanced growth and overcoming of the Hayflick limit. In another embodiment, fibroblasts are transfected with genes in an acidic pH incubation medium prior to expansion in culture. Subsequent to transfection of cells, expansion in culture may be performed using standard cell culture techniques.

Adherent and non-adherent plates will be well known to one skilled in the art. Adherent plates are frequently coated to increase adherence, or made of plastics with high cellular adherence. Examples of such plates include, but are not limited to, classic tissue culture plates such as are sold by Fischer Scientific, Corning, Sigma Aldrich and others, gelatin-coated plates, and collagen coated plates. Non-adherent plates also refer to low-adherence plates such as are common for culturing spheroids.

The term “limiting dilution” as used herein refers to diluting the concentration of cells in a media such that there is a high probability that only a single cell will be present in each sample. In some embodiments, limiting dilution refers to diluting cells such that when plated into wells of a dish there is only one cell in every other well, every third well, every fifth well, or every tenth well. Each possibility represents a separate embodiment of the current invention.

After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37.0±2.0° C. with 5.0±1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask.

Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies.

Cells are incubated at 37.0±2.0° C. with 5.0±1.0% CO₂ and passaged every three to five days in the T-500 flask and every five to seven days in the ten layer cell stack (10 CS). Cells should not remain in the T-500 flask for more than 10 days prior to passaging. Quality Control (QC) release testing for safety of the Bulk Drug Substance includes sterility and endotoxin testing. When cell confluence in the T-500 flask is greater than or equal to 95%, cells are passaged to a 10 CS culture vessel. Alternately, two Five Layer Cell Stacks (5 CS) or a 10 Layer Cell Factory (10 CF) can be used in place of the 10 CS. 10 CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers.

Cells are then incubated at 37.0±2.0° C. with 5.0±1.0% CO₂ and fed with fresh Complete Growth Media every five to seven days. Cells should not remain in the 10 CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is greater than or equal to 95%, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

In one embodiment fibroblasts are transfected with genes after expansion in culture, such as 10-fold or more expansion. In another embodiment, fibroblasts are transfected with genes in an acidic pH incubation medium after expansion in culture, such as 10-fold or more expansion.

The cells express proteins characteristic of normal fibroblasts including the fibroblast-specific marker, CD90 (Thy-1), a 35 kDa cell-surface glycoprotein, and the extracellular matrix protein, collagen. In some embodiments, the fibroblast dosage formulation is an autologous cell therapy product composed of a suspension of autologous fibroblasts, grown from a biopsy of each individual's own skin using standard tissue culture procedures. In one embodiment the fibroblasts of the invention can also be used to create other cell types for tissue repair or regeneration.

In one embodiment, the disclosure concerns the use of activation of fibroblasts prior to therapeutic use, and/or administration of agents which act as “regenerative adjuvants” for said fibroblasts. The term “regenerative adjuvants” as used herein refers to agents which mobilize fibroblasts or enhance production of growth factors such as IGF, EGF, or PDGF. In specific embodiments, the activation is achieved by administration of G-CSF. In specific embodiments, the activation is achieved by administration of M-CSF. In specific embodiments, the activation is achieved by administration of GM-CSF. In specific embodiments, the activation is achieved by administration of Flt3 ligand.

In a particular embodiment, about 50 million to 500 million fibroblast cells are administered to the subject. For example, about 50 million to about 100 million fibroblast cells, about 50 million to about 200 million fibroblast cells, about 50 million to about 300 million fibroblast cells, about 50 million to about 400 million fibroblast cells, about 100 million to about 200 million fibroblast cells, about 100 million to about 300 million fibroblast cells, about 100 million to about 400 million fibroblast cells, about 100 million to about 500 million fibroblast cells, about 200 million to about 300 million fibroblast cells, about 200 million to about 400 million fibroblast cells, about 200 million to about 500 million fibroblast cells, about 300 million to about 400 million fibroblast cells, about 300 million to about 500 million fibroblast cells, about 400 million to about 500 million fibroblast cells, about 50 million fibroblast cells, about 100 million fibroblast cells, about 150 million fibroblast cells, about 200 million fibroblast cells, about 250 million fibroblast cells, about 300 million fibroblast cells, about 350 million fibroblast cells, about 400 million fibroblast cells, about 450 million fibroblast cells or about 500 million fibroblast cells may be administered to the subject.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Induction of Tumor Regression by Administration of Interferon Gamma Expressing Fibroblasts

Dermal fibroblasts were obtained from ATCC and cultured under manufacturer's recommendation. Briefly, subsequent to thawing and washing in phosphate buffered saline (PBS) cells were expanded in DMEM media with 10% fetal calf serum.

Generation of stable transfected fibroblasts was performed by cloning out the murine interferon gamma transcribed using the following primers:

(F-5′ AGCGGATCCGCCATGAATTATTCAAGCTACCTCTTAGC 3′
(SEQ ID NO: 1)
and
R-5′ TATCTCTAGATTACTGTGTGATTTTTCCTTGGCTTTT 3′;
SEQ ID NO: 2).

The amplicon was cloned into the pcDNA3.1 expression vector (Invitrogen) by using standard molecular biology procedures. The resulting construct was sequenced in both directions to ensure no errors were introduced during amplification or cloning. The construct (pcDNA3-IFN-γ) was transiently transfected into fibroblasts by using FuGENE transfection reagent (Promega). A construct-only control, transfection reagent-only control was also used. Prior to transfection, the cells were cultured in 6.5 pH media for 24 hours, and following transfection the cells were cultured in the 7 pH media.

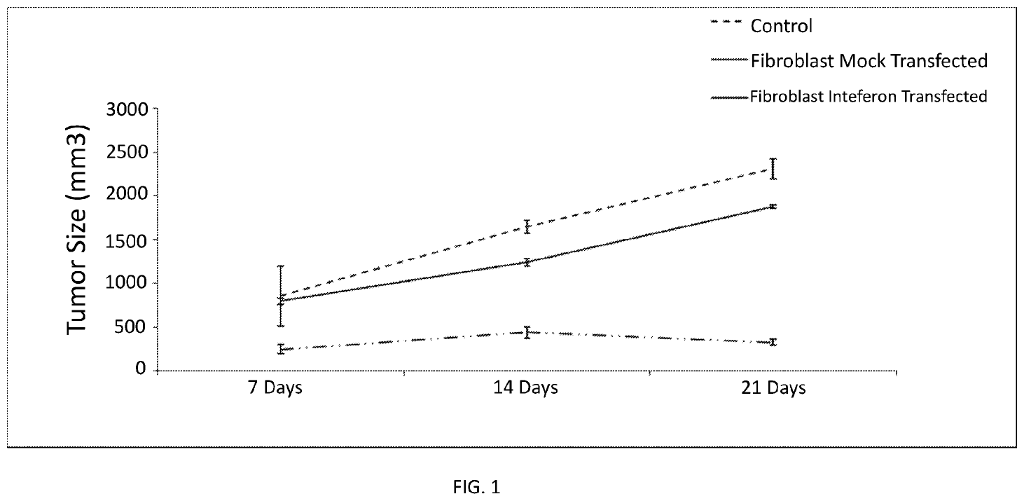

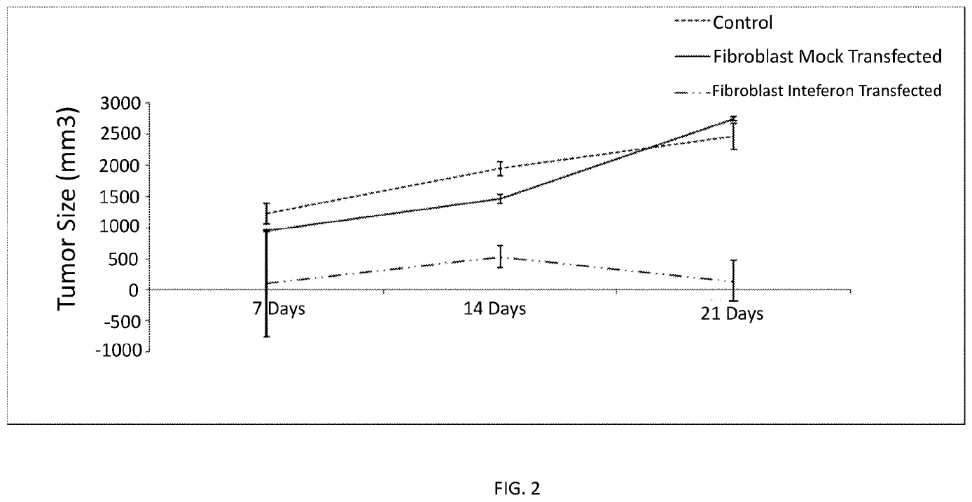

FIGS. 1-2 are demonstrations showing reduction in tumor size in a B16 melanoma model and a LLC lung cancer model following administration of interferon-transfected fibroblasts compared to controls.

Cells were injected at a concentration of 500,000 cells subcutaneously in the flank and quantified by calipers.

### REFERENCES

1. Qu, C., et al., Evaluation of growth, stemness, and angiogenic properties of dental pulp stem cells cultured in cGMP xeno-/serum-free medium. Cell Tissue Res, 2019.

2. Li, Q., et al., Wharton's jelly mesenchymal stem cell-based or umbilical vein endothelial cell-based serum-free coculture with cytokines supports the ex vivo expansion/maintenance of cord blood hematopoietic stem/progenitor cells. Stem Cell Res Ther, 2019. 10(1): p. 376.

3. Torres Chavez, A., et al., Expanding CAR T cells in human platelet lysate renders T cells with in vivo longevity. J Immunother Cancer, 2019. 7(1): p. 330.

4. Dong, G., et al., Serum-Free Culture System for Spontaneous Human Mesenchymal Stem Cell Spheroid Formation. Stem Cells Int, 2019. 2019: p. 6041816.

What is claimed is:

1. A method of preparing genetically modified fibroblasts, the method comprising culturing fibroblasts in an acidic pH incubation medium before genetic modification, concurrent with genetic modification, after genetic modification, or a combination thereof.

2. The method of claim 1, wherein the method comprises culturing fibroblasts in an acidic pH incubation medium before genetic modification.

3. The method of claim 1 or 2, wherein the method comprises culturing fibroblasts in an acidic pH incubation medium concurrent with genetic modification

4. The method of any one of claims 1 to 3, wherein the method comprises culturing fibroblasts in an acidic pH incubation medium after genetic modification.

5. The method of any one of claims 1 to 4, wherein the method comprises culturing fibroblasts in an acidic pH incubation medium before and after genetic modification.

6. The method of any one of claims 1 to 5, wherein said acidic pH is a pH of 4.6-6.5, 4.8-6.5, 5-6.5, 5.2-6.5, 5.4-6.5, 5.6-6.5, 5.8-6.5, 6-6.5, 4.6-6.4, 4.8-6.4, 5-6.4, 5.2-6.4, 5.4-6.4, 5.6-6.4, 5.8-6.4, 6-6.4, 4.6-6.3, 4.8-6.3, 5-6.3, 5.2-6.3, 5.4-6.3, 5.6-6.3, 5.8-6.3, 6-6.3, 4.6-6.2, 4.8-6.2, 5-6.2, 5.2-6.2, 5.4-6.2, 5.6-6.2, 5.8-6.2, 6-6.2, 4.6-6.1, 4.8-6.1. 5-6.1, 5.2-6.1, 5.4-6.1, 5.6-6.1, 5.8-6.1, 4.6-6, 4.8-6, 5.0-6, 5.2-6, 5.4-6, 5.6-6, or 5.8-6.

7. The method of any one of claims 1 to 6, wherein said acidic pH is a pH of 5-6.5.

8. The method of any one of claims 1 to 7, wherein said acidic pH is a pH of 5, 5.1, 5.2, 5.3, 5.4, 5.5, 5.6, 5.7, 5.8, 5.9, 6, 6.1, 6.2, 6.3, 6.4, or 6.5.

9. The method of any one of claims 1 to 8, wherein said acidic pH is a pH of 5.8-6.2.

10. The method of any one of claims 1 to 9, wherein said acidic pH is a pH of 5.8, 5.9, 6, 6.1, or 6.2.

11. The method of any one of claims 1 to 10, wherein said fibroblasts are cultured in an acidic pH incubation medium for 1 second-180 minutes, 1 second-8 minutes, 1 second-6 minutes, 1 second-4 minutes, 1-10 minutes, 1-8 minutes, 1-6 minutes, 1-4 minutes, 2-10 minutes, 2-8 minutes, 2-6 minutes, 2-4 minutes, 4-10 minutes, 4-8 minutes, or 4-6 minutes.

12. The method of any one of claims 1 to 11, wherein said fibroblasts are cultured in an acidic pH incubation medium for 10-180, 10-150, 10-120, 10-90, 10-60, 15-180, 15-150, 15-120, 15-90, 15-60, 20-180, 20-150, 20-120, 20-90, 20-60, 25-180, 25-150, 25-120, 25-90, 25-60, 30-180, 30-150, 30-120, 30-90, or 30-60, minutes.

13. The method of any one of claims 1 to 12, wherein said fibroblasts are cultured in an acidic pH incubation medium for 15-120 minutes.

14. The method of any one of claims 1 to 13, wherein said fibroblasts are cultured in an acidic pH incubation medium for 15, 20, 30, 40, 50, 60, 70, 80, 90, 100, 110, or 120 minutes.

15. The method of any one of claims 1 to 14, wherein said fibroblasts are cultured in a first acidic pH incubation medium and a second nonacidic pH incubation medium.

16. The method of any one of claims 1 to 15, wherein said fibroblasts are cultured in the first acidic pH incubation medium and the second nonacidic pH incubation medium before gene modification, concurrent with gene modification, after gene modification, or a combination thereof.

17. The method of any one of claims 1 to 16, wherein said fibroblasts are cultured in a second nonacidic incubation medium for 10 minutes to 3 weeks, 10 minutes to 2 weeks, 10 minutes to 1 week, 10 minutes to four days, 10 minutes to 2 days, 10 minutes to 1 day, 1 hour to 3 weeks, 1 hour to 2 weeks, 1 hour to 1 week, 1 hour to four days, 1 hour to two days, 1 hour to 1 day, 12 hours to 3 weeks, 12 hours to 2 weeks, 12 hours to 1 week, 12 hours to four days, 12 hours to 2 days, 12 hours to 1 day, 1 day to 3 weeks, 1 day to 2 weeks, 1 day to 1 week, three days to 3 weeks, 3 days to 2 weeks, 3 days to 1 week, 3 days to 4 days, 1 week to 3 weeks, 1 week to 2 weeks, or 2 weeks to three weeks.

18. The method of any one of claims 1 to 17, wherein said fibroblasts are cultured in a second nonacidic incubation medium for 10 minutes to 3 weeks.

19. The method of any one of claims 1 to 18, wherein said fibroblasts are cultured in a hypoxic condition.

20. The method of claim 19, wherein said hypoxic condition is at or below 4.5%, 4%, 3.5%, 3%, or 2.5%, oxygen.

21. The method of claim 19 or 20, wherein said hypoxic condition is at 1-4.5%, 1-4%, 1-3.5%, 1-3%, 1.5-4.5%, 1.5-4%, 1.5-3.5%, 1.5-3%, 2-4.5%, 2-4%, 2-3.5%, 2-3%, 2.5-4.5%, 2.5-4%, 2.5-3.5%, or 2.5-3%, oxygen.

22. The method of any one of claims 19 to 21, wherein said hypoxic condition is at 1-4.5% oxygen.

23. The method of any one of claims 19 to 22, wherein said hypoxic condition is at 2-4% oxygen.

24. The method of any one of claims 19 to 23, wherein said hypoxic condition is at 2, 2.5, 3, 3.5, or 4%, oxygen.

25. The method of any one of claims 19 to 24, wherein said hypoxic condition is at or below 3% oxygen.

26. The method of any one of claims 1 to 25, wherein said incubation medium is DMEM, RPMI, F12, IMDM, MEM, or a combination thereof.

27. The method of any one of claims 1 to 26, wherein said incubation medium comprises less than 5 g/L, 4 g/L, 3 g/L, 2 g/L, 1 g/L, or 0.5 g/L, glucose.

28. The method of any one of claims 1 to 27, wherein said incubation medium comprises less than 5 g/L glucose.

29. The method of any one of claims 1 to 28, wherein said incubation medium further comprises one or more mitogenic factors.

30. The method of claim 29, wherein said mitogenic factor is EGF, one or more FGFs, one or more cytokines, one or more ROCK inhibitors, one or more GSK3 inhibitors, one or more SHH inhibitors, one or more ERK activators, one or more MAO inhibitors, one or more PKC activators, one or more histone modifying enzyme inhibitors, Y-27632, CHIR99021, tranylcypromine, PMA, 3-deazaneplanocin A, 5-aza-2′-deoxycytidine, 4-phenylbutyrate, one or more PKC alpha activators, one or more PKC epsilon activators, one or more ERK activators, valproic acid, insulin, or a combination thereof.

31. The method of claim 29 or 30, wherein said mitogenic factor is EGF.

32. The method of any one of claims 29 to 31, wherein said incubation medium comprises at least 1, 2, 5, 7, 10, 12, or 15 ng/nl EGF.

33. The method of any one of claims 29 to 32, wherein said incubation medium comprises at most 5, 7, 10, 12, 15, 20, or 25 ng/nl EGF.

34. The method of any one of claims 29 to 33, wherein said incubation medium comprises 1-20, 2-20, 5-20, 7-20, 10-20, 12-20, 1-15, 2-15, 5-15, 7-15, 10-15, 12-15, 1-12, 2-12, 5-12, 7-12, or 10-12 ng/nl EGF.

35. The method of any one of claims 29 to 34, wherein said incubation medium comprises 5-20 ng/nl EGF.

36. The method of any one of claims 29 to 35, wherein said mitogenic factor is one or more FGFs.

37. The method of any one of claims 29 to 36, wherein said incubation medium comprises at least 1, 2, 5, 7, 10, 12, or 15 ng/nl of one or more FGFs.

38. The method of any one of claims 29 to 37, wherein said incubation medium comprises at most 5, 7, 10, 12, 15, 20, or 25 ng/nl of one or more FGFs.

39. The method of any one of claims 29 to 38, wherein said incubation medium comprises 1-20, 2-20, 5-20, 7-20, 10-20, 12-20, 1-15, 2-15, 5-15, 7-15, 10-15, 12-15, 1-12, 2-12, 5-12, 7-12, or 10-12 ng/nl of one or more FGFs.

40. The method of any one of claims 29 to 39, wherein said incubation medium comprises 5-20 ng/nl of one or more FGFs.

41. The method of any one of claims 29 to 40, wherein said mitogenic factor is one or more cytokines.

42. The method of claim 41, wherein said cytokine is one or more pro-inflammatory cytokines, one or more chemokines, one or more pro-fibrotic cytokines, or a combination thereof.

43. The method of claim 41 or 42, wherein said cytokine is IGF-1; IL-6, TGF-beta, TNF-alpha, CTGF, SPARC, SDF1, or a combination thereof.

44. The method of any one of claims 29 to 43, wherein said incubation medium comprises 0.1-100 ng/nl of one or more cytokines.

45. The method of any one of claims 29 to 44, wherein said incubation medium comprises 10-50 ng/nl IL-6.

46. The method of any one of claims 29 to 45, wherein said incubation medium comprises 0.1-30 ng/nl TGF-beta.

47. The method of any one of claims 29 to 46, wherein said incubation medium comprises 25-80 ng/nl TNF-alpha.

48. The method of any one of claims 29 to 47, wherein said incubation medium comprises 1-50 ng/nl CTGF.

49. The method of any one of claims 29 to 48, wherein said incubation medium comprises 10-50 ng/nl SPARK.

50. The method of any one of claims 29 to 49, wherein said incubation medium comprises 1-50 ng/nl SDF1.

51. The method of any one of claims 29 to 50, wherein said mitogenic factor is one or more ROCK inhibitors.

52. The method of claim 51, wherein said ROCK inhibitor is Y-27632, thiazovivin, fasudil, GSK429286A, or a combination thereof.

53. The method of any one of claims 29 to 52, wherein said mitogenic factor is one or more GSK3 inhibitors.

54. The method of claim 53, wherein said GSK3 inhibitor is CHIR99021, kenpaullone, SB-216763, indirubin, hymenidin, aloinin A, lithium chloride, or a combination thereof.

55. The method of any one of claims 29 to 54, wherein said mitogenic factor is one or more MAO inhibitors.

56. The method of claim 55, wherein said MAO inhibitor is tranylcypromine, 2-propynal, clorgyline hydrochloride, ethyl homovanillate, phenelzine sulfate salt, rasagiline, pimprinine, or a combination thereof.

57. The method of any one of claims 29 to 56, wherein said mitogenic factor is one or more PKC activators.

58. The method of claims 57, wherein said PKC activator is PKC alpha, PKC epsilon, PMA, alpha-amyloid precursor protein, phorbol-12, 13-dibutyrate, mezerein, ingenol 3-angelate, phorbol 12, 13-dihexanoate, or a combination thereof.

59. The method of any one of claims 29 to 58, wherein said mitogenic factor is one or more ERK activators.

60. The method of claim 59, wherein said ERK activator is Ceramide C6.

61. The method of any one of claims 29 to 60, wherein said mitogenic factor is one or more histone modifying enzyme inhibitors.

62. The method of claim 61, wherein said histone modifying enzyme inhibitor is 3-deazaneplanocin A, 5-aza-2′-deoxycytidine, 4-phenylbutyrate, TC-H 106, valproic acid, insulin, SIRT1 activator 3, or a combination thereof. Tom, are these all inhibitors?

63. The method of any one of claims 29 to 62, wherein said mitogenic factor is Y-27632, CHIR99021, tranylcypromine, PMA, 3-deazaneplanocin A, 5-aza-2′-deoxycytidine, 4-phenylbutyrate, one or more PKC alpha activators, one or more PKC epsilon activators, valproic acid, insulin, or a combination thereof.

64. The method of any one of claims 29 to 63, wherein said incubation medium further comprises 0.1-100 ng/ml of Y-27632, CHIR99021, tranylcypromine, PMA, 3-deazaneplanocin A, 5-aza-2′-deoxycytidine, 4-phenylbutyrate, one or more PKC alpha activators, one or more PKC epsilon activators, valproic acid, insulin, or a combination thereof.

65. The method of any one of claims 29 to 64, wherein said mitogenic factor is PMA.

66. The method of any one of claims 29 to 65, wherein said incubation medium further comprises 1-50 ng/ml PMA.

67. The method of any one of claims 1 to 66, wherein said incubation medium comprises an effective amount of an inhibitor to reduce expression or function of a protein or complex from 10 to 99%.

68. The method of any one of claims 1 to 67, wherein said incubation medium comprises an effective amount of an activator to increase expression or function of a protein or complex by 10 to 99%.

69. The method of any one of claims 1 to 68, further comprising the step of genetically modifying the fibroblasts.

70. The method of claim 69, wherein said genetic modification comprises transfecting the fibroblasts with one or more genes.

71. The method of claim 70, wherein a therapeutically effective amount fibroblasts genetically modified with said one or more genes are provided as a therapeutic application to an individual in need thereof.

72. The method of claim 71, wherein said therapeutic application is treatment of fibrosis, treatment of inflammation, acceleration of healing, treatment of clotting disorders, or promotion of angiogenesis.

73. The method of claim 71 or 72, wherein said therapeutic application comprises treatment of fibrosis, wherein said one or more genes comprises one or more anti-fibrotic factors.

74. The method of claim 73, wherein said antifibrotic factor is one or more cathepsins, calpin 1, calpin 2, chondroitinase ABC, chondroitinase AC, pancreatic elastase, elastase-2a, elastase-2b, neutrophil elastase, proteinase-3, endogenous vascular elastase, mast cell chymase, mast cell tryptase, plasmin, thrombin, granzyme B, hyaluronidase, chymopapain, chymotrypsin, legumain, collagenase, one or more matrix metalloproteinases, one or more aggrecanases, papain, subtilisin, subtilisin A, heparanase, one or more TGF-beta receptor antagonists, one or more angiotensin inhibitors, one or more angiotensin-converting enzyme inhibitors, one or more angiotensin-II receptor antagonists, one or more cannabinoid receptor, one or more interleukins, one or more interferons, one or more cytokines, one or more chemokines, one or more chemotactic molecules, relaxin, one or more hormones, or a combination thereof.

75. The method of claim 73 or 74, wherein said antifibrotic factor is one or more cathepsins.

76. The method of claim 74 or 75, wherein said cathepsin is cathepsin D, cathepsin E, cathepsin S, cathepsin K, cathepsin L, cathepsin B, cathepsin C, cathepsin H, cathepsin F, cathepsin G, cathepsin O, cathepsin R, cathepsin V, cathepsin W, cathepsin Z, or a combination thereof.

77. The method of any one of claims 73 to 76, wherein said antifibrotic factor is one or more metalloproteinases.

78. The method of claim 77, wherein said metalloproteinase is MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-23, MMP-24, MMP-25, MMP-26, MMP-23, MMP-27, MMP-28, or a combination thereof.

79. The method of any one of claims 73 to 78, wherein said anti-fibrotic factor is one or more aggrecanases.

80. The method of claim 79, wherein said aggrecanase is ADAMTS-1, ADAMTS-2, ADAMTS -3, ADAMTS -4, ADAMTS -5, ADAMTS -14, or a combination thereof.

81. The method of any one of claims 73 to 80, wherein said anti-fibrotic factor is one or more TGF-beta receptor antagonists.

82. The method of claim 81, wherein said TGF-beta receptor antagonist is decorin, one or more decorin-fusion proteins, one or more anti-TGF-beta receptor antibodies, one or more anti-TGF-beta receptor oligonucleotides, or a combination thereof.

83. The method of claim 81 or 82, wherein said TGF-beta receptor antagonist is one or more decorin-fusion proteins.

84. The method of claim 82 or 83, wherein said decorin-fusion protein is CAR-decorin.

85. The method of any one of claims 81 to 84, wherein said TGF-beta receptor antagonist is one or more anti-TGF-beta receptor antibodies.

86. The method of claim 85, wherein said anti-TGF-beta receptor antibody is fresolimumab, lerdelimumab, metelimumab, one or more monoclonal antibodies, one or more humanized antibodies, one or more human antibodies, one or more antibody fragments, or a combination thereof.

87. The method of any one of claims 81 to 86, wherein said TGF-beta receptor antagonist is one or more anti-TGF-beta receptor oligonucleotides.

88. The method of claim 87, wherein said anti-TGF-beta receptor oligonucleotide is trabedersen, one or more siRNAs targeting TGF-beta, or a combination thereof.

89. The method of claim 71 or 72, wherein said therapeutic application comprises treatment of inflammation, wherein said one or more genes comprises one or more anti-inflammatory factors.

90. The method of claim 89, wherein said anti-inflammatory factor is one or more inflammatory cytokine antagonists, one or more anti-microbial factors, one or more antibodies, one or more non-antibody anti-inflammatory factors, one or more soluble receptor fusion proteins, or a combination thereof.

91. The method of claim 89 or 90, wherein said anti-inflammatory factor is one or more inflammatory cytokine antagonists.

92. The method of claim 90 or 91, wherein said inflammatory cytokine antagonist is one or more IL-6 antagonists, one or more IL-10 antagonists, IL-10, LL-37, thymosin beta 4, or a combination thereof.

93. The method of any one of claims 89 to 92, wherein said anti-inflammatory factor is one or more anti-microbial factors.

94. The method of claim 93, wherein said anti-microbial factor is LL-37, thymosin beta 4, or a combination thereof.

95. The method of any one of claims 89 to 94, wherein said anti-inflammatory factor is one or more antibodies.

96. The method of claim 95, wherein said antibody is one or more anti-TNF-alpha antibodies, one or more anti-IL-6 antibodies, one or more anti-IL-10 antibodies, one or more monoclonal antibodies, one or more humanized antibodies, one or more human antibodies, one or more antibody fragments, or a combination thereof.

97. The method of claim 95 or 96, wherein said antibody is one or more anti-TNF-alpha antibodies.

98. The method of claim 96 or 97, wherein said anti-TNF-alpha antibody is infliximab, adalimumab, certolizumab pegol, golimumab, or combinations thereof.

99. The method of any one of claims 89 to 98, wherein said anti-inflammatory factor is one or more soluble receptor fusion proteins.

100. The method of claim 99, wherein said soluble receptor fusion protein is etanercept.

101. The method of any one of claims 89 to 100, wherein said anti-inflammatory factor is one or more non-antibody anti-inflammatory factors.

102. The method of claim 101, wherein said non-antibody anti-inflammatory factor excludes antibodies.

103. The method of any one of claims 101 to 102, wherein said non-antibody anti-inflammatory factor excludes monoclonal antibodies, human antibodies, humanized antibodies, or a combination thereof.

104. The method of any one of claims 101 to 103, wherein said non-antibody anti-inflammatory factor excludes TNF antibodies, alemtuzumab, afelimomab, aselizumab, atlizumab, atorolimumab, basiliximab, belimumab, bertilimumab, cedelizumab, clenoliximab, daclizumab, dorlimomab aritox, dorlixizumab, eculizumab, efalizumab, elsilimomab, erlizumab, faralimomab, fontolizumab, galiximab, gantenerumab, gavilimomab, golimumab, gomiliximab, ibalizumab, inolimomab, ipilimumab, keliximab, lebrilizumab, lerdelimumab, lumiliximab, maslimomab, mepolizumab, metelimumab, morolimumab, muromonab-CD3, natalizumab, nerelimomab, ocrelizumab, odulimomab, omalizumab, otelixizumab, pascolizumab, pexelizumab, rituxumab, reslizumab, rovelizumab, ruplizumab, siplizumab, talizumab, telimomab aritox, teneliximab, teplizumab, tocilizumab, toralizumab, vapaliximab, vepalimomab, visilizumab, zanolimumab, ziralimumab, zolimomab aritox, or a combination thereof.

105. The method of claim 71 or 72, wherein said therapeutic application comprises acceleration of healing, wherein said one or more genes comprises one or more extracellular matrix factors.

106. The method of claim 105, wherein said extracellular matrix factor is decorin, fibronectin, vitronectin, laminin, COM, tenascin-C, tenascin-X, elastin, TIMP-1, albumin, osteonectin, one or more collagens, one or more keratins, one or more thrombospondins, one or more proteoglycans, one or more glycosaminoglycans, one or more integrins, or a combination thereof.

107. The method of claim 105 or 106, wherein said extracellular matrix factor is one or more collagens.

108. The method of claim 106 or 107, wherein said collagen is collagen-I, collagen-III, collagen-VI, or a combination thereof.

109. The method of any one of claims 105 to 108, wherein said extracellular matrix factor is one or more keratins.

110. The method of claim 109, wherein said keratin is K6, K16, or a combination thereof.

111. The method of any one of claims 105 to 110, wherein said extracellular matrix factor is one or more thrombospondins.

112. The method of claim 111, wherein said thrombospondin is thrombospondin-1, thrombospondin-2, or a combination thereof.

113. The method of any one of claims 105 to 112, wherein said extracellular matrix factor is one or more proteoglycans.

114. The method of claim 113, wherein said proteoglycan is versican, syndecan, glypicans, perlecan, lumican, heparin sulfate, or a combination thereof.

115. The method of any one of claims 105 to 114, wherein said extracellular matrix factor is one or more glycosaminoglycans.

116. The method of claim 115, wherein said glycosaminoglycan is a combination of hyaluronan and hyaluronic acid.

117. The method of claim 71 or 72, wherein said therapeutic application comprises treatment of one or more clotting disorders, wherein said one or more genes comprises one or more clotting factors.

118. The method of claim 117, wherein said clotting factor is factor I, factor II, factor III, factor V, factor VII, factor VIII, factor IX, factor X, factor XI, factor XII, factor XIII, von Willebrand factor, prekallikrein, HMWK, fibronectin, antithrombin III, heparin cofactor II, protein C, protein S, protein Z, plasminogen, tPA, urokinase, or a combination thereof.

119. The method of claim 71 or 72, wherein said therapeutic application comprises promotion of angiogenesis, wherein said one or more genes comprises one or more angiogenic factors.

120. The method of claim 119, wherein said angiogenic factor is one or more VEGFs, one or more angiopoietins, one or more angiopoietin-like proteins, one or more FGFs, one or more TGFs, one or more TNFs, one or more CSFs, one or more NOS s, EGF, or a combination thereof.

121. The method of claim 1199 or 120, wherein said angiogenic factor is one or more VEGFs.

122. The method of claim 120 or 121, wherein said VEGF is VEGF-A, VEGF-B, VEGF-C, VEGF-D, VEGF-E, PIGF, or a combination thereof.

123. The method of any one of claims 119 to 122, wherein said angiogenic factor is one or more angiopoietins.

124. The method of claim 123, wherein said angiopoietin is ANGPT1, ANGPT2, ANGPT4, or a combination thereof.

125. The method of any one of claims 119 to 124, wherein said angiogenic factor is one or more angiopoietin-like proteins.

126. The method of claim 125, wherein said angiopoietin-like protein is ANGPTL1, ANGPTL2, ANGPTL3, ANGPTL4, ANGPTL5, ANGPTL6, ANGTPL7, or a combination thereof.

127. The method of any one of claims 119 to 126, wherein said angiogenic factor is one or more FGFs.

128. The method of claim 127, wherein said FGF is FGF-1, FGF-2, or a combination thereof.

129. The method of any one of claims 119 to 128, wherein said angiogenic factor is one or more TGFs.

130. The method of claim 129, wherein said TGF is TGF-alpha, TGF-beta, or combinations thereof.

131. The method of any one of claims 119 to 130, wherein said angiogenic factor is one or more TNFs.

132. The method of claim 131, wherein said TNF is TNF-alpha.

133. The method of any one of claims 119 to 1332, wherein said angiogenic factor is one or more CSFs.

134. The method of claim 133, wherein said CSF is GM-CSF, M-CSF, or combinations thereof.

135. The method of any one of claims 1 to 134, wherein fibroblasts are autologous, allogeneic, or xenogeneic with respect to a recipient.

136. The method of any one of claims 1 to 135, wherein the source of said fibroblasts comprises: a) fallopian tube; b) menstrual blood; c) endometrium; d) skin; e) nail cuticle; f) deciduous tooth; g) hair follicle; h) tonsil; i) omentum; j) bone marrow; k) placenta; l) umbilical cord; m) peripheral blood; n) adipose tissue; o) amniotic fluid; p) umbilical cord blood; q) embryonic fibroblasts; r) plastic surgery related by-product; or s) nail matrix.

137. The method of claim 136, wherein said peripheral blood is extracted after activation of fibroblasts.

138. The method of claim 137, wherein said activation is achieved by administration of G-CSF.

139. The method of claim 137 or 138 wherein said activation is achieved by administration of M-CSF.

140. The method of any one of claims 137 to 139, wherein said activation is achieved by administration of GM-CSF.

141. The method of any one of claims 137 to 140, wherein said activation is achieved by administration of Flt3 ligand.

142. The method of any one of claims 1 to 141, wherein said fibroblasts are transfected with one or more genes selected from a group comprising: Lin-28, STAT3, NFKB, CEBP/B, SOX2, OCT4, WNT5A, LIF, COX2, RUNX2 and NANOG.

143. The method of any one of claims 1 to 142, wherein said fibroblasts endogenously express at least one of CD184, CD193, CD235a, CD318, CD255, CD268, fMLP, ITGA2, or ITGA4.

144. The method of any one of claims 1 to 143, wherein said fibroblasts are transfected with and/or endogenously express one or more genes selected from the group consisting of 37135 (Sep-01); 37500 (Sep-02); 37865 (Sep-03); 38231 (Sep-04); 38596 (Sep-05); 38961 (Sep-06); 39326 (Sep-07); 40057 (Sep-09); 40422 (Sep-10); 40787 (Sep-11); 41153 (Sep-12); 41883 (Sep-14); A1BG; A1CF; A2M; A2ML1; A4GNT; AAAS; AACS; AADAC; AAGAB; AAK1; AANAT; AARD; AARS2; AARS; AASDH; AASDHPPT; AASS; AATF; AATK; ABAT; ABCA12; ABCA13; ABCA1; ABCA2; ABCA3; ABCA5; ABCA6; ABCA7; ABCA9; ABCB11; ABCB1; ABCB4; ABCB5; ABCB6; ABCB7; ABCB8; ABCB9; ABCC10; ABCC11; ABCC12; ABCC1; ABCC2; ABCC3; ABCC4; ABCC5; ABCC6; ABCC8; ABCC9; ABCD1; ABCD2; ABCD3; ABCD4; ABCE1; ABCF1; ABCF2; ABCG1; ABCG2; ABCG4; ABCG5; ABCG8; ABHD12B; ABHD12; ABHD16A; ABHD17B; ABHD2; ABHDS; ABHD6; ABI1; ABI2; ABI3BP; ABI3; ABL1; ABL2; ABLIM1; ABLIM2; ABO; ABRA; ABR; ABT1; ACAA1; ACAA2; ACACA; ACACB; ACAD10; ACAD8; ACAD9; ACADL; ACADSB; ACADS; ACADVL; ACAN; ACAT1; ACAT2; ACBD3; ACBD4; ACBD5; ACBD6; ACCS; ACD; ACE2; ACE; ACER2; ACER3; ACHE; ACINI; ACKR1; ACKR2; ACKR3; ACKR4; ACLY; ACMSD; ACO1; ACO2; ACOT13; ACOT1; ACOT2; ACOT9; ACOX1; ACOX2; ACOX3; ACOXL; ACP1; ACP2; ACP5; ACP6; ACPP; ACRBP; ACRC; ACR; ACRV1; ACSBG1; ACSBG2; ACSF3; ACSL1; ACSL3; ACSL5; ACSL6; ACSM1; ACSM2B; ACSM3; ACSS1; ACSS2; ACSS3; ACTA1; ACTA2; ACTB; ACTBL2; ACTC1; ACTG1; ACTG2; ACTL6A; ACTL6B; ACTL8; ACTL9; ACTN1; ACTN2; ACTN3; ACTN4; ACTR1A; ACTR1B; ACTR2; ACTR3B; ACTR3; ACTR5; ACTRT1; ACVR1B; ACVR1C; ACVR1; ACVR2A; ACVR2B; ACVRL1; ACYL; ACY3; ACYP2; ADAD1; ADA; ADAM10; ADAM11; ADAM12; ADAM15; ADAM17; ADAM19; ADAM20; ADAM21; ADAM22; ADAM23; ADAM28; ADAM29; ADAM2; ADAM33; ADAM7; ADAMS; ADAMS; ADAMDEC1; ADAMTS10; ADAMTS12; ADAMTS13; ADAMTS14; ADAMTS15; ADAMTS16; ADAMTS17; ADAMTS18; ADAMTS19; ADAMTS1; ADAMTS20; ADAMTS2; ADAMTS3; ADAMTS4; ADAMTS5; ADAMTS6; ADAMTS7; ADAMTS8; ADAMTS9; ADAMTSL1; ADAMTSL2; ADAMTSL3; ADAMTSL4; ADAMTSL5; ADAP1; ADAP2; ADARB1; ADARB2; ADAR; ADAT2; ADAT3; ADCK3; ADCK4; ADCY10; ADCY1; ADCY2; ADCY3; ADCYS; ADCY6; ADCY7; ADCY8; ADCY9; ADCYAP1; ADCYAP1R1; ADD1; ADD2; ADD3; ADGB; ADGRA1; ADGRA2; ADGRA3; ADGRB1; ADGRB2; ADGRB3; ADGRD1; ADGRD2; ADGRE1; ADGRE2; ADGRE5; ADGRF5; ADGRG1; ADGRG2; ADGRG3; ADGRG6; ADGRG7; ADGRL1; ADGRL3; ADGRV1; ADH1A; ADH1B; ADH4; ADH5; ADH6; ADH7; ADHFE1; AM; ADIG; ADIPOQ; ADIPOR1; ADIPOR2; ADIRF; ADK; ADM2; ADM; ADNP; ADO; ADORAL; ADORA2A; ADORA2B; ADPGK; ADPRH; ADPRHL1; ADPRHL2; ADRA1A; ADRA1B; ADRA1D; ADRA2A; ADRA2B; ADRA2C; ADRB1; ADRB2; ADRB3; ADRBK1; ADRBK2; ADRM1; ADSL; ADSS; ADSSL1; ADTRP; AEBP2; AEN; AES; AFAP1; AFAP1L1; AFAP1L2; AFF1; AFF2; AFF3; AFF4; AFG3L2; AFM; AFP; AGA; AGAP1; AGAP2; AGAP3; AGAP4; AGBL1; AGBL2; AGBL3; AGBL4; AGER; AGFG1; AGFG2; AGGF1; AGK; AGMO; AGO2; AGPAT1; AGPAT2; AGPAT3; AGPAT4; AGPAT9; AGPS; AGR2; AGR3; AGRN; AGRP; AGT; AGTPBP1; AGTR1; AGTR2; AGXT2; AGXT; AHCY; AHCYL2; AHI1; AHNAK; AHR; AHRR; AHSA1; AHSA2; AHSG; AICDA; AIDA; AIF1; AIFM1; AIFM2; AIFM3; AIG1; AIM1; AIM2; AIMP1; AIMP2; AIP; AIPL1; AIRE; AJAP1; AJUBA; AK1; AK3; AK6; AK7; AK8; AK9; AKAP10; AKAP12; AKAP13; AKAP17A; AKAP1; AKAP2; AKAP3; AKAP4; AKAP5; AKAP6; AKAP7; AKAP9; AKIP1; AKIRIN2; AKNA; AKR1B10; AKR1B1; AKR1C1; AKR1C2; AKR1C3; AKR1C4; AKR1D1; AKR1E2; AKR7A3; AKT1; AKT1S1; AKT2; AKT3; AKTIP; ALAD; ALAS1; ALAS2; ALB; ALCAM; ALDH16A1; ALDH18A1; ALDH1A1; ALDH1A2; ALDH1A3; ALDH1B1; ALDH1L1; ALDH2; ALDH3A1; ALDH3A2; ALDH3B1; ALDH4A1; ALDH5A1; ALDH6A1; ALDH7A1; ALDH9A1; ALDOA; ALDOB; ALDOC; ALG10B; ALG10; ALG11; ALG12; ALG13; ALG1; ALG1L; ALG2; ALG3; ALG8; ALG9; ALKBH1; ALKBH2; ALKBH3; ALKBH7; ALKBH8; ALK; ALLC; ALMS1; ALOX12B; ALOX12; ALOX15B; ALOX15; ALOX5AP; ALOX5; ALOXE3; ALPI; ALPK1; ALPK2; ALPK3; ALPP; ALPPL2; ALS2CL; ALS2CR12; ALS2; ALX1; ALX4; ALYREF; AMACR; AMBN; AMBP; AMBRA1; AMD1; AMELX; AMELY; AMER1; AMER2; AMER3; AMFR; AMH; AMHR2; AMICA1; AMIGO2; AMMECR1; AMN; AMOT; AMOTL1; AMPD2; AMPD3; AMPH; AMT; AMZ1; ANAPC10; ANAPC11; ANAPC13; ANAPC1; ANAPC2; ANAPC4; ANAPC5; ANAPC7; ANG; ANGPT1; ANGPT2; ANGPT4; ANGPTL1; ANGPTL2; ANGPTL4; ANGPTL6; ANK1; ANK2; ANK3; ANKFN1; ANKFY1; ANKHD1-EIF4EBP3; ANKHD1; ANKH; ANKK1; ANKLET; ANKLE2; ANKMY1; ANKRD10; ANKRD11; ANKRD12; ANKRD18A; ANKRD1; ANKRD23; ANKRD26; ANKRD28; ANKRD2; ANKRD30A; ANKRD36B; ANKRD36; ANKRD37; ANKRD44; ANKRD45; ANKRD46; ANKRD50; ANKRD55; ANKRD6; ANKRD7; ANKS1A; ANKS1B; ANKS4B; ANKS6; ANLN; AN010; ANO1; ANO2; ANO3; ANO4; ANDS; ANO6; ANO7; ANP32A; ANP32B; ANP32D; ANPEP; ANTXR1; ANTXR2; ANXA10; ANXA11; ANXA13; ANXA1; ANXA2; ANXA2R; ANXA3; ANXA4; ANXA5; ANXA6; ANXA7; ANXA8; ANXA8L1; AOAH; AOC1; AOC2; AOC3; AOX1; AP1AR; AP1B1; AP1G1; AP1M1; AP1M2; AP1S1; AP1S2; AP1S3; AP2A1; AP2B1; AP2M1; AP2S1; AP3B1; AP3D1; AP3M2; AP3S1; AP3S2; AP4B1; AP4E1; AP4M1; AP4S1; AP5M1; AP5Z1; APAF1; APBA1; APBA2; APBA3; APBB1; APBB1IP; APBB2; APBB3; APC2; APCDD1; APCDD1L; APC; APCS; APEH; APEX1; APEX2; APH1B; APIS; APIP; APLN; APLNR; APLP1; APLP2; APOA1BP; APOA2; APOA4; APOA5; APOBEC1; APOBEC2; APOBEC3A_B; APOBEC3B; APOBEC3C; APOBEC3F; APOBEC3G; APOBEC3H; APOB; APOBR; APOC1; APOC2; APOC3; APOC4; APOD; APOE; APOF; APOH; APOL1; APOL2; APOL3; APOL4; APOL6; APOLD1; APOM; APOO; APOPT1; APPBP2; APP; APPL1; APPL2; APRT; APTX; AQP10; AQP1; AQP2; AQP3; AQP4; AQP5; AQP6; AQP7; AQP8; AQP9; ARAF; ARAP1; ARAP3; ARC; ARCN1; AREG; ARF1; ARF3; ARF4; ARF6; ARFGAP1; ARFGAP2; ARFGAP3; ARFGEF1; ARFGEF2; ARFGEF3; ARFIP1; ARFRP1; ARG1; ARGLU1; ARHGAP10; ARHGAP11A; ARHGAP11B; ARHGAP15; ARHGAP18; ARHGAP1; ARHGAP20; ARHGAP21; ARHGAP22; ARHGAP23; ARHGAP24; ARHGAP25; ARHGAP26; ARHGAP27; ARHGAP28; ARHGAP30; ARHGAP31; ARHGAP32; ARHGAP35; ARHGAP42; ARHGAP4; ARHGAP5; ARHGAP6; ARHGAP9; ARHGD1A; ARHGD1B; ARHGEF10; ARHGEF10L; ARHGEF11; ARHGEF12; ARHGEF15; ARHGEF16; ARHGEF17; ARHGEF1; ARHGEF25; ARHGEF26; ARHGEF28; ARHGEF2; ARHGEF38; ARHGEF3; ARHGEF4; ARHGEF5; ARHGEF6; ARHGEF7; ARHGEF9; AR; ARID1B; ARID2; ARID3A; ARID3B; ARID4A; ARID4B; ARID5B; ARIH1; ARL11; ARL13A; ARL13B; ARL14EP; ARL14; ARL15; ARL1; ARL2BP; ARL2; ARL3; ARL4A; ARL4C; ARL4D; ARL5A; ARL5B; ARL6; ARL6IP1; ARL6IP5; ARMC10; ARMC1; ARMC2; ARMC3; ARMC4; ARMC5; ARMC8; ARMC9; ARMCX1; ARMS2; ARMT1; ARNT2; ARNT; ARNTL2; ARNTL; ARPC1A; ARPC1B; ARPC2; ARPC3; ARPC5; ARPIN; ARPP21; ARR3; ARRB1; ARRB2; ARRDC2; ARRDC3; ARRDC4; ARSA; ARSB; ARSD; ARSE; ARSF; ARSG; ARSH; ARSI; ARSJ; ARSK; ART1; ART3; ART4; ARVCF; ARX; AS3MT; ASAH1; ASAH2; ASAP1; ASAP2; ASB10; ASB13; ASB15; ASB18; ASB1; ASB2; ASB6; ASB7; ASCC1; ASCC2; ASCC3; ASCL1; ASCL2; ASCL4; ASF1A; ASF1B; ASH1L; ASH2L; ASIC1; ASIC2; ASIC3; ASIC4; ASIC5; ASIP; ASL; ASMT; ASMTL; ASNA1; ASNS; ASPA; ASPG; ASPH; ASPM; ASPN; ASPRV1; ASPSCR1; ASRGL1; ASS1; ASTN2; ASUN; ASXL1; ASXL2; ASXL3; ASZ1; ATAD2; ATAD3B; ATAD3C; ATAD5; ATAT1; ATCAY; ATE1; ATFL; ATF2; ATF3; ATF4; ATF5; ATF6B; ATF6; ATF7; ATF7IP; ATG10; ATG12; ATG16L1; ATG2B; ATG3; ATG4A; ATG4B; ATG4C; ATG5; ATG7; ATG9A; ATIC; ATL1; ATL2; ATL3; ATM; ATN1; ATOH1; ATOH7; ATOX1; ATP10A; ATP10B; ATP10D; ATP11A; ATP11AUN; ATP11B; ATP12A; ATP13A3; ATP13A4; ATP1A2; ATP1A3; ATP1A4; ATP1B1; ATP1B2; ATP2A1; ATP2A2; ATP2A3; ATP2B1; ATP2B2; ATP2B3; ATP2B4; ATP2C1; ATP2C2; ATP4A; ATP4B; ATP5A1; ATP5B; ATP5C1; ATP5D; ATP5E; ATP5G1; ATP5G2; ATP5G3; ATP5H; ATP5J2; ATP5J; ATP5L; ATP5O; ATP6AP1; ATP6AP1L; ATP6AP2; ATP6V0A1; ATP6V0A2; ATP6V0A4; ATP6V0C; ATP6V0D1; ATP6V0E1; ATP6V0E2; ATP6V1B1; ATP6V1B2; ATP6V1C1; ATP6V1D; ATP6V1E1; ATP6V1F; ATP6V1G1; ATP6V1G2; ATP6V1G3; ATP7A; ATP7B; ATP8A1; ATP8A2; ATP8B1; ATP8B3; ATP8B4; ATP9B; ATPAF2; ATRAID; ATR; ATRIP; ATRN; ATRNL1; ATRX; ATXN10; ATXN1; ATXN1L; ATXN2; ATXN2L; ATXN3; ATXN7; ATXN7L3B; AUH; AURKA; AURKB; AURKC; AUTS2; AVEN; AVP; AVPI1; AVPR1A; AVPR1B; AVPR2; AWAT1; AXDND1; AXIN1; AXIN2; AXL; AZGP1; AZI2; AZIN1; AZIN2; AZUL; B2M; B3GALNT1; B3GALNT2; B3GALT2; B3GALT4; B3GALT5; B3GALTL; B3GAT1; B3GAT2; B3GAT3; B3GNT2; B3GNT3; B3GNT5; B3GNT6; B3GNT8; B3GNTL1; B4GALNT1; B4GALNT2; B4GALNT3; B4GALT1; B4GALT3; B4GALT4; B4GALT5; B4GALT6; B4GALT7; B4GAT1; B9D1; B9D2; BAALC; BAAT; BABAM1; BACE1; BACE2; BACH1; BACH2; BAD; BAG1; BAG3; BAG4; BAG5; BAG6; BAIAP2L1; BAIAP3; BAK1; BAMBI; BANF1; BANK1; BANP; BAP1; BARD1; BARHL1; BARX1; BARX2; BASP1; BATF2; BATF; BAX; BAZ1A; BAZ1B; BAZ2A; BAZ2B; BBC3; BBIP1; BBOX1; BBS10; BBS12; BBS1; BBS2; BBS4; BBS5; BBS7; BBS9; BBX; BCAM; BCAN; BCAP29; BCAP31; BCAR1; BCAS1; BCAS3; BCAS4; BCAT1; BCAT2; BCCIP; BCDIN3D; BCHE; BCKDHA; BCKDHB; BCKDK; BCL10; BCL11A; BCL11B; BCL2A1; BCL2; BCL2L10; BCL2L11; BCL2L12; BCL2L13; BCL2L14; BCL2L1; BCL2L2; BCL2L2-PABPN1; BCL3; BCL6B; BCL6; BCL7A; BCL7B; BCL7C; BCL9L; BCLAF1; BCO1; BCO2; BCOR; BCORL1; BCR; BCS1L; BDH1; BDH2; BDKRB1; BDKRB2; BDNF; BDP1; BEAN1; BEGAIN; BEND3; BEND4; BEST1; BEST2; BET1; BET1L; BEX1; BEX2; BEX4; BFAR; BFSP1; BFSP2; BGLAP; BGN; BHLHA15; BHLHA9; BHLHB9; BHLHE22; BHLHE23; BHLHE40; BHLHE41; BHMT2; BHMT; BICC1; BICD1; BICD2; BID; BIN1; BIN2; BIN3; BIRC2; BIRC3; BIRC5; BIRC6; BIRC7; BIVM; BLCAP; BLID; BLK; BLMH; BLM; BLNK; BLOC1S2; BLOC1S3; BLOC1S4; BLOC1S5; BLOC1S6; BLVRA; BLVRB; BLZF1; BMF; BMI1; BMP10; BMP15; BMP1; BMP2; BMP2K; BMP3; BMP4; BMP5; BMP6; BMP7; BMP8B; BMPER; BMPR1A; BMPR1B; BMPR2; BMS1; BMX; BNC1; BNC2; BNIP1; BNIP2; BNIP3; BNIP3L; BNIPL; BOC; BOD1L2; BOK; BOLA3; BOLL; BOP1; BORA; BPGM; BPIFA1; BPIFA2; BPIFA3; BPIFB1; BPIFB2; BPIFC; BPI; BPNT1; BPTF; BPY2; BRAF; BRAP; BRAT1; BRCA1; BRCA2; BRCC3; BRD1; BRD2; BRD3; BRD4; BRD7; BRD8; BRE; BRF1; BRF2; BRI3BP; BRI3; BRINP1; BRINP2; BRINP3; BRIP1; BRK1; BRMS1; BRMS1L; BRS3; BRSK1; BRSK2; BRWD1; BRWD3; BSCL2; BSG; BSN; BSPH1; BSPRY; BST1; BST2; BSX; BTBD10; BTBD11; BTBD16; BTBD1; BTBD2; BTBD3; BTBD9; BTC; BTD; BTF3; BTG1; BTG2; BTG3; BTG4; BTK; BTLA; BTN1A1; BTN2A1; BTN2A2; BTN3A1; BTN3A2; BTN3A3; BTNL2; BTRC; BUB1B; BUB1; BUB3; BUD13; BUD31; BVES; BYSL; BZRAP1; BZW1; C10orf107; C10orf10; C10orf113; C10orf11; C10orf2; C10orf32; C10orf35; C10orf54; C10orf67; C10orf82; C10orf88; C10orf90; C11orf21; C11orf30; C11orf53; C11orf65; C11orf68; C11orf73; C11orf74; C11orf80; C11orf87; C11orf95; C12orf10; C12orf42; C12orf43; C12orf50; C12orf57; C12orf5; C12orf65; C12orf66; C12orf75; C12orf77; C14orf166; C14orf177; C14orf1; C15orf32; C15orf41; C15orf48; C15orf53; C15orf59; C16orf72; C16orf74; C16orf78; C16orf95; C17orf51; C17orf53; C17orf64; C17orf96; C18orf54; C18orf8; C19orf12; C19orf18; C19orf24; C19orf26; C19orf33; C19orf40; C19orf45; C19orf48; C19orf57; C19orf68; C1D; C1GALT1C1; C1GALT1; C1orf106; C1orf109; C1orf110; C1orf112; C1orf115; C1orf127; C1orf141; C1orf167; C1orf204; C1orf226; C1orf228; C1orf27; C1orf61; C1orf86; C1QB; C1QBP; C1QL1; C1QL3; C1QTNF1; C1QTNF3; C1QTNF5; C1QTNF6; C1QTNF7; C1QTNF9B-AS1; C1R; C1RL; C1S; C20orf194; C20orf196; C20orf27; C20orf85; C21orf2; C21orf33; C21orf59; C21orf62; C21orf91; C22orf29; C2CD3; C2CD4A; C2CD4B; C2CD5; C2; C2orf16; C2orf40; C2orf43; C2orf47; C2orf57; C2orf61; C3AR1; C3; C3orf17; C3orf18; C3orf20; C3orf35; C3orf56; C3orf58; C3orf67; C3orf79; C4A; C4B_2; C4B; C4BPA; C4BPB; C4orf22; C4orf26; C4orf27; C4orf32; C4orf33; C4orf36; C4orf48; C4orf51; C5AR1; C5AR2; C5; C5orf22; C5orf30; C5orf34; C5orf38; C5orf42; C5orf63; C6; C6orf106; C6orf10; C6orf15; C6orf25; C6orf47; C6orf48; C6orf89; C7; C7orf49; C7orf57; C7orf60; C7orf62; C7orf65; C7orf69; C7orf72; C8orf34; C8orf37; C8orf46; C8orf48; C8orf4; C8orf86; C9; C9orf106; C9orf135; C9orf152; C9orf156; C9orf170; C9orf171; C9orf3; C9orf43; C9orf66; C9orf72; C9orf85; C9orf91; C9orf9; CA10; CA11; CA12; CA13; CA1; CA2; CA3; CA4; CASA; CA6; CA8; CAB39; CAB39L; CABIN1; CABLES1; CABP2; CABP4; CABS1; CABYR; CACNA1A; CACNA1B; CACNA1C; CACNA1D; CACNA1E; CACNA1F; CACNA1G; CACNA1H; CACNA1I; CACNA1S; CACNA2D1; CACNA2D2; CACNA2D3; CACNA2D4; CACNB1; CACNB2; CACNB3; CACNB4; CACNG2; CACNG3; CACNG4; CACNG5; CACNG6; CACUL1; CACYBP; CAD; CADM1; CADM2; CADM3; CADM4; CADPS2; CADPS; CAGE1; CALB1; CALB2; CALCA; CALCB; CALCOCO1; CALCOCO2; CALCR; CALCRL; CALD1; CALHM1; CALHM2; CALHM3; CALM2; CALML3; CALML5; CALN1; CALR3; CALR; CALU; CALY; CAMK1D; CAMK1G; CAMK1; CAMK2A; CAMK2B; CAMK2D; CAMK2G; CAMK4; CAMKK1; CAMKK2; CAMKMT; CAMLG; CAMP; CAMSAP1; CAMSAP2; CAMTA1; CAND1; CAND2; CANT1; CANX; CAP1; CAP2; CAPG; CAPN10; CAPN13; CAPN14; CAPN1; CAPN2; CAPN3; CAPN5; CAPN6; CAPN7; CAPN9; CAPNS1; CAPRIN1; CAPRIN2; CAPS2; CAPS; CAPSL; CAPZA2; CAPZA3; CARD10; CARD11; CARD14; CARD16; CARD6; CARDS; CARDS; CARF; CARKD; CARM1; CARS; CARTPT; CASC1; CASC3; CASC4; CASC5; CASD1; CASK; CASP10; CASP14; CASP1; CASP2; CASP3; CASP4; CASP5; CASP6; CASP7; CASP8AP2; CASP8; CASP9; CASQ1; CASR; CASS4; CAST; CASZ1; CAT; CATSPER1; CATSPER2; CAV1; CAV2; CAV3; CBFA2T2; CBFA2T3; CBFB; CBLB; CBL; CBLL1; CBLN1; CBLN2; CBLN4; CBR1; CBR3; CBR4; CBS; CBX1; CBX2; CBX3; CBX4; CBX5; CBX6; CBX7; CBX8; CBY1; CC2D1A; CC2D1B; CC2D2A; CCAR1; CCAR2; CCBE1; CCBL1; CCDC101; CCDC102B; CCDC103; CCDC105; CCDC108; CCDC114; CCDC115; CCDC121; CCDC122; CCDC129; CCDC130; CCDC134; CCDC136; CCDC140; CCDC141; CCDC148; CCDC151; CCDC167; CCDC169-SOHLH2; CCDC170; CCDC171; CCDC175; CCDC176; CCDC178; CCDC180; CCDC181; CCDC185; CCDC22; CCDC28A; CCDC39; CCDC3; CCDC40; CCDC42B; CCDC42; CCDC50; CCDC54; CCDC60; CCDC62; CCDC63; CCDC65; CCDC66; CCDC67; CCDC68; CCDC6; CCDC78; CCDC80; CCDC83; CCDC85A; CCDC86; CCDC88A; CCDC88C; CCDC8; CCDC91; CCDC94; CCDC97; CCHCR1; CCKAR; CCKBR; CCK; CCL11; CCL13; CCL14; CCL15; CCL16; CCL17; CCL18; CCL19; CCL1; CCL20; CCL21; CCL22; CCL23; CCL24; CCL25; CCL26; CCL27; CCL28; CCL2; CCL3; CCL3L3; CCL4; CCL4L1; CCL4L2; CCL5; CCL7; CCL8; CCM2; CCNA1; CCNA2; CCNB1; CCNB2; CCNB3; CCNC; CCND1; CCND2; CCND3; CCNDBP1; CCNE1; CCNE2; CCNF; CCNG1; CCNG2; CCNH; CCNI; CCNJ; CCNJL; CCNK; CCNL1; CCNL2; CCNO; CCNT1; CCNY; CCP110; CCR10; CCR1; CCR2; CCR3; CCR4; CCR5; CCR6; CCR7; CCR8; CCR9; CCRL2; CCRN4L; CCSER1; CCS; CCT2; CCT3; CCT4; CCT5; CCT6A; CCT6B; CCT7; CD109; CD14; CD151; CD163; CD163L1; CD164; CD177; CD180; CD19; CD1A; CD1B; CD1C; CD1D; CD1E; CD200; CD200R1; CD207; CD209; CD226; CD22; CD244; CD247; CD248; CD24; CD274; CD276; CD27; CD28; CD2AP; CD300A; CD300C; CD300LF; CD302; CD320; CD33; CD34; CD36; CD37; CD38; CD30; CD3EAP; CD3E; CD3G; CD40; CD40LG; CD44; CD46; CD47; CD48; CD4; CD55; CD59; CD5; CD5L; CD63; CD68; CD69; CD6; CD72; CD74; CD79A; CD79B; CD7; CD80; CD81; CD82; CD83; CD84; CD86; CD8A; CD8B; CD93; CD96; CD99; CD9; CDADC1; CDAN1; CDC123; CDC14A; CDC14B; CDC16; CDC20B; CDC23; CDC25A; CDC25B; CDC25C; CDC27; CDC34; CDC37; CDC37L1; CDC42BPA; CDC42BPB; CDC42BPG; CDC42EP1; CDC42EP3; CDC42SE2; CDC45; CDC5L; CDC6; CDC73; CDC7; CDCA2; CDCA3; CDCA5; CDCA7; CDCA7L; CDCP1; CDH10; CDH11; CDH13; CDH15; CDH16; CDH17; CDH18; CDH19; CDH1; CDH20; CDH22; CDH23; CDH26; CDH2; CDH3; CDH4; CDH5; CDH6; CDH7; CDH8; CDH9; CDHR1; CDHR2; CDHR3; CDHR5; CDIPT; CDK10; CDK11B; CDK12; CDK13; CDK14; CDK15; CDK16; CDK17; CDK18; CDK19; CDK1; CDK20; CDK2AP1; CDK2AP2; CDK2; CDK3; CDK4; CDK5; CDK5R1; CDK5R2; CDK5RAP1; CDK5RAP2; CDK5RAP3; CDK6; CDK7; CDK8; CDK9; CDKAL1; CDKL1; CDKL2; CDKL3; CDKL4; CDKL5; CDKN1A; CDKN1B; CDKN1C; CDKN2A; CDKN2AIP; CDKN2B; CDKN2C; CDKN20; CDKN3; CDNF; CDO1; CDON; CDR1; CDR2; CDS2; CDT1; CDV3; CDX1; CDX2; CDX4; CDY1B; CDY2B; CDYL2; CDYL; CEACAM16; CEACAM19; CEACAM1; CEACAM21; CEACAM3; CEACAM4; CEACAM5; CEACAM6; CEACAM7; CEACAM8; CEBPA; CEBPB; CEBPD; CEBPE; CEBPG; CEBPZ; CECR1; CECR2; CELA1; CELA3B; CELF1; CELF2; CELF4; CELF5; CELF6; CEL; CELSR1; CELSR3; CEMIP; CEMP1; CEND1; CENPA; CENPB; CENPC; CENPE; CENPF; CENPH; CENPJ; CENPK; CENPN; CENPO; CENPQ; CENPU; CENPV; CENPW; CEP112; CEP120; CEP128; CEP131; CEP135; CEP152; CEP162; CEP164; CEP170; CEP192; CEP19; CEP250; CEP290; CEP41; CEP55; CEP57; CEP63; CEP68; CEP72; CEP76; CEP83; CEP85L; CEP89; CERT; CERK; CERKL; CERS1; CERS2; CERS3; CERS4; CERS6; CES1; CES2; CES3; CETN1; CETN2; CETN3; CETP; CFAP126; CFAP36; CFAP44; CFAP52; CFAP53; CFAP57; CFAP58; CFAP61; CFAP69; CFAP97; CFB; CFC1B; CFC1; CFD; CFDP1; CFH; CFHR1; CFHR2; CFHR3; CFHR4; CFHR5; CFI; CFL1; CFL2; CFLAR; CFP; CFTR; CGA; CGB1; CGB2; CGB5; CGB7; CGN; CGNL1; CGRRF1; CH.sub.25H; CHAC1; CHAD; CHAF1A; CHAF1B; CHAMP1; CHAT; CHCHD10; CHCHD1; CHCHD3; CHCHD5; CHCHD6; CHCHD7; CHD1; CHD2; CHD3; CHD4; CHD6; CHD7; CHD8; CHDH; CHEK1; CHEK2; CHERP; CHFR; CHGA; CHGB; CHI3L1; CHIC1; CHIC2; CHIT1; CHKA; CHKB; CHL1; CHM; CHML; CHMP1A; CHMP1B; CHMP2B; CHMP3; CHMP4A; CHMP4B; CHMP4C; CHMP5; CHN1; CHN2; CHODL; CHORDC1; CHP1; CHP2; CHPF; CHPT1; CHRAC1; CHRD; CHRDL1; CHRFAM7A; CHRM1; CHRM2; CHRM3; CHRM4; CHRM5; CHRNA10; CHRNA1; CHRNA2; CHRNA3; CHRNA4; CHRNA5; CHRNA6; CHRNA7; CHRNB1; CHRNB2; CHRNB3; CHRNB4; CHRND; CHRNE; CHRNG; CHST10; CHST11; CHST12; CHST13; CHST14; CHST15; CHST1; CHST2; CHST3; CHST4; CHST5; CHST6; CHST8; CHST9; CHSY1; CHSY3; CHTF18; CHTOP; CHURC1; CIAO1; CIAPIN1; CIB1; CIB2; CIC; CIDEB; CIDEC; CITTA; CILP2; CILP; CINP; CIPC; CIR1; CIRBP; CIRH1A; CISD1; CISD2; CISH; CITED1; CITED2; CIT; CIZ1; CKAP2; CKAP2L; CKAP4; CKAP5; CKB; CKLF; CKM; CKMT1A; CKMT1B; CKMT2; CKS1B; CKS2; CLASP1; CLASP2; CLCA2; CLCA4; CLCF1; CLC; CLCN1; CLCN2; CLCN3; CLCN4; CLCN5; CLCN6; CLCN7; CLCNKB; CLDN10; CLDN11; CLDN14; CLDN15; CLDN16; CLDN18; CLDN1; CLDN23; CLDN2; CLDN5; CLDN6; CLDN7; CLDN8; CLDN9; CLEC10A; CLEC11A; CLEC12A; CLEC14A; CLEC16A; CLEC1B; CLEC2A; CLEC2B; CLEC20; CLEC2L; CLEC3B; CLEC4A; CLEC4C; CLEC40; CLEC4E; CLEC4G; CLEC4M; CLEC5A; CLEC6A; CLEC7A; CLEC9A; CLECL1; CLGN; CLIC1; CLIC3; CLIC5; CLIC6; CLINT1; CLIP1; CLIP2; CLK1; CLK2; CLLU1; CLMN; CLMP; CLN3; CLN5; CLN6; CLN8; CLNK; CLNS1A; CLOCK; CLP1; CLPP; CLPS; CLPTM1; CLPTM1L; CLRN1; CLSPN; CLSTN1; CLSTN2; CLTA; CLTC; CLTCL1; CLUAP1; CLU; CLUL1; CLVS1; CLYBL; CMA1; CMAS; CMCJ; CMC2; CMC4; CMIP; CMKLR1; CMPK2; CMSS1; CMTM3; CMTM5; CMTM7; CMTM8; CMTR1; CMTR2; CMYA5; CNBD1; CNBP; CNDP1; CNDP2; CNGA1; CNGA3; CNGB1; CNGB3; CNIH3; CNKSR1; CNKSR2; CNKSR3; CNN1; CNN2; CNNM1; CNNM2; CNNM4; CNOT1; CNOT2; CNOT3; CNOT4; CNOT6; CNOT6L; CNOT7; CNOT8; CNP; CNPY2; CNPY3; CNR1; CNR2; CNRIP1; CNST; CNTF; CNTFR; CNTLN; CNTN1; CNTN2; CNTN3; CNTN4; CNTN5; CNTN6; CNTNAP1; CNTNAP2; CNTNAP3; CNTNAP4; CNTNAP5; CNTRL; CNTROB; COAL; COA5; COASY; COBL; COBLL1; COCH; COG1; COG2; COG3; COG4; COG5; COG6; COG7; COG5; COIL; COL10A1; COL11A1; COL11A2; COL12A1; COL13A1; COL14A1; COL15A1; COL16A1; COL17A1; COL18A1; COL1A1; COL1A2; COL20A1; COL21A1; COL22A1; COL23A1; COL24A1; COL25A1; COL26A1; COL27A1; COL28A1; COL2A1; COL3A1; COL4A1; COL4A2; COL4A3BP; COL4A3; COL4A4; COL4A5; COL4A6; COL5A1; COL5A2; COL5A3; COL6A1; COL6A2; COL6A3; COL6A5; COL7A1; COL8A1; COL8A2; COL9A1; COL9A2; COL9A3; COLCA1; COLCA2; COLEC10; COLEC11; COLEC12; COLGALT2; COLQ; COMMD10; COMMD1; COMMD3-BMI1; COMMD5; COMMD7; COMP; COMT; COPA; COPB1; COPB2; COPE; COPG2; COPRS; COPS2; COPS3; COPS4; COPS5; COPSE; COPS7A; COPSE; COPZ2; COQ2; COQ3; COQ4; COQ5; COQ6; COQ7; COQ9; CORIN; CORO1A; CORO1B; CORO1C; CORO2A; CORO2B; CORO6; CORO7; CORO7-PAM16; COTL1; COX10; COX11; COX14; COX15; COX16; COX17; COX18; COX19; COX4I1; COX4I2; COX5A; COX5B; COX6A1; COX6A2; COX6B1; COX6C; COX7A1; COX7A2; COX7A2L; COX7B2; COX7B; COX7C; COX8A; CPA1; CPA2; CPA3; CPA4; CPA6; CPAMD8; CPB1; CPB2; CPD; CPEB1; CPEB3; CPEB4; CPED1; CPE; CP; CPLX1; CPLX2; CPLX3; CPLX4; CPM; CPN1; CPN2; CPNE1; CPNE2; CPNE3; CPNE4; CPNE7; CPNE8; CPO; CPDX; CPPED1; CPQ; CPS1; CPSF1; CPSF2; CPSF3; CPSF3L; CPSF4; CPSF6; CPSF7; CPT1A; CPT1B; CPT1C; CPVL; CPXCR1; CPZ; CR1; CR2; CRABP1; CRABP2; CRACR2A; CRADD; CRAMP1L; CRAT; CRB1; CRB2; CRB3; CRBN; CRCP; CRCT1; CREB1; CREB3; CREB3L1; CREB3L2; CREB3L3; CREB3L4; CREB5; CREBBP; CREBRF; CREBZF; CREG1; CRELD1; CRELD2; CREM; CRHBP; CRH; CRHR1; CRHR2; CRIM1; CRIP2; CRIP3; CRIPAK; CRIPT; CRISP1; CRISP2; CRISP3; CRISPLD1; CRISPLD2; CRK; CRKL; CRLF1; CRLF2; CRLF3; CRLS1; CRMP1; CRNKL1; CRNN; CROCC; CROT; CRP; CRTAC1; CRTAM; CRTAP; CRTC1; CRTC2; CRTC3; CRX; CRY1; CRY2; CRYAB; CRYBA1; CRYBA2; CRYBA4; CRYBB1; CRYBB2; CRYBB3; CRYGB; CRYGC; CRYGD; CRYGS; CRYL1; CRYM; CRYZL1; CSAD; CSAG3; CSE1L; CSF1R; CSF2; CSF2RA; CSF2RB; CSF3; CSGALNACT1; CSGALNACT2; CSH1; CSH2; CSHL1; CS; CSK; CSMD1; CSMD2; CSMD3; CSN1S1; CSN2; CSN3; CSNK1A1; CSNK1D; CSNK1G3; CSNK2A1; CSNK2A2; CSNK2B; CSPG4; CSPG5; CSPP1; CSRNP1; CSRNP3; CSRP1; CSRP2BP; CSRP2; CSRP3; CST1; CST2; CST3; CST4; CST5; CST6; CST7; CST8; CST9; CST9L; CSTA; CSTB; CSTF1; CSTF2; CSTF2T; CSTL1; CT45A1; CT55; CT83; CTAG1A; CTAG2; CTAGE1; CTAGE5; CTBP1; CTBP2; CTC1; CTCF; CTCFL; CTDP1; CTDSP1; CTDSP2; CTDSPL; CTF1; CTGF; CTHRC1; CTIF; CTLA4; CTNNA1; CTNNA2; CTNNA3; CTNNAL1; CTNNB1; CTNNBL1; CTNND1; CTNND2; CTNS; CTPS1; CTR9; CTRB1; CTRB2; CTRC; CTRL; CTSA; CTSB; CTSC; CTSD; CTSE; CTSF; CTSG; CTSH; CTSL; CTSO; CTSV; CTSW; CTSZ; CTTNBP2; CTTN; CTU1; CTXN3; CUBN; CUEDC1; CUEDC2; CUL1; CUL2; CUL3; CUL4A; CUL4B; CUL5; CULT; CUL9; CUTA; CUX1; CUX2; CUZD1; CWC22; CWC27; CWF19L1; CWF19L2; CWH43; CX3CL1; CX3CR1; CXADR; CXCL10; CXCL11; CXCL12; CXCL13; CXCL14; CXCL16; CXCL17; CXCL1; CXCL2; CXCL3; CXCL5; CXCL6; CXCL8; CXCL9; CXCR1; CXCR2; CXCR3; CXCR4; CXCR5; CXCR6; CXorf36; CXorf66; CXorf67; CXXC1; CXXC4; CYB561D2; CYB561; CYB5A; CYB5B; CYB5R3; CYB5R4; CYBA; CYBB; CYBRD1; CYC1; CYCS; CYFIP1; CYFIP2; CYGB; CYLC1; CYLC2; CYLD; CYP11A1; CYP11B1; CYP11B2; CYP17A1; CYP19A1; CYP1A1; CYP1A2; CYP1B1; CYP21A2; CYP24A1; CYP26A1; CYP26B1; CYP26C1; CYP27A1; CYP27B1; CYP27C1; CYP2A13; CYP2A6; CYP2A7; CYP2B6; CYP2C18; CYP2C19; CYP2C8; CYP2C9; CYP2D6; CYP2E1; CYP2F1; CYP2R1; CYP2S1; CYP2U1; CYP2W1; CYP39A1; CYP3A43; CYP3A4; CYP3A5; CYP3A7-CYP3A51P; CYP3A7; CYP46A1; CYP4A11; CYP4B1; CYP4F11; CYP4F12; CYP4F22; CYP4F2; CYP4F3; CYP4F8; CYP4V2; CYP51A1; CYP7A1; CYP7B1; CYP8B1; CYST; CYSLTR1; CYTH1; CYTH3; CYTIP; CYTL1; CYYR1; D2HGDH; DAAM1; DAAM2; DAB2; DAB2IP; DACH1; DACH2; DACT1; DACT2; DACT3; DAD1; DAG1; DAND5; DAOA; DAO; DAP3; DAP; DAPK1; DAPK2; DAPK3; DARS2; DARS; DAW1; DAXX; DAZ1; DAZ2; DAZ3; DAZ4; DAZAP1; DAZAP2; DAZL; DBF4; DBH; DBI; DBN1; DBNL; DBP; DBR1; DBX1; DCAF12; DCAF13; DCAF17; DCAF4; DCAF5; DCAF6; DCAF7; DCAF8; DCANP1; DCBLD1; DCBLD2; DCC; DCDC2C; DCDC2; DCD; DCHS1; DCHS2; DCK; DCLK1; DCLK2; DCLK3; DCLRE1A; DCLRE1C; DCN; DCP1A; DCP1B; DCPS; DCST1; DCST2; DCSTAMP; DCTD; DCT; DCTN1; DCTN2; DCTN3; DCTN4; DCTN5; DCTN6; DCUN101; DCX; DCXR; DDAH1; DDAH2; DDB1; DDB2; DDC; DDHD1; DDHD2; DDI1; DDIAS; DDIT3; DDIT4; DDIT4L; DDN; DDO; DDR1; DDR2; DDRGK1; DDT; DDTL; DDX10; DDX11; DDX17; DDX18; DDX19A; DDX1; DDX21; DDX25; DDX27; DDX28; DDX31; DDX39A; DDX39B; DDX3X; DDX3Y; DDX41; DDX42; DDX43; DDX46; DDX4; DDX50; DDX51; DDX52; DDX53; DDX54; DDX56; DDX58; DDX59; DDX5; DDX60; DDX6; DEAF1; DEC1; DECR1; DEDD; DEF6; DEFA1B; DEFA3; DEFA4; DEFA5; DEFA6; DEFB103B; DEFB104B; DEFB105A; DEFB106A; DEFB108B; DEFB112; DEFB125; DEFB126; DEFB1; DEFB4A; DEGS1; DEGS2; DEK; DENND1A; DENND1B; DENND2A; DENND4A; DENR; DEPDC1B; DEPDC5; DEPTOR; DERA; DERL1; DERL2; DERL3; DES; DESI2; DEXI; DFFB; DFNA5; DFNB31; DFNB59; DGAT1; DGAT2; DGCR2; DGCR6L; DGCR8; DGKA; DGKB; DGKD; DGKE; DGKG; DGKH; DGKI; DGKK; DGKQ; DGKZ; DGUOK; DHCR7; DHDH; DHFR; DHFRL1; DHH; DHODH; DHPS; DHRS11; DHRS2; DHRS4; DHRS7C; DHRS9; DHTKD1; DHX15; DHX16; DHX32; DHX34; DHX36; DHX38; DHX40; DHX58; DHX8; DHX9; DIABLO; DIAPH1; DIAPH2; DIAPH3; DICER1; DIDO1; DIEXF; DIP2A; DIP2B; DIP2C; DIRAS1; DIRAS2; DIRC1; DIRC2; DIS3; DIS3L2; DIS3L; DISCI; DISP1; DIXDC1; DKC1; DKK1; DKK2; DKK3; DKK4; DKKL1; DLAT; DLC1; DLD; DLEC1; DLEU7; DLG1; DLG2; DLG3; DLG4; DLG5; DLGAP1; DLGAP2; DLGAP3; DLGAP5; DLK1; DLL1; DLL3; DLL4; DLST; DLX1; DLX2; DLX3; DLX4; DLX5; DLX6; DMBT1; DMC1; DMD; DMGDH; DMKN; DMP1; DMPK; DMRT1; DMRT2; DMRT3; DMRTA1; DMTF1; DMTN; DMWD; DMXL1; DMXL2; DNA2; DNAAF1; DNAAF2; DNAAF3; DNAAF5; DNAH11; DNAH12; DNAH17; DNAH1; DNAH2; DNAH3; DNAH5; DNAH6; DNAH7; DNAH8; DNAH9; DNAI1; DNAI2; DNAJA1; DNAJA2; DNAJA3; DNAJB11; DNAJB13; DNAJB1; DNAJB2; DNAJB5; DNAJB6; DNAJB7; DNAJB8; DNAJB9; DNAJC10; DNAJC12; DNAJC13; DNAJC14; DNAJC15; DNAJC18; DNAJC19; DNAJC1; DNAJC27; DNAJC28; DNAJC2; DNAJC3; DNAJC5; DNAJC7; DNAL1; DNASE1; DNASE1L2; DNASE1L3; DNASE2B; DNASE2; DND1; DNER; DNHD1; DNLZ; DNM1; DNM1L; DNM2; DNM3; DNMBP; DNMT1; DNMT3A; DNMT3B; DNMT3L; DNPEP; DNTT; DOC2A; DOC2B; DOCK10; DOCK11; DOCK1; DOCK2; DOCK3; DOCK4; DOCK5; DOCK6; DOCK7; DOCK8; DOCK9; DOHH; DOK1; DOK2; DOK3; DOK4; DOK5; DOK6; DOK7; DOLK; DONSON; DOPEY2; DOT1L; DPAGT1; DPCD; DPCR1; DPEP1; DPEP2; DPEP3; DPF1; DPF3; DPH1; DPH3; DPH6; DPH7; DPMI; DPM2; DPM3; DPP10; DPP3; DPP4; DPP6; DPP7; DPP8; DPP9; DPPA2; DPPA3; DPPA4; DPT; DPY19L2; DPY19L3; DPY30; DPYS; DPYSL2; DPYSL3; DPYSL4; DPYSL5; DRAM1; DRAP1; DRC1; DRD1; DRD2; DRD3; DRD4; DRD5; DRG1; DRGX; DROSHA; DRP2; DSC1; DSC2; DSC3; DSCAM; DSCAML1; DSCC1; DSCR4; DSE; DSEL; DSG1; DSG2; DSG3; DSG4; DSP; DSPP; DST; DSTN; DSTYK; DTD1; DTHD1; DTL; DTNA; DTNB; DTNBP1; DTX1; DTX2; DTX3; DTX4; DTYMK; DUOX1; DUOX2; DUOXA1; DUOXA2; DUPD1; DUS2; DUSP10; DUSP11; DUSP12; DUSP13; DUSP14; DUSP15; DUSP16; DUSP18; DUSP1; DUSP21; DUSP22; DUSP23; DUSP27; DUSP28; DUSP2; DUSP3; DUSP4; DUSP5; DUSP6; DUSP7; DUSP8; DUSP9; DUT; DUX4; DVL2; DVL3; DXO; DYM; DYNAP; DYNC1H1; DYNC1I1; DYNC1I2; DYNC1LI1; DYNC2H1; DYNLL1; DYNLL2; DYNLRB1; DYNLT1; DYNLT3; DYRK1A; DYRK1B; DYRK2; DYRK3; DYSF; DYX1C1; DZIP1; E2F1; E2F3; E2F4; E2F5; E2F6; E2F7; E2F8; E4F1; EAF1; EAF2; EARS2; EBAG9; EBF1; EBF2; EBF3; EBF4; EBI3; EBP; EBPL; ECD; ECE2; ECEL1; ECHDC1; ECHDC3; ECHS1; ECI1; ECI2; ECSCR; ECSIT; ECT2; EDA2R; EDA; EDARADD; EDAR; EDC4; EDEM1; EDIL3; EDN1; EDN3; EDNRA; EDNRB; EEA1; EED; EEF1A1; EEF1A2; EEF1B2; EEF1D; EEF1E1; EEF1G; EEF2; EEFSEC; EFCAB11; EFCAB1; EFEMP1; EFEMP2; EFHB; EFHC1; EFHC2; EFHD1; EFNA1; EFNA2; EFNA3; EFNA4; EFNA5; EFNB1; EFNB2; EFNB3; EFR3A; EFR3B; EFS; EFTUD1; EFTUD2; EGF; EGFL6; EGFL7; EGFL8; EGFLAM; EGFR; EGLN1; EGLN2; EGLN3; EGR1; EGR2; EGR3; EGR4; EHBP1; EHD1; EHD2; EHD3; EHD4; EHF; EHHADH; EHMT1; EHMT2; EI24; EID1; EIF1AD; EIF1AX; EIF1AY; EIF1; EIF2A; EIF2AK1; EIF2AK2; EIF2AK3; EIF2AK4; EIF2B1; EIF2B2; EIF2B3; EIF2B4; EIF2B5; EIF2S1; EIF2S2; EIF2S3; EIF3A; EIF3B; EIF3C; EIF3E; EIF3F; EIF3H; EIF3J; EIF3K; EIF3M; EIF4A1; EIF4A2; EIF4A3; EIF4B; EIF4E2; EIF4E3; EIF4EBP1; EIF4EBP2; EIF4EBP3; EIF4E; EIF4EIF1; EIF4G1; EIF4G2; EIF4H; EIF5A2; EIF5A; EIF5; EIF6; ELAC1; ELAC2; ELANE; ELAVL1; ELAVL2; ELAVL3; ELAVL4; ELF1; ELF2; ELF3; ELF4; ELF5; ELK1; ELK3; ELK4; ELL2; ELL; ELMO1; ELMO2; ELMOD1; ELMOD2; ELMOD3; ELMSAN1; ELN; ELOF1; ELOVL2; ELOVL4; ELOVL5; ELOVL6; ELOVL7; ELP2; ELP3; ELP4; ELP6; ELSPBP1; EMB; EMC10; EMC2; EMC3; EMC7; EMC8; EMCN; EMD; EME1; EMG1; EMILIN1; EMILIN2; EMILIN3; EML1; EML2; EML4; EML5; EML6; EMP1; EMP2; EMX1; EMX2; EN1; EN2; ENAH; ENAM; ENC1; ENDOG; ENDOU; ENDOV; ENGASE; ENG; ENHO; ENO2; ENO3; ENO4; ENOPH1; ENOSF1; ENOX1; ENOX2; ENPEP; ENPP1; ENPP2; ENPP3; ENPP5; ENPP7; ENTHD2; ENTPD1; ENTPD2; ENTPD4; ENTPD5; ENTPD6; ENTPD7; EOGT; EOMES; EP300; EP400; EPAS1; EPB41L1; EPB41L2; EPB41L3; EPB41L4A; EPB41L4B; EPB42; EPC1; EPC2; EPCAM; EPDR1; EPG5; EPGN; EPHA1; EPHA3; EPHA4; EPHA5; EPHA6; EPHA7; EPHB1; EPHB3; EPHB4; EPHB6; EPHX1; EPHX2; EPHX3; EPM2A; EPM2AIP1; EPN1; EPO; EPOR; EPPIN; EPPIN-WFDC6; EPPK1; EPRS; EPS15L1; EPS8; EPS8L2; EPSTI1; EPX; EPYC; ERAL1; ERAP1; ERAP2; ERAS; ERBB2; ERBB2IP; ERBB3; ERBB4; ERCT; ERC2; ERCC1; ERCC2; ERCC3; ERCC4; ERCC5; ERCC6; ERCC6L2; ERCC6-PGBD3; ERCC8; EREG; ERF; ERG; ERGIC1; ERGIC2; ERGIC3; ERI3; ERICH5; ERICH6B; ERLEC1; ERLIN1; ERLIN2; ERMAP; ERMARD; ERMP1; ERN1; ERN2; ERO1LB; ERO1L; ERP29; ERP44; ERV3-1; ERVW-1; ESAM; ESCO1; ESCO2; ESD; ESF1; ESM1; ESPL1; ESPN; ESR1; ESR2; ESRP1; ESRP2; ESRRA; ESRRB; ESRRG; ESX1; ESYT1; ESYT2; ESYT3; ETAA1; ETF1; ETFA; ETFB; ETFDH; ETHE1; ETNK1; ETNK2; ETNPPL; ETS1; ETS2; ETV1; ETV3; ETV4; ETV5; ETV6; ETV7; EVA1A; EVA1C; EVC2; EVC; EVI2A; EVI2B; EVI5; EVL; EVPL; EVX1; EVX2; EWSR1; EXD2; EXD3; EXO1; EXOC1; EXOC2; EXOC3L1; EXOC3L2; EXOC4; EX005; EXOC7; EXOG; EXOSC1; EXOSC2; EXOSC3; EXOSC4; EXOSC5; EXOSC6; EXOSC7; EXOSC8; EXPH5; EXT1; EXT2; EXTL2; EXTL3; EYA1; EYA2; EYA4; EYS; EZH1; EZH2; EZR; F10; F11; F11R; F12; F13A1; F13B; F2; F2R; F2RL1; F2RL2; F2RL3; F5; F7; F8; F9; FA2H; FAAH2; FABP1; FABP2; FABP4; FABP5; FABP6; FABP7; FABP9; FADD; FADS1; FADS2; FADS3; FAF2; FAHD2A; FAH; FAIM2; FAIM3; FAM102A; FAM103A1; FAM105A; FAM107A; FAM107B; FAM109A; FAM110A; FAM110B; FAM110C; FAM111B; FAM114A1; FAM120B; FAM120C; FAM124B; FAM126A; FAM129A; FAM129B; FAM134B; FAM135A; FAM135B; FAM136A; FAM13A; FAM13C; FAM149A; FAM150B; FAM155A; FAM160B1; FAM161A; FAM163A; FAM163B; FAM167A; FAM168A; FAM168B; FAM169B; FAM170A; FAM172A; FAM173B; FAM174A; FAM175A; FAM175B; FAM177A1; FAM177B; FAM178A; FAM178B; FAM179B; FAM180A; FAM184A; FAM184B; FAM187B; FAM188A; FAM188B; FAM189B; FAM193B; FAM196A; FAM196B; FAM198B; FAM19A1; FAM19A2; FAM19A4; FAM19A5; FAM204A; FAM205A; FAM20A; FAM20B; FAM20C; FAM210B; FAM213A; FAM214A; FAM216A; FAM220A; FAM227B; FAM32A; FAM3A; FAM3B; FAM3C; FAM3D; FAM46A; FAM46D; FAM49A; FAM49B; FAM50B; FAM53B; FAM57A; FAM58A; FAM60A; FAM63B; FAM64A; FAM65B; FAM69C; FAM71F1; FAM71F2; FAM72B; FAM78B; FAM81B; FAM83A; FAM83B; FAM83D; FAM83H; FAM84A; FAM84B; FAM89A; FAM92A1; FAM92B; FAM9B; FAN1; FANCA; FANCB; FANCC; FANCD2; FANCE; FANCF; FANCG; FANCI; FANCL; FANCM; FANK1; FAP; FAR1; FARP1; FARP2; FARS2; FARSA; FAS; FASLG; FASN; FASTKD2; FASTK; FAT1; FAT2; FAT3; FAT4; FATE1; FAU; FBF1; FBL; FBLIM1; FBLN1; FBLN2; FBLN5; FBN1; FBN2; FBN3; FBP1; FBP2; FBRS; FBXL15; FBXL17; FBXL19; FBXL20; FBXL2; FBXL3; FBXL4; FBXL5; FBXL7; FBXO10; FBXO11; FBXO15; FBXO17; FBXO18; FBXO28; FBXO30; FBXO31; FBXO32; FBXO33; FBXO38; FBXO3; FBXO40; FBXO47; FBXO4; FBXO5; FBXO7; FBXO8; FBXO9; FBXW11; FBXW4; FBXW7; FBXW8; FCAR; FCER1A; FCER1G; FCER2; FCGBP; FCGR2A; FCGR2B; FCGR3A; FCGR3B; FCGRT; FCH01; FCHO2; FCHSD2; FCN1; FCN2; FCRL1; FCRL2; FCRL3; FCRL4; FCRL5; FCRL6; FCRLA; FCRLB; FDFT1; FDPS; FDX1; FDX1L; FDXR; FECH; FEM1A; FEM1B; FEM1C; FEN1; FERD3L; FER; FERMT1; FERMT2; FERMT3; FES; FEV; FEZ1; FEZ2; FEZF1; FEZF2; FFAR1; FFAR2; FFAR3; FFAR4; FGA; FGB; FGD1; FGD2; FGD3; FGD4; FGD5; FGD6; FGF10; FGF11; FGF12; FGF13; FGF14; FGF16; FGF17; FGF18; FGF19; FGF1; FGF20; FGF21; FGF23; FGF2; FGF3; FGF4; FGF5; FGF6; FGF7; FGF8; FGF9; FGFBP1; FGFBP2; FGFBP3; FGFR1; FGFR10P2; FGFR10P; FGFR2; FGFR3; FGFR4; FGFRL1; FGG; FGGY; FGL1; FGL2; FGR; FHDC1; FH; FHIT; FHL1; FHL2; FHL5; FHOD1; FHOD3; FIBP; FIG4; FIGF; FIGLA; FIGN; FILIP1; FILIP1L; FIP1L1; FIST; FITM1; FITM2; FJX1; FKBP10; FKBP11; FKBP14; FKBP15; FKBP1A; FKBP1B; FKBP2; FKBP3; FKBP4; FKBP5; FKBP6; FKBP7; FKBP8; FKBP9; FKBPL; FKRP; FKTN; FLAD1; FLCN; FLG2; FLG; FLIT; FLII; FLNA; FLNB; FLNC; FLOT1; FLOT2; FLRT2; FLRT3; FLT1; FLT3; FLT3LG; FLT4; FLVCR1; FLVCR2; FMN1; FMN2; FMNL1; FMNL2; FMNL3; FM01; FMO2; FMO3; FMO4; FMOD; FMR1; FN1; FN3K; FN3KRP; FNBP1; FNDC1; FNDC3A; FNDC3B; FNDC4; FNTA; FNTB; FOCAD; FOLH1; FOLR1; FOLR2; FOLR3; FOPNL; FOSB; FOS; FOSL1; FOSL2; FOXA1; FOXA2; FOXA3; FOXB1; FOXC1; FOXC2; FOXD1; FOXE1; FOXF1; FOXF2; FOXG1; FOXH1; FOXI1; FOXJ1; FOXJ2; FOXK1; FOXK2; FOXL1; FOXL2; FOXM1; FOXN1; FOXN2; FOXN3; FOXO1; FOXO3; FOXO4; FOXO6; FOXP1; FOXP2; FOXP3; FOXP4; FOXQ1; FOXR1; FOXR2; FOXRED1; FPGS; FPR1; FPR2; FPR3; FRAS1; FRAT1; FRAT2; FREM1; FREM2; FREM3; FRG1; FRG2; FRK; FRMD3; FRMD4A; FRMD4B; FRMD5; FRMD6; FRMD7; FRMPD1; FRMPD2; FRMPD4; FRRS1L; FRS2; FRS3; FRY; FRYL; FRZB; FSBP; FSCB; FSCN1; FSCN2; FSD1; FSD1L; FSD2; FSHB; FSHR; FSIP1; FST; FSTL1; FSTL3; FSTL4; FSTL5; FTCD; FTH1; FTL; FTMT; FTO; FTSJ1; FTSJ2; FTSJ3; FUBP1; FUBP3; FUCA1; FUCA2; FUNDC1; FUNDC2; FURIN; FUS; FUT10; FUT11; FUT1; FUT2; FUT3; FUT4; FUT5; FUT6; FUT7; FUT8; FUT9; FUZ; FXN; FXR1; FXR2; FXYD1; FXYD2; FXYD3; FXYD5; FXYD6; FYB; FYCO1; FYN; FZD10; FZD1; FZD2; FZD3; FZD4; FZD5; FZD6; FZD7; FZD8; FZD9; FZR1; GOS2; G2E3; G3BP1; G6PC2; G6PC3; G6PC; G6PD; GAA; GAB1; GAB2; GAB3; GABARAP; GABARAPL1; GABARAPL2; GABBR1; GABBR2; GABPA; GABPB1; GABRA1; GABRA2; GABRA3; GABRA4; GABRA5; GABRA6; GABRB1; GABRB2; GABRB3; GABRE; GABRG1; GABRG2; GABRG3; GABRP; GABRQ; GABRR1; GABRR2; GAD1; GAD2; GADD45B; GADD45G; GADD45GIP1; GADL1; GAGE10; GAGE1; GAK; GALC; GAL; GALK1; GALK2; GALM; GALNS; GALNT12; GALNT13; GALNT14; GALNT15; GALNT18; GALNT1; GALNT2; GALNT3; GALNT4; GALNT5; GALNT6; GALNT7; GALNT8; GALNT9; GALNTL6; GALP; GALR1; GALR2; GALR3; GALT; GAMT; GANAB; GANC; GAN; GAP43; GAPDH; GAPDHS; GAR1; GAREM; GARNL3; GARS; GART; GAS1; GAS2; GAS2L1; GAS6; GAS7; GASB; GAST; GATA1; GATA2; GATA3; GATA4; GATA5; GATA6; GATAD1; GATAD2A; GATAD2B; GATB; GATM; GBA2; GBA; GBAS; GBE1; GBF1; GBGT1; GBX1; GBX2; GCA; GCC1; GCDH; GCFC2; GCG; GCGR; GCH1; GCHFR; GC; GCK; GCKR; GCLC; GCM1; GCM2; GCN1L1; GCNT1; GCNT2; GCNT3; GCNT7; GCOM1; GCSAM; GCSAML; GCSH; GDA; GDAP1; GDE1; GDF10; GDF11; GDF15; GDF1; GDF2; GDF3; GDF5; GDF6; GDF7; GDF9; GDI1; GDI2; GDNF; GDPD3; GDPD5; GEM; GEMIN2; GEMIN4; GEMIN6; GEN1; GET4; GFAP; GFER; GFI1B; GFI1; GFM1; GFPT1; GFPT2; GFRA1; GFRA2; GFRA3; GFRA4; GGA1; GGA3; GGACT; GGCT; GGCX; GGH; GGNBP2; GGN; GGPS1; GGT1; GGT2; GGT5; GGTLC1; GH1; GH2; GHITM; GHRH; GHR; GHRHR; GHRL; GHSR; GIDS; GIF; GIGYF1; GIGYF2; GIMAP5; GIMAP7; GIMAP8; GIN1; GINS1; GINS2; GIPC1; GIPC3; GIP; GIPR; GIT1; GIT2; GJA1; GJA3; GJA8; GJB1; GJB2; GJB6; GJC1; GJC2; GJC3; GJD2; GJD3; GK; GKN1; GKN2; GLA; GLB1; GLCCI1; GLCE; GLDC; GLDN; GLE1; GLG1; GLI1; GLI2; GLI3; GLIPR1; GLIPR2; GLIS2; GLIS3; GLMN; GL01; GLOD4; GLP1R; GLP2R; GLRA1; GLRA2; GLRA3; GLRB; GLRX2; GLRX3; GLRX5; GLRX; GLS2; GLS; GLT1D1; GLT6D1; GLT8D1; GLTSCR1; GLTSCR2; GLUD1; GLUD2; GLUL; GLYAT; GLYATL3; GLYCTK; GM2A; GMCL1; GMDS; GMFB; GMFG; GMIP; GML; GMNN; GMPPA; GMPPB; GMPR2; GMPR; GMPS; GNA11; GNA12; GNA13; GNA14; GNA15; GNAI1; GNAI2; GNAL; GNAO1; GNAQ; GNAS; GNAT1; GNAT3; GNAZ; GNB1L; GNB2L1; GNB3; GNB4; GNB5; GNE; GNG10; GNG11; GNG2; GNG4; GNG7; GNG8; GNGT1; GNGT2; GNL1; GNL3; GNL3L; GNLY; GNMT; GNPAT; GNPDA1; GNPDA2; GNPTAB; GNPTG; GNRH1; GNRH2; GNRHR; GNS; GOLGA1; GOLGA2; GOLGA3; GOLGA4; GOLGA5; GOLGA8B; GOLGB1; GOLM1; GOLPH3; GOLT1A; GOLT1B; GON4L; GOPC; GORAB; GORASP1; GORASP2; GOSR1; GOSR2; GOT1; GOT2; GP1BA; GP1BB; GP2; GPS; GP6; GP9; GPA33; GPAA1; GPALPP1; GPAM; GPANK1; GPAT2; GPATCH1; GPATCH2; GPATCH2L; GPATCH8; GPBAR1; GPC1; GPC2; GPC3; GPC4; GPC5; GPC6; PCPD1; GPD1; GPD1L; GPD2; GPER1; GPHA2; GPHN; GPIHBP1; GPI; GPKOW; GPLD1; GPM6A; GPM6B; GPN1; GPNMB; GPR101; GPR119; GPR12; GPR132; GPR135; GPR137C; GPR139; GPR143; GPR148; GPR149; GPR150; GPR151; GPR152; GPR153; GPR155; GPR156; GPR158; GPR15; GPR160; GPR161; GPR162; GPR171; GPR174; GPR176; GPR179; GPR17; GPR180; GPR182; GPR183; GPR19; GPR1; GPR20; GPR22; GPR26; GPR34; GPR35; GPR37; GPR37L1; GPR39; GPR4; GPR50; GPR52; GPR55; GPR65; GPR68; GPR6; GPR75; GPR78; GPR83; GPR87; GPRASP1; GPRC5A; GPRC5B; GPRC5C; GPRC5D; GPRC6A; GPRIN1; GPRIN2; GPRIN3; GPS2; GPSM1; GPSM2; GPSM3; GPT2; GPT; GPX5; GRAMD1B; GRAMD3; GRAMD4; GRAP2; GRAP; GRASP; GRB10; GRB14; GRB2; GRB7; GREB1; GREM1; GREM2; GRHL1; GRHL2; GRHPR; GRIA1; GRIA2; GRIA3; GRIA4; GRID1; GRID2; GRIK1; GRIK2; GRIK4; GRIK5; GRIN1; GRIN2A; GRIN2B; GRIN2C; GRIN2D; GRIN3A; GRIN3B; GRINA; GRIP1; GRK1; GRK4; GRK5; GRK6; GRK7; GRM1; GRM2; GRM3; GRM4; GRM5; GRM6; GRM7; GRM8; GRN; GRPEL1; GRP; GRPR; GRSF1; GRXCR1; GRXCR2; GSC2; GSC; GSDMA; GSDMB; GSDMC; GSDMD; GSE1; GSG1L; GSK3A; GSK3B; GSN; GSPT1; GSPT2; GSR; GSS; GSTA1; GSTA2; GSTA4; GSTA5; GSTCD; GSTK1; GSTM3; GSTO1; GSTO2; GSTP1; GSTT1; GSTT2B; GSTT2; GSTZ1; GSX1; GSX2; GTDC1; GTF2A1; GTF2A1L; GTF2A2; GTF2E1; GTF2E2; GTF2F1; GTF2F2; GTF2H1; GTF2H2C_2; GTF2H2C; GTF2H2; GTF2H3; GTF2H4; GTF2H5; GTF2I; GTF2IRD1; GTF2IRD2; GTF3A; GTF3C1; GTPBP1; GTPBP3; GTPBP4; GTSF1; GUCA1A; GUCA1B; GUCD1; GUCY1A2; GUCY1A3; GUCY1B3; GUCY2C; GUCY2D; GUCY2F; GUK1; GULP1; GUSB; GYG1; GYG2; GYLTL1B; GYPA; GYPB; GYPC; GYPE; GYS1; GYS2; GZF1; GZMA; GZMB; GZMH; GZMK; GZMM; H1F0; H1FX; H2AFJ; H2AFX; H2AFY2; H2AFY; H2AFZ; H2BFWT; H3F3B; HAAO; HABP2; HABP4; HACD1; HACD2; HACD4; HACE1; HACL1; HADHA; HADHB; HADH; HAGH; HAL; HAMP; HAND1; HAND2; HAO1; HAO2; HAP1; HAPLN1; HAPLN4; HARS2; HARS; HAST; HAS2; HAS3; HAT1; HAVCR1; HAVCR2; HAX1; HBA2; HBB; HBD; HBE1; HBEGF; HBG1; HBG2; HBM; HBP1; HBQ1; HBS1L; HBZ; HCAR1; HCAR2; HCAR3; HCCS; HCFC1; HCFC2; HCK; HCLS1; HCN1; HCN2; HCN3; HCN4; HCRT; HCRTR2; HCST; HDAC10; HDAC11; HDAC1; HDAC2; HDAC3; HDAC4; HDAC5; HDAC6; HDAC7; HDAC8; HDAC9; HDC; HDDC2; HDGF; HDGFL1; HDGFRP3; HDHD1; HDLBP; HEATR1; HEATR3; HEATR5B; HEATR6; HEBP1; HEBP2; HECA; HECTD2; HECTD4; HECW1; HECW2; HEG1; HELLS; HELQ; HELT; HELZ2; HELZ; HEMGN; HEPACAM; HEPH; HEPN1; HERC1; HERC2; HERC3; HERC5; HERC6; HERPUD1; HES1; HES2; HES6; HEST; HESX1; HEXA; HEXB; HEXDC; HEXIM1; HEY1; HEY2; HFE; HFM1; HGD; HGFAC; HGF; HGH1; HGS; HGSNAT; HHAT; HHATL; HHEX; HHIP; HHIPL1; HHLA1; HHLA2; HIAT1; HIBADH; HIBCH; HIC1; HIC2; HID1; HIF1A; HIF1AN; HIF3A; HIGD1A; HIGD1C; HIGD2A; HILPDA; HINFP; HINT1; HINT2; HIP1; HIP1R; HIPK1; HIPK2; HIPK3; HIRA; HIRIP3; HIST1H1A; HIST1H1B; HIST1H1C; HIST1H1D; HIST1H1E; HIST1H1T; HIST1H2AE; HIST1H2AH; HIST1H2BG; HIST1H2BH; HIST1H2BM; HIST1H3G; HIST1H4D; HIST3H3; HIVEP1; HIVEP2; HJURP; HK1; HK2; HK3; HKDC1; HLA-A; HLA-B; HLA-C; HLA-DMA; HLA-DMB; HLA-DOA; HLA-DOB; HLA-DPA1; HLA-DPB1; HLA-DQA1; HLA-DQA2; HLA-DQB1; HLA-DQB2; HLA-DRA; HLA-DRB1; HLA-DRB3; HLA-DRB4; HLA-DRB5; HLA-E; HLA-F; HLA-G; HLCS; HLF; HLTF; HLX; HM13; HMBOX1; HMBS; HMCN1; HMG20A; HMG20B; HMGA1; HMGA2; HMGB1; HMGB2; HMGB3; HMGCR; HMGCS1; HMGN1; HMGN4; HMGN5; HMGXB3; HMGXB4; HMHA1; HMMR; HMOX1; HMOX2; HMP19; HMSD; HMX1; HN1; HN1L; HNF1A; HNF1B; HNF4A; HNF4G; HNMT; HNRNPAO; HNRNPA1; HNRNPA2B1; HNRNPA3; HNRNPAB; HNRNPC; HNRNPD; HNRNPDL; HNRNPF; HNRNPH1; HNRNPH2; HNRNPK; HNRNPL; HNRNPM; HNRNPR; HNRNPU; HNRNPUL1; HOGA1; HOMER1; HOMER2; HOMER3; HOMEZ; HOOK2; HOOK3; HOPX; HORMAD2; HOXA10; HOXA11; HOXA13; HOXA1; HOXA2; HOXA3; HOXA4; HOXA5; HOXA6; HOXA7; HOXA9; HOXB13; HOXB1; HOXB2; HOXB3; HOXB4; HOXB5; HOXB6; HOXB7; HOXB8; HOXB9; HOXC10; HOXC11; HOXC12; HOXC13; HOXC4; HOXC5; HOXC6; HOXC8; HOXC9; HOXD10; HOXD11; HOXD12; HOXD13; HOXD1; HOXD3; HOXD4; HOXD8; HOXD9; HP1BP3; HPCAL1; HPD; HPGD; HPGDS; HP; HPN; HPR; HPRT1; HPS1; HPS3; HPS4; HPS5; HPS6; HPSE2; HPSE; HPX; HRAS; HRASLS; HRC; HRG; HRH1; HRH2; HRH3; HRH4; HR; HRK; HRNR; HRSP12; HS1BP3; HS3ST1; HS3ST2; HS3ST3A1; HS3ST3B1; HS3ST4; HS3ST5; HS3ST6; HS6ST1; HS6ST2; HS6ST3; HSBP1; HSD11B1; HSD11B1L; HSD11B2; HSD17B10; HSD17B11; HSD17B12; HSD17B13; HSD17B14; HSD17B1; HSD17B2; HSD17B3; HSD17B4; HSD17B6; HSD17B7; HSD17B8; HSD3B1; HSD3B7; HSDL1; HSDL2; HSF1; HSF2; HSF4; HSF5; HSFY2; HSH2D; HSP90AA1; HSP90AB1; HSP90B1; HSPA12A; HSPA12B; HSPA13; HSPA14; HSPA1A; HSPAlL; HSPA2; HSPA4; HSPA4L; HSPA5; HSPA6; HSPA8; HSPA9; HSPB1; HSPB2; HSPB3; HSPB6; HSPB7; HSPB8; HSPB9; HSPBAP1; HSPBP1; HSPD1; HSPE1; HSPG2; HSPH1; HTATIP2; HTN1; HTN3; HTR1A; HTR1B; HTR1E; HTR1F; HTR2A; HTR2B; HTR2C; HTR3A; HTR3B; HTR3C; HTR3D; HTR3E; HTR4; HTR5A; HTR7; HTRA1; HTRA2; HTRA3; HTRA4; HTT; HUNK; HUS1B; HUS1; HUWE1; HVCN1; HYAL1; HYAL2; HYAL3; HYAL4; HYDIN; HYKK; HYLS1; HYOU1; HYPM; IAPP; IARS2; IARS; IBA57; TBSP; IBTK; ICA1; ICAM1; ICAM2; ICAM3; ICAM4; ICAM5; ICE1; ICE2; ICK; ICMT; ICOS; ICOSLG; ID1; ID2; ID4; IDE; IDH1; IDH2; IDH3B; IDNK; IDOL; ID02; IDS; IDUA; IER2; IER3; IER3IP1; IER5; IFF01; IFF02; IFI16; IFI27; IFI30; IFI35; IFI44; IFIH1; IFIT1; IFIT2; IFIT3; IFIT5; IFITM1; IFITM2; IFITM3; IFITM5; IFNA10; IFNA13; IFNA14; IFNA16; IFNA17; IFNA1; IFNA21; IFNA2; IFNA4; IFNA5; IFNA6; IFNA7; IFNA8; IFNAR1; IFNAR2; IFNB1; IFNE; IFNG; IFNGR1; IFNGR2; IFNK; IFNL1; IFNL2; IFNL3; IFNW1; IFRD1; IFT122; IFT140; IFT172; IFT27; IFT43; IFT57; IFT74; IFT80; IFT88; IGBP1; IGDCC3; IGF1; IGF1R; IGF2BP1; IGF2BP2; IGF2BP3; IGF2; IGF2R; IGFALS; IGFBP1; IGFBP2; IGFBP3; IGFBP4; IGFBP5; IGFBP6; IGFBP7; IGFBPL1; IGFL1; IGFLR1; IGHMBP2; IGJ; IGLL1; IGSF10; IGSF11; IGSF1; IGSF5; IGSF6; IGSF8; IHH; IKBKAP; IKBKB; IKBKE; IKBKG; IK; IKZF1; IKZF2; IKZF3; IKZF4; IL10; IL10RA; IL10RB; IL11; IL11RA; IL12A; IL12B; IL12RB1; IL13; IL13RA1; IL13RA2; IL15; IL15RA; IL16; IL17A; IL17B; IL17C; IL170; IL17F; IL17RA; IL17RB; IL17RC; IL17RD; IL17RE; IL17REL; IL18BP; IL18; IL18R1; IL18RAP; IL19; IL1A; IL1B; IL1F10; IL1R1; IL1R2; IL1RAP; IL1RAPL1; IL1RAPL2; IL1RL1; IL1RL2; IL1RN; IL20; IL20RA; IL20RB; IL21; IL21R; IL22; IL22RA2; IL23A; IL23R; IL24; IL25; IL26; IL27; IL27RA; IL2; IL2RA; IL2RB; IL2RG; IL31; IL31RA; IL32; IL33; IL34; IL36A; IL36B; IL36G; IL36RN; IL37; IL3; IL3RA; IL4; IL4I1; IL4R; ILS; IL5RA; IL6; IL6R; IL6ST; IL7; IL7R; IL9; IL9R; ILDR1; ILDR2; ILF2; ILF3; ILK; IMMP1L; IMMP2L; IMMT; IMP3; IMPA1; IMPA2; IMPACT; IMPAD1; IMPDH1; IMPDH2; IMPG1; IMPG2; INADL; INA; INCENP; INF2; ING1; ING2; ING3; ING4; ING5; INHA; INHBA; INHBB; INHBC; INHBE; INO80B; INO80D; INO80; INPP1; INPP4A; INPP4B; INPP5A; INPP5B; INPP5D; INPP5E; INPP5F; INPP5K; INPPL1; INSC; INS; INSIG1; INSIG2; INS-IGF2; INSL3; INSL4; INSL6; INSM1; INSM2; INSR; INSRR; INTS12; INTS1; INTS2; INTS3; INTS4; INTS5; INTS6; INTS7; INTS8; INTU; INVS; IP6K1; IP6K2; IP6K3; IPCEF1; IPMK; IP011; IP013; IP07; IP08; IP09; IQCB1; IQCG; IQCH; IQCJ; IQCJ-SCHIP1; IQCK; IQGAP1; IQGAP2; IQGAP3; IQSEC1; IQSEC2; IQSEC3; IRAK1BP1; IRAK1; IRAK2; IRAK3; IRAK4; IREB2; IRF1; IRF2BP2; IRF2BPL; IRF2; IRF3; IRF4; IRF5; IRF6; IRF7; IRF8; IRF9; IRG1; IRGM; IRS1; IRS2; IRS4; IRX1; IRX2; IRX3; IRX4; IRX5; ISCU; ISG20; ISL1; ISM1; ISM2; ISPD; IST1; ISX; ISYNA1; ITCH; ITFG1; ITFG3; ITGA10; ITGA11; ITGA1; ITGA2B; ITGA2; ITGA3; ITGA4; ITGA5; ITGA6; ITGA7; ITGA8; ITGA9; ITGAD; ITGAE; ITGAL; ITGAM; ITGAV; ITGAX; ITGB1BP1; ITGB1; ITGB2; ITGB3; ITGB4; ITGB5; ITGB6; ITGB7; ITGB8; ITGBL1; ITIH1; ITIH2; ITIH3; ITIH4; ITIH5; ITK; ITLN1; ITLN2; ITM2A; ITM2B; ITM2C; ITPA; ITPK1; ITPKA; ITPKC; ITPR1; ITPR2; ITPR3; ITSN1; ITSN2; IVD; IVL; IVNS1ABP; IYD; IZUMO1; JADE1; JADE2; JAG1; JAG2; JAGN1; JAK2; JAK3; JAKMIP1; JAKMIP2; JAKMIP3; JAM2; JAM3; JARID2; JAZF1; JDP2; JMJD1C; JMJD6; JMY; JPH2; JPH3; JPH4; JRK; JSRP1; JTB; JUNB; JUND; JUP; KAL1; KALRN; KANK1; KANK2; KANK4; KANSL1; KANSL3; KARS; KAT2A; KAT2B; KAT5; KAT6A; KAT6B; KAT7; KATE; KATNA1; KATNAL2; KATNB1; KAZALD1; KAZN; KBTBD11; KBTBD13; KBTBD8; KCMF1; KCNA1; KCNA4; KCNA5; KCNA6; KCNA7; KCNAB1; KCNAB2; KCNAB3; KCNB1; KCNB2; KCNC1; KCNC2; KCNC3; KCND2; KCNE1; KCNE2; KCNE3; KCNE4; KCNE5; KCNG3; KCNG4; KCNH1; KCNH2; KCNH3; KCNH4; KCNH5; KCNH6; KCNH7; KCNH8; KCNIP1; KCNIP2; KCNIP3; KCNIP4; KCNJ10; KCNJ11; KCNJ12; KCNJ13; KCNJ14; KCNJ15; KCNJ16; KCNJ18; KCNJ1; KCND2; KCNJ3; KCNJ4; KCNJ5; KCNJ6; KCNJ8; KCNJ9; KCNK10; KCNK12; KCNK16; KCNK17; KCNK18; KCNK1; KCNK2; KCNK3; KCNK5; KCNK6; KCNK9; KCNMA1; KCNMB1; KCNMB2; KCNMB3; KCNMB4; KCNN1; KCNN2; KCNN3; KCNN4; KCNQ1; KCNQ2; KCNQ3; KCNQ5; KCNRG; KCNS1; KCNS3; KCNT1; KCNT2; KCNU1; KCNV1; KCNV2; KCTD10; KCTD11; KCTD12; KCTD13; KCTD15; KCTD16; KCTD1; KCTD21; KCTD2; KCTD3; KCTD6; KCTD7; KCTD9; KDELC1; KDELR1; KDM2A; KDM2B; KDM3A; KDM3B; KDM4B; KDM4C; KDM5A; KDM5B; KDM5C; KDM50; KDM6A; KDM6B; KDR; KDSR; KEAP1; KEL; KERA; KHDC3L; KHDRBS2; KHDRBS3; KHK; KHSRP; KIAA0020; KIAA0040; KIAA0100; KIAA0101; KIAA0196; KIAA0226; KIAA0226L; KIAA0232; KIAA0319; KIAA0319L; KIAA0355; KIAA0391; KIAA0586; KIAA0825; KIAA0907; KIAA0922; KIAA1024; KIAA1033; KIAA1109; KIAA1147; KIAA1161; KIAA1211; KIAA1211L; KIAA1217; KIAA1279; KIAA1324; KIAA1324L; KIAA1377; KIAA1456; KIAA1462; KIAA1468; KIAA1524; KIAA1549; KIAA1551; KIAA1598; KIAA1715; KIAA1804; KIAA1841; KIAA1919; KIAA2022; KIDINS220; KIF11; KIF13A; KIF14; KIF15; KIF16B; KIF17; KIF18A; KIF1A; KIF1C; KIF20A; KIF20B; KIF21A; KIF21B; KIF22; KIF23; KIF24; KIF25; KIF26A; KIF26B; KIF2A; KIF2B; KIF2C; KIF3A; KIF3B; KIF3C; KIF4A; KIF4B; KIF5A; KIF5B; KIF5C; KIF6; KIF7; KIF9; KIFAP3; KIFC1; KIFC3; KIN; KIR2DL1; KIR2DL2; KIR2DL3; KIR2DL4; KIR2DL5A; KIR2DL5B; KIR2DS1; KIR2DS2; KIR2DS3; KIR2DS4; KIR3DL1; KIR3DL2; KIR3DL3; KIRREL2; KIRREL3; KIRREL; KISS1; KISS1R; KIT; KITLG; KLB; KLC1; KLF10; KLF11; KLF12; KLF13; KLF14; KLF15; KLF16; KLF1; KLF2; KLF3; KLF4; KLF5; KLF6; KLF7; KLF8; KLF9; KLHDC1; KLHDC2; KLHDC8A; KLHDC8B; KLHL10; KLHL12; KLHL1; KLHL20; KLHL25; KLHL26; KLHL29; KLHL2; KLHL31; KLHL35; KLHL3; KLHL40; KLHL41; KLHL42; KLHL5; KLHL6; KLHL7; KLHL9; KL; KLK10; KLK11; KLK12; KLK13; KLK14; KLK15; KLK1; KLK2; KLK3; KLK4; KLK5; KLK6; KLK7; KLK8; KLK9; KLKB1; KLLN; KLRB1; KLRC1; KLRC2; KLRC4; KLRC4-KLRK1; KLRD1; KLRG1; KLRG2; KMO; KMT2A; KMT2B; KMT2C; KMT20; KMT2E; KNG1; KNSTRN; KNTC1; KPNA1; KPNA2; KPNA3; KPNA4; KPNA6; KPNA7; KPNB1; KPRP; KPTN; KRAS; KRBOX4; KREMEN1; KRIT1; KRT10; KRT12; KRT13; KRT14; KRT15; KRT16; KRT17; KRT18; KRT19; KRT1; KRT20; KRT23; KRT2; KRT31; KRT32; KRT34; KRT35; KRT3; KRT4; KRT5; KRT6A; KRT6B; KRT6C; KRT71; KRT72; KRT74; KRT75; KRT76; KRT78; KRT7; KRT80; KRT81; KRT82; KRT83; KRT85; KRT86; KRT8; KRT9; KRTAP11-1; KRTAP5-1; KRTAP5-9; KSR1; KSR2; KTN1; KYNU; L1CAM; L2HGDH; L3MBTL1; L3MBTL2; L3MBTL3; L3MBTL4; LACC1; LACE1; LACRT; LACTB; LAD1; LAG3; LAIR1; LAIR2; LALBA; LAMA1; LAMA2; LAMA3; LAMA4; LAMAS; LAMB1; LAMB2; LAMB3; LAMB4; LAMC1; LAMC2; LAMC3; LAMP1; LAMP2; LAMP3; LAMTOR1; LAMTOR2; LAMTOR3; LANCL1; LANCL2; LAP3; LAPTM4B; LARGE; LARP1; LARP4; LARP6; LARP7; LARS2; LARS; LAS1L; LASP1; LAT2; LAT; LATS1; LATS2; LAX1; LAYN; LBH; LBP; LBR; LBX1; LBX2; LCA5; LCAT; LCE1C; LCE3A; LCE3B; LCE3C; LCE30; LCE3E; LCE5A; LCK; LCLAT1; LCMT1; LCN1; LCN2; LCOR; LCP1; LCP2; LCT; LDB1; LDB2; LDB3; LDHA; LDHAL6B; LDHB; LDHC; LDHD; LDLRAD3; LDLRAD4; LDLR; LDOC1; LDOC1L; LEAP2; LECT1; LECT2; LEF1; LEFTY1; LEFTY2; LEKR1; LEMD2; LEMD3; LE01; LEP; LEPROTL1; LETM1; LETMD1; LEUTX; LFNG; LGALS12; LGALS13; LGALS14; LGALS16; LGALS1; LGALS2; LGALS3BP; LGALS3; LGALS4; LGALS7B; LGALS8; LGALS9; LGALSL; LGI1; LGI2; LGI4; LGMN; LGR4; LGR5; LGR6; LGSN; LHB; LHCGR; LHFP; LHFPL1; LHFPL2; LHFPL3; LHFPL4; LHFPL5; LHPP; LHX1; LHX2; LHX3; LHX4; LHX5; LHX6; LHX9; LIAS; LIF; LIFR; LIG1; LIG3; LIG4; LILRA1; LILRA2; LILRA3; LILRA4; LILRA5; LILRB1; LILRB2; LILRB3; LILRB4; LILRB5; LIM2; LIMA1; LIMCH1; LIMD1; LIMD2; LIMK1; LIMK2; LIMS1; LIMS2; LIN28B; LIN52; LIN7A; LIN7B; LIN7C; LIN9; LING01; LINGO2; LINGO4; LINS; LIPA; LIPC; LIPE; LIPF; LIPG; LIPH; LIPI; LIPN; LIPT1; LIPT2; LITAF; LIX1; LLGL1; LLGL2; LMAN1; LMAN1L; LMAN2L; LMBR1; LMBRD1; LMBRD2; LMCD1; LMF1; LMLN; LMNA; LMNB1; LMNB2; LMNTD1; LMO1; LMO2; LMO3; LMO7; LMOD1; LMOD3; LMTK2; LMTK3; LMX1A; LMX1B; LNPEP; LNX1; LNX2; LOC100288966; LOC101060321; LOC102723475; LOC102723996; LOC102724127; LOC102724560; LOC102724770; LOC102725016; LOC102725035; LOC400499; LOC400927-CSNK1E; LOC645177; LONP1; LONRF1; LOR; LOXHD1; LOX; LOXL1; LOXL2; LOXL3; LOXL4; LPA; LPAR1; LPAR2; LPAR3; LPAR6; LPCAT1; LPCAT2; LPCAT3; LPCAT4; LPGAT1; LPIN1; LPIN2; LPL; LPO; LPP; LPPR2; LPPR4; LPXN; LRAT; LRBA; LRCH1; LRCH4; LRFN2; LRFN5; LRG1; LRGUK; LRIF1; LRIG1; LRIG3; LRIT1; LRIT3; LRMP; LRP10; LRP12; LRP1B; LRP1; LRP2BP; LRP2; LRP4; LRP5; LRP6; LRPAP1; LRPPRC; LRR1; LRRC15; LRRC16A; LRRC16B; LRRC17; LRRC18; LRRC1; LRRC20; LRRC26; LRRC30; LRRC32; LRRC37A; LRRC37B; LRRC39; LRRC49; LRRC4B; LRRC4C; LRRC4; LRRC52; LRRC59; LRRC61; LRRC63; LRRC69; LRRC6; LRRC74A; LRRC7; LRRC8A; LRRC8C; LRRCC1; LRRFIP1; LRRFIP2; LRRK1; LRRK2; LRRN1; LRRN2; LRRN3; LRRN4; LRRTM1; LRRTM2; LRRTM3; LRRTM4; LRSAM1; LRTM1; LRTOMT; LSAMP; LSM11; LSM1; LSM2; LSM3; LSM4; LSM5; LSM6; LSM7; LSP1; LSR; LSS; LST1; LTA4H; LTA; LTB4R2; LTB4R; LTB; LTBP1; LTBP2; LTBP3; LTBP4; LTBR; LTC4S; LTF; LTK; LUC7L3; LUC7L; LUM; LURAP1L; LUZP4; LUZP6; LVRN; LXN; LY6D; LY6E; LY6G5B; LY6G5C; LY6G6C; LY6G6F; LY6K; LY75-CD302; LY75; LY86; LY96; LY9; LYL1; LYN; LYNX1; LYPD1; LYPD2; LYPD3; LYPD4; LYPD5; LYPD6; LYPD8; LYPLA1; LYPLAL1; LYRM1; LYRM4; LYRM7; LYRM9; LYST; LYVE1; LYZ; LYZL1; LYZL2; LYZL6; LZTFL1; LZTR1; LZTS1; M6PR; MAATS1; MAB21L1; MAB21L2; MAB21L3; MACC1; MACROD1; MACROD2; MAD1L1; MAD2L1BP; MAD2L1; MAD2L2; MADCAM1; MADD; MAEA; MAEL; MAF1; MAFA; MAFB; MAFF; MAFG; MAF; MAFK; MAGEA10; MAGEA11; MAGEA12; MAGEA1; MAGEA2B; MAGEA3; MAGEA4; MAGEA6; MAGEA9; MAGEB17; MAGEB1; MAGEB2; MAGEB6; MAGEC1; MAGEC2; MAGEC3; MAGED1; MAGED2; MAGED4B; MAGED4; MAGEE1; MAGEH1; MAGEL2; MAG; MAGI1; MAGI2; MAGI3; MAGT1; MAK16; MAK; MAL; MALL; MALRD1; MALT1; MAML1; MAML2; MAML3; MAMLD1; MAN1A1; MAN1B1; MAN2A1; MAN2A2; MAN2B1; MAN2C1; MANBA; MANEA; MANF; MANSC1; MAOA; MAOB; MAP10; MAP1A; MAP1B; MAP1LC3A; MAP1LC3B2; MAP1LC3B; MAP1S; MAP2; MAP2K1; MAP2K2; MAP2K3; MAP2K4; MAP2K5; MAP2K6; MAP2K7; MAP3K10; MAP3K11; MAP3K12; MAP3K13; MAP3K14; MAP3K19; MAP3K1; MAP3K2; MAP3K3; MAP3K4; MAP3K5; MAP3K6; MAP3K7CL; MAP3K7; MAP3K8; MAP3K9; MAP4; MAP4K1; MAP4K2; MAP4K3; MAP4K4; MAP4K5; MAP6; MAP7; MAPS; MAPK10; MAPK11; MAPK12; MAPK13; MAPK14; MAPK15; MAPK1; MAPK3; MAPK6; MAPK7; MAPK8; MAPK8IP1; MAPK8IP2; MAPK8IP3; MAPK9; MAPKAP1; MAPKAPK2; MAPKAPK3; MAPKBP1; MAPRE1; MAPRE2; MAPRE3; MAPT; MARC1; MARC2; MARCH1; MARCH2; MARCH5; MARCH6; MARCH7; MARCH8; MARCKS; MARCO; MARK1; MARK2; MARK3; MARK4; MARS2; MARS; MARVELD1; MARVELD2; MARVELD3; MAST; MASTL; MASP1; MAST2; MAST4; MASTL; MAT1A; MAT2A; MAT2B; MATK; MATN2; MATN3; MATR3; MAU2; MAVS; MAX; MAZ; MB21D1; MB21D2; MBD1; MBD2; MBD3; MBD3L2; MBD4; MBD5; MBD6; MB; MBIP; MBL2; MBNL1; MBNL2; MBOAT1; MBOAT2; MBOAT4; MBP; MBTD1; MBTPS1; MBTPS2; MC1R; MC2R; MC3R; MC4R; MC5R; MCAM; MCAT; MCCC1; MCCC2; MCCD1; MCC; MCEE; MCF2; MCF2L2; MCF2L; MCFD2; MCHR1; MCHR2; MCIDAS; MCM10; MCM2; MCM3AP; MCM3; MCM4; MCM5; MCM6; MCMI; MCM8; MCM9; MCMBP; MCOLN1; MCOLN3; MCPH1; MCRS1; MCTP1; MCTP2; MCTS1; MCU; MCUR1; MDC1; MDFIC; MDFI; MDGA1; MDGA2; MDH1; MDH2; MDK; MDM1; MDM2; MDM4; ME1; ME2; ME3; MEA1; MECOM; MECP2; MED12; MED12L; MED13; MED13L; MED14; MED15; MED16; MED17; MED19; MEDT; MED22; MED23; MED24; MED25; MED28; MED29; MED30; MED4; MED6; MEDS; MEF2A; MEF2B; MEF2BNB-MEF2B; MEF2C; MEF2D; MEFV; MEGF10; MEGF11; MEGF8; MEGF9; MEI1; MEIS1; MEIS2; MELK; MEMO1; MEN1; MEOX1; MEOX2; MEP1A; MEPE; MERTK; MESDC1; MESDC2; MESP2; MEST; METAP1D; METAP1; METAP2; MET; METRN; METTL13; METTL16; METTL17; METTL18; METTL1; METTL21A; METTL21B; METTL21C; METTL24; METTLE; METTL7A; METTL9; MEX3B; MEX3C; MEX3D; MFAP3; MFAP4; MFAP5; MFGE8; MFHAS1; MFI2; MFN1; MFRP; MFSD11; MFSD12; MFSD1; MFSD6; MFSD7; MFSD8; MGA; MGAM; MGARP; MGAT1; MGAT2; MGAT3; MGAT4A; MGAT4B; MGAT4C; MGAT5B; MGAT5; MGEA5; MGLL; MGME1; MGMT; MGP; MGRN1; MGST1; MGST2; MGST3; MIA2; MIA3; MIA; MIB1; MIB2; MICA; MICAL2; MICALCL; MICB; MICU1; MICU3; MID1; MID2; MIEN1; MIER2; MIER3; MIF; MILR1; MINA; MINK1; MINPP1; MIOX; MIPEP; MIP; MIPOL1; MIR1-1HG; MIS18BP1; MITF; MIXL1; MKI67; MKKS; MKL1; MKL2; MKLN1; MKNK1; MKNK2; MKRN1; MKRN3; MKS1; MLANA; MLC1; MLEC; MLF1; MLF2; MLH1; MLH3; MLKL; MLLT10; MLLT1; MLLT3; MLLT4; MLLT6; MLN; MLNR; MLPH; MLST8; MLX; MLXIP; MLXIPL; MLYCD; MMAA; MMAB; MMADHC; MMD2; MMD; MME; MMEL1; MMP10; MMP11; MMP12; MMP13; MMP14; MMP15; MMP16; MMP17; MMP19; MMPI; MMP20; MMP21; MMP24; MMP25; MMP26; MMP28; MMP2; MMP3; MMPI; MMP8; MMP9; MMRN1; MMRN2; MMS19; MMS22L; MN1; MNAT1; MNDA; MNS1; MNT; MNX1; MOAP1; MOB1A; MOB1B; MOB2; MOB3B; MOB4; MOBP; MOCOS; MOCS1; MOCS2; MOGAT1; MOGAT2; MOGAT3; MOG; MOGS; MOK; MON1A; MON1B; MON2; MORC3; MORF4L1; MORN1; MORN2; MORNS; MOS; MOV10L1; MPC1; MPC2; MPDU1; MPDZ; MPEG1; MPG; MPHOSPH10; MPHOSPH6; MPHOSPH8; MPHOSPH9; MPI; MPLKIP; MPO; MPP1; MPP2; MPP3; MPP5; MPP7; MPPE1; MPPED2; MPRIP; MPST; MPV17; MPV17L2; MPZ; MPZL1; MPZL2; MPZL3; MR1; MRAP2; MRAP; MRAS; MRC1; MRC2; MRE11A; MREG; MRFAP1; MRGBP; MRGPRF; MRGPRX1; MRGPRX3; MRGPRX4; MRI1; MRM1; MROH2B; MRO; MRPL10; MRPL11; MRPL13; MRPL15; MRPL17; MRPL19; MRPL1; MRPL23; MRPL28; MRPL33; MRPL36; MRPL3; MRPL40; MRPL41; MRPL44; MRPL49; MRPL52; MRPL9; MRPS11; MRPS12; MRPS16; MRPS18B; MRPS22; MRPS23; MRPS28; MRPS30; MRPS31; MRPS33; MRPS6; MRPS7; MRPS9; MRRF; MRS2; MRVI1; MS4Al2; MS4A1; MS4A2; MS4A3; MS4A4A; MS4A6A; MS4A8; MSANTD3-TMEFF1; MSC; MSGN1; MSH2; MSH3; MSHS; MSH6; MSI1; MSI2; MSLN; MSMB; MSMO1; MSMP; MSN; MSR1; MSRA; MSRB2; MSRB3; MST1; MST1R; MSTN; MSTO1; MSX1; MSX2; MT1A; MT1B; MT1E; MT1F; MT1G; MT1H; MT1M; MT1X; MT2A; MT3; MT4; MTA1; MTA2; MTA3; MTAP; MTBP; MTCH1; MTCH2; MTCL1; MTCP1; MTDH; MTERF1; MTF1; MTFMT; MTFP1; MTG1; MTHFD1; MTHFD1L; MTHFD2; MTHFR; MTHFS; MTIF2; MTIF3; MTL5; MTM1; MTMR11; MTMR12; MTMR14; MTMR1; MTMR2; MTMR3; MTMR4; MTMR6; MTMR7; MTMR8; MTMR9; MTNR1A; MTNR1B; MT01; MTPAP; MTPN; MTRF1; MTR; MTRNR2L7; MTRR; MTSS1; MTTP; MTURN; MTUS1; MTUS2; MTX1; MTX2; MUC12; MUC13; MUC15; MUC17; MUC19; MUC1; MUC20; MUC21; MUC22; MUC2; MUC3A; MUC4; MUC5AC; MUC5B; MUC6; MUC7; MUCL1; MUM1; MURC; MUS81; MUSK; MUT; MUTYH; MVB12B; MVD; MVK; MVP; MX1; MX2; MXD1; MXD3; MXD4; MXI1; MXRA5; MYADM; MYBBP1A; MYB; MYBL1; MYBL2; MYBPC1; MYBPC2; MYBPC3; MYBPH; MYCBP2; MYCBPAP; MYC; MYON; MYCT1; MYD88; MYDGF; MYEF2; MYEOV; MYF5; MYF6; MYH10; MYH11; MYH13; MYH14; MYH15; MYH1; MYH2; MYH3; MYH4; MYH6; MYH7B; MYH7; MYH8; MYH9; MYL12A; MYL12B; MYL1; MYL2; MYL3; MYL4; MYL6; MYL7; MYL9; MYLIP; MYLK2; MYLK3; MYLK; MYLPF; MYNN; MYO10; MYO15A; MYO16; MYO18B; MYO1A; MYO1B; MY01C; MYOCD; MYO1E; MYO1F; MYO3A; MYO3B; MYO5A; MYO5B; MY05C; MYO6; MYO7A; MY07B; MYO9A; MYO9B; MYOCD; MYOC; MYOD1; MYOF; MYOG; MYOM1; MYOM2; MYOT; MYOZ1; MYOZ2; MYOZ3; MYPN; MYRF; MYRFL; MYRIP; MYT1; MYT1L; MYZAP; MZB1; MZF1; N4BP1; N4BP2L1; N4BP2L2; NAA10; NAA15; NAA16; NAA20; NAA25; NAA30; NAA35; NAA40; NAA50; NAA60; NAAA; NAALAD2; NAALADL1; NAALADL2; NAB1; NAB2; NABP1; NABP2; NACA; NACC1; NACC2; NADSYN1; NAE1; NAF1; NAGA; NAGK; NAGLU; NAGPA; NAGS; NAIP; NALCN; NAMPT; NANOG; NANOS1; NANOS2; NANOS3; NANS; NAP1L1; NAP1L3; NAP1L4; NAP1L5; NAPA; NAPEPLD; NAPG; NAPRT; NAPSA; NARFL; NARR; NARS; NAT10; NAT14; NAT1; NAT2; NAT6; NAT8B; NAT8; NAT8L; NAV1; NAV2; NAV3; NBAS; NBEA; NBEAL1; NBEAL2; NBN; NBPF3; NBR1; NCALD; NCAM1; NCAM2; NCAN; NCAPD2; NCAPD3; NCAPG2; NCAPG; NCBP2; NCEH1; NCF1; NCF2; NCF4; NCK1; NCK2; NCKAP1; NCKAP1L; NCKAP5; NCKIPSD; NCL; NCOA1; NCOA2; NCOA3; NCOA4; NCOA5; NCOA6; NCOA7; NCOR1; NCOR2; NCR1; NCR2; NCR3; NCR3LG1; NCS1; NCSTN; NDC80; NDE1; NDEL1; NDFIP1; NDFIP2; NDNF; NDN; NDP; NDRG1; NDRG2; NDRG3; NDRG4; NDST1; NDST2; NDST3; NDST4; NDUFA10; NDUFA11; NDUFA12; NDUFA13; NDUFA1; NDUFA2; NDUFA5; NDUFA6; NDUFA9; NDUFAB1; NDUFAF1; NDUFAF2; NDUFAF3; NDUFAF4; NDUFAF5; NDUFAF6; NDUFB10; NDUFB11; NDUFB2; NDUFB3; NDUFB4; NDUFB5; NDUFB6; NDUFB8; NDUFB9; NDUFC2; NDUFS1; NDUFS2; NDUFS3; NDUFS4; NDUFS6; NDUFS7; NDUFS8; NDUFV1; NDUFV2; NDUFV3; NEB; NEBL; NECAB1; NECAB3; NECAP1; NEDD1; NEDD4; NEDD4L; NEDD8; NEDD9; NEFH; NEFM; NEIL1; NEIL2; NEIL3; NEK10; NEK11; NEK1; NEK2; NEK3; NEK4; NEK6; NEK7; NEK8; NEK9; NELFA; NELFB; NELFCD; NELFE; NELL1; NELL2; NEMF; NE01; NES; NETT; NET01; NETO2; NEU1; NEU3; NEURL1; NEURL2; NEUROD1; NEUROD2; NEUROD4; NEUROD6; NEUROG1; NEUROG2; NEUROG3; NF1; NF2; NFAM1; NFASC; NFAT5; NFATC1; NFATC2; NFATC3; NFATC4; NFE2; NFE2L1; NFE2L2; NFE2L3; NFIA; NFIB; NFIC; NFIL3; NFIX; NFKB1; NFKB2; NFKBIA; NFKBIB; NFKBIE; NFKBIL1; NFKBIZ; NFRKB; NFS1; NFU1; NFYA; NFYB; NFYC; NGB; NGDN; NGEF; NGF; NGFRAP1; NGFR; NGLY1; NHEJ1; NHLH1; NHLRC1; NHLRC3; NHP2; NHP2L1; NHS; NHSL1; NICN1; NID1; NID2; NIF3L1; NIM1K; NIN; NINJ1; NINJ2; NINL; NIPA1; NIPA2; NIPAL3; NIPAL4; NIPBL; NIPSNAP1; NIPSNAP3B; NISCH; NIT1; NIT2; NKAIN2; NKAIN3; NKAP; NKD1; NKD2; NKG7; NKIRAS1; NKIRAS2; NKRF; NKTR; NKX1-1; NKX1-2; NKX2-1; NKX2-2; NKX2-3; NKX2-5; NKX2-6; NKX2-8; NKX3-1; NKX6-1; NKX6-2; NLE1; NLGN1; NLGN2; NLGN3; NLGN4X; NLGN4Y; NLK; NLN; NLRC3; NLRC4; NLRC5; NLRP10; NLRP11; NLRP12; NLRP13; NLRP14; NLRP1; NLRP2; NLRP3; NLRP4; NLRP5; NLRP6; NLRP7; NLRP8; NLRP9; NMB; NMBR; NME1; NME1-NME2; NME2; NME3; NME4; NME5; NME6; NME7; NME8; NME9; NMI; NMNAT1; NMNAT2; NMNAT3; NMS; NMT1; NMT2; NMU; NMUR1; NMUR2; NNAT; NNMT; NNT; NOA1; NOB1; NOBOX; NOC3L; NOD1; NOD2; NODAL; NOG; NOL11; NOL3; NOL4; NOL6; NOL8; NOLC1; NOMI; NOMO1; NONO; NOP10; NOP14; NOP16; NOP2; NOP56; NOP9; NOS1AP; NOS1; NOS2; NOS3; NOSIP; NOSTRIN; NOTCH1; NOTCH2; NOTCH3; NOTCH4; NOTUM; NOVA1; NOVA2; NOV; NOX1; NOX3; NOX4; NOX5; NOXA1; NOXO1; NPAP1; NPAS2; NPAS3; NPAS4; NPAT; NPB; NPBWR1; NPC1; NPC1L1; NPC2; NPDC1; NPEPL1; NPEPPS; NPFF; NPFFR2; NPHP1; NPHP3; NPHP4; NPHS1; NPHS2; NPL; NPLOC4; NPM1; NPM2; NPNT; NPPC; NPR1; NPR2; NPR3; NPRL2; NPRL3; NPS; NPSR1; NPTN; NPTX1; NPTX2; NPTXR; NPVF; NPW; NPY1R; NPY2R; NPY5R; NPY; NQO1; NQO2; NROB1; NR101; NR102; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRAP; NRARP; NRBP1; NRBP2; NRCAM; NRD1; NRDE2; NREP; NRF1; NRG1; NRG2; NRG3; NRG4; NRGN; NRIP1; NRIP2; NRK; NRL; NRM; NRN1; NRP1; NRP2; NRSN1; NRSN2; NRTN; NRXN1; NRXN2; NRXN3; NSA2; NSD1; NSDHL; NSF; NSFL1C; NSG1; NSMAF; NSMCE2; NSMF; NSRP1; NSUN2; NSUN3; NSUN5; NSUN7; NT5C1B; NT5C1B-RDH14; NT5C2; NT5C3A; NT5C; NT5DC1; NT5DC3; NT5E; NT5M; NTAN1; NTF3; NTF4; NTHL1; NTM; NTN1; NTN4; NTNG1; NTNG2; NTPCR; NTRK1; NTRK2; NTRK3; NTS; NTSR1; NTSR2; NUAK1; NUAK2; NUB1; NUBP1; NUBP2; NUBPL; NUCB1; NUCB2; NUCKS1; NUDCD1; NUDCD3; NUDC; NUDT10; NUDT11; NUDT15; NUDT19; NUDT1; NUDT21; NUDT2; NUDT3; NUDT6; NUDT7; NUF2; NUFIP1; NUFIP2; NUGGC; NUMA1; NUMB; NUMBL; NUP153; NUP155; NUP205; NUP210; NUP214; NUP35; NUP37; NUP43; NUP50; NUP62; NUP85; NUP88; NUP93; NUP98; NUPL2; NUPR1; NUS1; NUSAP1; NUTF2; NUTM1; NUTM2A; NUTM2B; NVL; NWD1; NXF1; NXF2B; NXF3; NXF5; NXN; NXNL1; NXNL2; NXPE1; NXPE2; NXPE4; NXPH1; NXPH2; NXT1; NXT2; NYAP2; NYX; OARD1; OAS1; OAS2; OAS3; OASL; OAT; OAZ1; OAZ2; OBFC1; OBP2A; OBSCN; OBSL1; OC90; OCA2; OCIAD1; OCIAD2; OCLM; OCLN; OCM2; OCM; OCRL; ODAM; ODC1; ODF1; ODF3B; ODF4; OFCC1; OFD1; OGDH; OGDHL; OGFOD1; OGFR; OGG1; OGN; OGT; OIP5; OIT3; OLA1; OLFM1; OLFM2; OLFM4; OLFML2B; OLIG1; OLIG2; OLIG3; OLR1; OMD; OMG; OMP; ONECUT1; ONECUT2; OPA1; OPA3; OPCML; OPHN1; OPN1LW; OPN1MW2; OPN1SW; OPN3; OPN4; OPN5; OPRK1; OPRL1; OPRM1; OPTC; OPTN; OR10A2; OR10A4; OR10C1; OR10J1; OR10J3; OR10J5; OR10K1; OR10K2; OR10R2; OR10T2; OR10X1; OR10Z1; OR11A1; OR12D2; OR12D3; OR13C3; OR13C4; OR13F1; OR13G1; OR13J1; OR14J1; OR1A1; OR1C1; OR1D2; OR1E1; OR1E2; OR1J2; OR1K1; OR1L8; OR1M1; OR1N1; OR1N2; OR2A25; OR2AG1; OR2AK2; OR2AT4; OR2B2; OR2B3; OR2B6; OR2C1; OR2F2; OR2G2; OR2G3; OR2H2; OR2J2; OR2J3; OR2L2; OR2M3; OR2M4; OR2M7; OR2S2; OR2T10; OR2T12; OR2T1; OR2T2; OR2T33; OR2T4; OR2T5; OR2W1; OR2Y1; OR2Z1; OR3A1; OR4A15; OR4C12; OR4C13; OR4C6; OR4D10; OR4K13; OR4P4; OR4S2; OR51A2; OR51A7; OR51E1; OR51E2; OR51F2; OR51V1; OR52B2; OR52B4; OR52D1; OR52E6; OR5211; OR5212; OR52K1; OR52K2; OR52M1; OR5D18; OR5H2; OR5H6; OR5K1; OR5K2; OR5V1; OR6B1; OR6B2; OR6C1; OR6F1; OR6K2; OR6K3; OR6K6; OR6N1; OR6N2; OR6X1; OR6Y1; OR7C1; OR7D2; OR7E24; OR8S1; OR9K2; OR9Q2; ORAI1; ORAI3; ORA0V1; ORC2; ORC3; ORC4; ORC5; ORC6; ORM1; ORM2; ORMDL3; OS9; OSBP2; OSBP; OSBPL10; OSBPL11; OSBPL1A; OSBPL2; OSBPL3; OSBPL5; OSBPL6; OSBPL8; OSBPL9; OSCAR; OSER1; OSGEP; OSGIN1; OSM; OSMR; OSR1; OSR2; OSTF1; OSTM1; OTC; OTOA; OTOF; OTOG; OTOGL; OTOL1; OTOP1; OTOR; OTP; OTUB1; OTUD1; OTUD4; OTUD7A; OTUD7B; OTULIN; OTX1; OTX2; OVCA2; OVOL1; OVOL2; OXA1L; OXCT1; OXER1; OXGR1; OXR1; OXSR1; OXT; OXTR; P2RX1; P2RX2; P2RX3; P2RX4; P2RX5; P2RX6; P2RX7; P2RY11; P2RY12; P2RY13; P2RY14; P2RY1; P2RY2; P2RY4; P2RY6; P2RY8; P3H2; P3H3; P3H4; P4HA1; P4HA2; P4HB; P4HTM; PA2G4; PABPC1; PABPC3; PABPC4L; PABPN1; PACRG; PACS1; PACS2; PACSIN1; PACSIN2; PADI4; PAEP; PAF1; PAFAH1B1; PAFAH1B2; PAFAH2; PAG1; PAGE1; PAGE4; PAGES; PAGR1; PAH; PAICS; PAIP1; PAIP2; PAK1; PAK1IP1; PAK2; PAK3; PAK4; PAK6; PAK7; PALB2; PALD1; PALLD; PALM2-AKAP2; PAM16; PAM; PAMR1; PAN2; PAN3; PANK1; PANK2; PANX1; PANX2; PAOX; PAPD7; PAPL; PAPOLA; PAPOLG; PAPPA2; PAPPA; PAPSS1; PAPSS2; PAQR3; PAQR5; PAQR7; PARD3B; PARD3; PARD6A; PARD6B; PARG; PARK2; PARL; PARM1; PARN; PARP12; PARP14; PARP15; PARP1; PARP2; PARP3; PARP4; PARP6; PARP9; PARPBP; PARS2; PARVA; PARVB; PARVG; PASD1; PASK; PATE1; PATZ1; PAWR; PAX1; PAX2; PAX3; PAX4; PAX5; PAX6; PAX8; PAX9; PAXIP1; PBK; PBLD; PBOV1; PBRM1; PBX1; PBX2; PBX3; PBX4; PCBD1; PCBD2; PCBP1; PCBP2; PCBP3; PCBP4; PCCA; PCCB; PCDH10; PCDH11X; PCDH11Y; PCDH15; PCDH17; PCDH18; PCDH19; PCDH1; PCDH20; PCDH7; PCDH8; PCDH9; PCDHA1; PCDHA4; PCDHA6; PCDHB1; PCDHB2; PCDHB3; PCDHB8; PCDHGA11; PCDHGA3; PCDHGB4; PCDHGB6; PCDHGC3; PCED1B; PCF11; PCGF1; PCGF2; PCGF3; PCGF6; PC; PCID2; PCK1; PCK2; PCLO; PCM1; PCMT1; PCMTD1; PCNA; PCNT; PCNXL2; PCNXL4; PCOLCE2; PCOLCE; PCP2; PCP4; PCSK1; PCSK1N; PCSK2; PCSK4; PCSK5; PCSK6; PCSK7; PCTP; PCYT1A; PCYT1B; PDAP1; PDCD10; PDCD1; PDCD1LG2; PDCD2; PDCD4; PDCD5; PDCD6; PDCD6IP; PDCD7; PDC; PDCL2; PDCL3; PDE10A; PDE11A; PDE12; PDE1A; PDE1B; PDE1C; PDE2A; PDE3A; PDE3B; PDE4A; PDE4D; PDE5A; PDE6A; PDE6B; PDE6C; PDE6D; PDE6G; PDE6H; PDE7A; PDE7B; PDE8A; PDE8B; PDE9A; PDF; PDGFA; PDGFB; PDGFC; PDGFD; PDGFRA; PDGFRB; PDGFRL; PDHA1; PDHB; PDHX; PDIA2; PDIA3; PDIA4; PDIA5; PDIA6; PDK1; PDK2; PDK3; PDK4; PDLIM1; PDLIM2; PDLIM3; PDLIM4; PDLIM5; PDLIM7; PDP1; PDP2; PDPK1; PDPR; PDRG1; PDS5A; PDS5B; PDSS1; PDSS2; PDX1; PDXDC1; PDXK; PDXP; PDYN; PDZD2; PDZD4; PDZD7; PDZD8; PDZK1; PDZRN3; PDZRN4; PEA15; PEAK1; PEARL; PEBP1; PEBP4; PECAM1; PECR; PEG10; PEG3; PELI1; PELI2; PELP1; PEMT; PENK; PEPD; PER1; PER2; PER3; PERM1; PERP; PES1; PET100; PET117; PEX11A; PEX12; PEX13; PEX14; PEX16; PEX19; PEX1; PEX26; PEX2; PEX3; PEX5; PEX5L; PEX6; PEX7; PF4; PF4V1; PFDN4; PFDN5; PFDN6; PFKFB1; PFKFB2; PFKFB3; PFKFB4; PFKL; PFKM; PFKP; PFN1; PFN2; PGA3; PGA4; PGA5; PGAM1; PGAM2; PGAM4; PGAP1; PGAP2; PGAP3; PGBD1; PGBD5; PGC; PGD; PGF; PGGT1B; PGK1; PGK2; PGLS; PGLYRP1; PGLYRP2; PGLYRP3; PGLYRP4; PGM3; PGPEP1; PGP; PGR; PGRMC1; PGRMC2; PHACTR1; PHACTR2; PHACTR3; PHB2; PHB; PHC1; PHC2; PHC3; PHEX; PHF10; PHF11; PHF12; PHF19; PHF1; PHF20; PHF21A; PHF23; PHF2; PHF3; PHF5A; PHF6; PHF8; PHGDH; PHIP; PHKA1; PHKA2; PHKB; PHKG1; PHKG2; PHLDA1; PHLDA2; PHLDA3; PHLDB1; PHLPP1; PHLPP2; PHOX2A; PHOX2B; PHPT1; PHRF1; PHTF1; PHTF2; PHYH; PHYHIP; PHYKPL; PI15; PI16; PI3; PI4K2A; PI4K2B; PI4KA; PI4KB; PIAS1; PIAS2; PIAS4; PIBF1; PICALM; PICK1; PID1; PIDD1; PIEZO1; PIEZO2; PIF1; PIFO; PIGA; PIGF; PIGG; PIGL; PIGM; PIGN; PIGO; PIGP; PIGQ; PIGR; PIGT; PIGU; PIGW; PIGY; PIH101; PIK3AP1; PIK3C2A; PIK3C2B; PIK3C2G; PIK3C3; PIK3CA; PIK3CB; PIK3CD; PIK3CG; PIK3IP1; PIK3R1; PIK3R2; PIK3R3; PIK3R4; PIK3R5; PIKFYVE; PILRA; PIM1; PIM2; PIM3; PINI; PIN4; PINX1; PIP4K2A; PIP4K2B; PIP4K2C; PIPSK1A; PIPSK1B; PIPSK1C; PIPSKL1; PIP; PIPDX; PIR; PISD; PITPNA; PITPNM1; PITPNM3; PITRM1; PITX1; PITX2; PITX3; PIWIL1; PIWIL2; PIWIL3; PIWIL4; PJA1; PJA2; PKD1; PKD1L1; PKD1L2; PKD1L3; PKD2; PKD2L1; PKDCC; PKDREJ; PKHD1; PKIA; PKIB; PKLR; PKM; PKMYT1; PKN1; PKN2; PKN3; PKNOX1; PKNOX2; PKP1; PKP2; PKP3; PKP4; PLAZA; PLA2G10; PLA2G12A; PLA2G12B; PLA2G15; PLA2G16; PLA2G1B; PLA2G3; PLA2G4A; PLA2G4B; PLA2G4C; PLA2G4D; PLA2G5; PLA2G6; PLA2G7; PLA2R1; PLAA; PLAC1; PLACE; PLAG1; PLAGL1; PLAGL2; PLAT; PLAU; PLAUR; PLB1; PLBD1; PLCB1; PLCB2; PLCB3; PLCB4; PLCD1; PLCD3; PLCD4; PLCE1; PLCG1; PLCG2; PLCH1; PLCL1; PLCL2; PLCXD2; PLCXD3; PLCZ1; PLD1; PLD2; PLD3; PLD4; PLD5; PLEC; PLEK2; PLEKHA1; PLEKHA2; PLEKHAS; PLEKHA6; PLEKHA7; PLEKHB1; PLEKHD1; PLEKHF1; PLEKHF2; PLEKHG1; PLEKHG2; PLEKHG3; PLEKHG4; PLEKHG6; PLEKHH2; PLEKHM1; PLEKHO1; PLEKHG2; PLEK; PLG; PLGLB2; PLIN1; PLIN2; PLIN3; PLIN4; PLIN5; PLK1; PLK2; PLK4; PLK5; PLLP; PLN; PLOD2; PLP1; PLP2; PLRG1; PLS1; PLS3; PLSCR1; PLSCR3; PLSCR4; PLSCR5; PLTP; PLVAP; PLXDC1; PLXDC2; PLXNA1; PLXNA2; PLXNA3; PLXNA4; PLXNB1; PLXNB3; PLXNC1; PLXND1; PM20D1; PMAIP1; PMCH; PMEL; PMEPA1; PMF1-BGLAP; PMF1; PML; PMM1; PMM2; PMP22; PMPCA; PMPCB; PMS1; PMS2; PMVK; PNCK; PNKD; PNKP; PNLDC1; PNLIP; PNLIPRP2; PNMA1; PNMA2; PNMT; PNN; PNO1; PNOC; PNP; PNPLA1; PNPLA2; PNPLA3; PNPLA4; PNPLA5; PNPLA6; PNPLA8; PNPO; PNPT1; PNRC1; POC1A; POC1B; P005; PODXL; POF1B; POFUT1; POFUT2; POGK; POGLUT1; POLA1; POLA2; POLB; POLD1; POLD2; POLD3; POLD4; POLDIP3; POLE2; POLE3; POLE4; POLE; POLG2; POLG; POLH; POLI; POLK; POLL; POLM; POLN; POLQ; POLR1A; POLR1B; POLR1C; POLR1D; POLR2A; POLR2B; POLR2C; POLR2D; POLR2E; POLR2F; POLR2G; POLR2H; POLR2J; POLR2K; POLR2M; POLR3A; POLR3B; POLR3E; POLR3K; POLRMT; POM121; POMC; POMGNT1; POMGNT2; POMK; POMP; POMT1; POMT2; POMZP3; PON1; PON2; PON3; POP1; POP4; POPDC3; PORCN; POR; POSTN; POT1; POTED; POTEF; POTEG; POTEH; POTEM; POU1F1; POU2AF1; POU2F1; POU2F2; POU2F3; POU3F2; POU3F3; POU3F4; POU4F1; POU4F2; POU4F3; POU5F1B; POU5F1; POU6F1; POU6F2; PPA1; PPA2; PPAP2A; PPAP2C; PPAPDC1B; PPAPDC2; PPAPDC3; PPARA; PPARD; PPARGC1A; PPARGC1B; PPARG; PPAT; PPBP; PPCDC; PPEF1; PPEF2; PPFIA1; PPFIA2; PPFIA4; PPFIBP1; PPFIBP2; PPIA; PPIB; PPIC; PPID; PPIF; PPIG; PPIL1; PPIL2; PPIL3; PPIP5K1; PPL; PPM1A; PPM1B; PPM1D; PPM1E; PPM1F; PPM1G; PPM1H; PPM1K; PPM1L; PPM1M; PPME1; PPDX; PPP1CA; PPP1CC; PPP1R10; PPP1R11; PPP1R12A; PPP1R12B; PPP1R12C; PPP1R13B; PPP1R13L; PPP1R14A; PPP1R14B; PPP1R14C; PPP1R15A; PPP1R15B; PPP1R17; PPP1R18; PPP1R1A; PPP1R1B; PPP1R2; PPP1R3A; PPP1R3B; PPP1R3C; PPP1R42; PPP1R7; PPP1R9A; PPP2CA; PPP2CB; PPP2R1A; PPP2R1B; PPP2R2A; PPP2R2B; PPP2R2C; PPP2R3A; PPP2R3B; PPP2R4; PPP2R5A; PPP2R5B; PPP2R5C; PPP2R5D; PPP2R5E; PPP3CA; PPP3CB; PPP3CC; PPP3R1; PPP3R2; PPP4C; PPP4R1; PPP5C; PPP6C; PPP6R2; PPP6R3; PPRC1; PPT2; PPY; PQBP1; PQLC3; PRAC1; PRAC2; PRADC1; PRAF2; PRAM1; PRAME; PRAP1; PRB1; PRB2; PRB3; PRB4; PRC1; PRCC; PRCD; PRCP; PRDM10; PRDM11; PRDM13; PRDM14; PRDM1; PRDM4; PRDM5; PRDM6; PRDM7; PRDM8; PRDM9; PRDX2; PRDX3; PRDX4; PRDX5; PRDX6; PREB; PRELP; PREP; PREPL; PREX1; PREX2; PRF1; PRG2; PRG4; PRH1; PRH2; PRICKLE1; PRICKLE2; PRICKLE4; PRIM1; PRIMAL; PRIMPOL; PRKAA1; PRKAB1; PRKACA; PRKACB; PRKACG; PRKAG1; PRKAG2; PRKAG3; PRKAR1A; PRKAR1B; PRKAR2A; PRKAR2B; PRKCA; PRKCB; PRKCDBP; PRKCD; PRKCE; PRKCG; PRKCH; PRKCI; PRKCQ; PRKCSH; PRKCZ; PRKD1; PRKD3; PRKDC; PRKG1; PRKG2; PRKRA; PRKRIR; PRKX; PRLH; PRL; PRLHR; PRLR; PRM1; PRM2; PRM3; PRMT1; PRMT2; PRMT3; PRMT5; PRMT8; PRND; PRNP; PROC; PROCR; PRODH; PROK2; PROKR1; PROKR2; PROL1; PROM1; PROM2; PROP1; PROS1; PROSER1; PROX1; PROZ; PRPF19; PRPF31; PRPF38B; PRPF3; PRPF40A; PRPF4B; PRPF4; PRPF6; PRPF8; PRPH2; PRPH; PRPS1; PRPS1L1; PRPS2; PRPSAP1; PRPSAP2; PRR11; PRR13; PRR15; PRR16; PRR34; PRR3; PRR5; PRR9; PRRC1; PRRC2A; PRRC2C; PRRG4; PRRT1; PRRT2; PRRX1; PRRX2; PRSS12; PRSS16; PRSS1; PRSS21; PRSS22; PRSS23; PRSS27; PRSS2; PRSS33; PRSS35; PRSS3; PRSS50; PRSS53; PRSS55; PRSS57; PRSS58; PRSS8; PRTFDC1; PRTG; PRTN3; PRUNE2; PRUNE; PRX; PRY; PSAP; PSAT1; PSCA; PSD3; PSD4; PSD; PSEN1; PSEN2; PSENEN; PSG1; PSG2; PSG5; PSG6; PSG8; PSG9; PSIP1; PSKH1; PSMA1; PSMA2; PSMA3; PSMA4; PSMA6; PSMA7; PSMB10; PSMB1; PSMB4; PSMB5; PSMB6; PSMB7; PSMB8; PSMB9; PSMC1; PSMC2; PSMC3; PSMC3IP; PSMC4; PSMC5; PSMC6; PSMD10; PSMD12; PSMD13; PSMD14; PSMD1; PSMD2; PSMD3; PSMD4; PSMD6; PSMD7; PSMD8; PSMD9; PSME1; PSME2; PSME3; PSME4; PSMF1; PSMG1; PSMG2; PSMG3; PSORS1C1; PSORS1C2; PSPC1; PSPH; PSPN; PSTPIP1; PSTPIP2; PTBP1; PTBP2; PTCD1; PTCH1; PTCHD1; PTCHD4; PTCRA; PTDSS1; PTEN; PTER; PTF1A; PTGDR2; PTGDR; PTGDS; PTGER1; PTGER2; PTGER3; PTGER4; PTGES2; PTGES3; PTGES; PTGIR; PTGIS; PTGR1; PTGS1; PTGS2; PTH1R; PTH2; PTH2R; PTH; PTHLH; PTK2B; PTK2; PTK6; PTK7; PTMA; PTMS; PTN; PTOV1; PTP4A1; PTP4A3; PTPMT1; PTPN11; PTPN12; PTPN13; PTPN14; PTPN18; PTPN1; PTPN21; PTPN22; PTPN23; PTPN2; PTPN3; PTPN4; PTPN5; PTPN6; PTPN7; PTPN9; PTPRA; PTPRB; PTPRCAP; PTPRD; PTPRE; PTPRG; PTPRH; PTPRJ; PTPRK; PTPRM; PTPRN2; PTPRN; PTPRO; PTPRQ; PTPRR; PTPRS; PTPRT; PTPRZ1; PTRF; PTRH1; PTRH2; PTRHD1; PTS; PTTG1; PTTG1IP; PTTG2; PTX3; PUF60; PUM2; PURA; PURB; PUS10; PUS1; PVALB; PVR; PVRL1; PVRL2; PVRL3; PVRL4; PWP2; PWWP2B; PXDN; PXDNL; PXK; PXMP2; PXN; PXT1; PYCARD; PYCR1; PYDC1; PYDC2; PYGB; PYGL; PYGM; PYG01; PYGO2; PYHIN1; PYROXD1; PYY; PZP; QARS; QDPR; QKI; QPCT; QPCTL; QPRT; QRFP; QRFPR; QRSL1; QSOX1; QSOX2; QTRT1; R3HCC1; R3HCC1L; R3HDM1; R3HDML; RAB11A; RAB11B; RAB11FIP1; RAB11FIP2; RAB11FIP3; RAB11FIP4; RAB11FIP5; RAB12; RAB14; RAB15; RAB18; RAB1A; RAB1B; RAB20; RAB21; RAB22A; RAB23; RAB24; RAB25; RAB27A; RAB27B; RAB28; RAB29; RAB2A; RAB31; RAB32; RAB33B; RAB34; RAB35; RAB36; RAB37; RAB38; RAB39A; RAB39B; RAB3A; RAB3D; RAB3GAP1; RAB3GAP2; RAB3IL1; RAB3IP; RAB40AL; RAB40B; RAB40C; RAB4A; RAB4B; RAB5A; RAB5B; RAB5C; RAB6A; RAB6B; RAB6C; RAB7A; RAB8A; RAB8B; RAB9A; RABAC1; RABEP1; RABEP2; RABEPK; RABGAP1L; RABGEF1; RABIF; RABL6; RAC1; RAC2; RAC3; RACGAP1; RAD17; RAD18; RAD1; RAD21; RAD21L1; RAD23A; RAD23B; RAD50; RAD51AP1; RAD51B; RAD51C; RAD51D; RAD51; RAD52; RAD54B; RAD9A; RAD9B; RAE1; RAET1E; RAET1L; RAFT; RAG1; RAG2; RAI14; RAI1; RAI2; RALA; RALB; RALBP1; RALGAPA1; RALGAPA2; RALGAPB; RALGDS; RALGPS1; RALY; RALYL; RAMP1; RAMP2; RAMP3; RANBP10; RANBP17; RANBP1; RANBP2; RANBP3; RANBP3L; RANBP6; RANBP9; RANGAP1; RANGRF; RAN; RAP1B; RAP1GAP2; RAP1GDS1; RAP2A; RAP2B; RAPGEF1; RAPGEF2; RAPGEF3; RAPGEF4; RAPGEF5; RAPH1; RAPSN; RARA; RARB; RARG; RARRES1; RARRES2; RARRES3; RARS2; RARS; RASA1; RASA2; RASAL1; RASAL2; RASD1; RASD2; RASEF; RASGEF1A; RASGEF1C; RASGRF1; RASGRF2; RASGRP1; RASGRP2; RASGRP3; RASGRP4; RASIP1; RASL10A; RASL10B; RASL11A; RASL11B; RASL12; RASSF1; RASSF2; RASSF3; RASSF4; RASSF5; RASSF6; RASSF7; RASSF8; RAX2; RAX; RB1CC1; RB1; RBAK; RBBP5; RBBP6; RBBP7; RBBP8; RBBP9; RBCK1; RBFOX1; RBFOX2; RBFOX3; RBL1; RBL2; RBM10; RBM12; RBM14; RBM14-RBM4; RBM15; RBM17; RBM20; RBM25; RBM26; RBM27; RBM28; RBM38; RBM39; RBM3; RBM45; RBM46; RBM47; RBM4; RBMS; RBM6; RBM7; RBMS1; RBMS2; RBMS3; RBMX2; RBMX; RBMXL2; RBMY1A1; RBP1; RBP2; RBP3; RBP4; RBPJ; RBPMS2; RBSN; RBX1; RC3H1; RCAN1; RCAN2; RCBTB1; RCBTB2; RCHY1; RCL1; RCN1; RCN2; RCOR1; RCSD1; RCVRN; RD3; RDH10; RDH11; RDH12; RDH14; RDH16; RDH5; RDH8; RDM1; RDX; REC8; RECK; RECQL5; RECQL; REEP1; REEP2; REEP3; REEP5; REEP6; REG1A; REG1B; REG3A; RELA; RELB; REL; RELN; REM1; RENBP; REN; REPS1; REPS2; RERG; RERGL; REST; RET; RETN; RETNLB; RETSAT; REV1; REV3L; REX01; REXO2; REXO4; RFC1; RFC2; RFC3; RFC4; RFC5; RFFL; RFK; RFPL1; RFT1; RFTN1; RFTN2; RFWD2; RFWD3; RFX1; RFX2; RFX3; RFX4; RFX5; RFX6; RFX8; RFXANK; RFXAP; RGCC; RGL1; RGL2; RGL4; RGMA; RGMB; RGN; RGPD2; RGR; RGS10; RGS11; RGS12; RGS13; RGS14; RGS16; RGS17; RGS18; RGS19; RGS1; RGS20; RGS21; RGS22; RGS2; RGS3; RGS4; RGS5; RGS6; RGS7BP; RGS7; RGS8; RGS9BP; RGS9; RGSL1; RHAG; RHBDD2; RHBDD3; RHBDF1; RHBDF2; RHBDL1; RHBDL2; RHCE; RHCG; RHEB; RHEBL1; RHNO1; RHOB; RHOBTB1; RHOBTB2; RHOBTB3; RHOD; RHOF; RHOG; RHOH; RHO; RHOJ; RHOQ; RHOT1; RHOU; RHOV; RHOXF1; RHOXF2; RHPN1; RHPN2; RIBC2; RIC1; RIC3; RICTOR; RIFT; RILP; RILPL1; RIMBP2; RIMBP3C; RIMS1; RIMS2; RIMS4; RINI1; RIN2; RIN3; RING1; RINT1; RIOK1; RIOK2; RIOK3; RIPK1; RIPK2; RIPK3; RIPK4; RIPPLY2; RIT1; RITZ; RITA1; RLBP1; RLF; RLIM; RLN1; RLN2; RLN3; RMDN1; RMDN2; RMDN3; RMI1; RMI2; RMND1; RNASE11; RNASE12; RNASE13; RNASE1; RNASE2; RNASE3; RNASE4; RNASE6; RNASE7; RNASE8; RNASE9; RNASEH1; RNASEH2A; RNASEH2B; RNASEH2C; RNASEK; RNASEL; RNASET2; RND1; RND3; RNF103-CHMP3; RNF103; RNF111; RNF112; RNF114; RNF123; RNF125; RNF128; RNF130; RNF135; RNF138; RNF139; RNF144A; RNF144B; RNF146; RNF149; RNF14; RNF150; RNF152; RNF157; RNF167; RNF168; RNF170; RNF180; RNF182; RNF19A; RNF207; RNF20; RNF213; RNF214; RNF216; RNF24; RNF2; RNF31; RNF34; RNF39; RNF40; RNF41; RNF43; RNF44; RNF4; RNF5; RNF6; RNF7; RNF8; RNGTT; RNH1; RNLS; RNMT; RNPEP; RNPEPL1; RNPS1; ROBOT; ROBO2; ROBO3; ROBO4; ROCK1; ROCK2; ROGDI; ROM1; ROPN1B; ROPN1; ROPN1L; ROR1; ROR2; RORA; RORB; RORC; ROS1; RP1; RP1L1; RP2; RP9; RPA1; RPA2; RPA3; RPA4; RPAIN; RPAP1; RPE; RPGR; RPGRIP1; RPGRIP1L; RPH3A; RPH3AL; RPIA; RPL10A; RPL10; RPL10L; RPL12; RPL13; RPL14; RPL15; RPL17-C18orf32; RPL17; RPL18; RPL19; RPL21; RPL23A; RPL23; RPL24; RPL27A; RPL29; RPL30; RPL31; RPL34; RPL35; RPL36A; RPL36AL; RPL37A; RPL38; RPL39; RPL39L; RPL3; RPL41; RPL4; RPL6; RPL7A; RPL7; RPLP0; RPLP1; RPN1; RPN2; RPP14; RPP21; RPP25; RPP38; RPP40; RPRD1A; RPRD1B; RPRD2; RPRM; RPS10; RPS14; RPS15A; RPS16; RPS18; RPS19BP1; RPS20; RPS24; RPS27A; RPS27; RPS27L; RPS29; RPS2; RPS3A; RPS3; RPS4X; RPS4Y1; RPS6KA2; RPS6KA3; RPS6KA4; RPS6KA5; RPS6KA6; RPS6KB1; RPS6KB2; RPS9; RPSA; RPTOR; RQCD1; RRAD; RRAGA; RRAS2; RRAS; RRBP1; RREB1; RRH; RRM1; RRM2B; RRM2; RRN3; RRNAD1; RRP1B; RRP1; RRP9; RRS1; RS1; RSAD2; RSF1; RSL101; RSL24D1; RSPH1; RSPH4A; RSPH9; RSPO2; RSPO3; RSPO4; RSRC1; RSRC2; RSRP1; RSU1; RTCB; RTEL1; RTKN2; RTKN; RTL1; RTN1; RTN2; RTN3; RTN4; RTN4IP1; RTN4R; RTP3; RTP4; RTTN; RUFY1; RUFY3; RUNDC3B; RUNX1; RUNX1T1; RUNX2; RUNX3; RUVBL1; RUVBL2; RXFP1; RXFP2; RXFP3; RXRA; RXRB; RXRG; RYBP; RYK; RYR1; RYR2; RYR3; S100A10; S100A11; S100Al2; S100A13; S100A14; S100A16; S100A1; S100A2; S100A3; S100A4; S100A5; S100A6; S100A7A; S100A7; S100A8; S100A9; S100B; S100P; S100Z; S1PR2; S1PR3; S1PR4; S1PR5; SAA1; SAA2; SAA4; SAAL1; SAC3D1; SACM1L; SACS; SAE1; SAFB2; SAFB; SAGE1; SAG; SALL1; SALL2; SALL3; SALL4; SAMD14; SAMD1; SAMD4A; SAMD5; SAMD9; SAMD9L; SAMHD1; SAMM50; SAMSN1; SAP30BP; SAP30L; SAPCD1; SAPCD2; SAR1A; SAR1B; SARDH; SARM1; SARNP; SARS2; SART1; SART3; SASH1; SASH3; SAT1; SAT2; SATB1; SATB2; SATL1; SAV1; SBDS; SBF1; SBF2; SBNO1; SBNO2; SBSN; SC50; SCAF11; SCAF1; SCAF4; SCAF8; SCAI; SCAMP2; SCAMPS; SCAPER; SCAP; SCARA3; SCARA5; SCARB1; SCARB2; SCARF2; SCCPDH; SCD5; SCD; SCFD1; SCFD2; SCG2; SCG3; SCG5; SCGB1A1; SCGB1D1; SCGB1D2; SCGB2A1; SCGB2A2; SCGB2B2; SCGB3A1; SCGB3A2; SCGN; SCHIP1; SCIN; SCLT1; SCLY; SCML2; SCML4; SCN10A; SCN11A; SCN1A; SCN1B; SCN2A; SCN2B; SCN3A; SCN3B; SCN4A; SCN4B; SCN5A; SCN7A; SCN8A; SCN9A; SCNM1; SCNN1A; SCNN1B; SCNN1D; SCNN1G; SCO1; SCO2; SCP2D1; SCPEP1; SCRIB; SCRN1; SCT; SCTR; SCUBE1; SCUBE2; SCUBE3; SCYL1; SCYL3; SDC1; SDC2; SDC3; SDC4; SDCBP2; SDCBP; SDCCAG3; SDCCAG8; SDF2; SDF2L1; SDF4; SDHAF1; SDHAF2; SDHAF4; SDHA; SDHC; SDHD; SDK1; SDK2; SDPR; SDR42E1; SDR9C7; SDS; SEC11A; SEC11C; SEC13; SEC14L1; SEC14L2; SEC14L3; SEC16B; SEC23A; SEC23B; SEC23IP; SEC24A; SEC24B; SEC24C; SEC31A; SEC61A1; SEC61G; SEC62; SEC63; SECISBP2; SECISBP2L; SECTM1; SEL1L; SELE; SELENBP1; SELL; SELP; SELPLG; SEMA3A; SEMA3B; SEMA3C; SEMA3D; SEMA3E; SEMA3F; SEMA3G; SEMA4A; SEMA4B; SEMA4D; SEMA4F; SEMA4G; SEMA5A; SEMA5B; SEMA6A; SEMA6B; SEMA6D; SEMA7A; SEMG1; SEMG2; SENP1; SENP2; SENP3; SENP5; SENP6; SENP8; SEPHS1; SEPSECS; SERAC1; SERF1A; SERGEF; SERINC1; SERINC3; SERINC5; SERP1; SERP2; SERPINA10; SERPINA11; SERPINA12; SERPINA1; SERPINA3; SERPINA4; SERPINA5; SERPINA6; SERPINA7; SERPINA9; SERPINB10; SERPINB13; SERPINB1; SERPINB2; SERPINB3; SERPINB4; SERPINB5; SERPINB6; SERPINB7; SERPINB8; SERPINB9; SERPINC1; SERPIND1; SERPINE1; SERPINE2; SERPINE3; SERPINF1; SERPINF2; SERPING1; SERPINH1; SERPINI1; SERPINI2; SERTAD1; SERTAD2; SESN1; SESN3; SETBP1; SETD1A; SETD1B; SETD2; SETD3; SETD5; SETD7; SETD8; SETDB2; SET; SETMAR; SETX; SEZ6; SEZ6L2; SEZ6L; SF1; SF3A1; SF3B1; SF3B2; SF3B6; SFI1; SFMBT1; SFMBT2; SFR1; SFRP1; SFRP2; SFRP4; SFRP5; SFSWAP; SFT2D2; SFT2D3; SFTA2; SFTA3; SFTPA1; SFTPA2; SFTPB; SFTPC; SFTPD; SFXN1; SFXN2; SFXN4; SGCA; SGCB; SGCD; SGCE; SGCG; SGCZ; SGIP1; SGK1; SGK223; SGK2; SGK3; SGMS1; SGMS2; SGOL1; SGPL1; SGPP1; SGPP2; SGSH; SGSM2; SGSM3; SGTA; SH2B1; SH2B2; SH2B3; SH2D1A; SH2D2A; SH2D3A; SH2D3C; SH2D4A; SH2D4B; SH3BGR; SH3BGRL2; SH3BGRL; SH3BP1; SH3BP2; SH3BP4; SH3BP5; SH3D19; SH3GL1; SH3GL2; SH3GL3; SH3KBP1; SH3PXD2A; SH3PXD2B; SH3RF1; SH3RF3; SH3TC2; SH3YL1; SHANK1; SHANK2; SHANK3; SHARPIN; SHBG; SHB; SHC1; SHC2; SHC3; SHC4; SHCBP1; SHF; SHFM1; SHH; SHISA2; SHISA3; SHISA6; SHISA9; SHMT1; SHMT2; SHOC2; SHOX2; SHOX; SHPK; SHPRH; SHQ1; SHROOM2; SHROOM3; SHROOM4; SIAE; SIAH1; SIAH2; SIDT1; SIGIRR; SIGLEC11; SIGLEC14; SIGLEC1; SIGLECS; SIGLEC7; SIGLEC8; SIGLEC9; SIGMAR1; SI; SIK1; SIK2; SIK3; Sil1; SIM1; SIM2; SIN3A; SIN3B; SIPA1; SIPA1L2; SIPA1L3; SIRPA; SIRPB1; SIRPG; SIRT1; SIRT2; SIRT3; SIRT4; SIRTS; SIRT6; SIRT7; SITZ; SIVA1; SIX1; SIX2; SIX3; SIX4; SIX5; SIX6; SKA1; SKA2; SKAP1; SKAP2; SKIL; SKIV2L2; SKIV2L; SKOR1; SKOR2; SKP1; SKP2; SLA2; SLA; SLAIN2; SLAMF1; SLAMF6; SLAMF7; SLAMF8; SLBP; SLC10A1; SLC10A2; SLC10A6; SLC10A7; SLC11A1; SLC11A2; SLC12A1; SLC12A2; SLC12A3; SLC12A4; SLC12A5; SLC12A6; SLC12A7; SLC12A8; SLC12A9; SLC13A1; SLC13A2; SLC13A3; SLC13A5; SLC14A1; SLC14A2; SLC15A1; SLC15A2; SLC15A4; SLC16A10; SLC16A11; SLC16Al2; SLC16A13; SLC16A1; SLC16A2; SLC16A3; SLC16A6; SLC16A7; SLC16A8; SLC16A9; SLC17A1; SLC17A2; SLC17A3; SLC17A4; SLC17A5; SLC17A6; SLC17A7; SLC17A8; SLC18A1; SLC18A2; SLC18A3; SLC19A1; SLC19A2; SLC19A3; SLC1A1; SLC1A2; SLC1A3; SLC1A4; SLC1A5; SLC1A6; SLC20A1; SLC20A2; SLC22A11; SLC22Al2; SLC22A13; SLC22A14; SLC22A16; SLC22A17; SLC22A18AS; SLC22A18; SLC22A1; SLC22A23; SLC22A24; SLC22A2; SLC22A3; SLC22A4; SLC22A5; SLC22A6; SLC22A7; SLC22A8; SLC23A1; SLC23A2; SLC24A1; SLC24A2; SLC24A3; SLC24A4; SLC24A5; SLC25A10; SLC25Al2; SLC25A13; SLC25A14; SLC25A15; SLC25A16; SLC25A18; SLC25A19; SLC25A1; SLC25A20; SLC25A21; SLC25A22; SLC25A23; SLC25A25; SLC25A27; SLC25A2; SLC25A36; SLC25A37; SLC25A38; SLC25A3; SLC25A40; SLC25A41; SLC25A42; SLC25A43; SLC25A45; SLC25A46; SLC25A47; SLC25A4; SLC25A52; SLC25A5; SLC25A6; SLC26A1; SLC26A2; SLC26A3; SLC26A4; SLC26A5; SLC26A6; SLC26A7; SLC26A8; SLC26A9; SLC27A1; SLC27A2; SLC27A3; SLC27A4; SLC27A5; SLC28A1; SLC28A2; SLC28A3; SLC29A1; SLC29A2; SLC29A3; SLC29A4; SLC2A10; SLC2A11; SLC2Al2; SLC2A13; SLC2A14; SLC2A2; SLC2A3; SLC2A4RG; SLC2A5; SLC2A6; SLC2A8; SLC2A9; SLC30A10; SLC30A1; SLC30A3; SLC30A4; SLC30A5; SLC30A6; SLC30A7; SLC30A8; SLC30A9; SLC31A1; SLC31A2; SLC32A1; SLC33A1; SLC34A1; SLC34A2; SLC34A3; SLC35A1; SLC35A2; SLC35A3; SLC35A4; SLC35B2; SLC35B4; SLC35C1; SLC35D3; SLC35F1; SLC35F2; SLC35F3; SLC35F4; SLC35F6; SLC35G1; SLC35G2; SLC35G5; SLC35G6; SLC36A1; SLC36A2; SLC37A1; SLC37A2; SLC37A4; SLC38A1; SLC38A2; SLC38A4; SLC38A5; SLC38A6; SLC38A7; SLC38A8; SLC38A9; SLC39A10; SLC39A11; SLC39Al2; SLC39A13; SLC39A14; SLC39A1; SLC39A2; SLC39A3; SLC39A4; SLC39A6; SLC39A7; SLC39A8; SLC39A9; SLC3A1; SLC3A2; SLC40A1; SLC41A1; SLC43A1; SLC43A2; SLC43A3; SLC44A1; SLC44A2; SLC44A4; SLC44A5; SLC45A2; SLC45A3; SLC45A4; SLC46A1; SLC46A2; SLC47A1; SLC48A1; SLC4A10; SLC4A11; SLC4A1AP; SLC4A1; SLC4A2; SLC4A3; SLC4A4; SLC4A5; SLC4A7; SLC4A9; SLC50A1; SLC51A; SLC51B; SLC52A1; SLC52A2; SLC52A3; SLC5A11; SLC5Al2; SLC5A1; SLC5A2; SLC5A3; SLC5A4; SLC5A5; SLC5A6; SLC5A7; SLC5A8; SLC6A11; SLC6Al2; SLC6A13; SLC6A14; SLC6A15; SLC6A18; SLC6A19; SLC6A1; SLC6A20; SLC6A2; SLC6A4; SLC6A5; SLC6A6; SLC6A7; SLC6A8; SLC6A9; SLC7A10; SLC7A11; SLC7A13; SLC7A14; SLC7A1; SLC7A2; SLC7A3; SLC7A4; SLC7A5; SLC7A6; SLC7A7; SLC7A8; SLC7A9; SLC8A1; SLC8A2; SLC8A3; SLC9A1; SLC9A2; SLC9A3; SLC9A3R1; SLC9A3R2; SLC9A4; SLC9A5; SLC9A6; SLC9A7; SLC9A8; SLC9A9; SLC9B1; SLC9B2; SLC9C1; SLC9C2; SLCO1A2; SLCO1B1; SLCO1B3; SLCO1B7; SLCO1C1; SLCO2A1; SLCO2B1; SLCO3A1; SLCO4A1; SLCO4C1; SLCO5A1; SLCO6A1; SLFN12; SLFN12L; SLFN14; SLFN5; SLIT1; SLIT2; SLIT3; SLITRK1; SLITRK2; SLITRK3; SLITRK5; SLITRK6; SLK; SLMAP; SLMO2; SLN; SLPI; SLTM; SLUT; SLURP1; SLX4; SLX4IP; SMAD1; SMAD2; SMAD3; SMAD4; SMAD5; SMAD6; SMAD7; SMAD9; SMAGP; SMAP1; SMARCA1; SMARCA2; SMARCA4; SMARCA5; SMARCAD1; SMARCAL1; SMARCB1; SMARCC1; SMARCC2; SMARCD1; SMARCD3; SMARCE1; SMC1A; SMC1B; SMC2; SMC3; SMC4; SMC5; SMC6; SMCHD1; SMCO4; SMCP; SMEK1; SMEK2; SMG1; SMG6; SMG8; SMIM15; SMIM19; SMIM20; SMIM21; SMIM23; SMIM5; SMN2; SMOC1; SMOC2; SMO; SMOX; SMPD1; SMPD2; SMPD3; SMPDL3A; SMPDL3B; SMPX; SMR3B; SMS; SMTN; SMTNL1; SMU1; SMUG1; SMURF1; SMURF2; SMYD1; SMYD2; SMYD3; SMYD4; SMYD5; SNAI1; SNAI2; SNAI3; SNAP23; SNAP25; SNAP29; SNAP47; SNAP91; SNAPC1; SNAPC4; SNAPC5; SNCA; SNCAIP; SNCB; SNCG; SND1; SNED1; SNF8; SNIP1; SNRK; SNRNP200; SNRNP27; SNRNP70; SNRPA; SNRPB; SNRPC; SNRPD1; SNRPD3; SNRPE; SNRPF; SNRPN; SNTA1; SNTB1; SNTG1; SNTG2; SNUPN; SNORF; SNW1; SNX10; SNX12; SNX14; SNX16; SNX18; SNX19; SNX1; SNX20; SNX24; SNX25; SNX29; SNX2; SNX30; SNX3; SNX5; SNX9; SOAT1; SOAT2; SOBP; SOCS1; SOCS2; SOCS3; SOCS4; SOCS5; SOCS6; SOCS7; SOD1; SOD2; SOD3; SOHLH1; SOHLH2; SON; SORBS1; SORBS2; SORBS3; SORCS1; SORCS2; SORCS3; SORD; SORL1; SOS1; SOS2; SOSTDC1; SOST; SOX10; SOX11; SOX12; SOX13; SOX14; SOX15; SOX17; SOX18; SOX1; SOX21; SOX2; SOX3; SOX4; SOX5; SOX6; SOX7; SOX8; SOX9; SP100; SP110; SP140; SP1; SP2; SP3; SP4; SP5; SP6; SP7; SP8; SPA17; SPACA1; SPACA3; SPAG11A; SPAG11B; SPAG16; SPAG1; SPAG4; SPAG5; SPAG6; SPAG7; SPAG8; SPAG9; SPAM1; SPANXA2; SPANXB1; SPANXD; SPANXN4; SPARC; SPARCL1; SPAST; SPATA13; SPATA16; SPATA17; SPATA18; SPATA19; SPATA20; SPATA21; SPATA22; SPATA25; SPATA2; SPATA5; SPATA7; SPATA8; SPATA9; SPATC1; SPC24; SPC25; SPCS3; SPDEF; SPDL1; SPDYA; SPECC1; SPECC1L; SPEF2; SPEG; SPESP1; SPG11; SPG20; SPG21; SPG7; SPHK1; SPHK2; SPHKAP; SPIT; SPIB; SPIC; SPIDR; SPIN1; SPIN2A; SPINK1; SPINK2; SPINK4; SPINK5; SPINK6; SPINK7; SPINT1; SPINT2; SPNS1; SPNS2; SP011; SPOCK1; SPOCK2; SPOCK3; SPON1; SPON2; SPOP; SPP1; SPP2; SPPL2A; SPPL2B; SPPL2C; SPPL3; SPRED1; SPRED2; SPRED3; SPR; SPRN; SPRR1A; SPRR1B; SPRR2A; SPRR2B; SPRR3; SPRTN; SPRY1; SPRY2; SPRY3; SPRY4; SPRYD7; SPSB3; SPSB4; SPTA1; SPTAN1; SPTB; SPTBN1; SPTBN2; SPTBN4; SPTBN5; SPTLC1; SPTLC2; SPTLC3; SPTSSB; SPTY2D1; SPZ1; SQLE; SQRDL; SQSTM1; SRA1; SRBD1; SRCAP; SRC; SRCIN1; SRD5A1; SRD5A2; SRD5A3; SREBF1; SREBF2; SREK1; SREK1IP1; SRFBP1; SRF; SRGAP1; SRGAP2; SRGAP3; SRGN; SRI; SRL; SRMS; SRP14; SRP19; SRP68; SRP72; SRP9; SRPK1; SRPK2; SRPRB; SRPR; SRPX2; SRPX; SRR; SRRM1; SRRM2; SRRM4; SRRT; SRSF10; SRSF11; SRSF12; SRSF1; SRSF2; SRSF3; SRSF4; SRSF5; SRSF6; SRSF7; SRSF9; SRXN1; SRY; SS18; SS18L1; SSB; SSBP1; SSBP2; SSFA2; SSH1; SSH2; SSMEM1; SSNA1; SSPN; SSPO; SSR1; SSR2; SSRP1; SSSCA1; SST; SSTR1; SSTR2; SSTR3; SSTR4; SSTR5; SSUH2; SSX1; SSX2B; SSX2IP; SSX4B; SSX5; ST13; ST14; ST18; ST20; ST3GAL1; ST3GAL2; ST3GAL4; ST3GAL5; ST3GAL6; ST5; ST6GAL1; ST6GAL2; ST6GALNAC1; ST6GALNAC2; ST6GALNAC4; ST6GALNAC6; ST7; ST7L; ST8SIA1; ST8SIA2; ST8SIA3; ST8SIA4; ST8SIA6; STAB1; STAB2; STAC3; STAC; STAG1; STAG2; STAG3; STAM2; STAMBP; STAMBPL1; STAM; STAP1; STAP2; STARD10; STARD13; STARD3; STARD3NL; STARD5; STARD7; STARD8; STARD9; STAR; STAT1; STAT2; STAT3; STAT4; STAT5A; STAT5B; STATE; STATH; STAU2; STBD1; STC1; STC2; STEAP1; STEAP2; STEAP3; STEAP4; STH; STIL; STIM1; STIM2; STIP1; STK10; STK11; STK11IP; STK17A; STK17B; STK19; STK24; STK25; STK26; STK31; STK32A; STK32B; STK32C; STK33; STK35; STK38L; STK39; STK3; STK4; STMN2; STMN3; STMN4; STOM; STOML1; STOML2; STOML3; STON1-GTF2A1L; STON1; STOX1; STRA13; STRA6; STRA8; STRADA; STRADB; STRAP; STRC; STRN3; STRN4; STRN; STS; STT3A; STT3B; STUB1; STX11; STX16; STX17; STX18; STX1A; STX1B; STX2; STX3; STX4; STX5; STX6; STX8; STXBP1; STXBP2; STXBP4; STXBP5; STXBP5L; STXBP6; STYK1; STYX; SUB1; SUCLA2; SUCLG1; SUCLG2; SUCNR1; SUCO; SUDS3; SUFU; SUGCT; SUGP1; SUGT1; SULF1; SULF2; SULT1A1; SULT1A2; SULT1A4; SULT1B1; SULT1C2; SULT1E1; SULT2A1; SULT2B1; SULT4A1; SUMF1; SUMF2; SUMO1; SUMO2; SUMO3; SUMO4; SUN1; SUN2; SUN3; SUOX; SUPT20H; SUPT3H; SUPT4H1; SUPT7L; SUPV3L1; SURF1; SURF4; SUSD1; SUSD2; SUSD4; SUSD6; SUV39H1; SUV39H2; SUV420H2; SUZ12; SV2B; SV2C; SVEP1; SVIL; SVIP; SVOP; SWAP70; SWT1; SYBU; SYCE1; SYCE1L; SYCP2; SYCP2L; SYCP3; SYK; SYMPK; SYN1; SYN2; SYN3; SYNCRIP; SYNDIG1; SYNE1; SYNE2; SYNE4; SYNGAP1; SYNGR1; SYNGR2; SYNJ1; SYNJ2BP; SYNJ2; SYNM; SYNPO2; SYNPO; SYNPR; SYP; SYPL1; SYPL2; SYT11; SYT12; SYT13; SYT14; SYT1; SYT4; SYT6; SYT9; SYTL1; SYTL2; SYTL5; SYVN1; TAAR1; TAAR2; TAAR5; TAAR6; TAB1; TAB2; TAB3; TACT; TAC3; TAC4; TACC1; TACC2; TACC3; TACO1; TACR1; TACR2; TACR3; TADA1; TADA2A; TADA3; TAF15; TAF1B; TAF1C; TAF1D; TAF1; TAF2; TAF3; TAF4B; TAF4; TAF5L; TAF6; TAF7; TAF7L; TAF8; TAF9; TAGAP; TAGLN2; TAGLN; TALI; TAL2; TALDO1; TAMM41; TANC1; TANC2; TANGO2; TANK; TAOK1; TAOK2; TAOK3; TAP1; TAP2; TAPBP; TAPBPL; TARBP1; TARBP2; TARP; TARS; TARSL2; TAS2R10; TAS2R13; TAS2R14; TAS2R16; TAS2R1; TAS2R38; TAS2R50; TAS2R60; TAS2R9; TASP1; TATDN1; TAT; TAX1BP1; TAX1BP3; TAZ; TBATA; TBC1D15; TBC1D16; TBC1D1; TBC1D20; TBC1D22A; TBC1D22B; TBC1D24; TBC1D25; TBC1D2; TBC1D32; TBC1D3C; TBC1D3F; TBC1D4; TBC1D5; TBC1D7; TBC1D8; TBC1D9; TBCA; TBCC; TBCD; TBCE; TBCEL; TBK1; TBKBP1; TBL1X; TBL1XR1; TBL1Y; TBL2; TBL3; TBP; TBPL1; TBPL2; TBR1; TBRG1; TBX10; TBX18; TBX19; TBX1; TBX20; TBX21; TBX22; TBX2; TBX3; TBX4; TBX5; TBX6; TBXA2R; TBXAS1; TCAIM; TCAP; TCEA1; TCEA2; TCEA3; TCEAL1; TCEAL2; TCEAL4; TCEAL7; TCEB1; TCEB2; TCEB3C; TCERG1; TCERG1L; TCF12; TCF15; TCF19; TCF20; TCF21; TCF25; TCF3; TCF4; TCF7; TCF7L1; TCF7L2; TCFL5; TCHH; TCHP; TCIRG1; TCL1A; TCL1B; TCN1; TCN2; TCOF1; TCP10; TCP10L2; TCP11L1; TCP1; TCTA; TCTE1; TCTN1; TCTN2; TCTN3; TDGF1; TDG; TDO2; TCP1; TCP2; TDRD1; TDRD3; TDRD5; TDRD6; TDRD7; TDRD9; TDRKH; TDRP; TEAD1; TEAD2; TEAD3; TEAD4; TEC; TECPR2; TECR; TECRL; TECTA; TEF; TEFM; TEK; TEKT1; TEKT5; TELO2; TENM1; TENM2; TENM3; TENM4; TEP1; TEPP; TERF1; TERF2; TERF2IP; TERT; TESC; TES; TESPA1; TET1; TET2; TET3; TEX101; TEX11; TEX14; TEX15; TEX264; TEX29; TEX30; TEX35; TEX40; TFAM; TFAP2A; TFAP2B; TFAP2C; TFAP4; TFB1M; TFB2M; TFCP2; TFDP1; TFDP2; TFDP3; TFE3; TFEB; TFEC; TFF1; TFF2; TFF3; TFG; TF; TFIP11; TFPI2; TFPI; TFPT; TFR2; TFRC; TGFA; TGFB1; TGFB1I1; TGFB2; TGFB3; TGFBI; TGFBR1; TGFBR2; TGFBRAP1; TG; TGIF1; TGIF2-C20orf24; TGIF2; TGIF2LX; TGM1; TGM2; TGM3; TGM4; TGM5; TGM6; TGM7; TGOLN2; TGS1; THADA; THAP10; THAP11; THAP1; THAP2; THAP6; THBD; THBS1; THBS2; THBS3; THBS4; THEG; THEM4; THEM5; THEME; THEMIS2; THEMIS; THG1L; TH; THNSL1; THNSL2; THOC1; THOC2; TH005; THOC6; T; THOP1; THPO; THRA; THRB; THRSP; THSD1; THSD4; THSD7A; THYN1; TIAL; TIAF1; TIAL1; TIAM1; TIAM2; TICAM1; TICAM2; TICRR; TIFA; TIGD2; TIGIT; TIMD4; TIMELESS; TIMM10; TIMM17A; TIMM21; TIMM22; TIMM23; TIMM44; TIMM50; TIMM8A; TIMM8B; TIMMDC1; TIMP1; TIMP2; TIMP3; TIMP4; TINAG; TINF2; TIPARP; TIPIN; TIPRL; TIRAP; TJP1; TJP2; TJP3; TK1; TK2; TKT; TKTL1; TKTL2; TLDC1; TLE1; TLE2; TLE3; TLE4; TLE6; TLK1; TLK2; TLL1; TLL2; TLN1; TLN2; TLR10; TLR1; TLR2; TLR3; TLR4; TLR5; TLR6; TLR7; TLR8; TLR9; TLX1; TLX2; TLX3; TM4SF1; TM4SF20; TM4SF4; TM4SF5; TM6SF2; TM7SF2; TM9SF2; TM9SF4; TMBIM4; TMBIM6; TMC1; TMC2; TMC3; TMC5; TMC6; TMC8; TMCC1; TMCC2; TMCC3; TMCO1; TMCO4; TMCO5A; TMED10; TMED1; TMED2; TMED3; TMED4; TMED7; TMED7-TICAM2; TMED9; TMEFF1; TMEFF2; TMEM100; TMEM101; TMEM105; TMEM106B; TMEM108; TMEM114; TMEM115; TMEM117; TMEM11; TMEM126A; TMEM127; TMEM128; TMEM132A; TMEM132B; TMEM132C; TMEM1320; TMEM132E; TMEM134; TMEM135; TMEM138; TMEM150B; TMEM151A; TMEM151B; TMEM154; TMEM158; TMEM160; TMEM161B; TMEM163; TMEM165; TMEM169; TMEM170A; TMEM171; TMEM173; TMEM175; TMEM176B; TMEM178A; TMEM182; TMEM183A; TMEM184C; TMEM185A; TMEM187; TMEM189; TMEM189-UBE2V1; TMEM18; TMEM199; TMEM200A; TMEM205; TMEM207; TMEM209; TMEM213; TMEM215; TMEM216; TMEM217; TMEM219; TMEM220; TMEM229A; TMEM231; TMEM233; TMEM237; TMEM241; TMEM244; TMEM245; TMEM259; TMEM25; TMEM261; TMEM27; TMEM2; TMEM30A; TMEM30B; TMEM37; TMEM38A; TMEM38B; TMEM39A; TMEM40; TMEM43; TMEM45A; TMEM47; TMEM50B; TMEM55A; TMEM57; TMEM5; TMEM60; TMEM62; TMEM63A; TMEM67; TMEM70; TMEM74B; TMEM79; TMEM87A; TMEM88; TMEM89; TMEM8B; TMEM95; TMEM97; TMEM98; TMF1; TMIE; TMIGD2; TMIGD3; TMLHE; TMOD1; TMOD2; TMOD3; TMOD4; TMPO; TMPRSS11A; TMPRSS11B; TMPRSS110; TMPRSS11E; TMPRSS13; TMPRSS15; TMPRSS2; TMPRSS3; TMPRSS4; TMPRSS6; TMPRSS7; TMPRSS9; TMSB10; TMSB15B; TMSB4X; TMTC1; TMTC2; TMTC3; TMX1; TMX2; TMX3; TNC; TNFAIP1; TNFAIP2; TNFAIP3; TNFAIP6; TNFAIP8; TNFAIP8L2; TNFAIP8L3; TNF; TNFRSF10A; TNFRSF10B; TNFRSF10C; TNFRSF100; TNFRSF11A; TNFRSF11B; TNFRSF12A; TNFRSF13B; TNFRSF13C; TNFRSF14; TNFRSF19; TNFRSF1A; TNFRSF1B; TNFRSF21; TNFRSF6B; TNFRSF8; TNFSF10; TNFSF11; TNFSF12; TNFSF12-TNFSF13; TNFSF13B; TNFSF13; TNFSF14; TNFSF15; TNFSF18; TNFSF4; TNFSF8; TNFSF9; TNIK; TNIP1; TNIP2; TNIP3; TNK1; TNK2; TNKS2; TNKS; TNMD; TNNC1; TNN; TNNI1; TNNI2; TNNI3; TNNT1; TNNT2; TNNT3; TNP1; TNP2; TNPO1; TNPO2; TNPO3; TNRC18; TNRC6A; TNRC6B; TNR; TNS1; TNS2; TNS3; TNS4; TNXB; TOB1; TOB2; TOLLIP; TOM1; TOM1L1; TOMM20; TOMM34; TOMM40; TOMM70A; TONSL; TOP1; TOP2A; TOP2B; TOP3A; TOP3B; TOPBP1; TOPORS; TOR1A; TOR1AIP1; TOR1AIP2; TOR1B; TOR2A; TOX2; TOX3; TOX4; TOX; TP53AIP1; TP53BP1; TP53BP2; TP53; TP53I11; TP53I13; TP53I3; TP53INP1; TP53INP2; TP53RK; TP53TG3C; TP63; TP73; TPBG; TPCN1; TPCN2; TPD52; TPD52L1; TPD52L2; TPGS2; TPH1; TPH2; TPI1; TPK1; TPM1; TPM2; TPM3; TPM4; TPMT; TPO; TPP1; TPP2; TPPP2; TPPP3; TPPP; TPRG1; TPR; TPRN; TPSAB1; TPSB2; TPSD1; TPSG1; TPT1; TPTE2; TPTE; TPX2; TRA2A; TRA2B; TRABD2A; TRABD; TRADD; TRAF1; TRAF2; TRAF3; TRAF3IP1; TRAF3IP2; TRAF4; TRAF5; TRAF6; TRAF7; TRAFD1; TRAK1; TRAK2; TRAM1; TRAM1L1; TRAM2; TRAP1; TRAPPC10; TRAPPC11; TRAPPC1; TRAPPC2; TRAPPC4; TRAPPC9; TRAT1; TRDMT1; TRDN; TREH; TREM1; TREM2; TREML1; TREML2; TRERF1; TREX1; TREX2; TRHDE; TRH; TRHR; TRIAP1; TRIB1; TRIB2; TRIB3; TRIM10; TRIM11; TRIM13; TRIM15; TRIM16; TRIM17; TRIM21; TRIM22; TRIM23; TRIM24; TRIM25; TRIM26; TRIM27; TRIM28; TRIM29; TRIM2; TRIM31; TRIM32; TRIM34; TRIM35; TRIM36; TRIM37; TRIM38; TRIM39; TRIM3; TRIM40; TRIM42; TRIM44; TRIM50; TRIM56; TRIM58; TRIM59; TRIM5; TRIM62; TRIM66; TRIM68; TRIM69; TRIM6-TRIM34; TRIM71; TRIM72; TRIM73; TRIM74; TRIM8; TRIM9; TRIOBP; TRIO; TRIP10; TRIP11; TRIP13; TRIP4; TRIPE; TRIQK; TRMT10A; TRMT12; TRMT1; TRMT44; TRMT5; TRMU; TRO; TROVE2; TRPA1; TRPC1; TRPC3; TRPC4AP; TRPC4; TRPC5; TRPC6; TRPC7; TRPM1; TRPM2; TRPM3; TRPM4; TRPM5; TRPM6; TRPM7; TRPM8; TRPS1; TRPV1; TRPV2; TRPV3; TRPV4; TRPV5; TRPV6; TRRAP; TSACC; TSC1; TSC22D1; TSC22D3; TSC22D4; TSC2; TSEN2; TSEN34; TSEN54; TSFM; TSG101; TSGA10; TSHR; TSHZ1; TSHZ2; TSHZ3; TSLP; TSNAX; TSN; TSPAN10; TSPAN11; TSPAN12; TSPAN13; TSPAN14; TSPAN16; TSPAN18; TSPAN31; TSPAN32; TSPAN33; TSPAN4; TSPAN6; TSPAN7; TSPAN8; TSPAN9; TSPEAR; TSPO2; TSPO; TSPY10; TSPY1; TSPY3; TSPY4; TSPYL1; TSPYL2; TSPYL4; TSPYL5; TSR1; TSSC1; TSSK1B; TSSK2; TSSK4; TSTA3; TSTD1; TST; TTBK1; TTBK2; TTC12; TTC17; TTC19; TTC1; TTC21B; TTC28; TTC29; TTC37; TTC39A; TTC39B; TTC3; TTC5; TTC6; TTC7A; TTC7B; TTC8; TTC9B; TTC9C; TTC9; TTF1; TTF2; TTI1; TTI2; TTK; TTL; TTLL10; TTLL11; TTLL12; TTLL1; TTLL3; TTLL4; TTLL5; TTLL6; TTLL7; TTLL8; TTLL9; TTPA; TTR; TTYH1; TTYH2; TUBA1A; TUBA1B; TUBA1C; TUBA30; TUBA4A; TUBAE; TUBB1; TUBB2A; TUBB2B; TUBB3; TUBB4A; TUBB4B; TUBB6; TUBB; TUBD1; TUBE1; TUBG1; TUBG2; TUBGCP2; TUBGCP3; TUBGCP4; TUBGCP5; TUBGCP6; TUB; TUFM; TUFT1; TULP1; TULP2; TULP3; TULP4; TUSC1; TUSC2; TUSC3; TUSC5; TUT1; TVP23B; TWF1; TWIST1; TWIST2; TWSG1; TXK; TXLNG; TXN2; TXNDC15; TXNDC16; TXNDC17; TXNDC5; TXN; TXNL1; TXNRD2; TXNRD3NB; TYK2; TYMP; TYMS; TYR; TYRO3; TYROBP; TYRP1; TYSND1; TYW1B; U2AF1; U2AF2; UACA; UAP1; UBA1; UBA2; UBA3; UBA7; UBAC1; UBAC2; UBAP1; UBAP2; UBASH3A; UBASH3B; UBB; UBC; UBD; UBE2A; UBE2B; UBE2C; UBE2D1; UBE2D2; UBE2D3; UBE2E1; UBE2E2; UBE2E3; UBE2G1; UBE2G2; UBE2H; UBE2I; UBE2J1; UBE2K; UBE2L3; UBE2L6; UBE2M; UBE2N; UBE2Q2; UBE2QL1; UBE2R2; UBE2S; UBE2T; UBE2U; UBE2V1; UBE2V2; UBE2Z; UBE3A; UBE3B; UBE3C; UBE4A; UBE4B; UBIAD1; UBL3; UBL4A; UBL5; UBL7; UBLCP1; UBN1; UBOX5; UBP1; UBQLN1; UBQLN2; UBQLNL; UBR1; UBR3; UBR4; UBR5; UBR7; UBTD2; UBTF; UBXN1; UBXN2A; UBXN2B; UBXN4; UCHL1; UCHL3; UCHL5; UCK1; UCK2; UCKL1; UCMA; UCN2; UCN3; UCN; UCP1; UCP2; UCP3; UEVLD; UFD1L; UFL1; UFM1; UGCG; UGDH; UGGT1; UGGT2; UGP2; UGT1A10; UGT1A1; UGT1A3; UGT1A4; UGT1A5; UGT1A6; UGT1A7; UGT1A8; UGT1A9; UGT2A1; UGT2A2; UGT2A3; UGT2B10; UGT2B11; UGT2B15; UGT2B28; UGT2B4; UGT2B7; UGT3A2; UGT8; UHMK1; UHRF1BP1; UHRF1; UHRF2; UIMC1; ULBP1; ULBP2; ULBP3; ULK1; ULK2; ULK3; ULK4; UMOD; UMODL1; UMPS; UNC119; UNC13A; UNC13B; UNC13C; UNC13D; UNC45A; UNC45B; UNC5A; UNC5B; UNC5C; UNC5D; UNC79; UNC93A; UNC93B1; UNG; UPB1; UPF1; UPF2; UPF3A; UPF3B; UPK1A; UPK1B; UPK2; UPK3A; UPP1; UPP2; UPRT; UQCC1; UQCC2; UQCRB; UQCRC1; UQCRC2; UQCRFS1; UQCRQ; URB2; URGCP; URI1; URM1; UROC1; UROD; UROS; USB1; USE1; USF1; USF2; USH1C; USH1G; USH2A; USO1; USP10; USP11; USP12; USP13; USP14; USP15; USP17L2; USP17L30; USP18; USP20; USP22; USP25; USP26; USP28; USP2; USP32; USP33; USP36; USP37; USP3; USP40; USP42; USP43; USP44; USP46; USP48; USP49; USP4; USP5; USP6; USP6NL; USP7; USPS; USP9X; USP9Y; USPL1; UST; UTF1; UTP14A; UTP14C; UTP20; UTRN; UTS2B; UTS2; UTS2R; UTY; UVRAG; UVSSA; UXS1; UXT; VAC14; VAMP1; VAMP2; VAMP4; VAMP7; VAMP8; VANGL2; VAPA; VAPB; VARS2; VARS; VASH1; VASH2; VASP; VAT1; VAT1L; VAV1; VAV2; VAV3; VAX1; VAX2; VBP1; VCAN; VCL; VCP; VCX2; VCX3A; VCX3B; VCX; VCY; VDAC1; VDAC2; VDR; VEGFA; VEGFB; VEGFC; VENTX; VEPH1; VEZT; VGF; VGLL1; VGLL2; VGLL3; VGLL4; VHL; VHLL; VIL1; VIM; VIPAS39; VIP; VIPR1; VIPR2; VIT; VKORC1; VLDLR; VMA21; VMO1; VMP1; VN1R2; VN1R4; VNN1; VNN2; VNN3; VOPP1; VPRBP; VPREB1; VPREB3; VPS11; VPS13A; VPS13B; VPS13C; VPS26A; VPS28; VPS33A; VPS33B; VPS35; VPS36; VPS37A; VPS37B; VPS37C; VPS39; VPS41; VPS4A; VPS4B; VPS51; VPS52; VPS53; VPS54; VPS72; VPS8; VRK1; VRK2; VSIG10; VSIG1; VSIG2; VSIG4; VSNL1; VSTM1; VSX1; VSX2; VTA1; VTCN1; VTI1A; VTI1B; VTN; VWA2; VWA3A; VWA3B; VWA5A; VWA5B1; VWA7; VWA8; VWCE; VWDE; VWF; WAPAL; WARS2; WARS; WASF1; WASF3; WASH1; WAS; WASL; WBP1L; WBP2; WBSCR17; WBSCR22; WDFY2; WDFY4; WDHD1; WDPCP; WDR11; WDR12; WDR17; WDR19; WDR1; WDR20; WDR26; WDR31; WDR34; WDR35; WDR36; WDR37; WDR43; WDR45B; WDR45; WDR46; WDR48; WDR49; WDR4; WDR55; WDR5; WDR60; WDR62; WDR64; WDR66; WDR70; WDR72; WDR74; WDR76; WDR78; WDR7; WDR81; WDR83; WDR86; WDR93; WEE1; WFDC1; WFDC2; WFS1; WHSC1; WHSC1L1; WIF1; WIPF1; WIPF2; WIPF3; WIPI1; WIPI2; WISP1; WISP2; WISP3; WLS; WNK1; WNK2; WNK3; WNK4; WNT10A; WNT10B; WNT11; WNT16; WNT1; WNT2B; WNT2; WNT3A; WNT3; WNT4; WNT5A; WNT5B; WNT6; WNT7A; WNT7B; WNT8A; WNT8B; WNT9A; WNT9B; WRAP53; WRB; WRN; WRNIP1; WSB1; WSCD1; WSCD2; WT1; WTAP; WTIP; WWC1; WWC2; WWOX; WWP1; WWP2; WWTR1; XAB2; XAF1; XAGE1B; XAGE1E; XBP1; XCL1; XCL2; XCR1; XDH; XG; XIAP; XIRP1; XIRP2; XK; XKR4; XKR6; XKR9; XPA; XPC; XPNPEP1; XPNPEP2; XPNPEP3; XPO1; XPO4; XPO5; XPO6; XPO7; XPR1; XRCC1; XRCC2; XRCC3; XRCC4; XRCC5; XRCC6BP1; XRCC6; XRN1; XRN2; XRRA1; XXYLT1; XYLB; XYLT1; XYLT2; YAE1D1; YAP1; YARS2; YBX2; YBX3; YDJC; YEATS4; YES1; YIF1A; YIPF1; YIPF3; YIPF5; YKT6; YLPM1; YME1L1; YPEL1; YPEL2; YPEL3; YPEL4; YPEL5; YTHDC1; YTHDC2; YWHAB; YWHAE; YWHAG; YWHAH; YWHAQ; YWHAZ; YY1AP1; YY1; ZACN; ZAK; ZAP70; ZAR1; ZAR1L; ZASP; ZBED1; ZBED4; ZBED5; ZBP1; ZBTB10; ZBTB12; ZBTB14; ZBTB16; ZBTB17; ZBTB18; ZBTB20; ZBTB21; ZBTB22; ZBTB24; ZBTB2; ZBTB32; ZBTB33; ZBTB34; ZBTB38; ZBTB41; ZBTB46; ZBTB48; ZBTB49; ZBTB4; ZBTB5; ZBTB7C; ZBTB9; ZC2HC1B; ZC3H10; ZC3H11A; ZC3H12C; ZC3H12D; ZC3H14; ZC3H15; ZC3H3; ZC3H4; ZC3H7A; ZC3H7B; ZC3HAV1; ZC3HC1; ZC4H2; ZCCHC11; ZCCHC12; ZCCHC14; ZCCHC2; ZCCHC3; ZCCHC6; ZCCHC8; ZCRB1; ZCWPW1; ZDBF2; ZDHHC11; ZDHHC12; ZDHHC13; ZDHHC14; ZDHHC15; ZDHHC17; ZDHHC1; ZDHHC2; ZDHHC7; ZDHHC8; ZDHHC9; ZEB1; ZEB2; ZFAND3; ZFAND5; ZFAND6; ZFAT; ZFC3H1; ZFHX2; ZFHX3; ZFHX4; ZFP1; ZFP30; ZFP36; ZFP36L1; ZFP36L2; ZFP37; ZFP42; ZFP57; ZFP64; ZFP82; ZFP91; ZFPM1; ZFPM2; ZFR2; ZFR; ZFX; ZFY; ZFYVE19; ZFYVE21; ZFYVE26; ZFYVE27; ZFYVE28; ZFYVE9; ZG16B; ZGLP1; ZGPAT; ZHX1; ZHX2; ZIC1; ZIC2; ZIC3; ZIC4; ZIC5; ZIK1; ZIM2; ZKSCAN1; ZKSCAN3; ZKSCAN7; ZMAT3; ZMAT4; ZMIZ1; ZMYM2; ZMYM3; ZMYM4; ZMYM5; ZMYND10; ZMYND11; ZMYND8; ZNF106; ZNF107; ZNF10; ZNF112; ZNF121; ZNF131; ZNF132; ZNF133; ZNF141; ZNF143; ZNF146; ZNF148; ZNF154; ZNF160; ZNF169; ZNF175; ZNF177; ZNF182; ZNF184; ZNF185; ZNF189; ZNF197; ZNF202; ZNF205; ZNF20; ZNF212; ZNF214; ZNF215; ZNF217; ZNF224; ZNF22; ZNF230; ZNF236; ZNF239; ZNF23; ZNF248; ZNF24; ZNF253; ZNF260; ZNF263; ZNF264; ZNF266; ZNF267; ZNF268; ZNF273; ZNF274; ZNF276; ZNF277; ZNF280B; ZNF280D; ZNF281; ZNF282; ZNF286B; ZNF296; ZNF2; ZNF300; ZNF311; ZNF318; ZNF320; ZNF322; ZNF32; ZNF330; ZNF331; ZNF334; ZNF335; ZNF343; ZNF350; ZNF354A; ZNF35; ZNF365; ZNF366; ZNF367; ZNF382; ZNF383; ZNF384; ZNF385A; ZNF385B; ZNF385D; ZNF391; ZNF395; ZNF398; ZNF407; ZNF410; ZNF415; ZNF419; ZNF41; ZNF423; ZNF430; ZNF432; ZNF433; ZNF438; ZNF443; ZNF444; ZNF44; ZNF451; ZNF45; ZNF462; ZNF469; ZNF483; ZNF490; ZNF492; ZNF496; ZNF501; ZNF507; ZNF512B; ZNF512; ZNF513; ZNF516; ZNF519; ZNF521; ZNF536; ZNF555; ZNF559; ZNF568; ZNF569; ZNF577; ZNF580; ZNF581; ZNF582; ZNF583; ZNF585B; ZNF592; ZNF596; ZNF606; ZNF607; ZNF608; ZNF615; ZNF618; ZNF627; ZNF629; ZNF639; ZNF644; ZNF645; ZNF646; ZNF652; ZNF654; ZNF664; ZNF667; ZNF668; ZNF674; ZNF676; ZNF678; ZNF683; ZNF687; ZNF689; ZNF703; ZNF704; ZNF706; ZNF711; ZNF716; ZNF717; ZNF746; ZNF74; ZNF750; ZNF763; ZNF764; ZNF765; ZNF76; ZNF774; ZNF776; ZNF778; ZNF784; ZNF79; ZNF7; ZNF800; ZNF804A; ZNF804B; ZNF80; ZNF812; ZNF813; ZNF816; ZNF81; ZNF823; ZNF827; ZNF829; ZNF831; ZNF91; ZNF92; ZNF93; ZNF98; ZNFX1; ZNHIT2; ZNHIT3; ZNRD1; ZNRF3; ZP1; ZP4; ZPBP2; ZPLD1; ZPR1; ZRANB3; ZRSR2; ZSCAN18; ZSCAN22; ZSCAN26; ZSCAN31; ZSCAN32; ZSCAN9; ZSWIM2; ZSWIM6; ZW10; ZWILCH; ZWINT; ZYX; ZZEF1; ZZZ3, and a combination thereof.
